(19) United States
(12) Patent Application Publication
(10) Pub. No.: US 2022/0332708 A1
(43) Pub. Date: Oct. 20, 2022

(54) SULFONAMIDE COMPOUNDS AS CARDIAC SARCOMERE ACTIVATORS

(71) Applicant: Cytokinetics, Inc., South San Francisco, CA (US)

(72) Inventors: Jeffrey GARDINA, Rocklin, CA (US); Aroop CHANDRA, Carmel, IN (US); Luke W. ASHCRAFT, San Francisco, CA (US); Scott COLLIBEE, San Carlos, CA (US); Chihyuan CHUANG, Millbrae, CA (US); Alex MUCI - DECEASED, South San Francisco, CA (US); Bradley P. MORGAN, Oakland, CA (US); Antonio ROMERO, San Mateo, CA (US); Michael JOHNSON, South San Francisco, CA (US); David MOEBIUS, South San Francisco, CA (US); Hanmo ZHANG, South San Francisco, CA (US); Felix GONZALEZ, South San Francisco, CA (US)

(21) Appl. No.: 17/713,946

(22) Filed: Apr. 5, 2022

Related U.S. Application Data
(60) Provisional application No. 63/171,469, filed on Apr. 6, 2021.

(51) Int. Cl. C07D 405/14; C07D 403/12; C07D 403/14; C07D 239/96; C07D 401/12; C07D 401/14

(52) U.S. Cl. CPC C07D 405/14; C07D 403/12; C07D 403/14; C07D 239/96; C07D 401/12; C07D 401/14

(57) ABSTRACT

The present disclosure relates to sulfonamide compounds and pharmaceutically acceptable salts thereof as cardiac sarcomere activators.

### CROSS REFERENCE TO RELATED APPLICATIONS

This application claims the priority benefit of U.S. Provisional Application No. 63/171,469, filed on Apr. 6, 2021, which is incorporated herein by reference in its entirety.

### FIELD OF THE INVENTION

The present invention relates to sulfonamide-containing compounds, and pharmaceutical salts thereof, as cardiac sarcomere activators.

### BACKGROUND OF THE INVENTION

The “sarcomere” is an elegantly organized cellular structure found in cardiac and skeletal muscle made up of interdigitating thin and thick filaments; it comprises nearly 60% of cardiac cell volume. The thick filaments are composed of “myosin,” the protein responsible for transducing chemical energy (ATP hydrolysis) into force and directed movement. Myosin and its functionally related cousins are called motor proteins. The thin filaments are composed of a complex of proteins. One of these proteins, “actin” (a filamentous polymer) is the substrate upon which myosin pulls during force generation. Bound to actin are a set of regulatory proteins, the “troponin complex” and “tropomyosin,” which make the actin-myosin interaction dependent on changes in intracellular Ca²⁺ levels. With each heartbeat, Ca²⁺ levels rise and fall, initiating cardiac muscle contraction and then cardiac muscle relaxation. Each of the components of the sarcomere contributes to its contractile response.

Myosin is the most extensively studied of all the motor proteins. Of the thirteen distinct classes of myosin in human cells, the myosin-II class is responsible for contraction of skeletal, cardiac, and smooth muscle. This class of myosin is significantly different in amino acid composition and in overall structure from myosin in the other twelve distinct classes. Myosin-II consists of two globular head domains linked together by a long alpha-helical coiled-coiled tail that assembles with other myosin-IIs to form the core of the sarcomere's thick filament. The globular heads have a catalytic domain where the actin binding and ATP functions of myosin take place. Once bound to an actin filament, the release of phosphate (cf. ATP to ADP) leads to a change in structural conformation of the catalytic domain that in turn alters the orientation of the light-chain binding lever arm domain that extends from the globular head; this movement is termed the powerstroke. This change in orientation of the myosin head in relationship to actin causes the thick filament of which it is a part to move with respect to the thin actin filament to which it is bound. Un-binding of the globular head from the actin filament (also Ca²⁺ modulated) coupled with return of the catalytic domain and light chain to their starting conformation/orientation completes the contraction and relaxation cycle.

Cardiac troponin is a heterotrimeric protein (cTnC, cTnI, cTnT) that, together with tropomyosin, is bound to actin and forms the thin filament of the cardiac sarcomere. Troponin regulates contractility by sensing intracellular calcium levels and gating the contraction and relaxation of the sarcomere by myosin.

Mammalian heart muscle consists of two forms of cardiac myosin, alpha and beta, and they are well characterized. The beta form is the predominant form (>90 percent) in adult human cardiac muscle. Both have been observed to be regulated in human heart failure conditions at both transcriptional and translational levels, with the alpha form being down-regulated in heart failure.

The sequences of all of the human skeletal, cardiac, and smooth muscle myosins have been determined. While the cardiac alpha and beta myosins are very similar (93% identity), they are both considerably different from human smooth muscle (42% identity) and more closely related to skeletal myosins (80% identity). Conveniently, cardiac muscle myosins are incredibly conserved across mammalian species. For example, both alpha and beta cardiac myosins are >96% conserved between humans and rats, and the available 250-residue sequence of porcine cardiac beta myosin is 100% conserved with the corresponding human cardiac beta myosin sequence. Such sequence conservation contributes to the predictability of studying myosin based therapeutics in animal based models of heart failure.

The components of the cardiac sarcomere present targets for the treatment of heart failure, for example by increasing contractility or facilitating complete relaxation to modulate systolic and diastolic function, respectively.

Congestive heart failure (“CHF”) is not a specific disease, but rather a constellation of signs and symptoms, all of which are caused by an inability of the heart to adequately respond to exertion by increasing cardiac output. The dominant pathophysiology associated with CHF is systolic dysfunction, an impairment of cardiac contractility (with a consequent reduction in the amount of blood ejected with each heartbeat). Systolic dysfunction with compensatory dilation of the ventricular cavities results in the most common form of heart failure, “dilated cardiomyopathy,” which is often considered to be one in the same as CHF. The counterpoint to systolic dysfunction is diastolic dysfunction, an impairment of the ability to fill the ventricles with blood, which can also result in heart failure even with preserved left ventricular function. Congestive heart failure is ultimately associated with improper function of the cardiac myocyte itself, involving a decrease in its ability to contract and relax.

Many of the same underlying conditions can give rise to systolic and/or diastolic dysfunction, such as atherosclerosis, hypertension, viral infection, valvular dysfunction, and genetic disorders. Patients with these conditions typically present with the same classical symptoms: shortness of breath, edema and overwhelming fatigue. In approximately half of the patients with dilated cardiomyopathy, the cause of their heart dysfunction is ischemic heart disease due to coronary atherosclerosis. These patients have had either a single myocardial infarction or multiple myocardial infarctions; here, the consequent scarring and remodeling results in the development of a dilated and hypocontractile heart. At times the causative agent cannot be identified, so the disease is referred to as “idiopathic dilated cardiomyopathy.” Irrespective of ischemic or other origin, patients with dilated cardiomyopathy share an abysmal prognosis, excessive morbidity and high mortality.

The prevalence of CHF has grown to epidemic proportions as the population ages and as cardiologists have become more successful at reducing mortality from ischemic heart disease, the most common prelude to CHF. Roughly 4.6 million people in the United States have been diagnosed with CHF; the incidence of such diagnosis is approaching 10 per 1000 after 65 years of age. Hospitalization for CHF is usually the result of inadequate outpatient therapy. Hospital discharges for CHF rose from 377,000 (in 1979) to 970,000 (in 2002) making CHF the most common discharge diagnosis in people age 65 and over. The five-year mortality from CHF approaches 50%. Hence, while therapies for heart disease have greatly improved and life expectancies have extended over the last several years, new and better therapies continue to be sought, particularly for CHF.

“Acute” congestive heart failure (also known as acute “decompensated” heart failure) involves a precipitous drop in cardiac function resulting from a variety of causes. For example in a patient who already has congestive heart failure, a new myocardial infarction, discontinuation of medications, and dietary indiscretions may all lead to accumulation of edema fluid and metabolic insufficiency even in the resting state. A therapeutic agent that increases cardiac function during such an acute episode could assist in relieving this metabolic insufficiency and speeding the removal of edema, facilitating the return to the more stable “compensated” congestive heart failure state. Patients with very advanced congestive heart failure particularly those at the end stage of the disease also could benefit from a therapeutic agent that increases cardiac function, for example, for stabilization while waiting for a heart transplant. Other potential benefits could be provided to patients coming off a bypass pump, for example, by administration of an agent that assists the stopped or slowed heart in resuming normal function. Patients who have diastolic dysfunction (insufficient relaxation of the heart muscle) could benefit from a therapeutic agent that modulates relaxation.

Inotropes are drugs that increase the contractile ability of the heart. As a group, all current inotropes have failed to meet the gold standard for heart failure therapy, i.e., to prolong patient survival. In addition, current agents are poorly selective for cardiac tissue, in part leading to recognized adverse effects that limit their use. Despite this fact, intravenous inotropes continue to be widely used in acute heart failure (e.g., to allow for reinstitution of oral medications or to bridge patients to heart transplantation) whereas in chronic heart failure, orally given digoxin is used as an inotrope to relieve patient symptoms, improve the quality of life, and reduce hospital admissions.

Current inotropic therapies improve contractility by increasing the calcium transient via the adenylyl cyclase pathway, or by delaying cAMP degradation through inhibition of phosphodiesterase (PDE), which can be detrimental to patients with heart failure.

New approaches are needed to improve cardiac function in congestive heart failure. There remains a need for agents that exploit different mechanisms of action and may have better outcomes in terms of relief of symptoms, safety, and patient mortality, both short-term and long-term.

### BRIEF SUMMARY OF THE INVENTION

Provided herein are compounds and salts thereof which are useful as an active ingredient for pharmaceutical compositions, in particular, pharmaceutical compositions for treating a disease or condition responsive to modulation of the contractility of the cardiac sarcomere.

The present invention provides novel compounds which are expected to be used as an active ingredient in a pharmaceutical composition, and in particular, in a pharmaceutical composition for preventing or treating a disease or condition responsive to modulation of the contractility of the cardiac sarcomere.

In one aspect, provided is a compound of Formula (I)

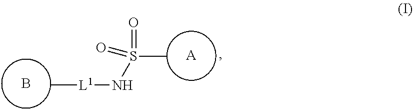

or a pharmaceutically acceptable salt thereof, wherein:
A is selected from the group consisting of

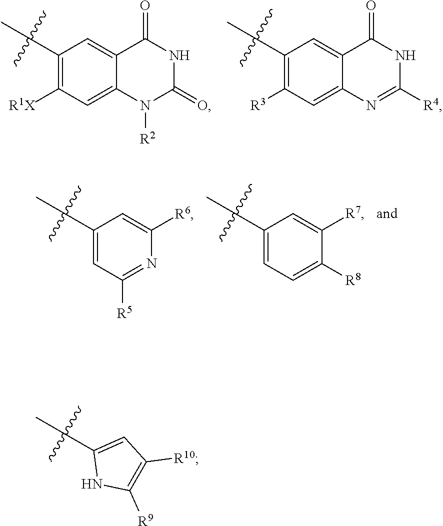

L¹ is a bond, C₁₋₆alkylene, or —NH—C₁₋₆alkylene-; and
B is selected from the group consisting of C₄₋₆cycloalkyl, tetrahydrofuranyl, pipiridinyl, phenyl, pyridyl, indolyl,

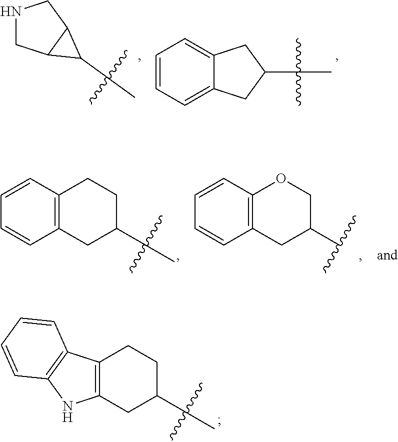

wherein
X is O or NH;
R¹, R², R⁴, and R⁵ are each independently C₁₋₆alkyl;
R³ is H, C₁₋₆alkyl, —NH—C₁₋₆alkyl, or —O—C₁₋₆alkyl, wherein the —O—C₁₋₆alkyl of R³ is optionally substituted with heterocyclyl;
R⁶ is 4- to 5-membered nitrogen-containing heterocyclyl substituted with one or more independently selected —CN, —OH, C₁₋₆alkyl, or —O—C₁₋₆alkyl substituents, wherein each C₁₋₆alkyl or —O—C₁₋₆alkyl substituent is optionally substituted with one or more independently selected halo substituents;
R⁷ is —CN or —C(O)—NH₂;
R⁸ is —NH—C₁₋₆alkyl or —N(C₁₋₆alkyl)₂;
R⁹ is C₁₋₆alkyl;
R¹⁰ is —C(O)—R^{a},

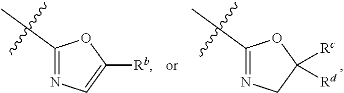

wherein
   R^{a} is selected from the group consisting of —O—C₁₋₆alkyl, —NR^{a1}R^{a2}, and a 4- to 7-membered nitrogen-containing heterocyclyl optionally substituted with one or more independently selected halo, —OH, —CN, C₁₋₆alkyl, C₁₋₆haloalkyl, or —O—C₁₋₆haloalkyl substituents;
   R^{a1} is H or C₁₋₆alkyl;
   R^{a2} is H or C₁₋₆alkyl optionally substituted with one or more independently selected halo, —OH, C₁₋₆haloalkyl, —O—C₁₋₆alkyl, or —NH—C₁₋₆haloalkyl substituents;
   R^{b}, R^{c}, and R^{d} are independently selected C₁₋₆ alkyl; and
B is optionally substituted with one or more substituents independently selected from the group consisting of: halo; —OH; C₁₋₆alkyl optionally substituted with phenyl, wherein the phenyl is optionally substituted with one or more independently selected halo substituents; C₁₋₆haloalkyl; C₃₋₆cycloalkyl; 3- to 6-membered heterocyclyl optionally substituted with one or more independently selected C₁₋₆alkyl substituents; phenyl optionally substituted with one or more independently selected halo, C₁₋₆alkyl, or C₁₋₆haloalkyl substituents; —NH-phenyl optionally substituted with one or more independently selected halo substituents; pyrazolyl optionally substituted with one or more independently selected C₁₋₆alkyl or C₁₋₆haloalkyl substituents; and pyridyl optionally substituted with one or more independently selected C₁₋₆alkyl or C₁₋₆haloalkyl substituents.

Also provided is a pharmaceutical composition comprising a pharmaceutically acceptable excipient, carrier or adjuvant and at least one compound of formula (I) or subformulae thereof.

Also provided is a packaged pharmaceutical composition, comprising a pharmaceutical composition comprising a pharmaceutically acceptable excipient, carrier or adjuvant and at least one compound of formula (I) or subformulae thereof, and instructions for using the composition to treat a patient suffering from a heart disease.

Also provided is a method of treating heart disease in a mammal which method comprises administering to a mammal in need thereof a therapeutically effective amount of at least one compound of formula (I) or subformulae thereof or a pharmaceutical composition comprising a pharmaceutically acceptable excipient, carrier or adjuvant and at least one compound of formula (I) or subformulae thereof.

Also provided is a method for modulating the cardiac sarcomere in a mammal which method comprises administering to a mammal in need thereof a therapeutically effective amount of at least one compound of formula (I) or subformulae thereof or a pharmaceutical composition comprising a pharmaceutically acceptable excipient, carrier or adjuvant and at least one compound of formula (I) or subformulae thereof.

Also provided is a method for potentiating Troponin C, Troponin I or the interface of Troponin C and Troponin I to increase activity of the cardiac sarcomere in a mammal which method comprises administering to a mammal in need thereof a therapeutically effective amount of at least one compound of formula (I) or subformulae thereof or a pharmaceutical composition comprising a pharmaceutically acceptable excipient, carrier or adjuvant and at least one compound of formula (I) or subformulae thereof.

Also provided is the use, in the manufacture of a medicament for treating heart disease, of at least one compound of formula I or subformulae thereof.

### DETAILED DESCRIPTION OF THE INVENTION

The following description is not intended to limit the scope of the present disclosure but rather provides a description of exemplary embodiments.

### Definitions

The term “halo” or “halogen” means fluoro, chloro, bromo, or iodo; in some embodiments, fluoro, chloro, or bromo; in some embodiments, fluoro or chloro.

The term “alkyl” refers to linear or branched fully saturated carbon chain. Accordingly, “C₁₋₆alkyl” is linear or branched alkyl having 1 to 6 carbon atoms, and specific examples thereof include methyl, ethyl, n-propyl, isopropyl, n-butyl, isobutyl, sec-butyl, tert-butyl, n-pentyl, or n-hexyl; in some embodiments, a group selected from the group consisting of methyl and ethyl. In some embodiments, “alkyl” may encompass C₁₋₆alkyl, C₂₋₆alkyl, C₃₋₆alkyl, C₁₋₂alkyl, C₁₋₃alkyl, C₁₋₄alkyl, C₂₋₄alkyl, or C₃₋₄alkyl. The term “alkylene” refers to a bivalent alkyl.

The term “haloalkyl” refers to an alkyl group substituted with one or more halo groups. Accordingly, “C₁₋₆haloalkyl” is linear or branched alkyl having 1 to 6 carbon atoms and one or more halo substituents. In some embodiments, a C₁₋₆alkyl substituted with one to three independently selected fluoro or chloro groups.

The term “cycloalkyl” refers to a non-aromatic, fully saturated carbocycle having the indicated number of annular carbon atoms, for example, 3 to 6, 4 to 6, 3 to 4, or 4 to 5 ring carbon atoms. Cycloalkyl groups may be monocyclic or polycyclic (e.g., bicyclic). A cycloalkyl substituent may be attached by a single covalent bond to one ring carbon, or a cycloalkyl substituent may be fused and share two or more ring carbons with the molecule to which it is attached. A cycloalkyl group comprising more than one ring may be fused, bridged, spiro, or any combination thereof. Examples of cycloalkyl groups include cyclopropyl, cyclobutyl, cyclopentyl, cyclohexyl, and bicyclo[1.1.1]pentanyl.

The term “heterocycle”, “heterocyclic”, or “heterocyclyl” refers to a saturated or partially unsaturated non-aromatic cyclic group having at least one annular heteroatom, including but not limited to heteroatoms such as nitrogen, oxygen, and sulfur. A heterocyclyl group may have a single ring or multiple condensed rings. A heterocyclyl group comprising more than one ring may be fused, bridged, spiro, or any combination thereof. Examples of heterocyclyl groups include, but are not limited to, tetrahydrofuranyl, piperidinyl,

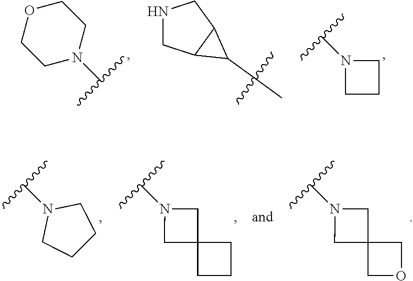 [SMILES: *N1CCOCC1 ; *C1C2CNCC12 ; *N1CCC1 ; *N1CCCC1 ; *N1CC2(CCC2)C1 ; *N1CC2(COC2)C1]

The term “optionally substituted” unless otherwise specified means that a group may be unsubstituted or substituted by one or more (e.g., 1, 2, 3, 4 or 5) of the substituents listed for that group in which the substituents may be the same of different. In some embodiments, an optionally substituted group has one substituent. In some embodiments, an optionally substituted group has two substituents. In some embodiments, an optionally substituted group has three substituents. In some embodiments, an optionally substituted group has four substituents. In some embodiments, an optionally substituted group has 1 to 2, 1 to 3, 1 to 4, 1 to 5, 2 to 3, 2 to 4, or 2 to 5 substituents. In some embodiments, an optionally substituted group is unsubstituted.

The term “pharmaceutically acceptable salts” are those salts which retain at least some of the biological activity of the free (non-salt) compound and which can be administered as drugs or pharmaceuticals to a subject. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

Any formula given herein, such as Formula (I) or (Ia), is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 has two or more stereocenters, also provided are any enantiomer or diastereomer of the compound. For example, if a compound of Table 1 contains a first stereocenter and a second stereocenter with “R” and “R” stereochemical configurations, respectively, also provided are stereoisomers of the compound having first and second stereocenters with “S” and “S” stereochemical configurations, respectively, “S” and “R” stereochemical configurations, respectively, and “R” and “S” stereochemical configurations, respectively. If a compound of Table 1 contains a first stereocenter and a second stereocenter with “S” and “S” stereochemical configurations, respectively, also provided are stereoisomers of the compound having first and second stereocenters with “R” and “R” stereochemical configurations, respectively, “S” and “R” stereochemical configurations, respectively, and “R” and “S” stereochemical configurations, respectively. If a compound of Table 1 contains a first stereocenter and a second stereocenter with “S” and “R” stereochemical configurations, respectively, also provided are stereoisomers of the compound having first and second stereocenters with “R” and “S” stereochemical configurations, respectively, “R” and “R” stereochemical configurations, respectively, and “S” and “S” stereochemical configurations, respectively. Similarly, if a compound of Table 1 contains a first stereocenter and a second stereocenter with “R” and “S” stereochemical configurations, respectively, also provided are stereoisomers of the compound having first and second stereocenters with “S” and “R” stereochemical configurations, respectively, “R” and “R” stereochemical configurations, respectively, and “S” and “S” stereochemical configurations, respectively. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

The compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms are replaced by an atom having the atomic mass or mass number different from the predominant atomic mass or mass number usually found in nature for the atom. All isotopes of any particular atom or element as specified are contemplated within the scope of the compounds of the present disclosure and include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I.

In some variations, any of the compounds described herein, such as a compound of Formula (I) or (Ia), or any variation thereof, or a compound of Table 1 may be deuterated (e.g., a hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art. Indication of an atom indicates isotopic variants of said atom, for instance, an explicit or implicit hydrogen includes deuterium at that position.

### Compounds

In one aspect, provided is a compound of Formula (I)

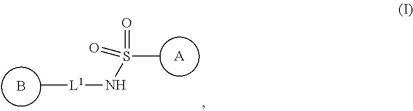

or a pharmaceutically acceptable salt thereof, wherein:
A is selected from the group consisting of

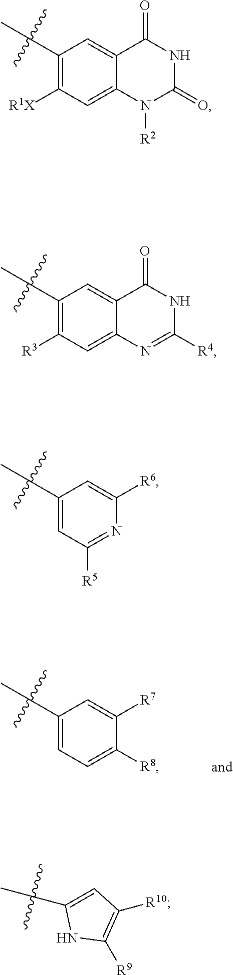

L¹ is a bond, C₁₋₆alkylene, or —NH—C₁₋₆alkylene-; and
B is selected from the group consisting of C₄₋₆cycloalkyl, tetrahydrofuranyl, pipiridinyl, phenyl, pyridyl, indolyl,

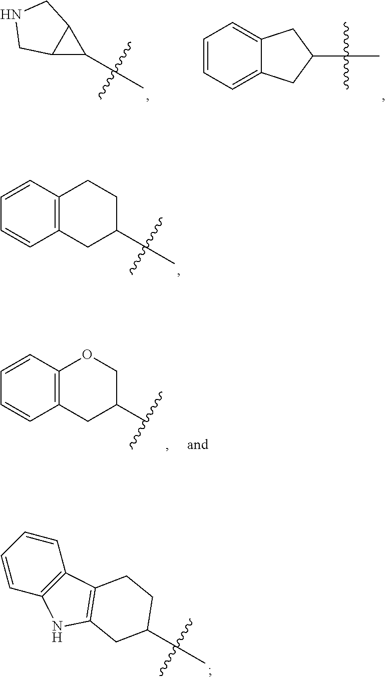

wherein
X is O or NH;
R¹, R², R⁴, and R⁵ are each independently C₁₋₆alkyl;
R³ is H, C₁₋₆alkyl, —NH—C₁₋₆alkyl, or —O—C₁₋₆alkyl, wherein the —O—C₁₋₆alkyl of R³ is optionally substituted with heterocyclyl;
R⁶ is 4- to 5-membered nitrogen-containing heterocyclyl substituted with one or more independently selected —CN, —OH, C₁₋₆alkyl, or —O—C₁₋₆alkyl substituents, wherein each C₁₋₆alkyl or —O—C₁₋₆alkyl substituent is optionally substituted with one or more independently selected halo substituents;
R⁷ is —CN or —C(O)—NH₂;
R⁸ is —NH—C₁₋₆alkyl or —N(C₁₋₆alkyl)₂;
R⁹ is C₁₋₆alkyl;
R¹⁰ is —C(O)—R^{a},

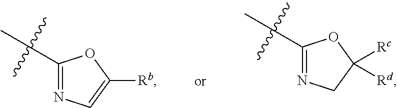

wherein
   R^{a} is selected from the group consisting of —O—C₁₋₆alkyl, —NR^{a1}R^{a2}, and a 4- to 7-membered nitrogen-containing heterocyclyl optionally substituted with one or more independently selected halo, —OH, —CN, C₁₋₆alkyl, C₁₋₆haloalkyl, or —O—C₁₋₆haloalkyl substituents;
   R^{a1} is H or C₁₋₆alkyl;
   R^{a2} is H or C₁₋₆alkyl optionally substituted with one or more independently selected halo, —OH, C₁₋₆haloalkyl, —O—C₁₋₆alkyl, or —NH—C₁₋₆haloalkyl substituents;
   R^{b}, R^{c}, and R^{d} are independently selected C₁₋₆ alkyl; and
B is optionally substituted with one or more substituents independently selected from the group consisting of: halo; —OH; C₁₋₆alkyl optionally substituted with phenyl, wherein the phenyl is optionally substituted with one or more independently selected halo substituents; C₁₋₆haloalkyl; C₃₋₆cycloalkyl; 3- to 6-membered heterocyclyl optionally substituted with one or more independently selected C₁₋₆alkyl substituents; phenyl optionally substituted with one or more independently selected halo, C₁₋₆alkyl, or C₁₋₆haloalkyl substituents; —NH-phenyl optionally substituted with one or more independently selected halo substituents; pyrazolyl optionally substituted with one or more independently selected C₁₋₆alkyl or C₁₋₆haloalkyl substituents; and pyridyl optionally substituted with one or more independently selected C₁₋₆alkyl or C₁₋₆haloalkyl substituents.

In another aspect, provided is a compound of Formula (Ia)

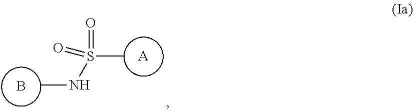

or a pharmaceutically acceptable salt thereof, wherein:
A is selected from the group consisting of

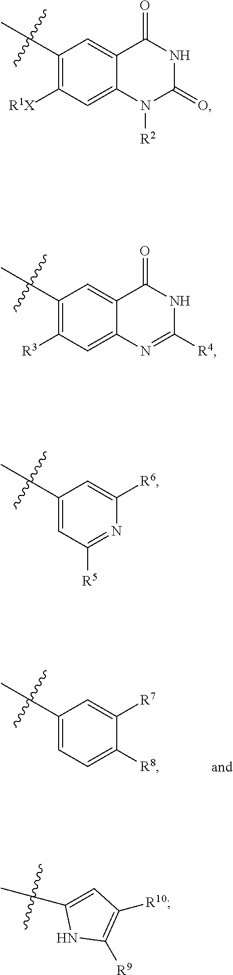

and
B is selected from the group consisting of C₄₋₆cycloalkyl, tetrahydrofuranyl, pipiridinyl, phenyl, pyridyl,

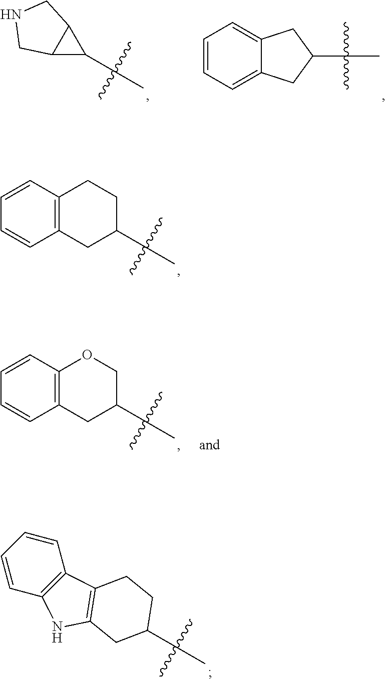

wherein
X is O or NH;
R¹, R², R⁴, and R⁵ are each independently C₁₋₆alkyl;
R³ is H, C₁₋₆alkyl, —NH—C₁₋₆alkyl, or —O—C₁₋₆alkyl, wherein the —O—C₁₋₆alkyl of R³ is optionally substituted with heterocyclyl;
R⁶ is 4- to 5-membered nitrogen-containing heterocyclyl substituted with one or more independently selected —CN, —OH, C₁₋₆alkyl, or —O—C₁₋₆alkyl substituents, wherein each C₁₋₆alkyl or —O—C₁₋₆alkyl substituent is optionally substituted with one or more independently selected halo substituents;
R⁷ is —CN or —C(O)—NH₂;
R⁸ is —NH—C₁₋₆alkyl or —N(C₁₋₆alkyl)₂;
R⁹ is C₁₋₆alkyl;
R¹⁰ is —C(O)—R^{a},

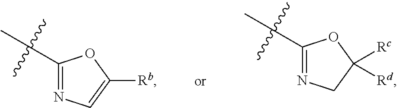

wherein
   R^{a} is selected from the group consisting of —O—C₁₋₆alkyl, —NR^{a1}R^{a2}, and a 4- to 7-membered nitrogen-containing heterocyclyl optionally substituted with one or more independently selected halo, —OH, —CN, C₁₋₆alkyl, C₁₋₆haloalkyl, or —O—C₁₋₆haloalkyl substituents;
   R^{a1} is H or C₁₋₆alkyl;
   R^{a2} is H or C₁₋₆alkyl optionally substituted with one or more independently selected halo, —OH, C₁₋₆haloalkyl, —O—C₁₋₆alkyl, or —NH—C₁₋₆haloalkyl substituents;
   R^{b}, R^{c}, and R^{d} are independently selected C₁₋₆ alkyl; and
B is optionally substituted with one or more substituents independently selected from the group consisting of: halo; —OH; C₁₋₆alkyl optionally substituted with phenyl, wherein the phenyl is optionally substituted with one or more independently selected halo substituents; C₁₋₆haloalkyl; C₃₋₆cycloalkyl; 3- to 6-membered heterocyclyl optionally substituted with one or more independently selected C₁₋₆alkyl substituents; phenyl optionally substituted with one or more independently selected halo, C₁₋₆alkyl, or C₁₋₆haloalkyl substituents; —NH-phenyl optionally substituted with one or more independently selected halo substituents; pyrazolyl optionally substituted with one or more independently selected C₁₋₆alkyl or C₁₋₆haloalkyl substituents; and pyridyl optionally substituted with one or more independently selected C₁₋₆alkyl or C₁₋₆haloalkyl substituents.

In some embodiments of a compound of Formula (I) or (Ia), A is selected from the group consisting of

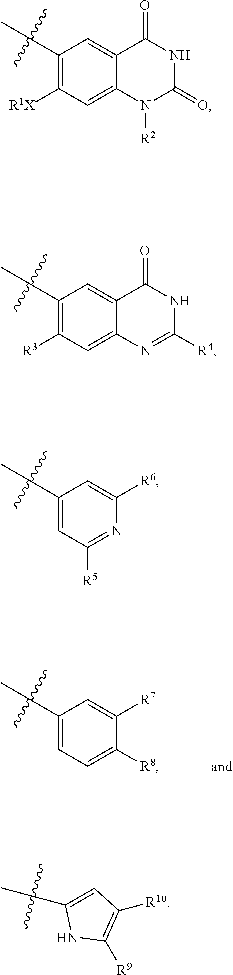

In some embodiments, A is selected from the group consisting of

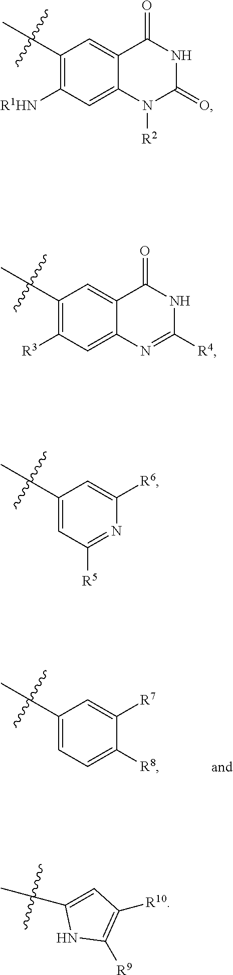

In some embodiments, A is selected from the group consisting of

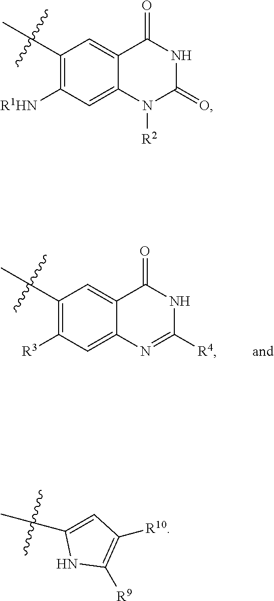

In some embodiments of a compound of Formula (I) or (Ia), A is

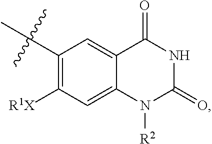

X is O or NH, and R¹ and R² are each independently C₁₋₆alkyl. In some embodiments, X is O or NH, and R¹ and R² are each methyl. In some embodiments, X is O, and R¹ and R² are each methyl. In some embodiments, X is NH, and R¹ and R² are each methyl.

In some embodiments of a compound of Formula (I) or (Ia), A is

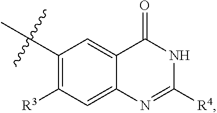

R³ is selected from the group consisting of H, —CH₃, —OCH₃, —NHCH₃, and —O—(CH₂)₂—N(CH₂CH₂)₂O, and R⁴ is C₁₋₆alkyl. In some embodiments, R³ is selected from the group consisting of H, —CH₃, —OCH₃, —NHCH₃, and —O—(CH₂)₂—N(CH₂CH₂)₂O, and R⁴ is methyl.

In some embodiments of a compound of Formula (I) or (Ia), A is

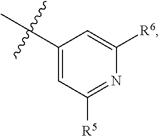

R⁵ is C₁₋₆alkyl, and R⁶ is 4- to 5-membered nitrogen-containing heterocyclyl substituted with one or more independently selected —CN, —OH, C₁₋₆alkyl, or —O—C₁₋₆alkyl substituents, wherein each C₁₋₆alkyl or —O—C₁₋₆alkyl substituent is optionally substituted with one or more independently selected halo substituents. In some embodiments, R⁵ is methyl. In some embodiments, R⁶ is a 5-membered nitrogen-containing heterocyclyl substituted with one or two independently selected —CN, —OH, C₁₋₆alkyl, or —O—C₁₋₆alkyl substituents. In some embodiments, R⁶ is a 5-membered nitrogen-containing heterocyclyl substituted with one —CN substituent. In some embodiments, R⁶ is a 4-membered nitrogen-containing heterocyclyl substituted with one or two independently selected —CN, —OH, C₁₋₆alkyl, or —O—C₁₋₆alkyl substituents, wherein each C₁₋₆alkyl or —O—C₁₋₆alkyl substituent is optionally substituted with one or more independently selected halo substituents. In some embodiments, R⁶ is a 4-membered nitrogen-containing heterocyclyl substituted with one or two independently selected —CN, —OH, C₁₋₆alkyl, or —O—C₁₋₆alkyl substituents, wherein each C₁₋₆alkyl or —O—C₁₋₆alkyl substituent is optionally substituted with two fluoro substituents. In some embodiments, R⁶ is a 4-membered nitrogen-containing heterocyclyl substituted with one substituent selected from the group consisting of —CN, —OH, C₁₋₆alkyl, and —O—C₁₋₆alkyl, wherein each C₁₋₆alkyl or —O—C₁₋₆alkyl substituent is optionally substituted with two fluoro substituents. In some embodiments, R⁶ is

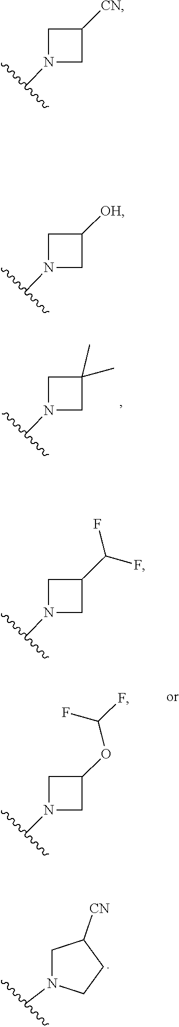

In some embodiments of a compound of Formula (I) or (Ia), A is

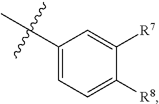

—CN or —C(O)—NH₂, and R⁸ is —NH—C₁₋₆alkyl or —N(C₁₋₆alkyl)₂. In some embodiments, R⁷ is CN, and R⁸ is —NH—C₁₋₆alkyl or —N(C₁₋₆alkyl)₂. In some embodiments, R⁷ is CN, R⁸ is —NH(CH₃) or —N(CH₃)₂. In some embodiments, R⁷ is —C(O)—NH₂, and R⁸ is —NH—C₁₋₆alkyl. In some embodiments, R⁷ is —C(O)—NH₂ and R⁸ is —NH—CH₃.

In some embodiments of a compound of Formula (I) or (Ia), A is

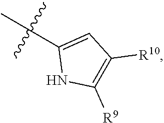

R⁹ is C₁₋₆alkyl, and R¹⁰ is —C(O)—R^{a}, wherein R^{a} is selected from the group consisting of —O—C₁₋₆alkyl, —NR^{a1}R^{a2}, and 4- to 7-membered nitrogen-containing heterocyclyl optionally substituted with one or more independently selected halo, —OH, —CN, C₁₋₆alkyl, C₁₋₆haloalkyl, or —O—C₁₋₆haloalkyl substituents. In some embodiments, R⁹ is methyl. In some embodiments, R^{a} is selected from the group consisting of —O—C₁₋₆alkyl, —NH₂, —NH—C₁₋₆alkyl, and —N(C₁₋₆alkyl)₂, wherein the —NH—C₁₋₆alkyl is optionally substituted with one or more independently selected halo, —OH, —O—C₁₋₆alkyl substituents. In some embodiments, R^{a} is selected from the group consisting of —O—C₁₋₃alkyl, —NH₂, —NH—C₁₋₃alkyl, and —N(C₁₋₃alkyl)₂, wherein the —NH—C₁₋₃alkyl is optionally substituted with one or more independently selected fluoro, —OH, or —O—C₁₋₃alkyl substituents. In some embodiments, R^{a} is 4- to 7-membered nitrogen-containing heterocyclyl optionally substituted with one or more independently selected halo, —OH, —CN, C₁₋₆alkyl, C₁₋₆haloalkyl, or —O—C₁₋₆haloalkyl substituents. In some embodiments, R^{a} is 4- to 7-membered nitrogen-containing heterocyclyl selected from the group consisting of

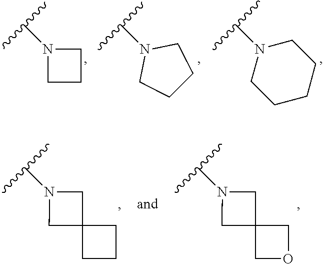

wherein each 4- to 7-membered nitrogen-containing heterocyclyl of R^{a} is optionally substituted with one or more independently selected fluoro, —OH, —CN, —CH₃, —CF₃, or —OCF₃ substituents. In some embodiments, R^{a} is unsubstituted

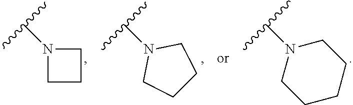 [SMILES: *N1CCC1 ; *N1CCCC1 ; *N1CCCCC1]

In some embodiments, R^{a} is

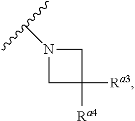

wherein R^{a3} and R^{a4} are independently selected H, fluoro, —OH, —CN, —CH₃, —CF₃, or —OCF₃ substituents. In some embodiments, R^{a} is

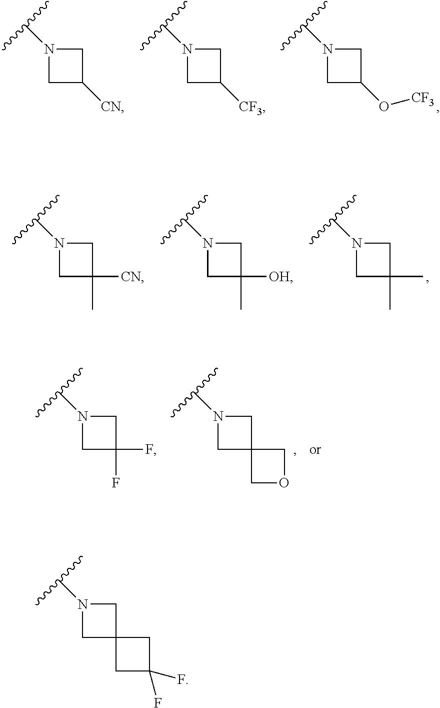

In some embodiments of a compound of Formula (I) or (Ia), A is

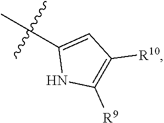

R⁹ is C₁₋₆alkyl, and R¹⁰ is

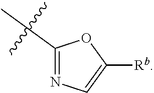

In some embodiments, R⁹ is CH₃. In some embodiments, R^{b} is C₁₋₆ alkyl; in some embodiments, R^{b} is methyl.

In some embodiments of a compound of Formula (I) or (Ia), A is

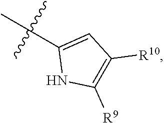

R⁹ is C₁₋₆alkyl, and R¹⁰ is

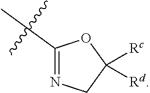

In some embodiments, R⁹ is methyl. In some embodiments, R^{c} and R^{d} are independently selected C₁₋₆ alkyl. In some embodiments, R^{c} and R^{d} are each methyl.

In some embodiments of a compound of Formula (I), B is selected from the group consisting of C₄₋₆cycloalkyl, tetrahydrofuranyl, pipiridinyl, phenyl, pyridyl, indolyl

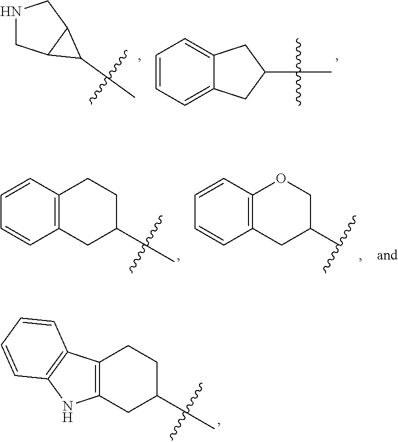

wherein B is optionally substituted with one or more substituents independently selected from the group consisting of: halo; —OH; C₁₋₆alkyl optionally substituted with phenyl, wherein the phenyl is optionally substituted with one or more independently selected halo substituents; C₁₋₆haloalkyl; C₃₋₆cycloalkyl; 3- to 6-membered heterocyclyl optionally substituted with one or more independently selected C₁₋₆alkyl substituents; phenyl optionally substituted with one or more independently selected halo, C₁₋₆alkyl, or C₁₋₆haloalkyl substituents; —NH-phenyl optionally substituted with one or more independently selected halo substituents; pyrazolyl optionally substituted with one or more independently selected C₁₋₆alkyl or C₁₋₆haloalkyl substituents; and pyridyl optionally substituted with one or more independently selected C₁₋₆alkyl or C₁₋₆haloalkyl substituents.

In some embodiments of a compound of Formula (I) or (Ia), B is selected from the group consisting of C₄₋₆cycloalkyl, tetrahydrofuranyl, pipiridinyl, phenyl, pyridyl,

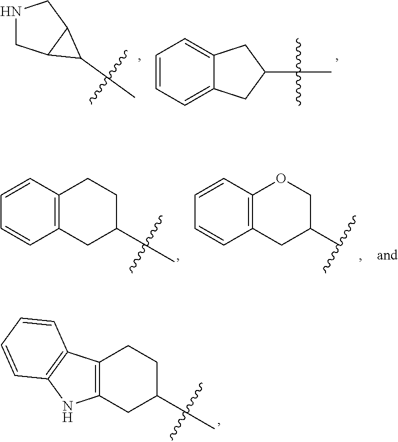

wherein B is optionally substituted with one or more substituents independently selected from the group consisting of: halo; —OH; C₁₋₆alkyl optionally substituted with phenyl, wherein the phenyl is optionally substituted with one or more independently selected halo substituents; C₁₋₆haloalkyl; C₃₋₆cycloalkyl; 3- to 6-membered heterocyclyl optionally substituted with one or more independently selected C₁₋₆alkyl substituents; phenyl optionally substituted with one or more independently selected halo, C₁₋₆alkyl, or C₁₋₆haloalkyl substituents; —NH-phenyl optionally substituted with one or more independently selected halo substituents; pyrazolyl optionally substituted with one or more independently selected C₁₋₆alkyl or C₁₋₆haloalkyl substituents; and pyridyl optionally substituted with one or more independently selected C₁₋₆alkyl or C₁₋₆haloalkyl substituents. In some embodiments, B is selected from the group consisting of C₄₋₆cycloalkyl, pyridyl,

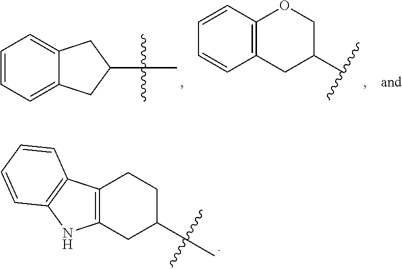

In some embodiments of a compound of Formula (I) or (Ia), B is selected from the group consisting of:

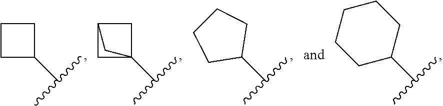

wherein B is substituted with one or two substituents from the group consisting of: C₁₋₆alkyl; phenyl optionally substituted with one or more independently selected halo or C₁₋₆haloalkyl substituents; —NH-phenyl optionally substituted with one or more independently selected halo substituents; pyrazolyl substituted with one or more independently selected C₁₋₆alkyl or C₁₋₆haloalkyl substituents; and pyridyl substituted with one or more independently selected C₁₋₆haloalkyl substituents. In some embodiments, B is substituted with one or two substituents from the group consisting of: 1-pyrazolyl substituted with one or more independently selected C₁₋₆alkyl or C₁₋₆haloalkyl substituents; and 2-pyridyl substituted with one or more independently selected C₁₋₆haloalkyl substituents.

In some embodiments of a compound of Formula (I) or (Ia), B is

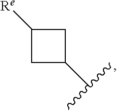

wherein R^{e} is selected from the group consisting of: phenyl optionally substituted with one or more independently selected halo or C₁₋₆haloalkyl substituents; —NH-phenyl optionally substituted with one or more independently selected halo substituents; pyrazolyl substituted with one or more independently selected C₁₋₆alkyl or C₁₋₆haloalkyl substituents; and pyridyl substituted with one or more independently selected C₁₋₆haloalkyl substituents. In some embodiments, B is substituted with one or two substituents from the group consisting of: 1-pyrazolyl substituted with one or more independently selected C₁₋₆alkyl or C₁₋₆haloalkyl substituents; and 2-pyridyl substituted with one or more independently selected C₁₋₆haloalkyl substituents.

In some embodiments of a compound of Formula (I) or (Ia), B is tetrahydrofuranyl substituted with C₁₋₆alkyl, wherein the C₁₋₆alkyl is optionally substituted with phenyl, wherein the phenyl is optionally substituted with one or more independently selected halo substituents. In some embodiments, B is tetrahydrofuranyl substituted with —C₁₋₆alkyl-C₆H₄Cl. In some embodiments B is tetrahydrofuranyl substituted with —(CH₂)—C₆H₄Cl. In some embodiments, B is

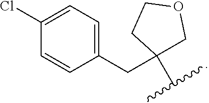 [SMILES: *C1(Cc2ccc(Cl)cc2)CCOC1]

In some embodiments of a compound of Formula (I) or (Ia), B is pipiridinyl substituted with phenyl, wherein the phenyl is optionally substituted with one or more independently selected halo substituents. In some embodiments, B is pipiridinyl substituted with phenyl, wherein the phenyl is substituted with one halo substituent. In some embodiments, B is pipiridinyl substituted with phenyl, wherein the phenyl is substituted with one fluoro.

In some embodiments of a compound of Formula (I) or (Ia), B is phenyl optionally substituted with phenyl. In some embodiments, B is unsubstituted phenyl. In some embodiments, B is unsubstituted phenyl and L¹ is —NH—C₁₋₆alkylene-. In some embodiments, B is biphenyl.

In some embodiments of a compound of Formula (I) or (Ia), B is pyridyl optionally substituted with one or more 3- to 6-membered heterocyclyl substituents, wherein the 3- to 6-membered heterocyclyl is optionally substituted with one or more independently selected C₁₋₆alkyl substituents. In some embodiments, B is pyridyl substituted with one or more independently selected 3- to 6-membered heterocyclyl, wherein the 3- to 6-membered heterocyclyl is optionally substituted with one or more independently selected C₁₋₆alkyl substituents. In some embodiments, B is pyridyl substituted with a pipiridinyl group, wherein the pipiridinyl group is substituted with two independently selected C₁₋₆alkyl substituents. In some embodiments, B is

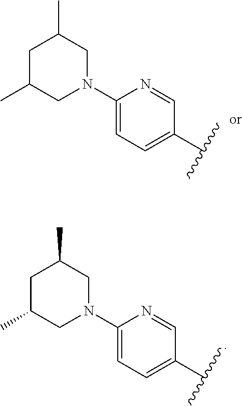 [SMILES: *c1ccc(N2CC(C)CC(C)C2)nc1 ; *c1ccc(N2C[C@H](C)C[C@@H](C)C2)nc1]

In some embodiments of a compound of Formula (I), B is indolyl optionally substituted with one or more independently selected halo or C₁₋₆alkyl substituents. In some embodiments, B is indolyl optionally substituted with one halo or C₁₋₆alkyl substituent. In some embodiments, B is 2-indolyl optionally substituted with one halo or C₁₋₆alkyl substituent. In some embodiments, B is 2-indolyl optionally substituted with one fluoro or methyl. In some embodiments of the foregoing, L¹ is —C₁₋₆alkylene-. In some embodiments, L¹ is —C₂₋₃alkylene-.

In some embodiments of a compound of Formula (I) or (Ia), B is

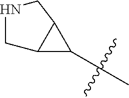 [SMILES: *C1C2CNCC12]

optionally substituted with one or more independently selected phenyl substituents, wherein each phenyl is optionally substituted with one or more independently selected halo, C₁₋₆alkyl, or C₁₋₆haloalkyl substituents. In some embodiments, B is

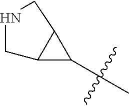 [SMILES: *C1C2CNCC12]

substituted with one phenyl substituent, wherein the phenyl is optionally substituted with one or more independently selected halo substituents. In some embodiments, B is

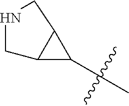 [SMILES: *C1C2CNCC12]

substituted with one phenyl substituent, wherein the phenyl is substituted with one fluoro.

In some embodiments of a compound of Formula (I) or (Ia), B is

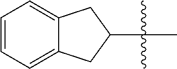 [SMILES: *C1Cc2ccccc2C1]

optionally substituted with one or more substituents independently selected from the group consisting of halo, —OH, C₁₋₆alkyl, and C₁₋₆haloalkyl. In some embodiments, B is

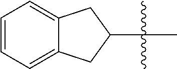 [SMILES: *C1Cc2ccccc2C1]

optionally substituted with one to four substituents independently selected from the group consisting of halo, —OH, C₁₋₆alkyl, and C₁₋₆haloalkyl. In some embodiments, B is

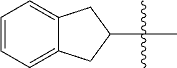 [SMILES: *C1Cc2ccccc2C1]

optionally substituted with one to four substituents independently selected from the group consisting of F, Cl, —OH, methyl, and CF₃. In some embodiments, B is

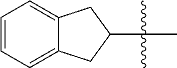 [SMILES: *C1Cc2ccccc2C1]

substituted with one to four substituents independently selected from the group consisting of F, Cl, —OH, methyl, and CF₃.

In some embodiments of a compound of Formula (I) or (Ia), B is

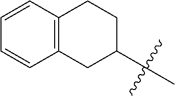 [SMILES: *C1CCc2ccccc2C1]

optionally substituted with one or more independently selected halo substituents. In some embodiments, B is

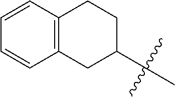 [SMILES: *C1CCc2ccccc2C1]

substituted with one halo substituent. In some embodiments, B is

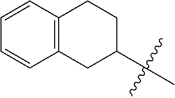 [SMILES: *C1CCc2ccccc2C1]

substituted with one chloro.

In some embodiments of a compound of Formula (I) or (Ia), B is

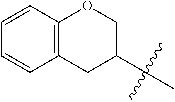 [SMILES: *C1COc2ccccc2C1]

optionally substituted with one or more substituents independently selected from the group consisting of halo, —OH, C₁₋₆alkyl, and C₃₋₆cycloalkyl. In some embodiments, B is

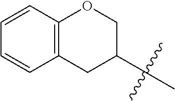 [SMILES: *C1COc2ccccc2C1]

substituted with one to three substituents independently selected from the group consisting of halo, —OH, C₁₋₆alkyl, and C₃₋₆cycloalkyl. In some embodiments, B is

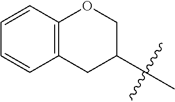 [SMILES: *C1COc2ccccc2C1]

substituted with one to three substituents independently selected from the group consisting of fluor, chloro, —OH, methyl, and cyclopropyl. In some embodiments, B is

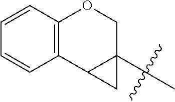 [SMILES: *C12COc3ccccc3C1C2]

substituted with one to two independently selected F or Cl substituents.

In some embodiments of a compound of Formula (I) or (Ia), B is unsubstituted

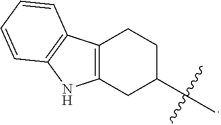 [SMILES: *C1CCc2c([nH]c3ccccc23)C1]

In some embodiments of a compound of Formula (I),

A is

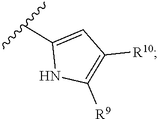

L¹ is a bond; and

B is selected from the group consisting of C₄₋₆cycloalkyl,

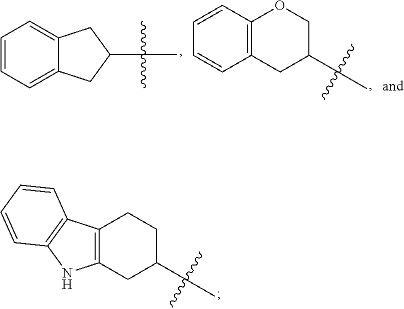

wherein

B is optionally substituted with one or more substituents independently selected from the group consisting of: halo; —OH; C₁₋₆alkyl; C₁₋₆haloalkyl; C₃₋₆cycloalkyl; phenyl optionally substituted with one or more independently selected halo or C₁₋₆haloalkyl substituents; pyrazolyl optionally substituted with one or more independently selected C₁₋₆alkyl or C₁₋₆haloalkyl substituents; and pyridyl optionally substituted with one or more independently selected C₁₋₆haloalkyl substituents.

In some embodiments of a compound of Formula (I),
A is

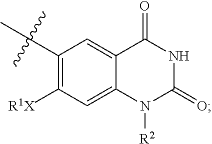

L¹ is a bond; and
B is C₄₋₆cycloalkyl; wherein
X is NH or O;
R¹ is CH₃;
R² is CH₃; and
B is optionally substituted with one or more substituents from the group consisting of: C₁₋₆alkyl; unsubstituted phenyl; —NH-phenyl optionally substituted with one or more independently selected halo substituents; pyrazolyl substituted with one or more independently selected C₁₋₆alkyl or C₁₋₆haloalkyl substituents; and pyridyl substituted with one or more independently selected C₁₋₆haloalkyl substituents.

In some embodiments of a compound of Formula (Ia),

A is

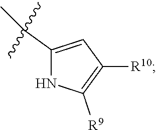

and

B is selected from the group consisting of C₄₋₆cycloalkyl, and

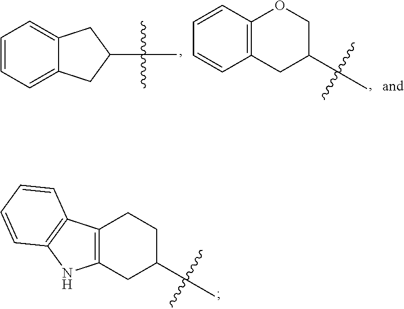

wherein

B is optionally substituted with one or more substituents independently selected from the group consisting of: halo; —OH; C₁₋₆alkyl; C₁₋₆haloalkyl; C₃₋₆cycloalkyl; phenyl optionally substituted with one or more independently selected halo or C₁₋₆haloalkyl substituents; pyrazolyl optionally substituted with one or more independently selected C₁₋₆alkyl or C₁₋₆haloalkyl substituents; and pyridyl optionally substituted with one or more independently selected C₁₋₆haloalkyl substituents.

In some embodiments of a compound of Formula (Ia),
A is

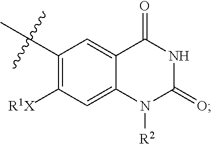

and
B is C₄₋₆cycloalkyl; wherein
X is NH or O;
R¹ is CH₃;
R² is CH₃; and
B is optionally substituted with one or more substituents from the group consisting of: C₁₋₆alkyl; unsubstituted phenyl; —NH-phenyl optionally substituted with one or more independently selected halo substituents; pyrazolyl substituted with one or more independently selected C₁₋₆alkyl or C₁₋₆haloalkyl substituents; and pyridyl substituted with one or more independently selected C₁₋₆haloalkyl substituents.

In some embodiments, provided is a compound as shown in Table 1, or a pharmaceutically acceptable salt thereof.

TABLE 1

| | |
|---|---|
| 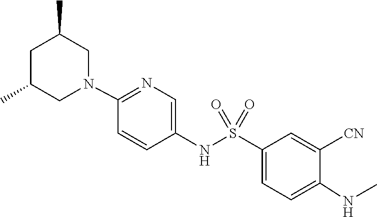 | 1 |
| 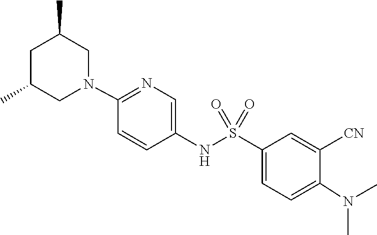 | 2 |
| 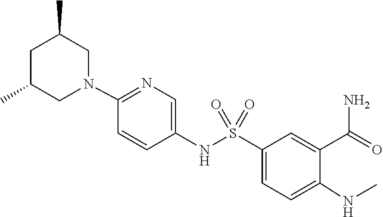 [SMILES: CNc1ccc(S(=O)(=O)Nc2ccc(N3C[C@H](C)C[C@@H](C)C3)nc2)cc1C(N)=O] | 3 |
| 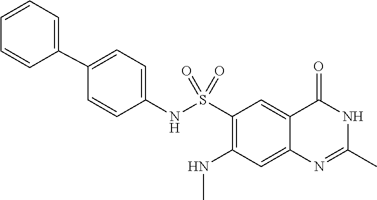 [SMILES: CNc1cc2nc(C)[nH]c(=O)c2cc1S(=O)(=O)Nc1ccc(-c2ccccc2)cc1] | 4 |
| 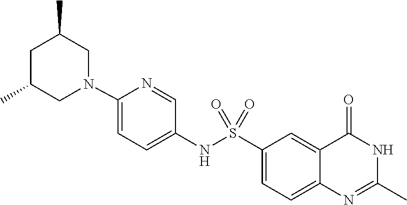 | 5 |
| 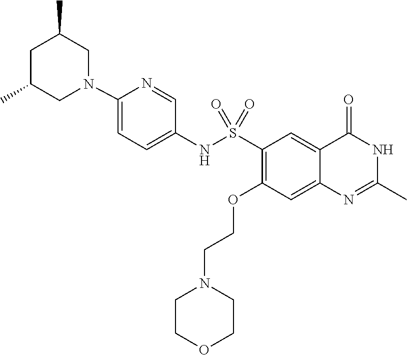 | 6 |
| 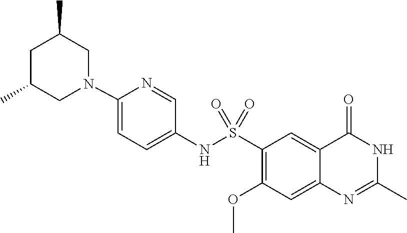 | 7 |
| 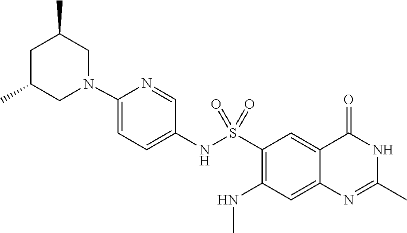 | 8 |
| 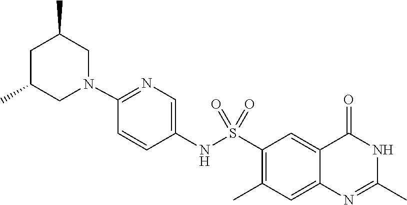 | 9 |
| 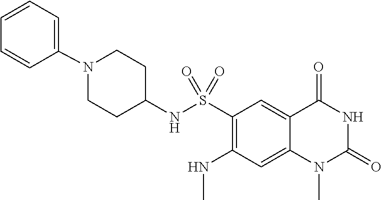 [SMILES: CNc1cc2c(cc1S(=O)(=O)NC1CCN(c3ccccc3)CC1)c(=O)[nH]c(=O)n2C] | 10 |
| 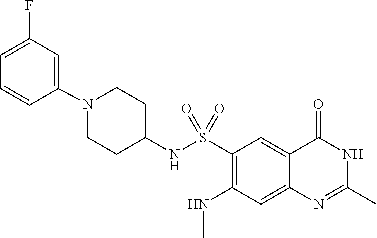 [SMILES: CNc1cc2nc(C)[nH]c(=O)c2cc1S(=O)(=O)NC1CCN(c2cccc(F)c2)CC1] | 11 |
| 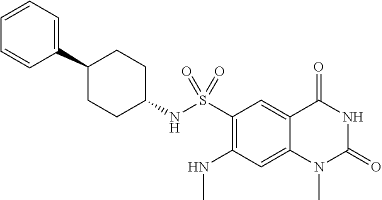 [SMILES: CNc1cc2c(cc1S(=O)(=O)N[C@H]1CC[C@H](c3ccccc3)CC1)c(=O)[nH]c(=O)n2C] | 12 |
| 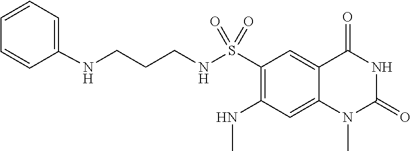 [SMILES: CNc1cc2c(cc1S(=O)(=O)NCCCNc1ccccc1)c(=O)[nH]c(=O)n2C] | 13 |
| 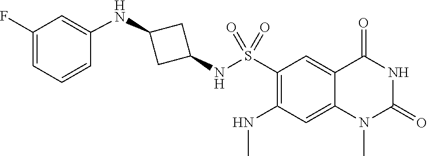 [SMILES: CNc1cc2c(cc1S(=O)(=O)N[C@H]1C[C@@H](Nc3cccc(F)c3)C1)c(=O)[nH]c(=O)n2C] | 14 |
| 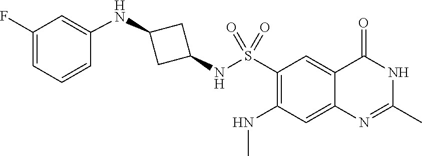 [SMILES: CNc1cc2nc(C)[nH]c(=O)c2cc1S(=O)(=O)N[C@H]1C[C@@H](Nc2cccc(F)c2)C1] | 15 |
| 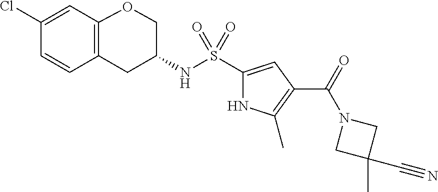 [SMILES: Cc1[nH]c(S(=O)(=O)N[C@H]2COc3cc(Cl)ccc3C2)cc1C(=O)N1CC(C)(C#N)C1] | 16 |
| 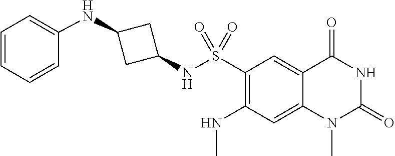 [SMILES: CNc1cc2c(cc1S(=O)(=O)N[C@H]1C[C@@H](Nc3ccccc3)C1)c(=O)[nH]c(=O)n2C] | 17 |
| 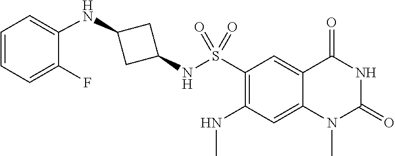 [SMILES: CNc1cc2c(cc1S(=O)(=O)N[C@H]1C[C@@H](Nc3ccccc3F)C1)c(=O)[nH]c(=O)n2C] | 18 |
| 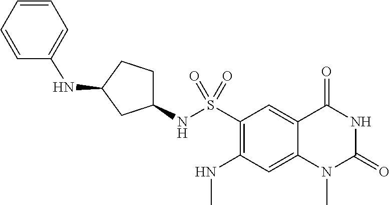 [SMILES: CNc1cc2c(cc1S(=O)(=O)N[C@@H]1CC[C@H](Nc3ccccc3)C1)c(=O)[nH]c(=O)n2C] | 19 |
| 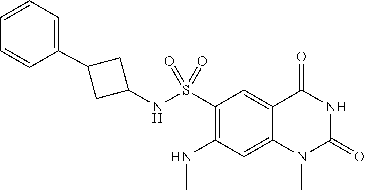 [SMILES: CNc1cc2c(cc1S(=O)(=O)NC1CC(c3ccccc3)C1)c(=O)[nH]c(=O)n2C] | 20 |
| 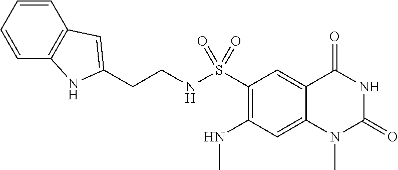 [SMILES: CNc1cc2c(cc1S(=O)(=O)NCCc1cc3ccccc3[nH]1)c(=O)[nH]c(=O)n2C] | 21 |
| 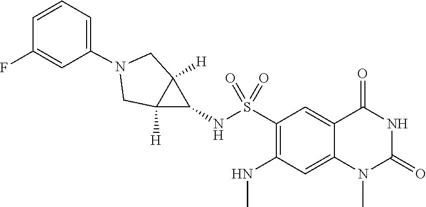 [SMILES: CNc1cc2c(cc1S(=O)(=O)N[C@H]1[C@@H]3CN(c4cccc(F)c4)C[C@@H]31)c(=O)[nH]c(=O)n2C] | 22 |
| 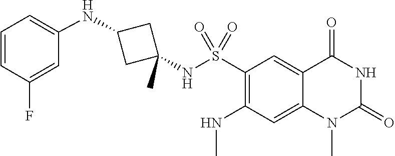 [SMILES: CNc1cc2c(cc1S(=O)(=O)N[C@]1(C)C[C@H](Nc3cccc(F)c3)C1)c(=O)[nH]c(=O)n2C] | 23 |
| 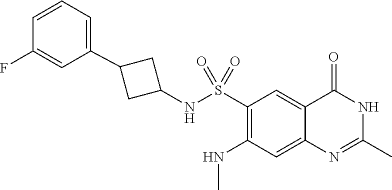 [SMILES: CNc1cc2nc(C)[nH]c(=O)c2cc1S(=O)(=O)NC1CC(c2cccc(F)c2)C1] | 24 |
| 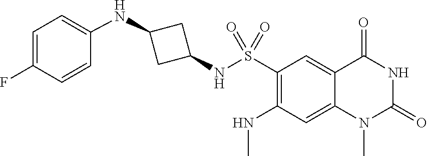 [SMILES: CNc1cc2c(cc1S(=O)(=O)N[C@H]1C[C@@H](Nc3ccc(F)cc3)C1)c(=O)[nH]c(=O)n2C] | 25 |
| 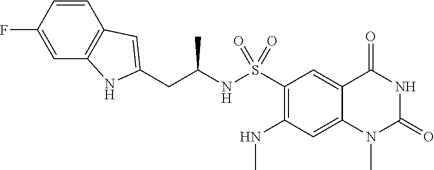 [SMILES: CNc1cc2c(cc1S(=O)(=O)N[C@H](C)Cc1cc3ccc(F)cc3[nH]1)c(=O)[nH]c(=O)n2C] | 26 |
| 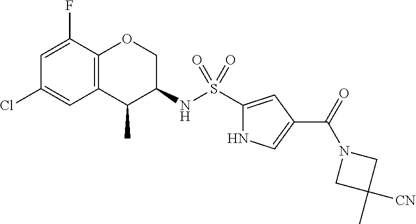 [SMILES: C[C@H]1c2cc(Cl)cc(F)c2OC[C@H]1NS(=O)(=O)c1cc(C(=O)N2CC(C)(C#N)C2)c[nH]1] | 27 |
| 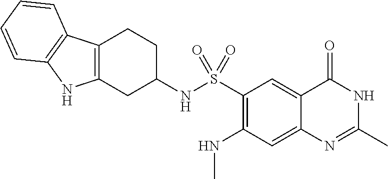 [SMILES: CNc1cc2nc(C)[nH]c(=O)c2cc1S(=O)(=O)NC1CCc2c([nH]c3ccccc23)C1] | 28 |
| 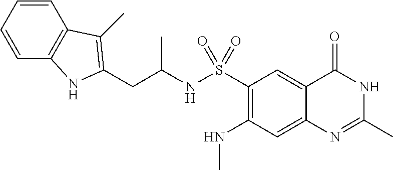 [SMILES: CNc1cc2nc(C)[nH]c(=O)c2cc1S(=O)(=O)NC(C)Cc1[nH]c2ccccc2c1C] | 29 |
| 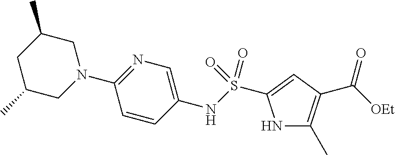 [SMILES: CCOC(=O)c1cc(S(=O)(=O)Nc2ccc(N3C[C@H](C)C[C@@H](C)C3)nc2)[nH]c1C] | 30 |
| 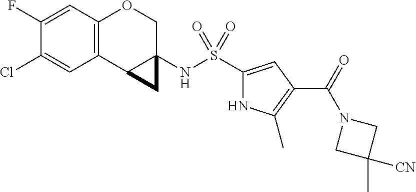 [SMILES: Cc1[nH]c(S(=O)(=O)N[C@@]23COc4cc(F)c(Cl)cc4[C@@H]2C3)cc1C(=O)N1CC(C)(C#N)C1] | 31 |
| 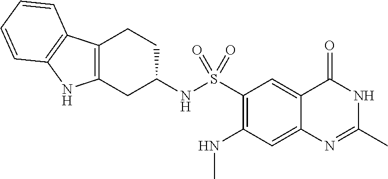 [SMILES: CNc1cc2nc(C)[nH]c(=O)c2cc1S(=O)(=O)N[C@H]1CCc2c([nH]c3ccccc23)C1] | 32 |
| 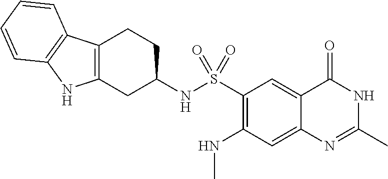 [SMILES: CNc1cc2nc(C)[nH]c(=O)c2cc1S(=O)(=O)N[C@@H]1CCc2c([nH]c3ccccc23)C1] | 33 |
| 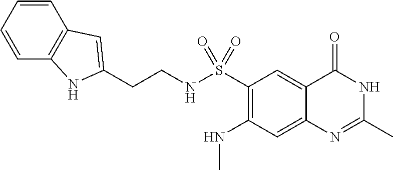 [SMILES: CNc1cc2nc(C)[nH]c(=O)c2cc1S(=O)(=O)NCCc1cc2ccccc2[nH]1] | 34 |
| 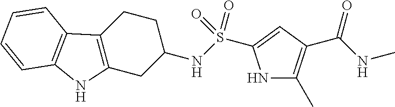 [SMILES: CNC(=O)c1cc(S(=O)(=O)NC2CCc3c([nH]c4ccccc34)C2)[nH]c1C] | 35 |
| 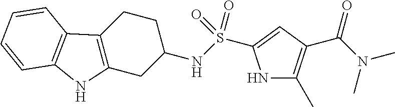 [SMILES: Cc1[nH]c(S(=O)(=O)NC2CCc3c([nH]c4ccccc34)C2)cc1C(=O)N(C)C] | 36 |
| 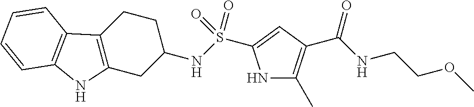 [SMILES: COCCNC(=O)c1cc(S(=O)(=O)NC2CCc3c([nH]c4ccccc34)C2)[nH]c1C] | 37 |
| 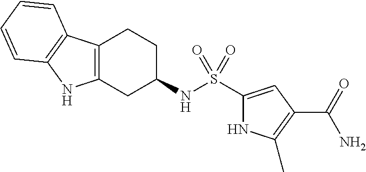 [SMILES: Cc1[nH]c(S(=O)(=O)N[C@@H]2CCc3c([nH]c4ccccc34)C2)cc1C(N)=O] | 38 |
| 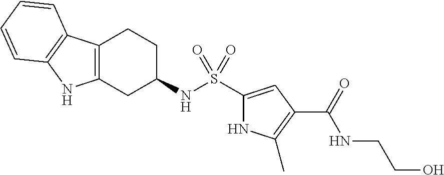 [SMILES: Cc1[nH]c(S(=O)(=O)N[C@@H]2CCc3c([nH]c4ccccc34)C2)cc1C(=O)NCCO] | 39 |
| 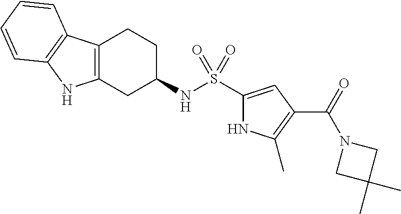 [SMILES: Cc1[nH]c(S(=O)(=O)N[C@@H]2CCc3c([nH]c4ccccc34)C2)cc1C(=O)N1CC(C)(C)C1] | 40 |
| 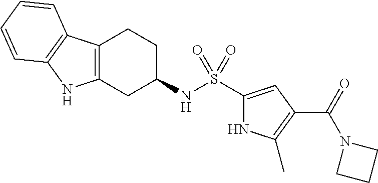 [SMILES: Cc1[nH]c(S(=O)(=O)N[C@@H]2CCc3c([nH]c4ccccc34)C2)cc1C(=O)N1CCC1] | 41 |
| 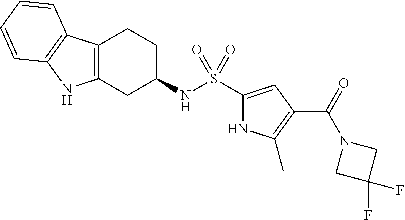 [SMILES: Cc1[nH]c(S(=O)(=O)N[C@@H]2CCc3c([nH]c4ccccc34)C2)cc1C(=O)N1CC(F)(F)C1] | 42 |
| 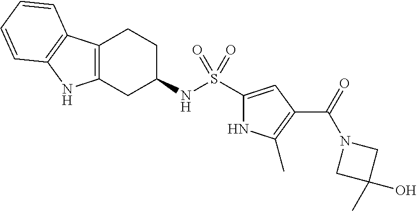 [SMILES: Cc1[nH]c(S(=O)(=O)N[C@@H]2CCc3c([nH]c4ccccc34)C2)cc1C(=O)N1CC(C)(O)C1] | 43 |
| 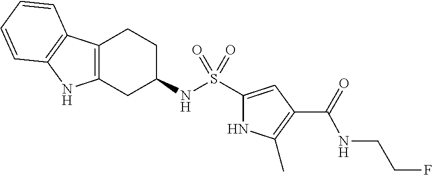 [SMILES: Cc1[nH]c(S(=O)(=O)N[C@@H]2CCc3c([nH]c4ccccc34)C2)cc1C(=O)NCCF] | 44 |
| 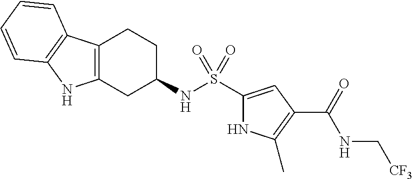 [SMILES: Cc1[nH]c(S(=O)(=O)N[C@@H]2CCc3c([nH]c4ccccc34)C2)cc1C(=O)NCC(F)(F)F] | 45 |
| 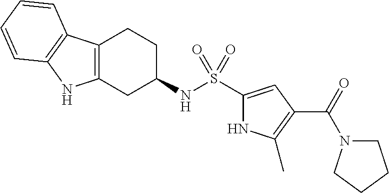 [SMILES: Cc1[nH]c(S(=O)(=O)N[C@@H]2CCc3c([nH]c4ccccc34)C2)cc1C(=O)N1CCCC1] | 46 |
| 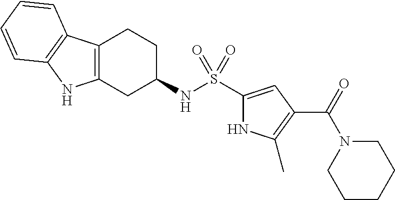 [SMILES: Cc1[nH]c(S(=O)(=O)N[C@@H]2CCc3c([nH]c4ccccc34)C2)cc1C(=O)N1CCCCC1] | 47 |
| 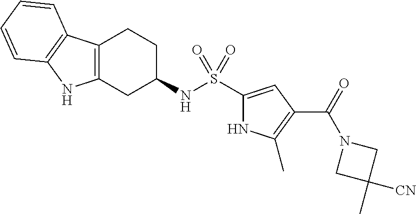 [SMILES: Cc1[nH]c(S(=O)(=O)N[C@@H]2CCc3c([nH]c4ccccc34)C2)cc1C(=O)N1CC(C)(C#N)C1] | 48 |
| 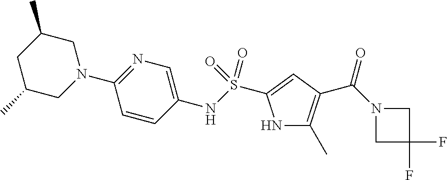 [SMILES: Cc1[nH]c(S(=O)(=O)Nc2ccc(N3C[C@H](C)C[C@@H](C)C3)nc2)cc1C(=O)N1CC(F)(F)C1] | 49 |
| 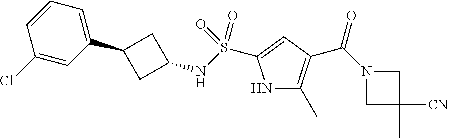 [SMILES: Cc1[nH]c(S(=O)(=O)N[C@H]2C[C@H](c3cccc(Cl)c3)C2)cc1C(=O)N1CC(C)(C#N)C1] | 50 |
| 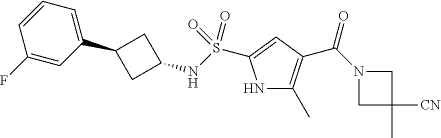 [SMILES: Cc1[nH]c(S(=O)(=O)N[C@H]2C[C@H](c3cccc(F)c3)C2)cc1C(=O)N1CC(C)(C#N)C1] | 51 |
| 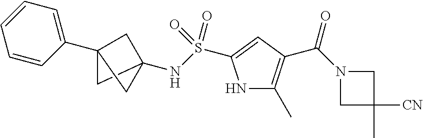 [SMILES: Cc1[nH]c(S(=O)(=O)NC23CC(c4ccccc4)(C2)C3)cc1C(=O)N1CC(C)(C#N)C1] | 52 |
| 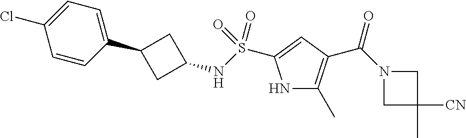 [SMILES: Cc1[nH]c(S(=O)(=O)N[C@H]2C[C@H](c3ccc(Cl)cc3)C2)cc1C(=O)N1CC(C)(C#N)C1] | 53 |
| 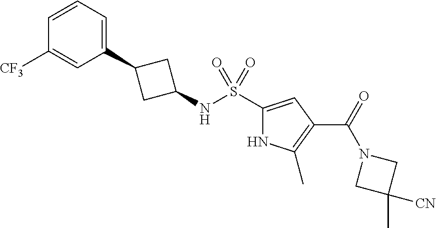 [SMILES: Cc1[nH]c(S(=O)(=O)N[C@H]2C[C@@H](c3cccc(C(F)(F)F)c3)C2)cc1C(=O)N1CC(C)(C#N)C1] | 54 |
| 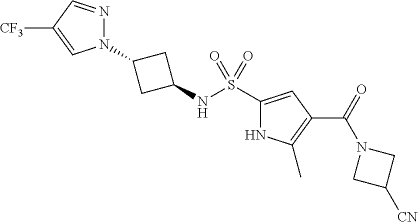 [SMILES: Cc1[nH]c(S(=O)(=O)N[C@H]2C[C@H](n3cc(C(F)(F)F)cn3)C2)cc1C(=O)N1CC(C#N)C1] | 55 |
| 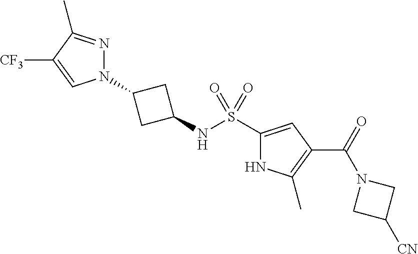 [SMILES: Cc1nn([C@H]2C[C@H](NS(=O)(=O)c3cc(C(=O)N4CC(C#N)C4)c(C)[nH]3)C2)cc1C(F)(F)F] | 56 |
| 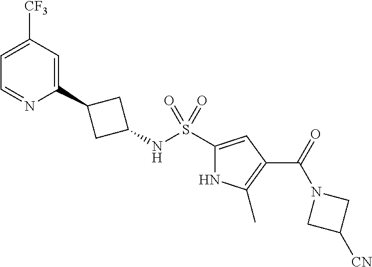 [SMILES: Cc1[nH]c(S(=O)(=O)N[C@H]2C[C@H](c3cc(C(F)(F)F)ccn3)C2)cc1C(=O)N1CC(C#N)C1] | 57 |
| 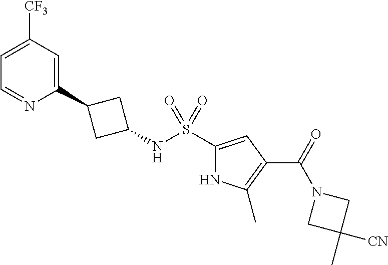 [SMILES: Cc1[nH]c(S(=O)(=O)N[C@H]2C[C@H](c3cc(C(F)(F)F)ccn3)C2)cc1C(=O)N1CC(C)(C#N)C1] | 58 |
| 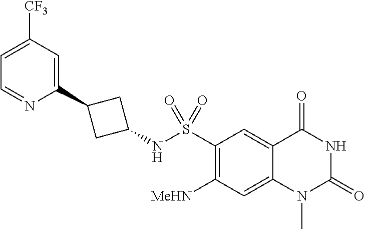 [SMILES: CNc1cc2c(cc1S(=O)(=O)N[C@H]1C[C@H](c3cc(C(F)(F)F)ccn3)C1)c(=O)[nH]c(=O)n2C] | 59 |
| 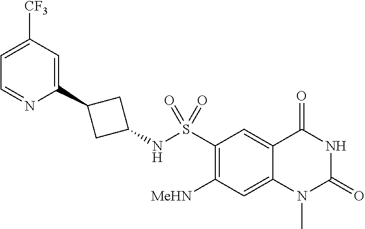 [SMILES: CNc1cc2c(cc1S(=O)(=O)N[C@H]1C[C@H](c3cc(C(F)(F)F)ccn3)C1)c(=O)[nH]c(=O)n2C] | 60 |
| 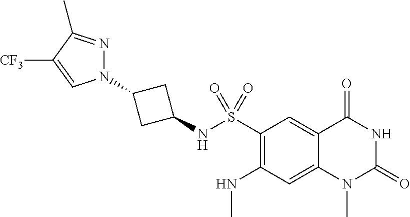 [SMILES: CNc1cc2c(cc1S(=O)(=O)N[C@H]1C[C@H](n3cc(C(F)(F)F)c(C)n3)C1)c(=O)[nH]c(=O)n2C] | 61 |
| 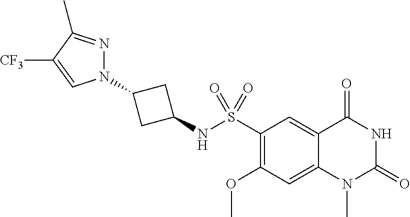 [SMILES: COc1cc2c(cc1S(=O)(=O)N[C@H]1C[C@H](n3cc(C(F)(F)F)c(C)n3)C1)c(=O)[nH]c(=O)n2C] | 62 |
| 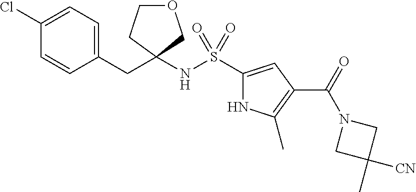 [SMILES: Cc1[nH]c(S(=O)(=O)N[C@]2(Cc3ccc(Cl)cc3)CCOC2)cc1C(=O)N1CC(C)(C#N)C1] | 63 |
| 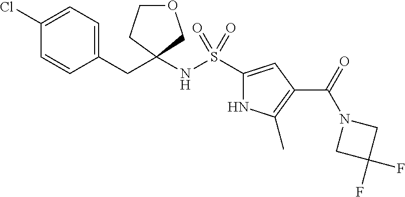 [SMILES: Cc1[nH]c(S(=O)(=O)N[C@]2(Cc3ccc(Cl)cc3)CCOC2)cc1C(=O)N1CC(F)(F)C1] | 64 |
| 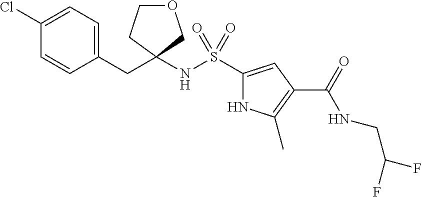 [SMILES: Cc1[nH]c(S(=O)(=O)N[C@]2(Cc3ccc(Cl)cc3)CCOC2)cc1C(=O)NCC(F)F] | 65 |
| 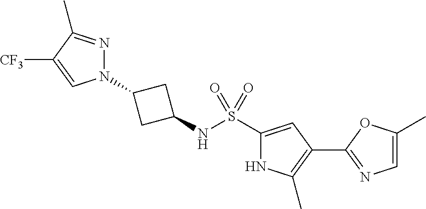 [SMILES: Cc1cnc(-c2cc(S(=O)(=O)N[C@H]3C[C@H](n4cc(C(F)(F)F)c(C)n4)C3)[nH]c2C)o1] | 66 |
| 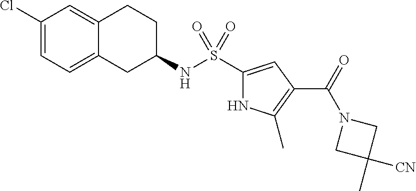 [SMILES: Cc1[nH]c(S(=O)(=O)N[C@@H]2CCc3cc(Cl)ccc3C2)cc1C(=O)N1CC(C)(C#N)C1] | 67 |
| 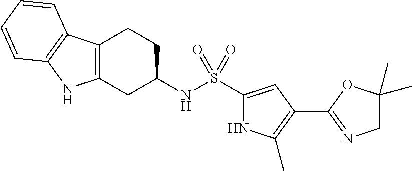 [SMILES: Cc1[nH]c(S(=O)(=O)N[C@@H]2CCc3c([nH]c4ccccc34)C2)cc1C1=NCC(C)(C)O1] | 68 |
| 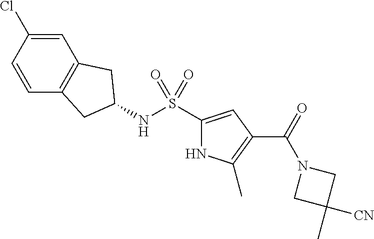 [SMILES: Cc1[nH]c(S(=O)(=O)N[C@@H]2Cc3ccc(Cl)cc3C2)cc1C(=O)N1CC(C)(C#N)C1] | 69 |
| 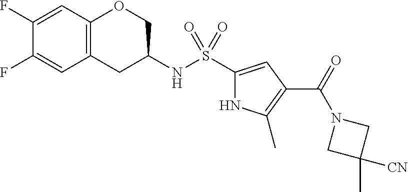 [SMILES: Cc1[nH]c(S(=O)(=O)N[C@@H]2COc3cc(F)c(F)cc3C2)cc1C(=O)N1CC(C)(C#N)C1] | 70 |
| 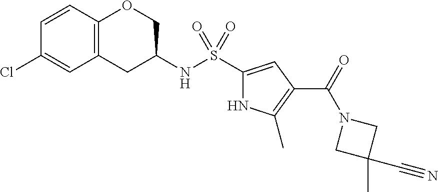 [SMILES: Cc1[nH]c(S(=O)(=O)N[C@@H]2COc3ccc(Cl)cc3C2)cc1C(=O)N1CC(C)(C#N)C1] | 71 |
| 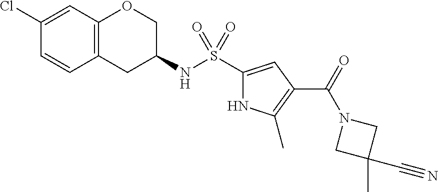 [SMILES: Cc1[nH]c(S(=O)(=O)N[C@@H]2COc3cc(Cl)ccc3C2)cc1C(=O)N1CC(C)(C#N)C1] | 72 |
| 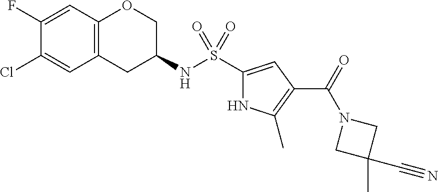 [SMILES: Cc1[nH]c(S(=O)(=O)N[C@@H]2COc3cc(F)c(Cl)cc3C2)cc1C(=O)N1CC(C)(C#N)C1] | 73 |
| 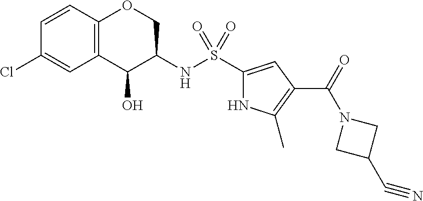 [SMILES: Cc1[nH]c(S(=O)(=O)N[C@@H]2COc3ccc(Cl)cc3[C@@H]2O)cc1C(=O)N1CC(C#N)C1] | 74 |
| 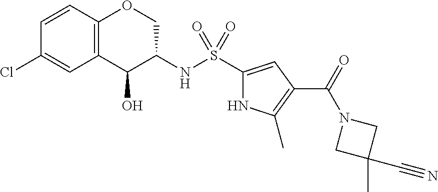 [SMILES: Cc1[nH]c(S(=O)(=O)N[C@H]2COc3ccc(Cl)cc3[C@@H]2O)cc1C(=O)N1CC(C)(C#N)C1] | 75 |
| 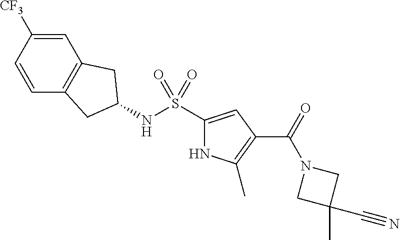 [SMILES: Cc1[nH]c(S(=O)(=O)N[C@@H]2Cc3ccc(C(F)(F)F)cc3C2)cc1C(=O)N1CC(C)(C#N)C1] | 76 |
| 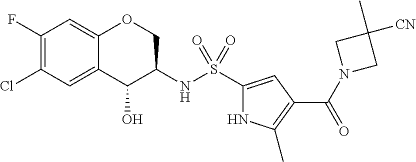 [SMILES: Cc1[nH]c(S(=O)(=O)N[C@@H]2COc3cc(F)c(Cl)cc3[C@H]2O)cc1C(=O)N1CC(C)(C#N)C1] | 77 |
| 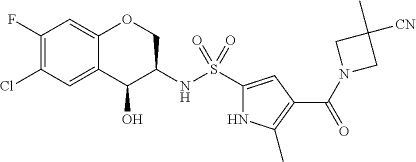 [SMILES: Cc1[nH]c(S(=O)(=O)N[C@@H]2COc3cc(F)c(Cl)cc3[C@@H]2O)cc1C(=O)N1CC(C)(C#N)C1] | 78 |
| 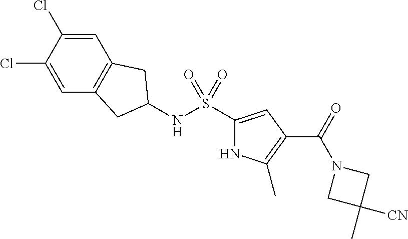 [SMILES: Cc1[nH]c(S(=O)(=O)NC2Cc3cc(Cl)c(Cl)cc3C2)cc1C(=O)N1CC(C)(C#N)C1] | 79 |
| 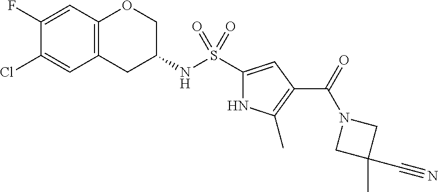 [SMILES: Cc1[nH]c(S(=O)(=O)N[C@H]2COc3cc(F)c(Cl)cc3C2)cc1C(=O)N1CC(C)(C#N)C1] | 80 |
| 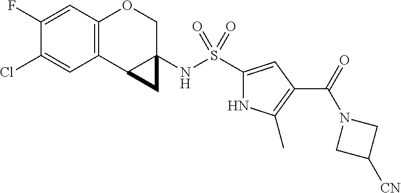 [SMILES: Cc1[nH]c(S(=O)(=O)N[C@@]23COc4cc(F)c(Cl)cc4[C@@H]2C3)cc1C(=O)N1CC(C#N)C1] | 81 |
| 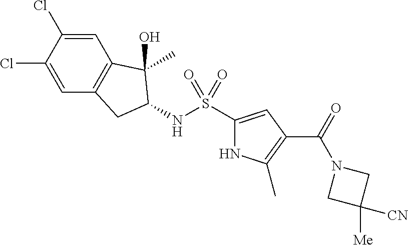 [SMILES: Cc1[nH]c(S(=O)(=O)N[C@@H]2Cc3cc(Cl)c(Cl)cc3[C@@]2(C)O)cc1C(=O)N1CC(C)(C#N)C1] | 82 |
| 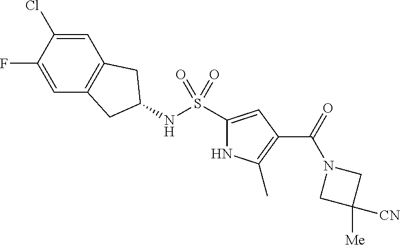 [SMILES: Cc1[nH]c(S(=O)(=O)N[C@@H]2Cc3cc(F)c(Cl)cc3C2)cc1C(=O)N1CC(C)(C#N)C1] | 83 |
| 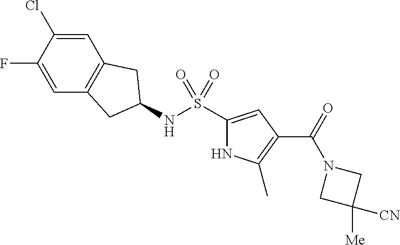 [SMILES: Cc1[nH]c(S(=O)(=O)N[C@H]2Cc3cc(F)c(Cl)cc3C2)cc1C(=O)N1CC(C)(C#N)C1] | 84 |
| 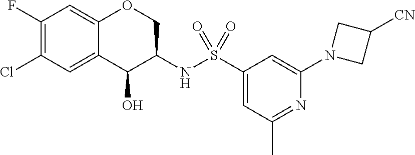 [SMILES: Cc1cc(S(=O)(=O)N[C@@H]2COc3cc(F)c(Cl)cc3[C@@H]2O)cc(N2CC(C#N)C2)n1] | 85 |
| 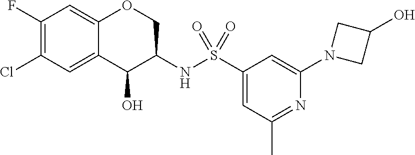 [SMILES: Cc1cc(S(=O)(=O)N[C@@H]2COc3cc(F)c(Cl)cc3[C@@H]2O)cc(N2CC(O)C2)n1] | 86 |
| 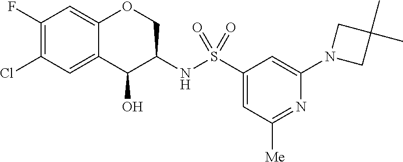 [SMILES: Cc1cc(S(=O)(=O)N[C@@H]2COc3cc(F)c(Cl)cc3[C@@H]2O)cc(N2CC(C)(C)C2)n1] | 87 |
| 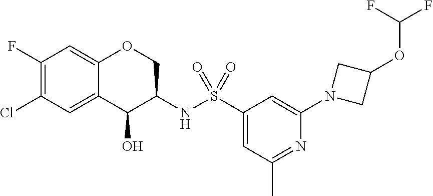 [SMILES: Cc1cc(S(=O)(=O)N[C@@H]2COc3cc(F)c(Cl)cc3[C@@H]2O)cc(N2CC(OC(F)F)C2)n1] | 88 |
| 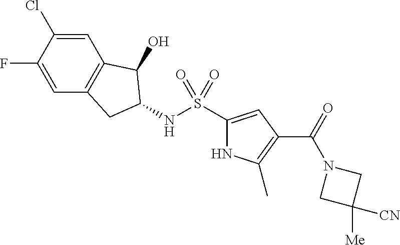 [SMILES: Cc1[nH]c(S(=O)(=O)N[C@@H]2Cc3cc(F)c(Cl)cc3[C@H]2O)cc1C(=O)N1CC(C)(C#N)C1] | 89 |
| 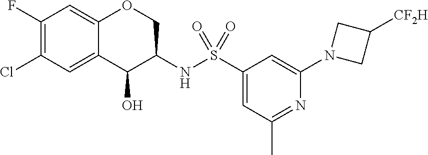 [SMILES: Cc1cc(S(=O)(=O)N[C@@H]2COc3cc(F)c(Cl)cc3[C@@H]2O)cc(N2CC(C(F)F)C2)n1] | 90 |
| 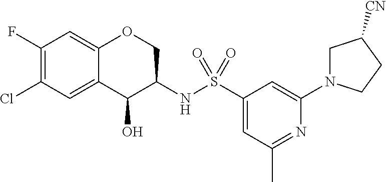 [SMILES: Cc1cc(S(=O)(=O)N[C@@H]2COc3cc(F)c(Cl)cc3[C@@H]2O)cc(N2CC[C@@H](C#N)C2)n1] | 91 |
| 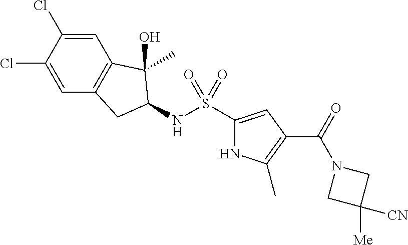 [SMILES: Cc1[nH]c(S(=O)(=O)N[C@H]2Cc3cc(Cl)c(Cl)cc3[C@@]2(C)O)cc1C(=O)N1CC(C)(C#N)C1] | 92 |
| 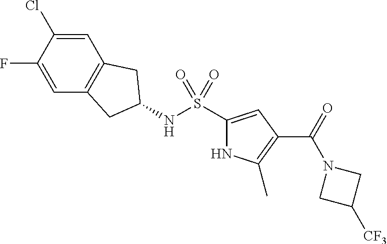 [SMILES: Cc1[nH]c(S(=O)(=O)N[C@@H]2Cc3cc(F)c(Cl)cc3C2)cc1C(=O)N1CC(C(F)(F)F)C1] | 93 |
| 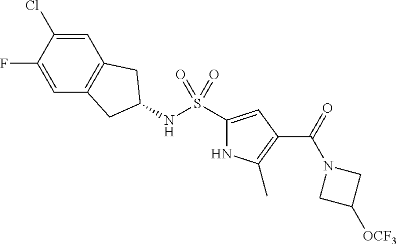 [SMILES: Cc1[nH]c(S(=O)(=O)N[C@@H]2Cc3cc(F)c(Cl)cc3C2)cc1C(=O)N1CC(OC(F)(F)F)C1] | 94 |
| 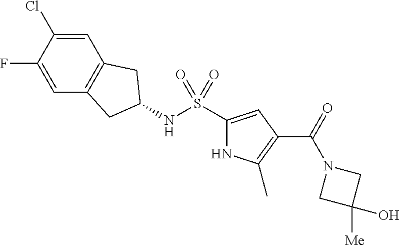 [SMILES: Cc1[nH]c(S(=O)(=O)N[C@@H]2Cc3cc(F)c(Cl)cc3C2)cc1C(=O)N1CC(C)(O)C1] | 95 |
| 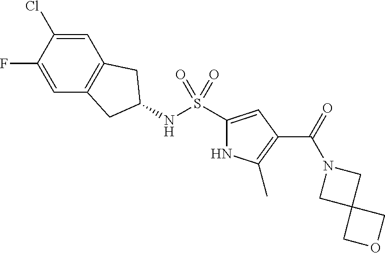 [SMILES: Cc1[nH]c(S(=O)(=O)N[C@@H]2Cc3cc(F)c(Cl)cc3C2)cc1C(=O)N1CC2(COC2)C1] | 96 |
| 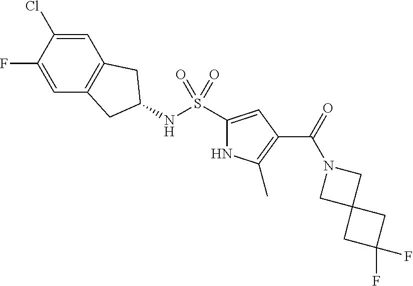 [SMILES: Cc1[nH]c(S(=O)(=O)N[C@@H]2Cc3cc(F)c(Cl)cc3C2)cc1C(=O)N1CC2(C1)CC(F)(F)C2] | 97 |
| 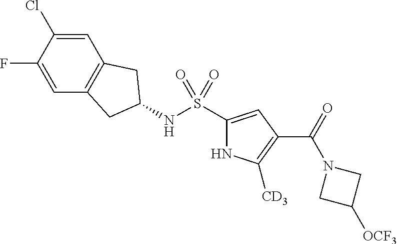 [SMILES: [2H]C([2H])([2H])c1[nH]c(S(=O)(=O)N[C@@H]2Cc3cc(F)c(Cl)cc3C2)cc1C(=O)N1CC(OC(F)(F)F)C1] | 98 |
| 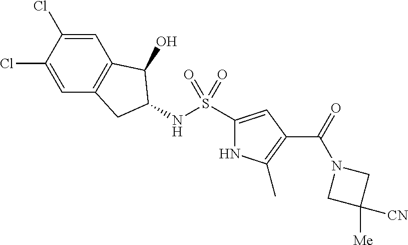 [SMILES: Cc1[nH]c(S(=O)(=O)N[C@@H]2Cc3cc(Cl)c(Cl)cc3[C@H]2O)cc1C(=O)N1CC(C)(C#N)C1] | 100 |
| 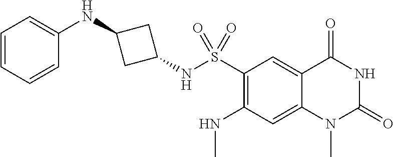 [SMILES: CNc1cc2c(cc1S(=O)(=O)N[C@H]1C[C@H](Nc3ccccc3)C1)c(=O)[nH]c(=O)n2C] | 101 |

### Compositions

Also provided are compositions, such as pharmaceutical compositions, that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, provided are compositions, such as pharmaceutical compositions that contain one or more compounds described herein, or a pharmaceutically acceptable salt thereof.

In some embodiments, provided is a pharmaceutically acceptable composition comprising a compound disclosed and/or described herein, or a pharmaceutically acceptable salt thereof. In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds or conjugates that are substantially pure.

Also provided are packaged pharmaceutical compositions, comprising a pharmaceutical composition as described herein and instructions for using the composition to treat a patient suffering from a disease or condition described herein.

### Methods of Use

The compounds disclosed herein in free form or in pharmaceutically acceptable salt form, exhibit valuable pharmacological properties, e.g. cardiac sarcomere modulating properties and more particularly cardiac sarcomere activating properties e.g. as indicated in in vitro and in vivo tests as provided in the next sections and are therefore indicated for therapy.

The present invention provides methods of treating a disease or disorder associated with heart muscle contractility by administering to a subject in need thereof an effective amount of a compound disclosed herein. In certain aspects, methods are provided for the treatment of diseases associated with increasing activity of the cardiac sarcomere.

In a specific embodiment, the present invention provides a method of treating or preventing heart failure by administering to a subject in need thereof an effective amount of a compound disclosed herein. In certain embodiments, patients who are currently asymptomatic but are at risk of developing heart failure are suitable for administration with a compound of the invention. The methods of treating or preventing heart failure include, but are not limited to, methods of treating or preventing systolic heart failure.

In some embodiments, the present invention provides methods of treating a disease or disorder associated with decreased ejection fraction from the heart, e.g., heart failure by administering to a subject in need thereof an effective amount of a compound disclosed herein. Examples of known heart failure patient populations associated with reduced or compromised ejection fraction include systolic heart failure.

In some embodiments, the compounds disclosed herein are used in the treatment or prevention of heart failure with reduced ejection fraction (HFrEF) or systolic heart failure, dilated cardiomyopathy, postpartum cardiomyopathy, idiopathic cardiomyopathy, pediatric HFrEF, chemotherapy-induced heart failure, heart failure associated with muscular dystrophy, bi-ventricular HFrEF, HFrEF with pulmonary hypertension, heart failure with preserved ejection fraction (HFpEF) with right ventricular dysfunction, pulmonary hypertension with right ventricular dysfunction, scleroderma with pulmonary hypertension, right ventricular dysfunction, Chagas disease, or myocarditis. In some embodiments, provided herein are methods of treating or preventing heart failure with reduced ejection fraction or systolic heart failure, dilated cardiomyopathy, postpartum cardiomyopathy, idiopathic cardiomyopathy, pediatric HFrEF, chemotherapy-induced heart failure, heart failure associated with muscular dystrophy, bi-ventricular HFrEF, HFrEF with pulmonary hypertension, heart failure with preserved ejection fraction (HFpEF) with right ventricular dysfunction, pulmonary hypertension with right ventricular dysfunction, scleroderma with pulmonary hypertension, right ventricular dysfunction, Chagas disease, or myocarditis, which methods comprise administering to a subject in need thereof an effective amount of one or more compounds disclosed herein. Also provided herein is the use of one or more compounds disclosed herein in the manufacture of a medicament for the treatment or prevention of heart failure with reduced ejection fraction or systolic heart failure, dilated cardiomyopathy, postpartum cardiomyopathy, idiopathic cardiomyopathy, pediatric HFrEF, chemotherapy-induced heart failure, heart failure associated with muscular dystrophy, bi-ventricular HFrEF, HFrEF with pulmonary hypertension, heart failure with preserved ejection fraction (HFpEF) with right ventricular dysfunction, pulmonary hypertension with right ventricular dysfunction, scleroderma with pulmonary hypertension, right ventricular dysfunction, Chagas disease, or myocarditis.

In some embodiments, the dilated cardiomyopathy is selected from the group consisting of genetic dilated cardiomyopathy, peripartum cardiomyopathy (e.g., post-partum cardiomyopathy), idiopathic dilated cardiomyopathy, post-infectious dilated cardiomyopathy, toxin-induced dilated cardiomyopathy, and nutritional deficiency dilated cardiomyopathy. In some embodiments, the pediatric HFrEF occurs in pediatric patients with univentricular hearts or a single ventricle or patients post Fontan or Fontan-Kreutzer procedure. In some embodiments, the pediatric HFrEF is pediatric heart failure associated with congenital heart disease. In some embodiments, the chemotherapy-induced heart failure is selected from the group consisting of chemotherapy-induced left ventricular dysfunction, radiation-induced heart failure, heart failure resulting from anthracycline treatment (including but not limited to doxorubicin, epirubicin, and daunorubicin), heart failure resulting from antiERBB2 treatment (including but not limited to trastuzumab and lapatinib), heart failure resulting from VEGF inhibitor treatment (including but not limited to bevacizumab), and heart failure resulting from tyrosine-kinase inhibitor treatment (including but not limited to imatinib, dasatinib, nilotinim, sorafenib, and sunitinib). In some embodiments, the heart failure associated with muscular dystrophy is selected from the group consisting of heart failure associated with Duchenne muscular dystrophy, heart failure associated with Becker muscular dystrophy, heart failure associated with myotonic dystrophy (e.g., Steinert's disease), heart failure associated with laminopathies such as Emery-Dreifuss muscular dystrophy (EDMD), including both X-linked EDMD and autosomal dominant EDMD, heart failure associated with facioscapulohumeral muscular dystrophy (FSHMD), heart failure associated with Limb-girdle muscular dystrophy, including sarcoglycanopathies and the autosomal dominant form of the disease, and heart failure associated with congenital muscular dystrophy. In some embodiments, the pulmonary hypertension with right ventricular dysfunction is associated with high left ventricular (diastolic) pressure in HFrEF or high left ventricular (diastolic) pressure in HFpEF.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

The activity of a compound according to the present invention can be assessed by in vitro & in vivo methods, such as those described in the examples below.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound disclosed herein and at least one other therapeutic agent as a combined preparation for simultaneous, separate or sequential use in therapy. In one embodiment, the therapy is the treatment of a disease or condition mediated by the cardiac sarcomere. In preferred aspects, the therapy is a treatment for heart failure having reduced or compromised ejection fraction. Products provided as a combined preparation include a composition comprising the compound disclosed herein and the other therapeutic agent(s) together in the same pharmaceutical composition, or the compound disclosed herein and the other therapeutic agent(s) in separate form, e.g. in the form of a kit.

In one embodiment, the invention provides a pharmaceutical composition comprising a compound as disclosed herein and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound disclosed herein. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. The compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound as disclosed herein for treating a disease or condition mediated by the cardiac sarcomere wherein the medicament is prepared for administration with another therapeutic agent. The invention also provides the use of another therapeutic agent for treating a disease or condition mediated by the cardiac sarcomere, wherein the medicament is administered with a compound as disclosed herein. In another aspect, the invention provides the use of a compound as disclosed herein for treating a heart failure having reduced or compromised ejection fraction wherein the medicament is prepared for administration with another therapeutic agent. The invention also provides the use of another therapeutic agent for treating heart failure having reduced or compromised ejection fraction, wherein the medicament is administered with a compound as disclosed herein.

The invention also provides a compound as disclosed herein for use in a method of treating a disease or condition mediated by the cardiac sarcomere or in the treating of heart failure having reduced or compromised ejection fraction, wherein the compound is prepared for administration with another therapeutic agent. The invention also provides another therapeutic agent for use in a method of treating a disease or condition mediated by the cardiac sarcomere or in the treating of heart failure having reduced or compromised ejection fraction, wherein the other therapeutic agent is prepared for administration with a compound as disclosed herein. The invention also provides a compound as disclosed herein for use in a method of treating a disease or condition mediated by the cardiac sarcomere or in the treating of heart failure having reduced or compromised ejection fraction, wherein the compound is administered with another therapeutic agent. The invention also provides another therapeutic agent for use in a method of treating a disease or condition mediated by the cardiac sarcomere or in the treating of heart failure having reduced or compromised ejection fraction, wherein the other therapeutic agent is administered with a compound as disclosed herein.

The invention also provides the use of a compound as disclosed herein for treating a disease or condition mediated by the cardiac sarcomere or in the treating of heart failure having reduced or compromised ejection fraction wherein the patient has previously (e.g. within 24 hours) been treated with another therapeutic agent. The invention also provides the use of another therapeutic agent for treating a disease or condition mediated by the cardiac sarcomere or in the treating of heart failure having reduced or compromised ejection fraction wherein the patient has previously (e.g. within 24 hours) been treated with a compound as disclosed herein.

The pharmaceutical compositions can be administered alone or in combination with other molecules known to have a beneficial effect on heart failure including molecules capable of increasing the contractility of the heart and/or increasing the ejection fraction in patients suffering from or susceptible to heart failure.

A combination therapy regimen may be additive, or it may produce synergistic results (e.g., increases in cardiac contractility or increased cardiac ejection fraction which is more than expected for the combined use of the two agents). In some embodiments, the present invention provide a combination therapy for preventing and/or treating heart failure or more particularly systolic heart failure disease as described above with a compound of the invention and a second therapeutic agent. Suitable additional active agents include, for example: therapies that retard the progression of heart failure by down-regulating neurohormonal stimulation of the heart and attempt to prevent cardiac remodeling (e.g., ACE inhibitors or β-blockers); therapies that improve cardiac function by stimulating cardiac contractility (e.g., positive inotropic agents, such as the β-adrenergic agonist dobutamine or the phosphodiesterase inhibitor milrinone); therapies that reduce cardiac preload (e.g., diuretics, such as furosemide), agents that reduce afterload such as nephrilysin inhibitors/angiotensin receptor blockers, as well as drugs that slow heart rate, such as ivabradine; angiotensin receptor blockers (e.g., without nephrilysin inhibitors); aldosterone antagonists (e.g. spironolactone, eplerenone); hydralizine-nitrates; and digoxin. Suitable additional active agents also include, for example, agents that improve mitochondrial function.

In one embodiment, the invention provides a method of modulating activity of the cardiac sarcomere in a subject, wherein the method comprises administering to the subject a therapeutically effective amount of the compound according to the definition of Formula (I). The invention further provides methods of modulating the activity of the cardiac sarcomere in a subject by administering a compound as disclosed herein which bind to the Troponin C/Troponin I interface to increase activity of the cardiac sarcomere, wherein the method comprises administering to the subject a therapeutically effective amount of the compound as disclosed herein.

In one embodiment, the invention provides a compound as disclosed herein, for use as a medicament.

In one embodiment, the invention provides the use of a compound as disclosed herein for the treatment of a disorder or disease in a subject characterized by reduced cardiac function. In particular, the invention provides the use of a compound as disclosed herein for the treatment of a disorder or disease mediated by reduced cardiac sarcomere function, e.g., heart failure or more particularly systolic heart failure.

In one embodiment, the invention provides the use of a compound as disclosed herein in the manufacture of a medicament for the treatment of a disorder or disease in a subject characterized by reduced cardiac function. More particularly in the manufacture of a medicament for the treatment of a disease or disorder in a subject characterized by reduced cardiac sarcomere function, e.g., heart failure or more particularly systolic heart failure.

In one embodiment, the invention provides the use of a compound as disclosed herein for the treatment of a disorder or disease in a subject characterized by reduced cardiac function. More particularly, the invention provides uses of the compounds provided herein in the treatment of a disease or disorder characterized by reduced cardiac sarcomere function, e.g., heart failure or more particularly systolic heart failure. In certain embodiments, the use is in the treatment of a disease or disorder is selected from heart failure or systolic heart failure.

In a specific embodiment, the present invention provides use of the compounds of the invention for treating or preventing heart failure or systolic heart failure. In certain embodiments, patients who are currently asymptomatic but are at risk of developing a symptomatic heart failure or systolic heart failure are suitable for administration with a compound of the invention. The use in treating or preventing heart failure or systolic heart failure include, but are not limited to, uses in treating or preventing one or more symptoms or aspects of heart failure selected from reduced heart contractility and reduced ejection fraction.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure materials.

### SYNTHETIC EXAMPLES

### Example I-1. Intermediate Synthesis 1

### Preparation of 6-((trans)-3,5-dimethylpiperidin-1-yl)pyridin-3-amine (Intermediate 1.1)

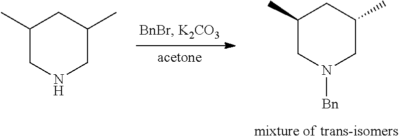

### Step 1: trans-3,5-dimethylpiperidine

BnBr (1.25 kg, 7.4 mol, 1.8 equiv.) was added dropwise to a stirring solution of 3,5-dimethylpiperidine (459 g, 4.1 mmol, 1 equiv.) and K₂CO₃ (1.69 kg, 12 mol, 3 equiv.) in acetone (2.5 L) at a rate capable of keeping the internal temperature below 40° C. After 1 h, the reaction began to solidify and was diluted with acetone (500 mL). After 1 h, the reaction was filtered, the cake washed with acetone and EtOAc, and the mother liquor concentrated by rotary evaporation. The mother liquor was then diluted with 50% EtOAc/hex, filtered, and the mother liquor concentrated by rotary evaporation. The residue was resolved by silica chromatography (0-5% Et₂O/hexanes) to give the desired mixture of trans-products (80 g, 10%). ¹H NMR (400 MHz, Chloroform-d) δ 7.34-7.15 (m, 5H), 3.54-3.28 (m, 2H), 2.37 (d, J=9.1 Hz, 2H), 2.13-1.97 (m, 2H), 1.90 (ddp, J=10.0, 6.3, 3.6 Hz, 2H), 1.28 (t, J=5.8 Hz, 2H), 0.95 (d, J=6.8 Hz, 6H). LC/MS (APCI) m/z calcd. for C₁₄H₂₂N⁺ [M+H]⁺: 204.1; 204.1 found.

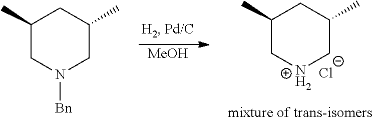

### Step 2: trans-3,5-dimethylpiperidin-1-ium chloride

trans-3,5-Dimethylpiperidine (80 g, 393 mmol, 1 equiv.) and Pd/C (4 g, 7.5 mmol, 0.02 equiv., 20% Pd by mass) were suspended in MeOH (300 mL) before being stirred under H₂ (60 psi) at 40° C. for 2 days. Additional Pd/C (6 g, 11 mmol, 0.03 equiv., 20% Pd by mass) was added and the reaction continued to stir at under H₂ (60 psi) at 40° C. for 2 days. The reaction was then cooled to rt, filtered, acidified with 4M HCl in dioxanes (200 mL), and solvent removed by rotary evaporation to give the product as a clear semi-solid (59 g, 99%). ¹H NMR (400 MHz, Methanol-d₄) δ 3.14 (dd, J=12.6, 4.0 Hz, 2H), 2.83 (dd, J=12.5, 7.0 Hz, 2H), 2.20-2.06 (m, 2H), 1.55 (t, J=5.8 Hz, 2H), 1.07 (d, J=7.1 Hz, 6H).

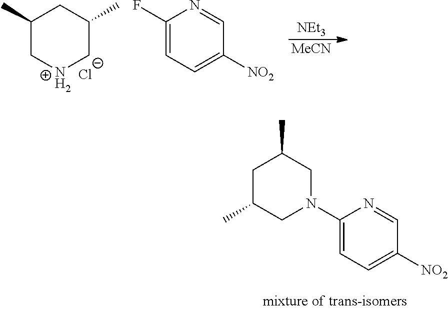

### Step 3: 2-(trans-3,5-dimethylpiperidin-1-yl)-5-nitropyridine

(3S,5S)-3,5-dimethylpiperidin-1-ium chloride (59 g, 394 mmol, 1.1 equiv.) 2-fluoro-5-nitropyridine (52 g, 366 mmol, 1 equiv.), and NEt₃ (100 mL, 740 mmol, 2.1 were suspended in MeCN (500 mL) before being heated to 70° C. for 1 day. The reaction was then stirred at rt for 3 days before being filtered, the cake washed with MeCN, and the mother liquor concentrated by rotary evaporation. The residue was suspended in water, extracted with EtOAc, the organic layer washed with brine, dried over sodium sulfate, filtered, and solvent removed by rotary evaporation. The resulting solids were dissolved in minimum EtOAc, 20% EtOAc/hexanes added (50 mL), followed by slow addition of hexanes until precipitation was observed, the reaction suspension was then stirred at rt for 14 h. The product was then filtered and washed with 20% EtOAc/hexanes before being dried under high vacuum to give the desired product as a yellow tinged solid (55 g, 71%). ¹H NMR (400 MHz, Chloroform-d) δ 9.01 (d, J=2.8 Hz, 1H), 8.14 (ddd, J=9.6, 2.9, 0.6 Hz, 1H), 6.54 (d, J=9.6 Hz, 1H), 3.82 (d, J=12.3 Hz, 2H), 3.38 (dd, J=13.2, 7.1 Hz, 2H), 2.03 (ddp, J=10.4, 6.4, 4.0 Hz, 2H), 1.54 (t, J=5.9 Hz, 2H), 0.96 (d, J=6.8 Hz, 6H). LC/MS (APCI) m/z calcd. for C₁₂H₁₈N₃O₂+ [M+H]+: 236.1; 236.1 found.

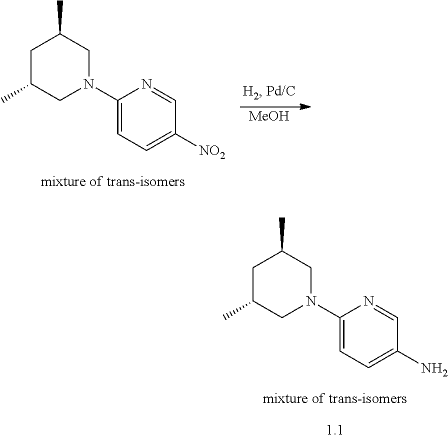 [SMILES: "MeOH" ; C[C@@H]1C[C@@H](C)CN(c2ccc([N+](=O)[O-])cn2)C1 ; C[C@@H]1C[C@@H](C)CN(c2ccc(N)cn2)C1]

### Step 3: 6-(trans-3,5-Dimethylpiperidin-1-yl)pyridin-3-amine

2-(trans-3,5-Dimethylpiperidin-1-yl)-5-nitropyridine (4.5 g, 19.1 mmol, 1 equiv.) and Pd/C (70 mg, 0.13 mmol, 0.007 equiv.) were suspended in EtOH (65 mL) and CH₂Cl₂ (10 mL) before being stirred under H₂ for 1 h. The reaction was then filtered and solvent removed by rotary evaporation to give the product (Intermediate 1.1) as a yellow solid. LC/MS (APCI) m/z calcd. for C₁₂H₁₉N₃⁺ [M+H]⁺: 206.2; 206.1 found.

### Preparation of 6-((3R,5R)-3,5-dimethylpiperidin-1-yl)pyridin-3-amine (Intermediate 1.2)

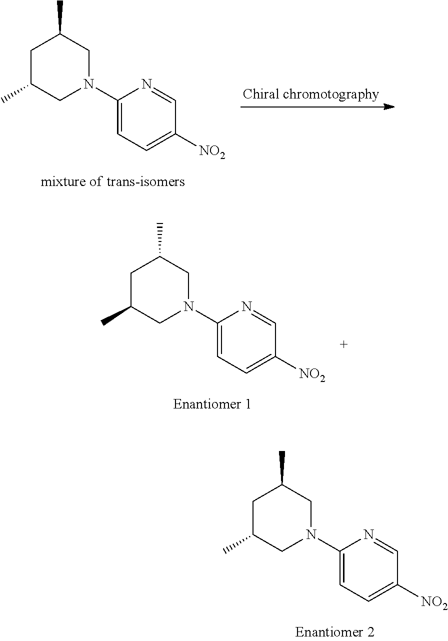 [SMILES: C[C@@H]1C[C@@H](C)CN(c2ccc([N+](=O)[O-])cn2)C1 ; C[C@H]1C[C@H](C)CN(c2ccc([N+](=O)[O-])cn2)C1 ; C[C@@H]1C[C@@H](C)CN(c2ccc([N+](=O)[O-])cn2)C1]

### Step 1: 2-((3R,5R)-3,5-dimethylpiperidin-1-yl)-5-nitropyridine

2-(trans-3,5-dimethylpiperidin-1-yl)-5-nitropyridine (˜1 g) was resolved into its respective enantiomers by chiral SFC (Chiralcel AD-H, 20% (1:1) isopropanol:MeCN/CO₂, 100 bar, 62 mL/min) to give enantiomer 1 (525 mg, [α]20/D=+41.4° [c 0.95, EtOAc) and enantiomer 2 (520 mg, [α]20/D=−45.0 (c 0.91, EtOAc). Enantiomers were numbered based on order of elution from stated conditions: Enantiomer 1-2-((3S,5S)-3,5-dimethylpiperidin-1-yl)-5-nitropyridine eluted first, Enantiomer 2-2-((3R,5R)-3,5-dimethylpiperidin-1-yl)-5-nitropyridine eluted second. Absolute stereochemistry was confirmed later through x-ray crystallography.

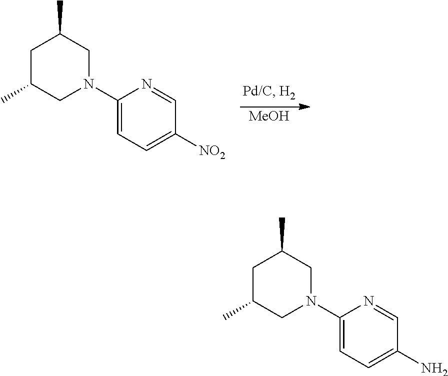 [SMILES: "MeOH" ; C[C@@H]1C[C@@H](C)CN(c2ccc([N+](=O)[O-])cn2)C1 ; C[C@@H]1C[C@@H](C)CN(c2ccc(N)cn2)C1]

### Step 2: 6-((3R,5R)-3,5-dimethylpiperidin-1-yl)pyridin-3-amine

2-((3R,5R)-3,5-dimethylpiperidin-1-yl)-5-nitropyridine (Enantiomer 2 from previous step, 350 mg, 1.49 mmol, 1 equiv.) and Pd/C (80 mg, 0.075 mmol, 0.05 equiv.) were suspended in MeOH (35 mL) before being stirred under H₂ (30 psi) for 1 h. The reaction was then filtered through a pad of celite and solvent removed by rotary evaporation to give the desired product (Intermediate 1.2). LC/MS (APCI) m/z calcd. for C₁₂H₁₉N₃⁺ [M+H]⁺: 206.2; 206.1.

### Example I-2. Intermediate Synthesis 2

### Preparation of (trans)-N¹-(3-fluorophenyl)cyclobutane-1,3-diamine hydrochloride (Intermediate 2.1)

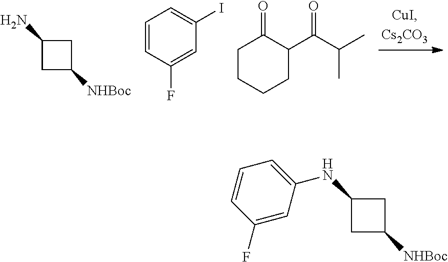 [SMILES: CC(C)(C)OC(=O)N[C@H]1C[C@@H](N)C1 ; Fc1cccc(I)c1 ; CC(C)C(=O)C1CCCCC1=O ; CC(C)(C)OC(=O)N[C@H]1C[C@@H](Nc2cccc(F)c2)C1]

### Step 1. tert-butyl (cis)-3-(3-fluorophenoxy)cyclobutyl)carbamate

tert-Butyl (cis)-3-aminocyclobutyl)carbamate (400 mg, 2.15, 1 equiv.), 3-fluoroiodobenzene (524 mg, 2.36 mmol, 1.1 equiv.), Cs₂CO₃ (1.4 g, 4.30 mmol, 2 equiv.), CuI (20 gm, 0.107 mmol, 0.05 equiv.), and 2-isobutyrylcyclohexanone (72 mg, 0.43 mmol, 0.2 equiv.) were suspended in DMF (3 mL) at rt. After 13 h, the reaction was diluted with water, extracted with EtOAc, the organic layer washed with brine, dried over sodium sulfate, filtered, and solvent removed by rotary evaporation. The product was isolated by silica chromatography (10->20% EtOAc/hexanes) as a colorless oil (300 mg, 50%). ¹H NMR (400 MHz, Chloroform-d) δ 7.10-6.90 (m, 1H), 6.33 (td, J=8.5, 2.4 Hz, 1H), 6.24 (dd, J=8.2, 2.2 Hz, 1H), 6.16 (dt, J=11.4, 2.4 Hz, 1H), 4.64-4.56 (m, 1H), 3.96-3.80 (m, 1H), 3.70 (dt, J=9.3, 4.8 Hz, 2H), 2.91-2.75 (m, 2H), 2.75-2.56 (m, 2H), 1.37 (s, 9H). LC/MS (APCI) m/z calcd. for C₁₅H₂₂FN₂O₂⁺ [M+H]⁺: 281.2; 281.1 found.

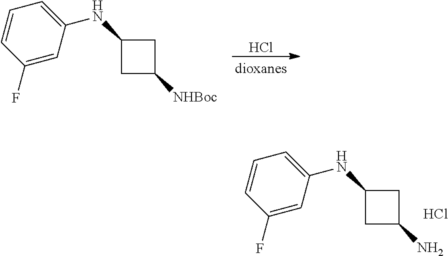 [SMILES: CC(C)(C)OC(=O)N[C@H]1C[C@@H](Nc2cccc(F)c2)C1 ; Cl ; N[C@H]1C[C@@H](Nc2cccc(F)c2)C1 ; Cl]

### Step 2: (cis)-N¹-(3-fluorophenyl)cyclobutane-1,3-diamine hydrochloride

tert-Butyl ((1s,3s)-3-(3-fluorophenoxy)cyclobutyl)carbamate (300 mg, 1.07 mmol, 1 equiv.) was suspended in EtOAc and 6M HCl. After 5 h, the reaction was concentrated by rotary evaporation and dried under high vacuum to give the desired product (Intermediate 2.1). LC/MS (APCI) m/z calcd. for C₁₀H₁₄FN₂⁺ [M+H]⁺: 181.1; 181.1 found.

TABLE I-2.1
The following compounds and intermediates were prepared according to synthetic
procedures as described in Intermediate Synthesis 2, step 1.

| Intermediate | Ar-I | Amine | Structure, Name and Data |
|---|---|---|---|
| 2.1 | 3- fluoroiodobenzene | tert-Butyl (cis)-3- aminocyclobutyl) carbamate | 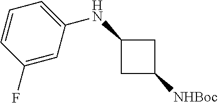 [SMILES: CC(C)(C)OC(=O)N[C@H]1C[C@@H](Nc2cccc(F)c2)C1] tert-butyl ((cis)-3-(3- fluorophenoxy) cyclobutyl)carbamate. ¹H NMR (400 MHz, Chloroform-d) δ 7.10- 6.90 (m, 1H), 6.33 (td, J = 8.5, 2.4 Hz, 1H), 6.24 (dd, J = 8.2, 2.2 Hz, 1H), 6.16 (dt, J = 11.4, 2.4 Hz, 1H), 4.64-4.56 (m, 1H), 3.96-3.80 (m, 1H), 3.70 (dt, J = 9.3, 4.8 Hz, 2H), 2.91-2.75 (m, 2H), 2.75- 2.56 (m, 2H), 1.37 (s, 9H). LC/MS (APCI) m/z calcd. for C₁₅H₂₂FN₂O₂⁺ [M + H]⁺: 281.2; 281.1 found. |
| 2.2 | iodobenzene | tert-Butyl (cis)-3- aminocyclobutyl) carbamate | 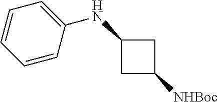 [SMILES: CC(C)(C)OC(=O)N[C@H]1C[C@@H](Nc2ccccc2)C1] tert-butyl ((cis)-3- (phenylamino)cyclobutyl) carbamate. LC/MS (APCI) m/z calcd. for C₁₅H₂₃N₂O₂⁺ [M + H]⁺: 263.2; 263.2 found. |
| 2.3 | 2- fluoroiodobenzene | tert-Butyl (cis)-3- aminocyclobutyl) carbamate | 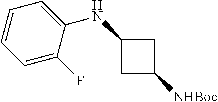 [SMILES: CC(C)(C)OC(=O)N[C@H]1C[C@@H](Nc2ccccc2F)C1] tert-butyl ((cis)-3-((2- fluorophenyl)amino)cyclobutyl) carbamate. ¹H NMR (400 MHz, DMSO-d₆) δ 7.10 (d, J = 8.0 Hz, 1H), 7.05-6.97 (m, 1H), 6.97- 6.92 (m, 1H), 6.65-6.58 (m, 1H), 6.58-6.49 (m, 1H), 5.55-5.41 (m, 1H), 3.71 (q, J = 8.2 Hz, 1H), 3.48 (h, J = 7.4 Hz, 1H), 2.73- 2.57 (m, 2H), 1.81 (qd, J = 8.8, 2.9 Hz, 2H), 1.38 (s, 9H). LC/MS (APCI) m/z calcd. for C₁₅H₂₂FN₂O₂⁺ [M + H]⁺: 281.2; 281.3 found. |
| 2.4 | 4- fluoroiodobenzene | tert-Butyl (cis)-3- aminocyclobutyl) carbamate | 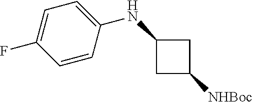 [SMILES: CC(C)(C)OC(=O)N[C@H]1C[C@@H](Nc2ccc(F)cc2)C1] tert-butyl ((cis)-3-((4- fluorophenyl)amino)cyclobutyl) carbamate. LC/MS (APCI) m/z calcd. for C₁₅H₂₂FN₂O₂⁺ [M + H]⁺: 281.2; 281.1 found. |
| 2.5 | Iodobenzene | tert-Butyl (cis)-3- aminocyclopentyl) carbamate | 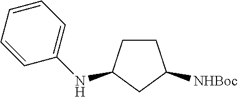 [SMILES: CC(C)(C)OC(=O)N[C@@H]1CC[C@H](Nc2ccccc2)C1] and |
| | | | 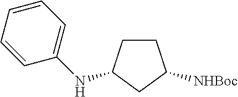 [SMILES: CC(C)(C)OC(=O)N[C@H]1CC[C@@H](Nc2ccccc2)C1] tert-butyl ((cis)-3- (phenylamino)cyclopentyl) carbamate. ¹H NMR (400 MHz, DMSO-d₆) δ 7.12-7.00 (m, 2H), 6.89 (d, J = 7.5 Hz, 1H), 6.59- 6.46 (m, 3H), 5.55 (d, J = 6.9 Hz, 1H), 3.79 (q, J = 7.3 Hz, 1H), 3.72- 3.55 (m, 1H), 2.31 (dq, J = 14.2, 7.4, 6.7 Hz, 2H), 1.93-1.75 (m, 2H), 1.51 (ddd, J = 10.7, 8.0, 5.8 Hz, 2H), 1.39 (s, 9H). LC/MS (APCI) m/z calcd. for C₁₆H₂₅N₂O₂⁺ [M + H]⁺: 277.2; 277.3 found. |

TABLE I-2.2
The following intermediates were prepared according to synthetic
procedures as described in Intermediate Synthesis 2, step 2.

| Inter- | Starting | |
|---|---|---|
| mediate | Intermediate | Structure, Name and Data |
| 2.6 | 2.1 | 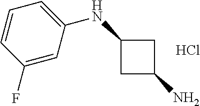 [SMILES: N[C@H]1C[C@@H](Nc2cccc(F)c2)C1 ; Cl] (cis)-N¹-(3-fluorophenyl)cyclobutane- 1,3-diaminehydrochloride. LC/MS (APCI) m/z calcd. for C₁₀H₁₄FN₂⁺ [M + H]⁺: 181.1; 181.1 found. |
| 2.7 | 2.2 | 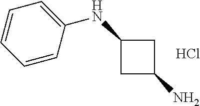 [SMILES: N[C@H]1C[C@@H](Nc2ccccc2)C1 ; Cl] (cis)-N¹-phenylcyclobutane-1,3- diamine hydrochloride. LC/MS (APCI) m/z calcd. for C₁₀H₁₅N₂⁺ [M + H]⁺: 163.1; 163.2 found. |
| 2.8 | 2.3 | 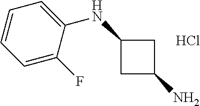 [SMILES: N[C@H]1C[C@@H](Nc2ccccc2F)C1 ; Cl] (cis)-N¹-(2-Fluorophenyl) cyclobutane-1,3-diamine hydrochloride. LC/MS (APCI) m/z calcd. for C₁₀H₁₄FN₂⁺ [M + H]⁺: 181.1; 181.2 found. |
| 2.9 | 2.4 | 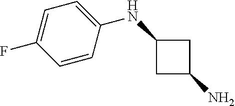 (cis)-N¹-(4-fluorophenyl) cyclobutane-1,3-diamine. LC/MS (APCI) m/z calcd. for C₁₅H₂₂FN₂O₂⁺ [M + H]⁺: 181.2; 181.1 found. |
| 2.10 | 2.5 | 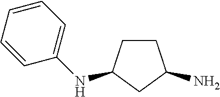 [SMILES: N[C@@H]1CC[C@H](Nc2ccccc2)C1] and |
| | | 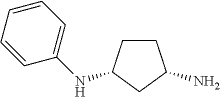 [SMILES: N[C@H]1CC[C@@H](Nc2ccccc2)C1] (cis)-N¹-phenylcyclopentane- 1,3-diamine. LC/MS (APCI) m/z calcd. for C₁₁H₁₇N₂⁺ [M + H]⁺: 177.1; 177.2 found. |

### Example 1-3. Intermediate Synthesis 3

### Preparation of (trans)-3-(3-methyl-4-(trifluoromethyl)-1H-pyrazol-1-yl)cyclobutan-1-amine (Intermediate 3.3)

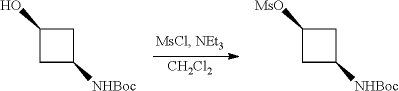 [SMILES: CC(C)(C)OC(=O)N[C@H]1C[C@@H](O)C1 ; "CH2Cl2" ; CC(C)(C)OC(=O)N[C@H]1C[C@@H](OS(C)(=O)=O)C1]

### Step 1: (cis)-3-((tert-butoxycarbonyl)amino)cyclobutyl methanesulfonate

Methanesulfonyl chloride (13.6 mL, 176 mmol, 1.1 equiv.) was added to a stirring solution of tert-butyl ((cis)-3-hydroxycyclobutyl)carbamate (30 g, 160 mmol, 1 equiv.) and NEt₃ (45 mL, 320 mmol, 2 equiv.) in CH₂Cl₂ (200 mL) at 0° C. The reaction was returned to rt over 14 h before being diluted with a saturated sodium bicarbonate solution, extracted with EtOAc, the organic layer washed with brine, dried over sodium sulfate, filtered, and solvent removed by rotary evaporation. The product was isolated by silica chromatography (0-10% EtOAc/CH₂Cl₂) as a white solid. ¹H NMR (400 MHz, DMSO-d₆) δ 7.24 (d, J=8.0 Hz, 1H), 4.68 (p, J=7.3 Hz, 1H), 3.62 (h, J=7.9 Hz, 1H), 3.14 (s, 3H), 2.67 (dh, J=12.1, 3.2 Hz, 2H), 2.13 (qd, J=8.9, 3.1 Hz, 2H), 1.37 (s, 9H). LC/MS (APCI) m/z calcd. for C₁₀H₂₀NO₅S⁺ [M+H]⁺: 266.1; 266.1 found.

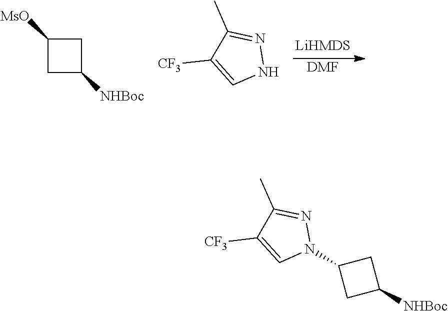

### Step 2: tert-Butyl ((trans)-3-(3-methyl-4-(trifluoromethyl)-1H-pyrazol-1-yl)cyclobutyl)carbamate (Intermediate 3.1)

LiHMDS (90 mL, 2M in THF, 0.18 mmol, 1.2 equiv.) was added to a stirring solution of (cis)-3-((tert-butoxycarbonyl)amino)cyclobutyl methanesulfonate (39.8 g, 150 mmol, 1 equiv.) and 3-methyl-4-(trifluoromethyl)-1H-pyrazole (29.3 g, 195 mmol, 1.3 equiv.) in DMF (100 mL) at rt before being heated to 70° C. for 14 h. The reaction was then cooled to rt, quenched with water, extracted with EtOAc, the organic layer washed with brine, dried over sodium sulfate, filtered, and solvent removed by rotary evaporation. The product was then obtained by silica chromatography (4% THF/CH₂Cl₂) as a 9:1 ratio of diastereomers (18 g, 38%). ¹H NMR (400 MHz, DMSO-d₆) δ 8.32 (s, 1H), 7.29 (dd, J=51.7, 7.9 Hz, 1H), 4.66 (p, J=7.4 Hz, 1H), 3.70-3.53 (m, 1H), 3.22 (d, J=76.3 Hz, 3H), 2.71-2.55 (m, 2H), 2.40 (ddd, J=13.2, 8.4, 5.5 Hz, 1H), 2.17-2.02 (m, 1H), 1.37 (d, J=6.9 Hz, 9H). LC/MS (APCI) m/z calcd. for C₁₄H₂₁F₃N₃O₂⁺ [M+H]⁺: 320.2; 320.2 found.

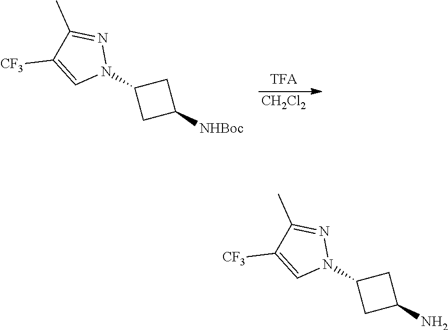 [SMILES: Cc1nn([C@H]2C[C@H](NC(=O)OC(C)(C)C)C2)cc1C(F)(F)F ; "CH2Cl2" ; Cc1nn([C@H]2C[C@H](N)C2)cc1C(F)(F)F]

### Step 3: (trans)-3-(3-methyl-4-(trifluoromethyl)-1H-pyrazol-1-yl)cyclobutan-1-amine (Intermediate 3.1)

TFA (80 mL) was added to a stirring solution of tert-butyl ((1r,3r)-3-(3-methyl-4-(trifluoromethyl)-1H-pyrazol-1-yl)cyclobutyl)carbamate (20 g, 62.6 mmol, 1 equiv.) in CH₂Cl₂ (100 mL) at rt. After 30 min, solvent was removed by rotary evaporation and dried under high vacuum to give the product which use used in subsequent steps without further processing. LC/MS (APCI) m/z calcd. for C₉H₁₃F₃N₃⁺ [M+H]⁺: 220.1; 220.1 found.

TABLE I-3.1
The following intermediates were prepared according to synthetic
procedures as described in Intermediate Synthesis 3, step 2.

| | Starting | | |
|---|---|---|---|
| Intermediate | cyclobutane | Heterocycle | Structure, Name and Data |
| 3.1 | tert-butyl ((cis)-3- hydroxycyclobutyl) carbamate | 3-methyl-4- (trifluoromethyl)- 1H-pyrazole | 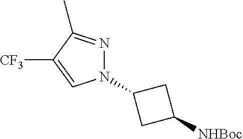 [SMILES: Cc1nn([C@H]2C[C@H](NC(=O)OC(C)(C)C)C2)cc1C(F)(F)F] tert-Butyl ((trans)-3-(3-methyl-4- (trifluoromethyl)-1H-pyrazol-1- yl)cyclobutyl)carbamate. ¹H NMR (400 MHz, DMSO-d₆) δ 8.32 (s, 1H), 7.29 (dd, J = 51.7, 7.9 Hz, 1H), 4.66 (p, J = 7.4 Hz, 1H), 3.70- 3.53 (m, 1H), 3.22 (d, J = 76.3 Hz, 3H), 2.71- 2.55 (m, 2H), 2.40 (ddd, J = 13.2, 8.4, 5.5 Hz, 1H), 2.17-2.02 (m, 1H), 1.37 (d, J = 6.9 Hz, 9H). LC/MS (APCI) m/z calcd. for C₁₄H₂₁F₃N₃O₂⁺ [M + H]⁺: 320.2; 320.2 found. |
| 3.2 | tert-butyl ((cis)-3- hydroxycyclobutyl) carbamate | 4- (trifluoromethyl)- 1H-pyrazole | 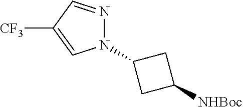 [SMILES: CC(C)(C)OC(=O)N[C@H]1C[C@H](n2cc(C(F)(F)F)cn2)C1] tert-butyl ((trans)-3-(4-(trifluoromethyl)- 1H-pyrazol-1-yl)cyclobutyl)carbamate. ¹H NMR (400 MHz, DMSO-d₆) δ 8.48 (d, J = 0.9 Hz, 1H), 7.92 (s, 1H), 7.37 (d, J = 7.3 Hz, 1H), 4.94 (ddd, J = 13.9, 8.5, 5.3 Hz, 1H), 4.19 (d, J = 7.6 Hz, 1H), 2.64 (dtd, J = 13.4, 5.2, 2.8 Hz, 2H), 2.43 (ddd, J = 13.3, 5.8, 2.8 Hz, 2H), 1.38 (s, 9H). LC/MS (APCI) m/z calcd. for C₁₃H₁₉F₃N₃O₂⁺ [M + H]⁺: 306.2; 206.1 found (M + H − Boc). |

TABLE I-3.2
The following intermediates were prepared according to synthetic
procedures as described in Intermediate Synthesis 3, step 3.

| | Starting | |
|---|---|---|
| Intermediate | Intermediate | Structure, Name and Data |
| 3.3 | 3.1 | 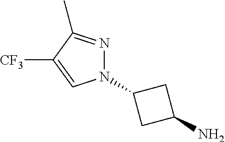 (trans)-3-(3-methyl-4-(trifluoromethyl)-1H-pyrazol-1- yl)cyclobutan-1-amine. LC/MS (APCI) m/z calcd. for C₉H₁₃F₃N₃⁺ [M + H]⁺: 220.1; 220.1 found. |
| 3.4 | 3.2 | 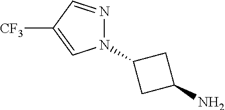 [SMILES: N[C@H]1C[C@H](n2cc(C(F)(F)F)cn2)C1] (trans)-3-(4-(Trifluoromethyl)-1H-pyrazol-1-yl)cyclobutan- 1-amine. LC/MS (APCI) m/z calcd. for C₈H₁₁F₃N₃⁺ [M + H]⁺: 206.1; 206.1 found. |

### Example I-4. Intermediate Synthesis 4

### Preparation of (trans)-3-(4-(trifluoromethyl)pyridin-2-yl)cyclobutan-1-amine (Intermediate 4)

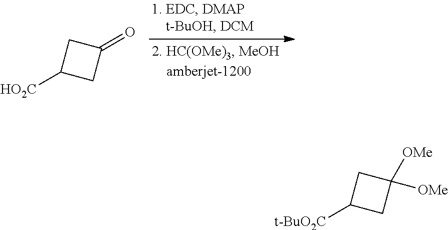

### Step 1: tert-Butyl 3,3-dimethoxycyclobutane-1-carboxylate

To a solution containing carboxylic acid (100 g, 876 mmol), t-BuOH (164 mL, 1.75 mol), and DMAP (107 g, 0.88 equiv.) in DCM (876 mL, 1.0 M) was added EDCI (185 g, 964 mmol). The reaction was stirred for 12 hours at rt. The reaction was quenched with saturated aqueous NH₄Cl solution and extracted with EtOAc (3×200 mL). The combined organic extracts were washed with 1N aqueous citric acid solution, saturated aqueous NaHCO₃ solution, and brine. The organic phase was dried over Na₂SO₄, filtered, and concentrated in vacuo to afford the intermediate ester (109 g) as colorless oil which was used in the step without further purification.

The ester (100 g, 588 mmol) obtained in the previous step was dissolved in MeOH (336 mL, 1.75 M) at rt. Trimethylorthoformate (149 g, 1.41 mol) and AMBERJET-1200H (10 g, 10% w/w) were added and the reaction stirred at 65° C. for 3 hours. The solution was cooled to rt and filtered over a pad of celite. The resulting filtrate was concentrated to afford the ketal as a colorless oil. ¹H NMR (400 MHz, Chloroform-d) δ 3.16 (s, 3H), 3.14 (s, 3H), 2.83-2.69 (m, 1H), 2.44-2.26 (m, 4H), 1.44 (s, 9H).

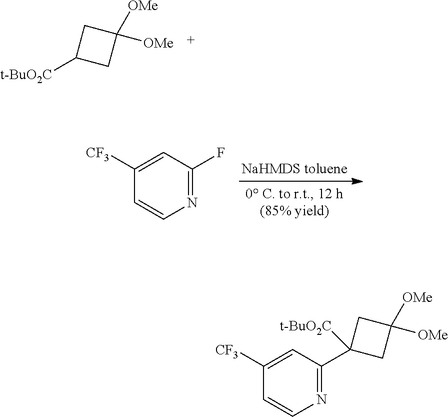

### Step 2: tert-Butyl 3,3-dimethoxy-1-(4-(trifluoromethyl)pyridin-2-yl)cyclobutane-1-carboxylate

A solution of 4-trifluoromethyl-2-fluoropyridine (38.1 g, 231 mmol) and tert-butyl 3,3-dimethoxy-1-(4-(trifluoromethyl)pyridin-2-yl)cyclobutane-1-carboxylate (50.0 g, 231 mmol) in toluene (690 mL, 0.33 M) was cooled to 0° C. A solution of sodium 1,1,1,3,3,3-hexamethyldisilazan-2-ide (2 M solution in THF, 115.5 mL, 231 mmol, 1.0 equiv) was added dropwise over 15 minutes. The reaction mixture was stirred for 60 minutes at the same temperature, and then allowed to warm to room temperature and stirred for 11 hours. The reaction was quenched with saturated aqueous NH₄Cl solution and extracted with EtOAc (3×200 mL). The combined organic extracts were washed with 1N aqueous citric acid solution and brine. The organic phase was dried over Na₂SO₄, filtered, and concentrated in vacuo to give a crude product as yellow oil. Purification by silica gel column chromatography (800 g biotage column) eluting with 0-25% EtOAc in hexanes provided the desired product (73.1 g, 85% yield) contaminated with starting tert-butyl 3,3-dimethoxy-1-(4-(trifluoromethyl)pyridin-2-yl)cyclobutane-1-carboxylate (10% by 1H NMR analysis). ¹H NMR (400 MHz, Chloroform-d) δ 8.59 (d, J=5.1 Hz, 1H), 7.39 (d, J=1.3 Hz, 1H), 7.29-7.15 (m, 1H), 3.06 (s, 3H), 2.98 (s, 3H), 2.92-2.83 (m, 2H), 2.68-2.59 (m, 2H), 1.25 (s, 9H).

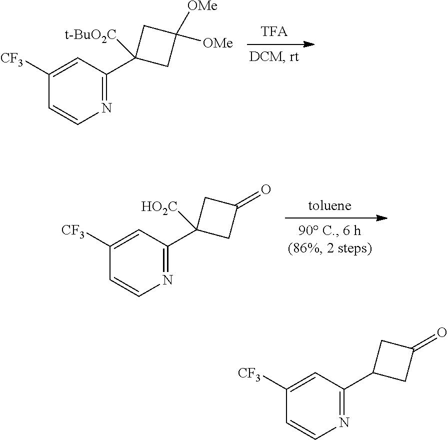

### Step 3: 3-(4-(trifluoromethyl)pyridin-2-yl)cyclobutan-1-one

To a solution of tert-butyl 3,3-dimethoxy-1-(4-(trifluoromethyl)pyridin-2-yl)cyclobutane-1-carboxylate (72.5 g, 217 mmol) in DCM (217 mL, 1.0 M) was added TFA (217 mL, 1.0 M) at room temperature under N₂. The resulting solution was stirred for 3 hours. The solvent was removed in vacuo to provide intermediate 3-oxo-1-(4-(trifluoromethyl)pyridin-2-yl)cyclobutane-1-carboxylic acid (60.1 g) which was taken through the next without any further purification.

3-Oxo-1-(4-(trifluoromethyl)pyridin-2-yl)cyclobutane-1-carboxylic acid (60.1 g) was dissolved in toluene (555 mL, 0.5 M). The resulting mixture was warmed up to 90° C. and stirred for 6 hours. The reaction mixture was cooled to room temperature and diluted with EtOAc and water. The organic phase was washed with saturated aqueous NaHCO₃ solution, then brine, dried over Na₂SO₄, filtered, and concentrated in vacuo to afford the crude product as a dark brown oil. The crude product was purified by silica gel column chromatography (800 g biotage column) eluting with 0-30% EtOAc in heptane resulting in the desired product as a pale-yellow oil (40.4 g, 86% yield). ¹H NMR (400 MHz, Chloroform-d) δ 8.77 (d, J=5.1 Hz, 1H), 7.45 (s, 1H), 7.40 (dd, J=5.1, 1.6 Hz, 1H), 3.88-3.69 (m, 1H), 3.47 (s, 2H), 3.46-3.43 (m, 2H).

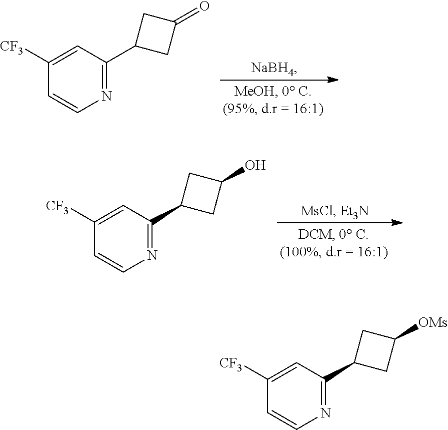 [SMILES: O=C1CC(c2cc(C(F)(F)F)ccn2)C1 ; O[C@H]1C[C@@H](c2cc(C(F)(F)F)ccn2)C1 ; CS(=O)(=O)O[C@H]1C[C@@H](c2cc(C(F)(F)F)ccn2)C1]

### Step 4a: (cis)-3-(4-(trifluoromethyl)pyridin-2-yl)cyclobutan-1-ol

To a solution of 3-(4-(trifluoromethyl)pyridin-2-yl)cyclobutan-1-one (23.0 g, 107 mmol) in MeOH (430 mL, 0.25 M) at 0° C. was added NaBH₄ (4.44 g, 117 mmol) portionwise over 5 minutes. The reaction was stirred for an hour at 0° C. and then warmed to rt and quenched with the sequential addition of H₂O (200 mL) and NaOH (200 mL, 1 M aq. sol). The solution was stirred for 10 minutes at rt and then concentrated. The resulting residue was dissolved in EtOAc (200 mL) and H₂O (200 mL). The organic layer was separated, and the aqueous layer extracted with EtOAc (2×30 mL). The organic fractions were combined, washed with brine, dried over MgSO₄, and concentrated. The crude cis-alcohol (22.1 g, 95% yield, dr 16:1) was directly taken through the next step without any additional purification.

### Step 4b: (cis)-3-(4-(trifluoromethyl)pyridin-2-yl)cyclobutyl methanesulfonate

(cis)-3-(4-(trifluoromethyl)pyridin-2-yl)cyclobutan-1-ol (22.4 g, 103 mmol), obtained from the previous step, was dissolved in DCM (410 mL, 0.25 M) and the solution was cooled to 0° C. Et₃N (154 mmol, 21.5 mL) and methanesulfonyl chloride (113 mmol, 8.73 mL) were subsequently added and the reaction stirred for an hour at 0° C. The reaction was quenched with the addition of satd aq. NH₄Cl (40 mL). The organic layer was separated, and the aqueous layer extracted with DCM (2×100 mL). The organic fractions were combined, dried over MgSO₄, concentrated to afford the corresponding mesylate (31.0 g, 100% yield, d.r 16:1, 95% purity). ¹H NMR (400 MHz, Chloroform-d) δ 8.71 (d, J=5.1 Hz, 1H), 7.35-7.24 (m, 2H), 5.02 (tt, J=8.2, 7.0 Hz, 1H), 3.23 (tt, J=9.9, 7.5 Hz, 1H), 2.96 (s, 3H), 2.89-2.77 (m, 2H), 2.73-2.57 (m, 2H).

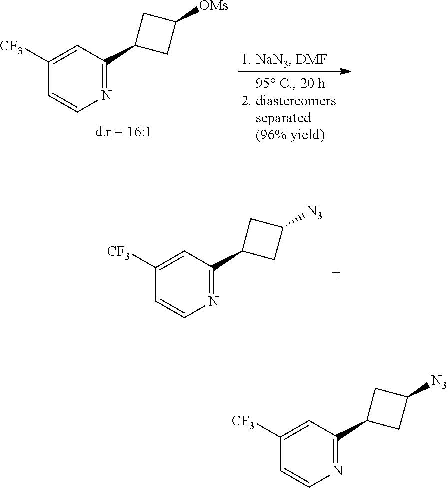 [SMILES: CS(=O)(=O)O[C@H]1C[C@@H](c2cc(C(F)(F)F)ccn2)C1 ; [N-]=[N+]=N[C@H]1C[C@H](c2cc(C(F)(F)F)ccn2)C1 ; [N-]=[N+]=N[C@H]1C[C@@H](c2cc(C(F)(F)F)ccn2)C1]

### Step 5: 2-(trans)-3-azidocyclobutyl)-4-(trifluoromethyl)pyridine

To a solution of (cis)-3-(4-(trifluoromethyl)pyridin-2-yl)cyclobutyl methanesulfonate (31.0 g, 105 mmol, 16:1 dr) in DMF (80 mL, 1.25 M) at rt was added NaN₃ (22.5 g, 346 mmol, 3.3 equiv.). The reaction was stirred for 15 min at rt and then warmed to 95° C. and stirred for 20 h. Upon reaction completion as evidenced by crude TLC analysis, the solution was quenched with addition of satd aq. NaHCO₃ (500 mL). The aqueous solution was extracted with EtOAc (3×300 mL). The organic fractions were combined and washed with brine, dried over MgSO₄, and concentrated. The crude azide was purified by chromatography (SiO₂, 0-10% EtOAC/Hexanes) to afford the corresponding diastereomers.

trans diasteromer (23.2 g, 91% yield). ¹H NMR (400 MHz, Chloroform-d) δ 8.69 (d, J=5.0 Hz, 1H), 7.29 (dd, J=5.0, 1.5 Hz, 1H), 7.26 (s, 1H), 3.85 (tt, J=9.0, 7.3 Hz, 1H), 3.30 (tt, J=10.0, 7.7 Hz, 1H), 2.68 (dddd, J=12.0, 7.5, 5.0, 2.8 Hz, 2H), 2.41 (qd, J=9.1, 2.8 Hz, 2H).

cis diastereomer (1.3 g, 5% yield). ¹H NMR (400 MHz, Chloroform-d) δ 8.70 (d, J=5.0 Hz, 1H), 7.29 (dd, J=5.1, 1.6 Hz, 1H), 7.26 (s, 1H), 4.26 (dddd, J=13.3, 7.3, 5.9, 1.1 Hz, 1H), 3.78-3.56 (m, 1H), 2.61 (dddd, J=12.7, 7.7, 5.3, 2.4 Hz, 2H), 2.55-2.33 (m, 2H).

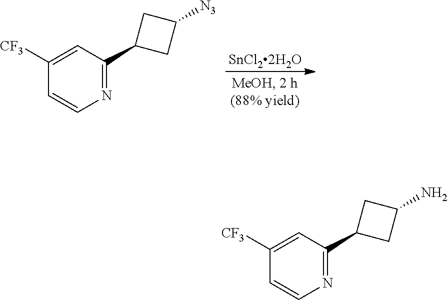 [SMILES: [N-]=[N+]=N[C@H]1C[C@H](c2cc(C(F)(F)F)ccn2)C1 ; N[C@H]1C[C@H](c2cc(C(F)(F)F)ccn2)C1]

### Step 6: (trans)-3-(4-(trifluoromethyl)pyridin-2-yl)cyclobutan-1-amine (Intermediate 4)

2-((trans)-3-azidocyclobutyl)-4-(trifluoromethyl)pyridine (6.3 g, 26.1 mmol) was dissolved in MeOH (130 mL, 0.2 M) and SnCl₂.H₂O (12.0 g, 53.4 mmol, 2.05 equiv.) was added. The reaction was stirred at rt for 2 hours, whereupon the solution was cooled to 0° C. NH₃ (7.0 M in MeOH) was added dropwise until a white precipitate crashed out. The solution was sonicated for 10 minutes and then filtered over a pad of celite. The filtrate was concentrated and dissolved in Et₂O and NaOH (1.0 M). The organic layer was separated, and the aqueous layer extracted with Et₂O (2×100 mL). The organic fractions were combined, dried over MgSO₄, and concentrated. The crude amine (Intermediate 4, 4.9 g, 88% yield) was used in the next step without further purification. ¹H NMR (400 MHz, Methanol-d₄) δ 8.68 (d, J=5.3 Hz, 1H), 7.48 (s, 1H), 7.42 (dd, J=5.3, 1.8 Hz, 1H), 3.77-3.61 (m, 2H), 2.53 (tt, J=7.8, 5.1 Hz, 2H), 2.23 (dtd, J=12.6, 6.3, 2.5 Hz, 2H).

### Example I-5. Intermediate Synthesis 5

### Preparation of (R)-2,3,4,9-tetrahydro-1H-carbazol-2-amine and (S)-2,3,4,9-tetrahydro-1H-carbazol-2-amine (Intermediates 5.3 and 5.4)

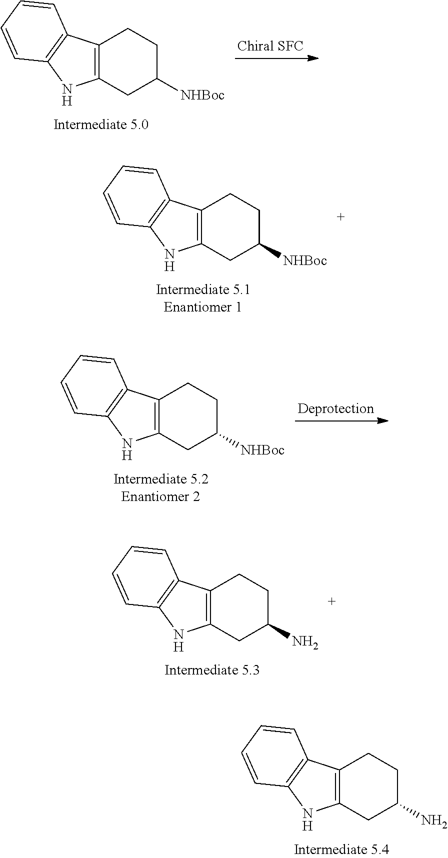 [SMILES: CC(C)(C)OC(=O)NC1CCc2c([nH]c3ccccc23)C1 ; CC(C)(C)OC(=O)N[C@@H]1CCc2c([nH]c3ccccc23)C1 ; CC(C)(C)OC(=O)N[C@H]1CCc2c([nH]c3ccccc23)C1 ; N[C@@H]1CCc2c([nH]c3ccccc23)C1 ; N[C@H]1CCc2c([nH]c3ccccc23)C1]

### Step 1: Chiral SFC separation of tert-butyl (2,3,4,9-tetrahydro-1H-carbazol-2-yl)carbamate

Racemic tert-butyl (2,3,4,9-tetrahydro-1H-carbazol-2-yl)carbamate (intermediate 5.0) (1 g) was subjected to chiral SFC separation using Chiralpak AD-H column (eluting with 25% methanol with 0.1% isopropylamine). The first-eluting enantiomer (0.45 g, 97.1% ee) was arbitrarily assigned as Intermediate 5.1. The second eluting enantiomer (0.46 g, 95.5% ee) was arbitrarily assigned as Intermediate 5.2.

### Step 2: Boc deprotection of tert-butyl (2,3,4,9-tetrahydro-H-carbazol-2-yl)carbamate

To a solution of Intermediate 5.1 (0.20 g, 0.70 mmol) was added 6N HCl (10 equiv) solution in 1,4-dioxane. The reaction mixture was stirred at 60° C. for 24 h. The resulting mixture was then cooled to 24° C. and concentrated under reduced pressure. The crude product was suspended in diethyl ether (50 mL), and the mixture was stirred for 30 m). The remaining solid was collected via filtration and washed with diethyl ether to afford pure (R)-2,3,4,9-tetrahydro-1H-carbazol-2-amine (Intermediate 5.3) as white solid (0.098 g, 75% yield). The absolute stereochemistry was arbitrarily assigned.

TABLE I-5.1
The following intermediates were prepared according to synthetic
procedures as described in Intermediate Synthesis 5, step 1.

| | Starting | |
|---|---|---|
| Intermediate | Carbamate | Structure, Name and Data |
| 5.1 | tert-butyl (2,3,4,9- tetrahydro-1H- carbazol-2- yl)carbamate | 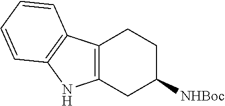 [SMILES: CC(C)(C)OC(=O)N[C@@H]1CCc2c([nH]c3ccccc23)C1] tert-butyl (R)-(2,3,4,9-tetrahydro-1H-carbazol-2- yl)carbamate. 1H NMR (400 MHz, DMSO-d6) δ 10.62 (s, 1H), 7.33 (d, J = 7.7 Hz, 1H), 7.24 (d, J = 7.9 Hz, 1H), 7.03-6.96 (m, 2H), 6.92 (t, J = 7.3 Hz, 1H), 3.77 (t, J = 8.3 Hz, 1H), 2.94 (dd, J = 16.0, 5.4 Hz, 1H), 2.82-2.69 (m, 1H), 2.69-2.56 (m, 2H), 2.01 (dt, J = 12.7, 3.2 Hz, 1H), 1.67 (ddq, J = 16.6, 11.0, 5.7 Hz, 1H), 1.42 (s, 9H). LC/MS (APCI) m/z calcd. for C17H23N2O2+ [M + H]+: 287.1; 287.1 found. Stereochemistry arbitrarily assigned. |
| 5.2 | tert-butyl (2,3,4,9- tetrahydro-1H- carbazol-2- yl)carbamate | 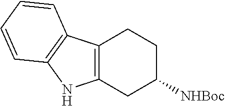 [SMILES: CC(C)(C)OC(=O)N[C@H]1CCc2c([nH]c3ccccc23)C1] tert-butyl (S)-(2,3,4,9-tetrahydro-1H-carbazol-2- yl)carbamate. LC/MS (APCI) m/z calcd. for C17H23N2O2+ [M + H]+: 287.1; 287.1 found. Stereochemistry arbitrarily assigned. |

TABLE I-5.2
The following intermediates were prepared according to synthetic
procedures as described in Intermediate Synthesis 5, step 2.

| Inter- | Starting | |
|---|---|---|
| mediate | Carbamate | Structure, Name and Data |
| 5.3 | 5.1 | 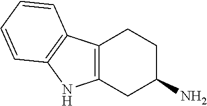 [SMILES: N[C@@H]1CCc2c([nH]c3ccccc23)C1] (R)-2,3,4,9-tetrahydro-1H- carbazol-2-amine. LC/MS (APCI) m/z calcd. for C12H15N2+ [M + H]+: 187.1; 187.1 found. Stereochemistry arbitrarily assigned. |
| 5.4 | 5.2 | 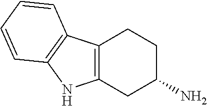 [SMILES: N[C@H]1CCc2c([nH]c3ccccc23)C1] (S)-2,3,4,9-tetrahydro-1H- carbazol-2-amine. LC/MS (APCI) m/z calcd. for C12H15N2+ [M + H]+: 187.1; 187.1 found. Stereochemistry arbitrarily assigned. |

### Example 1-6. Intermediate Synthesis 6

### Preparation of 7-Chlorochroman-3-amine (Intermediate 6.0)

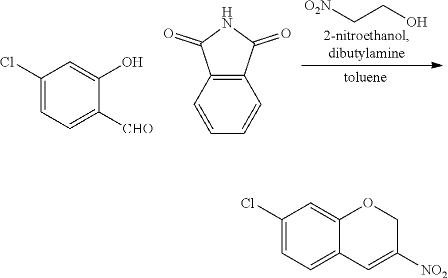 [SMILES: O=[N+]([O-])CCO ; O=Cc1ccc(Cl)cc1O ; O=C1NC(=O)c2ccccc21 ; O=[N+]([O-])C1=Cc2ccc(Cl)cc2OC1]

### Step 1: 7-chloro-3-nitro-2H-chromene

To 4-chloro-2-hydroxybenzaldehyde (5 g, 31.9 mmol, 1 equiv), dibutylamine (2.69 mL, 15.97 mmol, 0.5 equiv), phthalic anhydride (9.46 g, 63.87 mmol, 2 equiv) in toluene (250 mL) was added nitroethanol (6.18 mL, 86.2 mmol, 2.7 equiv). The round bottom flask was fitted with a dean stark apparatus and refluxed for 18 h. The mixture was cooled and another equivalent of nitroethanol was added, and the resulting mixture was further refluxed for 24 h. The reaction was poured into EtOAc, washed with water and brine, dried over sodium sulfate, filtered, and solvent removed by rotary evaporation. The product and remaining starting material were coeluted by silica chromatography (0->10% EtOAc/hexanes) as a white solid (1.7 g, 25%). ¹H NMR (400 MHz, DMSO-d₆) δ 8.10 (d, J=1.1 Hz, 1H), 7.56 (d, J=8.1 Hz, 1H), 7.15 (dd, J=8.2, 2.0 Hz, 1H), 7.12-7.07 (m, 1H), 5.29 (d, J=1.2 Hz, 2H). LC/MS (APCI) m/z calcd. for C₉H₆ClNO₃⁻ [M−H]⁻: 210.0; 210.1 found.

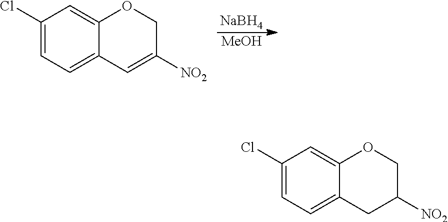 [SMILES: "MeOH" ; O=[N+]([O-])C1=Cc2ccc(Cl)cc2OC1 ; "NaBH4" ; O=[N+]([O-])C1COc2cc(Cl)ccc2C1]

### Step 2: 7-Chloro-3-nitrochromane

Sodium borohydride (0.912 g, 24.1 mmol, 3 equiv) was added to a stirring solution of 7-chloro-3-nitro-2H-chromene (1.7 g, 8.03 mmol, 1 equiv) in MeOH (100 mL) at rt. After 30 minutes, the reaction was quenched with AcOH (20 mL) and concentrated by rotary evaporation. The crude residue was dissolved in EtOAc, washed with sodium bicarbonate and brine, dried over sodium sulfate, filtered, and concentrated by rotary evaporation. The product was then isolated by silica chromatography (5->20% EtOAc/hex) as a yellow solid (419 mg, 24%). ¹H NMR (400 MHz, DMSO-d₆) δ 7.23 (d, J=8.2 Hz, 1H), 6.98 (dd, J=8.2, 2.2 Hz, 1H), 6.90 (d, J=2.2 Hz, 1H), 5.43-5.34 (m, 1H), 4.75 (dt, J=12.3, 2.8 Hz, 1H), 4.40 (dd, J=12.2, 2.2 Hz, 1H), 3.48-3.36 (m, 1H), 3.32 (s, 1H). LC/MS (APCI) m/z calcd. for C₉H₇ClNO₃⁻ [M−H]⁻: 212.0; 212.1 found.

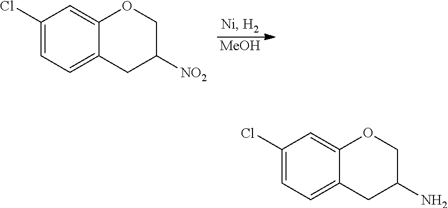 [SMILES: "MeOH" ; O=[N+]([O-])C1COc2cc(Cl)ccc2C1 ; NC1COc2cc(Cl)ccc2C1]

### Step 3: 7-Chlorochroman-3-amine (Intermediate 6.0)

Raney nickel (500 mg) was added to a solution of 7-chloro-3-nitrochromane (1.34 g, 6.27 mmol, 1 equiv) in MeOH (20 mL) before being stirred under H2 at rt. After 4 h, the reaction was filtered through a pad of celite and solvent removed by rotary evaporation to give the product as a yellow oil. LC/MS (APCI) m/z calcd. for C₉H₇ClNO₃⁺ [M+H]⁺: 184.1; 184.1 found.

TABLE I-6
The following intermediates were prepared according to synthetic
procedures as described in Intermediate Synthesis 6, steps 1-3.

| Inter- | Starting | |
|---|---|---|
| mediate | Aldehyde | Structure, Name, Data |
| 6.0 | 4-chloro-2- hydroxy- benzaldehyde | 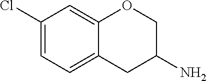 [SMILES: NC1COc2cc(Cl)ccc2C1] 7-Chlorochroman-3-amine. LC/MS (APCI) m/z calcd. for C₉H₇ClNO₃⁺ [M + H]⁺: 184.1; 184.1 found. |
| 6.1 | 5-chloro-2- hydroxy- benzaldehyde | 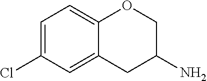 [SMILES: NC1COc2ccc(Cl)cc2C1] 6-Chlorochroman-3-amine. ¹H NMR (400 MHz, DMSO-d₆) δ 7.17-7.11 (m, 1H), 7.08 (dd, J = 8.7, 2.7 Hz, 1H), 6.75 (d, J = 8.7 Hz, 1H), 4.14-4.03 (m, 2H), 3.60 (dd, J = 10.3, 8.5 Hz, 1H), 3.09 (tdd, J = 8.6, 5.2, 3.4 Hz, 1H), 2.88 (ddd, J = 16.3, 5.2, 1.8 Hz, 1H), 1.68 (s, 2H). LC/MS (APCI) m/z calcd. for C₉H₇ClNO₃⁺ [M + H]⁺: 184.1; 184.1 found. |

### Example I-7. Intermediate Synthesis 7

### Preparation of (cis)-6-chloro-8-fluoro-4-methylchroman-3-amine (Intermediate 7)

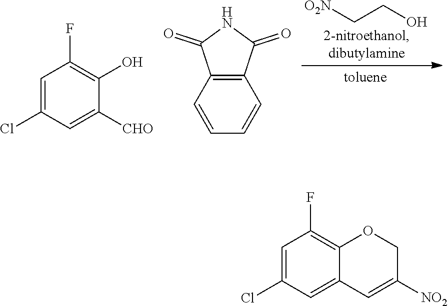 [SMILES: O=[N+]([O-])CCO ; O=Cc1cc(Cl)cc(F)c1O ; O=C1NC(=O)c2ccccc21 ; O=[N+]([O-])C1=Cc2cc(Cl)cc(F)c2OC1]

### Step 1: Synthesis of 6-chloro-8-fluoro-3-nitro-2H-chromene

To 3,fluoro-5-chloro-2-hydroxybenzaldehyde (4.5 g, 25.8 mmol), dibutylamine (1.66 g, 12.9 mmol), phthalic anhydride (7.64 g, 7.64 mmol) in toluene (200 mL) was added nitroethanol (2.34 g, 25.78 mmol). The round bottom flask was fitted with a dean stark apparatus and refluxed for 18 h. The mixture was cooled and another equivalent of nitroethanol was added, and the resulting mixture was further refluxed for 24 h. The reaction was evaporated down to approximately 30 mL and purified by silica chromatography (10% EtOAc/hexanes). The unreacted starting material and product coeluted. The fraction containing the product and SM were combined and washed with 2% NaOH solution. The organic layers were combined, dried over sodium sulfate, filtered, and solvent removed by rotary evaporation. The product was recrystallized from EtOAc/Hexanes (10%) to provide product (2.1 g, 9.2 mmol, 35.4% yield) as yellow crystalline solid. LC/MS (APCI) m/z calcd. for C₉H₆ClFNO₃⁺ [M+H]⁺: 230.0; 230.2 found.

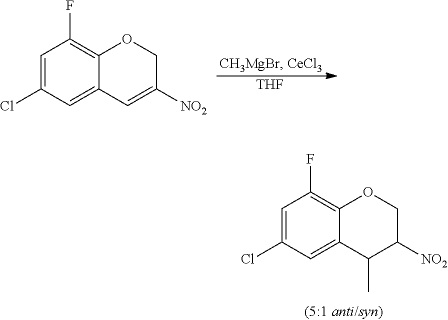 [SMILES: O=[N+]([O-])C1=Cc2cc(Cl)cc(F)c2OC1 ; CC1c2cc(Cl)cc(F)c2OCC1[N+](=O)[O-] ; "THF"]

### Step 2: Synthesis of 6-chloro-8-fluoro-4-methyl-3-nitrochromane

MeMgBr in Et₂O (3 M, 2.82 mL, 8.46 mmol) was added to a suspension of CuI (3.0 g, 8.46 mmol) in THF (30 mL) at −0° C. After 90 min, the solution was cooled to −40° C. and a solution of 6-chloro-8-fluoro-3-nitro-2H-chromene (986 mg, 4.3 mmol) in THF (10 ml) was added dropwise under vigorous stirring. After 10 min, the reaction was quenched with glacial AcOH (10 eq) and left to stir under for 5 min −40° C. before being diluted with water and extracted with EtOAc. The organic layers were combined, dried sodium sulfate, filtered, and solvent removed by rotary evaporation. The crude was purified by silica chromatography (10% EtOAc/hexanes) to provide the product (0.82 g, 3.34 mmol, 77.7% yield, 5:1 anti/syn) as a pale yellow oil. R_{f}=0.18 (SiO₂, 5% EtOAc/hexanes). LC/MS (APCI) m/z calcd. for C₁₀H₁₀ClFNO₃⁺ [M+H]⁺: 246.0; 246.2 found.

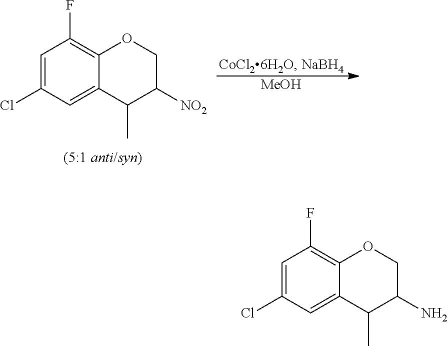 [SMILES: "MeOH" ; CC1c2cc(Cl)cc(F)c2OCC1[N+](=O)[O-] ; CC1c2cc(Cl)cc(F)c2OCC1N]

### Step 3: Synthesis of 6-chloro-8-fluoro-4-methylchroman-3-amine (Intermediate 7)

A solution of the 6-chloro-8-fluoro-4-methyl-3-nitrochromane (0.8 g, 3.26 mmol) and cobalt(II) chloride (0.77 g, 3.26 mmol) in MeOH (0.1 M) was cooled to 0° C. followed by addition of sodium borohydride (0.62 g, 16.2 mmol). The resulting black suspension was stirred at 0° C. for 15 minutes and then at room temperature until complete (monitored by LCMS, 60 mins). The reaction was quenched by the dropwise addition of 3 M aq HCl until pH 2 was reached. Then 1 M aq NH₄OH was added dropwise until the solution attained pH 9. Methanol was removed, and the aqueous layer was extracted with ethyl acetate. The combined organic extracts were dried over magnesium sulfate and concentrated to afford the product (0.625 g, 2.90 mmol, 89% yield) as a yellow oil. LC/MS (APCI) m/z calcd. for C₁₀H₁₂ClFNO⁺ [M+H]⁺: 216.1; 216.2 found.

### Example I-8. Intermediate Synthesis 8

### Preparation of 5,6-Dichloro-2,3-dihydro-1H-inden-2-amine (Intermediate 8.0)

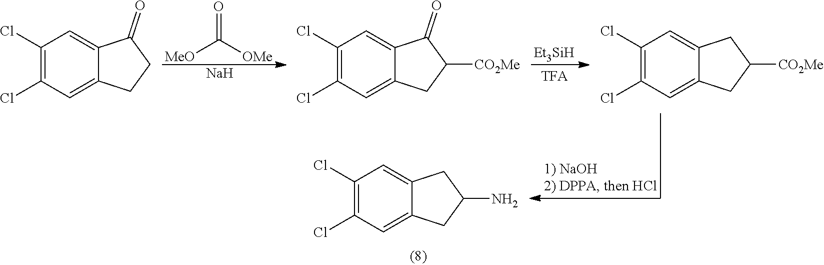

### Step 1: Synthesis of 5,6-dichloro-1-oxo-2,3-dihydro-1H-indene-2-carboxylate

To a 100-mL round-bottomed flask was added 5,6-dichloro-1-indanone (1.0 g, 5.0 mmol), dimethyl carbonate (0.67 ml, 8.0 mmol) and NaH (60% dispersion in mineral oil, 7.5 mmol). The reaction mixture was stirred at 80° C. for 4 h, before it was cooled to 22° C. The solvent was evaporated, and the residue was absorbed onto a plug of silica gel and purified by chromatography through a Redi-Sep pre-packed silica gel column (12 g), eluting with a gradient of 0-15% EtOAc/EtOH (3:1) in heptane, to provide methyl 5,6-dichloro-1-oxo-2,3-dihydro-1H-indene-2-carboxylate (0.62 g, 2.4 mmol, 48.1% yield) as tan solid: ¹H NMR (400 MHz, CHLOROFORM-d) δ 10.15-10.35 (m, 1H), 7.72 (s, 1H), 7.57 (s, 1H), 3.88 (s, 3H), 3.51 (d, J=0.73 Hz, 2H); LCMS-ESI (POS) m/z: 259.0 (M+H)⁺.

### Step 2: Synthesis of 5,6-Dichloro-2,3-dihydro-1H-indene-2-carboxylate

To a 100-mL round-bottomed flask was added methyl 5,6-dichloro-3-hydroxy-1H-indene-2-carboxylate (1.4 g, 5.6 mmol) and triethylsilane (4.4 ml, 27.8 mmol) in trifluoroacetic acid (15.9 ml). The reaction mixture was stirred at 22° C. for 48 h. The solvent was evaporated, and the crude material was absorbed onto a plug of silica gel and purified by chromatography through a Redi-Sep pre-packed silica gel column (12 g), eluting with a gradient of 0-10% EtOAc/EtOH (3:1) in heptane, to provide methyl 5,6-dichloro-2,3-dihydro-1H-indene-2-carboxylate (0.9 g, 3.7 mmol, 66.1% yield) as yellow powder: ¹H NMR (400 MHz, DMSO-d₆) δ 7.49 (s, 2H), 3.64 (s, 3H), 3.39-3.47 (m, 1H), 3.03-3.23 (m, 4H).

### Step 3: 5,6-Dichloro-2,3-dihydro-1H-inden-2-amine (Intermediate 8.0)

To a 100-mL round-bottomed flask was added methyl 5,6-dichloro-2,3-dihydro-1H-indene-2-carboxylate (0.8 g, 3.3 mmol) and lithium hydroxide (0.39 g, 16.3 mmol) in a mixed solvent of THF (19.6 ml), methanol (6.5 ml), and water (6.5 ml). The reaction mixture was stirred at 22° C. for 1 h, before it was diluted with water and acidified to pH=3. The mixture was extracted with DCM, and the combined organic layers were dried over MgSO₄ and concentrated under reduced pressure. The resulting crude product was directly used in the subsequent reaction without further purification. A mixture of crude 5,6-dichloro-2,3-dihydro-1H-indene-2-carboxylic acid (0.35 g, 1.5 mmol), diphenyl phosphorazidate (0.49 ml, 2.3 mmol) and triethylamine (0.32 ml, 2.3 mmol) in acetonitrile (10.1 ml) was stirred at 80° C. for 1 h. It was cooled to 22° C. and treated with 1N HCl (2 mL). The reaction mixture was stirred at 80° C. for 24 h. The mixture was diluted with water and washed with EtOAc. The aqueous fraction was basified with 6N NaOH and extracted with DCM. The combined organic layers were dried over MgSO₄ and concentrated under reduced pressure to afford crude 5,6-dichloro-2,3-dihydro-1H-inden-2-amine: LCMS-ESI (POS) m/z: 202.0 (M+H)⁺.

TABLE I-8
The following intermediates were prepared according to synthetic
procedures as described in Intermediate Synthesis 8, steps 1-3.

| Inter- | Starting | |
|---|---|---|
| mediate | indanone | Structure, Name, Data |
| 8.0 | 5,6- dichloro- 1-indanone | 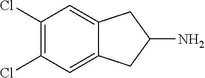 [SMILES: NC1Cc2cc(Cl)c(Cl)cc2C1] 5,6-Dichloro-2,3-dihydro-1H-inden-2- amine: LCMS-ESI (POS) m/z: 202.0 (M + H)⁺. |
| 8.1 | 5-chloro-6- fluoro-1- indanone | 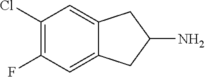 [SMILES: NC1Cc2cc(F)c(Cl)cc2C1] 5-Chloro-6-fluoro-2,3-dihydro-1H-inden-2- amine: ¹H NMR (500 MHz, DMSO-d₆) δ 8.26 (br s, 2H), 7.51 (d, J = 7.14 Hz, 1H), 7.36 (d, J = 9.47 Hz, 1H), 3.95-4.09 (m, 1H), 3.21- 3.30 (m, 2H), 2.90-3.01 (m, 2H); LCMS-ESI (POS) m/z: 186.2 (M + H)⁺. |

### Example I-9. Intermediate Synthesis 9

### Preparation of (R)-5-Chloro-6-fluoro-2,3-dihydro-1H-inden-2-amine and (S)-5-Chloro-6-fluoro-2,3-dihydro-1H-inden-2-amine (Intermediates 9.1 and 9.2)

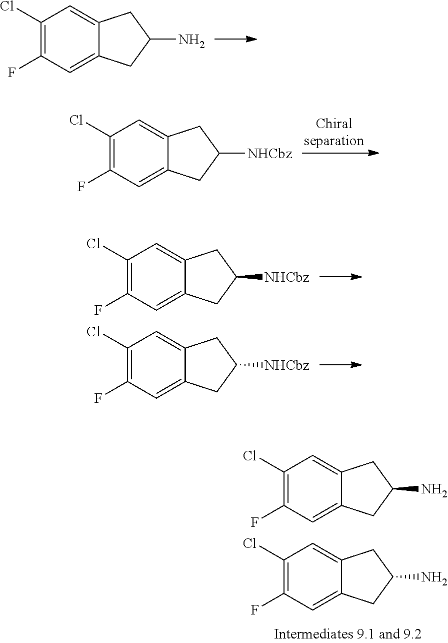

Racemic benzyl (5-chloro-6-fluoro-2,3-dihydro-1H-inden-2-yl)carbamate (50.8 g, 159 mmol) was prepared from 5-chloro-6-fluoro-2,3-dihydro-1H-inden-2-amine hydrochloride and then subjected to chiral SFC separation using Chiralcel OJ-H column (eluting with 25% isopropanol).

The first-eluting enantiomer (21.1 g, 66.0 mmol, 99% ee) was dissolved in a 6N HCl solution in 1,4-dioxane (55.0 mL, 330.0 mmol). The reaction mixture was stirred at 110° C. for 60 h; it was then cooled to 22° C. and concentrated under reduced pressure. The resulting crude product was suspended in diethyl ether (50 mL), and the mixture was stirred for 30 min. The remaining solid was collected via filtration and washed with diethyl ether to afford pure compound (Intermediate 9.1) as white solid (14.3 g, 64.3 mmol, 97% yield, >99% ee).

Cbz-deprotection of the second-eluting enantiomer (22.7 g, 71.0 mmol, 97% ee) obtained from the above mentioned chiral separation using the same procedures described above afforded pure compound (Intermediate 9.2) as white solid (12.0 g, 54.2 mmol, 82% yield, >99% ee).

### Example I-10. Intermediate Synthesis 10

### Preparation of 5-(Trifluoromethyl)-2,3-dihydro-1H-inden-2-amine hydrochloride (Intermediate 10.0)

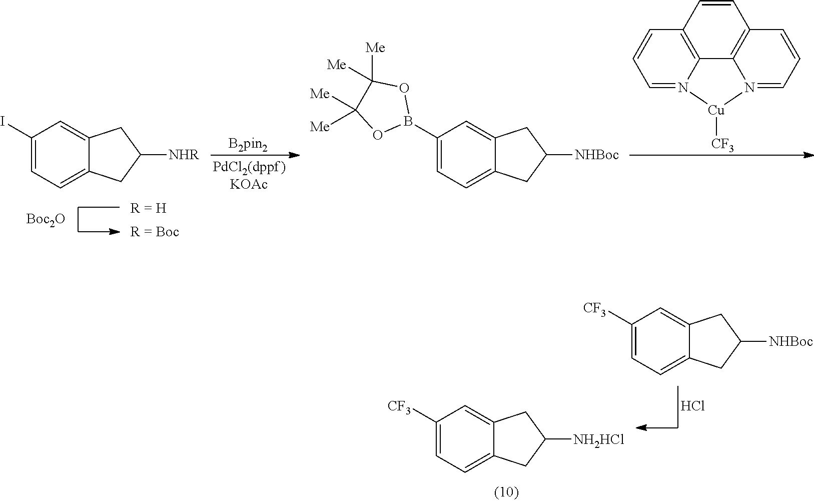

### Step 1: Synthesis of Tert-butyl (5-iodo-2,3-dihydro-1H-inden-2-yl)carbamate

A solution of 5-iodo-2,3-dihydro-1H-inden-2-amine (5.20 g, 20.1 mmol) and di-tert-butyl dicarbonate (2.0 M in DCM, 20.1 mmol) was treated with 1-methylimidazole (2.1 ml, 26.1 mmol). The reaction mixture was allowed to stir at 22° C. for 1 h. The solvent was evaporated; purification of the crude residue by silica gel column chromatography (0-50% EtOAc/heptane) gave tert-butyl (5-iodo-2,3-dihydro-1H-inden-2-yl)carbamate (8.04 g, 20.0 mmol, 99% yield) with minor impurities: ¹H NMR (400 MHz, ACETONITRILE-d₃) δ 7.57-7.62 (m, 1H), 7.52 (td, J=0.86, 7.93 Hz, 1H), 7.03 (dd, J=0.93, 7.88 Hz, 1H), 5.50-5.65 (m, 1H), 4.26-4.37 (m, 1H), 3.11-3.24 (m, 2H), 2.69-2.84 (m, 2H), 2.13-2.18 (m, 9H); LCMS-ESI (POS) m/z: 382.0 (M+Na)⁺.

### Step 2: Synthesis of Tert-butyl (5-(4,4,5,5-tetramethyl-1,3,2-dioxaborolan-2-yl)-2,3-dihydro-1H-inden-2-yl)carbamate

A solution of bis(pinacolato)diboron (5.1 g, 19.9 mmol), tert-butyl (5-iodo-2,3-dihydro-1H-inden-2-yl)carbamate (6.5 g, 18.1 mmol), PdCl₂(dppf)-CH₂Cl₂ adduct (0.74 g, 0.91 mmol), and potassium acetate (7.1 g, 72.4 mmol) in DMF (30 mL) was heated to 100° C. for 12 h. The reaction mixture was purified directly by silica gel column chromatography (0-50% EtOAc/heptane) yielding tert-butyl (5-(4,4,5,5-tetramethyl-1,3,2-dioxaborolan-2-yl)-2,3-dihydro-1H-inden-2-yl)carbamate (6.0 g, 16.8 mmol, 93% yield): LCMS-ESI (POS) m/z: 382.2 (M+Na)⁺.

### Step 3: Synthesis of Tert-butyl (5-(trifluoromethyl)-2,3-dihydro-1H-inden-2-yl)carbamate

A solution of tert-butyl-(5-(4,4,5,5-tetramethyl-1,3,2-dioxaborolan-2-yl)-2,3-dihydro-1H-inden-2-yl)carbamate (6.0 g, 16.8 mmol) in 5:1 MeCN/DMF (120 mL) was heated to 80° C. in a crystalizing dish. Portion-wise addition of (1,10-phenanthroline)(trifluoromethyl)copper(I) (7.86 g, 25.1 mmol) was completed over 1 h. The reaction mixture was diluted with a saturated aqueous Rochelle's salt solution and was extracted with DCM. The organic layers were dried over MgSO₄ and concentrated under reduced pressure. Purification of the crude residue by silica gel column chromatography (0-50% EtOAc/heptane) gave tert-butyl (5-(trifluoromethyl)-2,3-dihydro-1H-inden-2-yl)carbamate (2.5 g, 8.2 mmol, 48.7% yield): ¹H NMR (400 MHz, DMSO-d₆) δ 7.54 (m, 1H), 7.48-7.19 (m, 3H), 4.24 (m, 1H), 3.32-3.16 (m, 2H), 2.85-2.80 (m, 2H), 1.39 (s, 9H); LCMS-ESI (POS) m/z: 202.0 (M-Boc)⁺.

### Step 4: Synthesis of 5-(Trifluoromethyl)-2,3-dihydro-1H-inden-2-amine hydrochloride

Tert-butyl (5-(trifluoromethyl)-2,3-dihydro-1H-inden-2-yl)carbamate (51.0 g, 169.0 mmol) was dissolved in a solution of HCl in 1,4-dioxane (4M, 500 mL), and the reaction mixture was stirred at 25° C. for 16 h. The solvent was evaporated under reduced pressure, and the crude product was collected by filtration and washed with petroleum ether to afford 5-(trifluoromethyl)-2,3-dihydro-1H-inden-2-amine hydrochloride (37.5 g, 158.0 mmol, 93% yield) as white solid: LCMS-ESI (POS) m/z: 202.2 (M+H)⁺.

### Example I-11. Intermediate Synthesis 11

### Preparation of 5-Chloro-2,3-dihydro-1H-inden-2-amine (Intermediate 11.0)

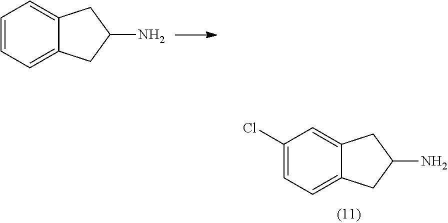 [SMILES: NC1Cc2ccccc2C1 ; NC1Cc2ccc(Cl)cc2C1]

To a solution of 2,3-dihydro-1H-inden-2-amine hydrochloride (5.0 g, 29.5 mmol) in water (50.0 mL) was added N-chlorosuccinimide (2.0 g, 14.7 mmol) and a concentrated HCl solution (29.5 mmol) at 22° C. The reaction mixture was stirred at 55° C. for 48 h. The reaction mixture was cooled to 22° C., before the removal of −20 mL of water under reduced pressure. The crude product was collected by filtration and then suspended in isopropanol. The slurry was stirred at 90° C. for 2 h, before it was cooled to 22° C. and filtered to afford 5-chloro-2,3-dihydro-1H-inden-2-amine hydrochloride (Intermediate 11.0) (2.3 g, 38.2% yield) as off-white powder: ¹H NMR (400 MHz, ACETONITRILE-d₃) δ 7.11-7.25 (m, 3H), 3.73-3.86 (m, 1H), 3.03-3.19 (m, 2H), 2.55-2.70 (m, 2H), 1.81-1.93 (m, 2H).

### Preparation of (S)-5-Chloro-2,3-dihydro-1H-inden-2-amine and (R)-5-Chloro-2,3-dihydro-1H-inden-2-amine (Intermediates 11.1 and 11.2)

Chiral SFC purification of racemic 5-chloro-2,3-dihydro-1H-inden-2-amine (4.0 g, 19.6 mmol) by Chiralpak AY-H column eluting with 10% isopropanol with 0.2% diethylamine afforded the following enantiomers that were arbitrarily assigned:

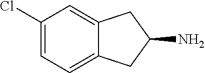 [SMILES: N[C@H]1Cc2ccc(Cl)cc2C1]

(S)-5-Chloro-2,3-dihydro-1H-inden-2-amine (Intermediate 11.1). The title compound was isolated as the first-eluting enantiomer (1.3 g, 7.9 mmol, 40.2% yield, 99% ee) as off-white solid.¹

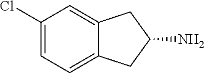 [SMILES: N[C@@H]1Cc2ccc(Cl)cc2C1]

(R)-5-Chloro-2,3-dihydro-1H-inden-2-amine (Intermediate 11.2). The title compound was isolated as the second-eluting enantiomer (1.1 g, 6.4 mmol, 32.4% yield, 94% ee) as off-white solid.

### Example I-12. Intermediate Synthesis 12

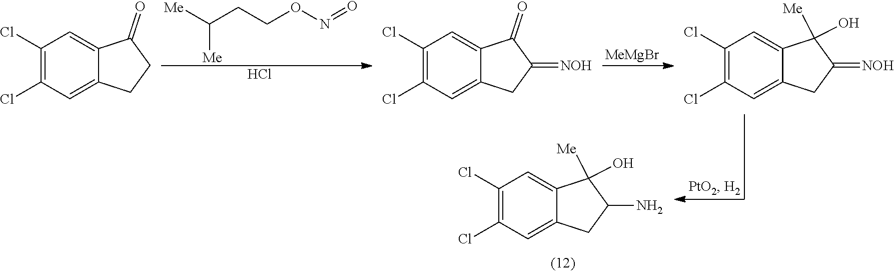

### Preparation of 2-Amino-5,6-dichloro-1-methyl-2,3-dihydro-1H-inden-1-ol (Intermediate 12.0)

### Step 1: 5,6-Dichloro-2-(hydroxyimino)-2,3-dihydro-1H-inden-1-one

A 2-L flask was equipped with an overhead stirrer, N₂ inlet, and a thermal couple, and was charged with 5,6-dichloro-2,3-dihydro-1H-inden-1-one (100.0 g, 497.0 mmol), isoamyl nitrite (200.0 mL, 1492.0 mmol) and methanol (500.0 mL). A 6 N aqueous HCl solution was added dropwise over a period of 30 min, and the reaction mixture was stirred at 50° C. for 2 h. Another 3 equiv of isoamyl nitrite (200.0 mL, 1492.0 mmol) was added, and the reaction mixture was stirred at 22° C. for 16 h. Water (1000.0 mL) was added, and the crude product was collected by filtration and then washed with a NaHCO₃ solution and more water to afford 5,6-dichloro-2-(hydroxyimino)-2,3-dihydro-1H-inden-1-one as light-yellow solid (98.6 g, 429.0 mmol, 86% yield): LCMS-ESI (POS) m/z: 252.0 (M+Na)⁺.

### Step 2: 5,6-Dichloro-1-hydroxy-1-methyl-1H-inden-2(3H)-one oxime

A 2-L flask was equipped with an overhead stirrer, N₂ inlet, and a thermal couple, and was charged with 5,6-dichloro-2-(hydroxyimino)-2,3-dihydro-1H-inden-1-one (89.0 g, 387.0 mmol) and tetrahydrofuran (900.0 mL). The reaction mixture was cooled to −78° C., followed by dropwise addition of methylmagnesium bromide (284.0 mL, 967.0 mmol). It was allowed to warm to 0° C. and stir for 2 h. The reaction mixture was cooled to −20° C., carefully treated with a saturated aqueous NH₄Cl solution (250.0 mL) and water (250.0 mL), and then extracted with EtOAc (500.0 mL). The organic layer was dried over MgSO₄ and concentrated under reduced pressure. The crude product was suspended in DCM (500.0 mL) that upon filtration afforded 5,6-dichloro-1-hydroxy-1-methyl-1H-inden-2(3H)-one oxime as purple solid (73.2 g, 297.0 mmol, 77% yield): LCMS-ESI (POS) m/z: 228.0 (M−OH)⁺.

### Step 3: 2-Amino-5,6-dichloro-1-methyl-2,3-dihydro-1H-inden-1-ol (Intermediate 12.0)

To a 250-mL pressure-proof vessel equipped with a stir bar was added 5,6-dichloro-1-hydroxy-1-methyl-1H-inden-2(3H)-one oxime (10.0 g, 40.6 mmol), platinium (iv)oxide (1.4 g, 6.1 mmol), and ethyl acetate (150.0 mL). The reaction vessel was evacuated and then backfilled with hydrogen gas (40 psi). The reaction mixture was stirred at 22° C. for 12 h. The catalyst was removed by filtration, and the filtrate was concentrated under reduced pressure. The crude product was suspended in DCM that upon filtration afforded 2-amino-5,6-dichloro-1-methyl-2,3-dihydro-1H-inden-1-ol in a mixture of cis- and trans-isomers as white solid (3.85 g, 40.8% yield): LCMS-ESI (POS) m/z: 232.2 (M+H)⁺.

### Example I-13. Intermediate Synthesis 13

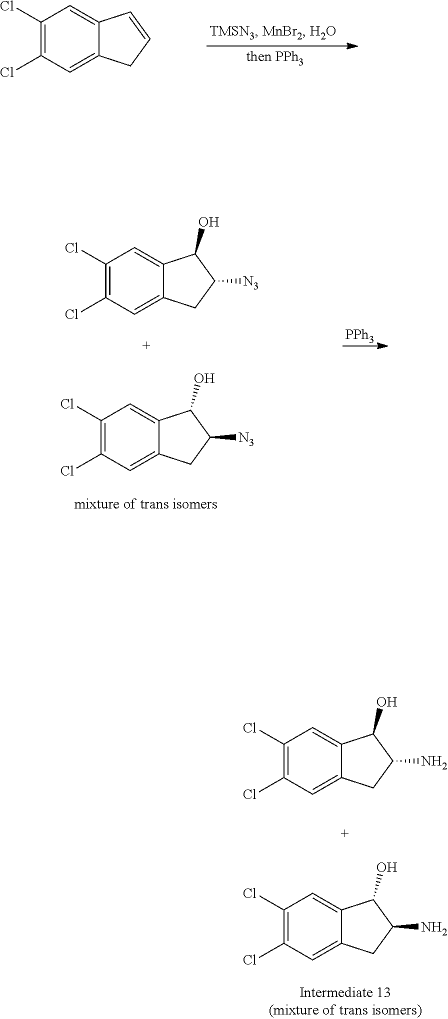 [SMILES: Clc1cc2c(cc1Cl)CC=C2 ; [N-]=[N+]=N[C@@H]1Cc2cc(Cl)c(Cl)cc2[C@H]1O ; [N-]=[N+]=N[C@H]1Cc2cc(Cl)c(Cl)cc2[C@@H]1O ; N[C@@H]1Cc2cc(Cl)c(Cl)cc2[C@H]1O ; N[C@H]1Cc2cc(Cl)c(Cl)cc2[C@@H]1O ; "PPh3"]

### Preparation of Trans-2-Amino-5,6-dichloro-1-methyl-2,3-dihydro-1H-inden-1-ol (Intermediate 13.0)

### Step 1: Trans-2-azido-5,6-dichloro-2,3-dihydro-1H-inden-1-ol

To a 15-mL vial was added 5,6-dichloro-1H-indene (0.78 g, 4.2 mmol) and manganese (II) bromide (91.0 mg, 0.42 mmol) in a mixed solvent of acetonitrile (28.4 ml) and water (0.78 mL). Trimethylsilyl azide (1.1 mL, 8.4 mmol) was slowly added, and the reaction mixture was stirred at 22° C. for 12 h. Triphenylphosphine (1.1 g, 4.2 mmol) was added, and the resulting mixture was stirred for 10 min. The solvent was evaporated under reduced pressure, and the residue was absorbed onto a plug of silica gel and purified by chromatography through a Redi-Sep pre-packed silica gel column (12 g), eluting with a gradient of 0-20% EtOAc/EtOH (3:1) in heptane, to provide trans-2-azido-5,6-dichloro-2,3-dihydro-1H-inden-1-ol (0.49 g, 2.0 mmol, 47.8% yield) as the major product as tan powder (along with 7% of trans-2-azido-5,6-dichloro-2,3-dihydro-1H-inden-1-ol as the minor product): ¹H NMR (400 MHz, CHLOROFORM-d) δ 7.47 (s, 1H), 7.33 (d, J=0.83 Hz, 1H), 5.07 (d, J=6.32 Hz, 1H), 4.10 (dt, J=6.27, 7.80 Hz, 1H), 3.23-3.35 (m, 1H), 2.78-2.90 (m, 1H).

### Step 2: Trans-2-amino-5,6-dichloro-2,3-dihydro-1H-inden-1-ol (Intermediate 13.0)

To a 100-mL round-bottomed flask was added trans-2-azido-5,6-dichloro-2,3-dihydro-1H-inden-1-ol (0.48 g, 2.0 mmol) and triphenylphosphine (1.0 g, 3.9 mmol) in tetrahydrofuran (20.0 mL). The reaction mixture was stirred at 22° C. for 6 h. Water (1 mL) was added, and resulting mixture was stirred for an additional 12 h. The mixture was diluted with 6N HCl and extracted with EtOAc. The aqueous fraction was basified with a 6N NaOH solution to pH=14 and then extracted with DCM. The combined organic layers were dried over MgSO₄ and concentrated under reduced pressure. This crude product was directly used without further purification: LCMS-ESI (POS) m/z: 218.0 (M+H)⁺.

TABLE I-13
The following intermediates were prepared according to synthetic
procedures as described in Intermediate Synthesis 13, steps 1-2.

| Inter- | Starting | |
|---|---|---|
| mediate | indene | Structure, Name, Data |
| 13.0 | 5,6- dichloro- 1H-indene | 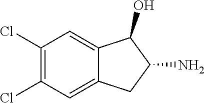 [SMILES: N[C@@H]1Cc2cc(Cl)c(Cl)cc2[C@H]1O] and |
| | | 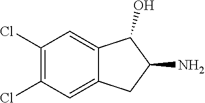 [SMILES: N[C@H]1Cc2cc(Cl)c(Cl)cc2[C@@H]1O] Trans-2-amino-5,6-dichloro-2,3-dihydro- 1H-inden-1-ol.: LCMS-ESI (POS) m/z: 218.0 (M + H)⁺. |
| 13.1 | 5-chloro- 6-fluoro- 1H-indene | 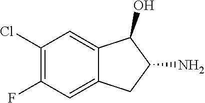 [SMILES: N[C@@H]1Cc2cc(F)c(Cl)cc2[C@H]1O] and |
| | | 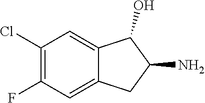 [SMILES: N[C@H]1Cc2cc(F)c(Cl)cc2[C@@H]1O] Trans-2-amino-6-chloro-5-fluoro-2,3- dihydro-1H-inden-1-ol.: ¹H NMR (400 MHz, DMSO-d₆) δ 7.33 (d, J = 7.26 Hz, 1H), 7.22 (d, J = 9.54 Hz, 1H), 5.52 (br s, 1H), 4.51 (br d, J = 6.22 Hz, 1H), 3.22-3.28 (m, 1H), 3.00 (dd, J = 7.26, 15.86 Hz, 1H), 2.41-2.48 (m, 1H), 1.85-2.25 (m, 2H); LCMS-ESI (POS) m/z: 202.2 (M + H)⁺. |

### Example I-14. Intermediate Synthesis 14

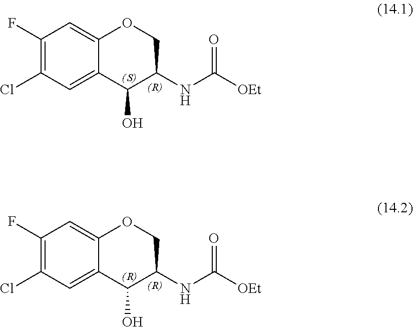

### Preparation of Ethyl ((3R,4S)-6-chloro-7-fluoro-4-hydroxychroman-3-yl)carbamate and ethyl ((3R,4R)-6-chloro-7-fluoro-4-hydroxychroman-3-yl)carbamate (Intermediates 14.1 and 14.2)

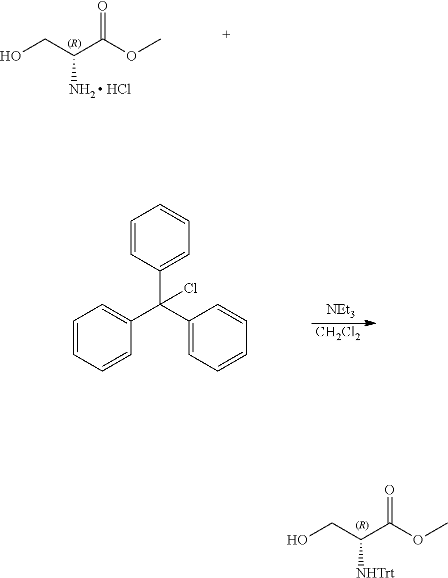

### Step 1: Methyl trityl-D-serinate

To methyl D-serinate hydrochloride (30.0 g, 193 mmol) in dichloromethane (250 mL) was added triethylamine (46.7 mL, 335 mmol). The resulting mixture was cooled to 0° C. with an ice bath and trityl chloride (46.7 g, 168 mmol) was added portionwise. The ice bath was removed, and the reaction stirred at room temperature for 2 h. It was diluted with saturated aqueous sodium bicarbonate (300 mL) and additional dichloromethane (300 mL). The layers were shaken and separated, and the organic phase was washed with saturated aqueous sodium bircarbonate (200 mL), brine (200 mL) and dried over sodium sulfate. Concentration under reduced pressure provided a viscous oil which was recrystallized from diethyl ether providing methyl trityl-D-serinate (25.0 g, 69.1 mmol) as a white crystalline solid. ¹H NMR (400 MHz, DMSO-d₆) δ 7.45-7.32 (m, 6H), 7.32-7.13 (m, 9H), 4.93 (t, J=6.0 Hz, 1H), 3.59 (m, 1H), 3.47-3.36 (m, 1H), 3.20 (m, 1H), 3.13 (s, 3H), 2.81 (d, J=10.0 Hz, 1H). LC/MS (APCI) m/z calcd. for C₂₃H₂₃NO₃⁺ [M+H]⁺: 362.2; 243.2 found (fragmentation).

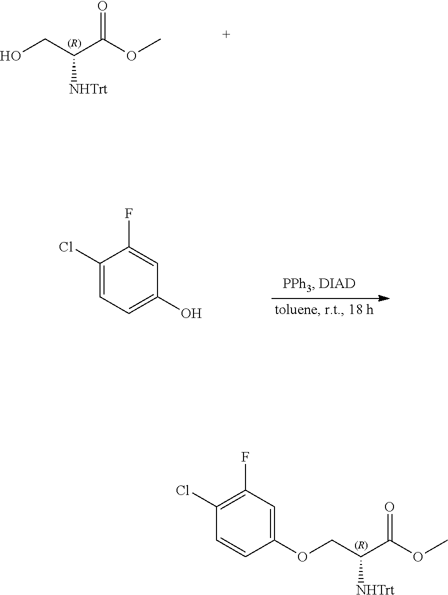

### Step 2: Methyl O-(4-chloro-3-fluorophenyl)-N-trityl-D-serinate

A 500 mL round bottom flask was charged with methyl trityl-D-serinate (14.8 g, 40.9 mmol). Anhydrous toluene (150 mL) was added, followed by 4-chloro-3-fluorophenol (7.2 g, 49.1 mmol) and then triphenylphosphine (11.8 g, 45.0 mmol). The resulting mixture was stirred at 21° C. for 20 min and then diisopropyl azodicarboxylate (8.9 mL, 45.0 mmol) was added dropwise over 15 min. The resulting yellow solution was stirred at 21° C. for 18 h. It was diluted with ethyl acetate (300 mL), washed with water (250 mL), 1 M aqueous NaOH (200 mL) and brine (200 mL). The organic phase was dried over sodium sulfate and concentrated to a viscous yellow oil. The oil was dissolved in diethyl ether (100 mL) and hexanes (350 mL) was added. The resulting precipitate was sonicated and then filtered. The filtered solid was discarded, and the filtrate was concentrated under reduced pressure. Drying under high vacuum provided 17.3 g of methyl O-(4-chloro-3-fluorophenyl)-N-trityl-D-serinate (43%, ˜50% purity) as a faintly yellow sticky solid. The product was carried on to the following step without further purification. ¹H NMR (400 MHz, DMSO-d₆) δ 7.34-7.18 (m, 16H), 7.03 (dd, J=11.4, 2.8 Hz, 1H), 6.78-6.62 (m, 2H), 4.21 (dd, J=10.2, 5.5 Hz, 1H), 4.08 (dd, J=10.2, 7.0 Hz, 1H), 3.54-3.46 (m, 1H), 3.17 (s, 3H). LC/MS (APCI) m/z calcd. for C₂₅H₂₉ClFNO₃⁺ [M+H]⁺: 491.2; 243.2 found (Trityl fragmentation).

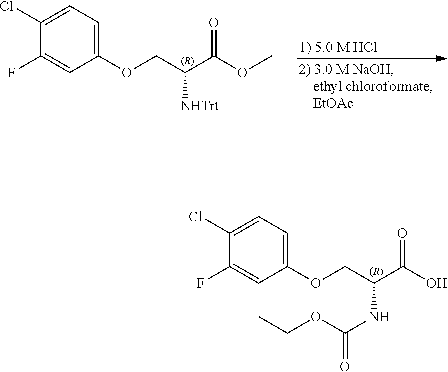

### Step 3: O-(4-chloro-3-fluorophenyl)-N-(ethoxycarbonyl)-D-serine

To methyl O-(4-chloro-3-fluorophenyl)-N-trityl-D-serinate (17.3 g, 17.6 mmol) was added methanol (50 mL) followed by 5.0 M hydrochloric acid (150 mL). The resulting mixture was heated at 80° C. for 16 h. The resulting suspension was cooled to room temperature and most of the methanol was evaporated under reduced pressure. The remaining aqueous phase was diluted with water (100 mL) and ethyl acetate (150 mL). The layers were shaken and separated, and the aqueous phase was washed with ethyl acetate (1×150 mL). The organic phases were discarded, and the aqueous phase was basified with 3.0 M NaOH (200 mL). The resulting mixture was stirred at room temperature for 30 min. The aqueous phase was concentrated to 200 mL under reduced pressure. Ethyl acetate (200 mL) was added, and the biphasic mixture was stirred vigorously while ethyl chloroformate (6.7 mL, 70.4 mmol) was added using a syringe. The mixture was stirred at room temperature for 30 min and the layers were separated. The pH of the aqueous phase was adjusted to 3 using 2.0 M HCl and extracted with additional ethyl acetate (2×150 mL). The organic phases were combined and dried over sodium sulfate. Concentration under reduced pressure provided a viscous oil which was partitioned between saturated aqueous sodium bicarbonate (200 mL) and ethyl acetate (200 mL). The layers were separated, and the aqueous phase was acidified using formic acid. The aqueous phase was extracted with ethyl acetate (2×150 mL). The organic extracts were combined, dried over sodium sulfate and concentrated to give 2.08 g (39%) of O-(4-chloro-3-fluorophenyl)-N-(ethoxycarbonyl)-D-serine as a white amorphous solid. ¹H NMR (400 MHz, DMSO-d₆) δ 12.94 (s, 1H), 7.60 (d, J=8.2 Hz, 1H), 7.47 (t, J=8.9 Hz, 1H), 7.08 (dd, J=11.4, 2.8 Hz, 1H), 6.84 (dd, J=8.9, 2.8 Hz, 1H), 4.40 (m, 1H), 4.23 (m, 2H), 4.01 (q, J=7.1 Hz, 2H), 1.16 (t, J=7.1 Hz, 3H). LC/MS (APCI) m/z calcd. for C₁₂H₁₄ClFNO₅ [M+H]⁺: 306.1; 306.1 found.

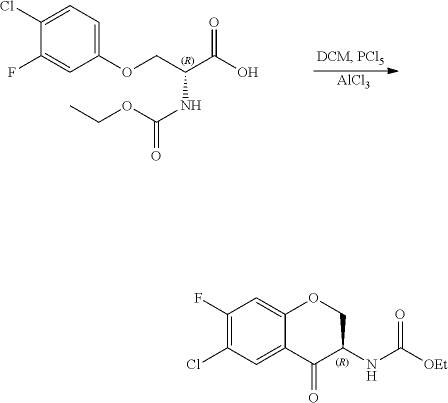

### Step 4: Ethyl (R)-(6-chloro-7-fluoro-4-oxochroman-3-yl)carbamate

O-(4-chloro-3-fluorophenyl)-N-(ethoxycarbonyl)-D-serine (2.0 g, 6.7 mmol) was dissolved in dichloromethane (30 mL) and cooled to 0° C. with an ice bath under an atmosphere of nitrogen. Phosphorous pentachloride (1.67 g, 8.02 mmol) was added and the resulting mixture was stirred at 0° C. for 30 min. The ice bath was removed, and the reaction was warmed to room temperature. Aluminum trichloride (3.56 g. 26.7 mmol) was added and the resulting mixture was stirred at room temperature for 1 h. The reaction mixture was added dropwise to 2.0 M HCl (50 mL) at 0° C. and stirred at 0° C. for 15 min and then diluted with additional dichloromethane (50 mL). The layers were separated, and the aqueous phase was extracted with additional dichloromethane (1×50 mL). The organic extracts were combined and washed with saturated aqueous sodium bicarbonate (75 mL), brine, dried over sodium sulfate and concentrated to a crude solid which was purified with silica gel using 15% ethyl acetate/hexanes, providing 1.2 g (62%) of ethyl (R)-(6-chloro-7-fluoro-4-oxochroman-3-yl)carbamate as a white solid. ¹H NMR (400 MHz, DMSO-d₆) δ 7.88 (d, J=8.5 Hz, 1H), 7.53 (d, J=8.1 Hz, 1H), 7.28 (d, J=10.4 Hz, 1H), 4.69 (ddd, J=12.8, 8.1, 5.9 Hz, 1H), 4.57 (dd, J=10.8, 5.9 Hz, 1H), 4.35 (dd, J=12.8, 10.8 Hz, 1H), 4.03 (q, J=7.1 Hz, 2H), 1.19 (t, J=7.1 Hz, 3H). LC/MS (APCI) m/z calcd. for C₁₂H₁₂ClFNO₄⁺ [M+H]⁺: 288.0; 288.0 found.

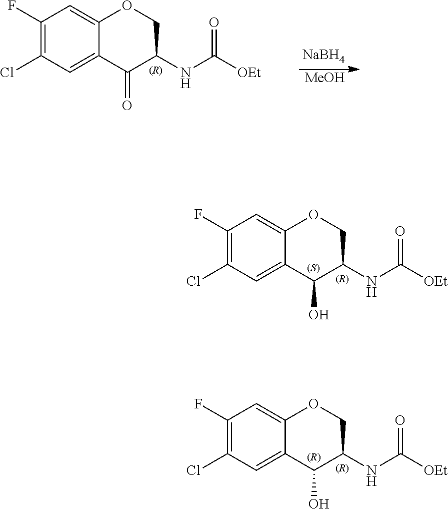

### Step 5: Ethyl ((3R,4S)-6-chloro-7-fluoro-4-hydroxychroman-3-yl)carbamate and ethyl ((3R,4R)-6-chloro-7-fluoro-4-hydroxychroman-3-yl)carbamate

Ethyl (R)-(6-chloro-7-fluoro-4-oxochroman-3-yl)carbamate (1.2 g, 4.1 mmol) was dissolved in methanol (10 mL) and cooled to 0° C. using an ice bath. Sodium borohydride (0.46 g, 12.2 mmol) was added portion wise, the ice bath was removed, and the resulting mixture was stirred at room temperature for 30 min. It was diluted with ethyl acetate (100 mL) and washed with saturated aqueous sodium bicarbonate (100 mL). The organic phase was washed with brine, dried over sodium sulfate and concentrated to a white amorphous solid which was purified with reverse phase HPLC using a 40 min gradient from 10-100% acetonitrile/water with 0.1% formic acid (Phenomenex Gemini 5 micron C18 column, 150×21 mm, Axia Pack) providing:

Ethyl ((3R,4S)-6-chloro-7-fluoro-4-hydroxychroman-3-yl)carbamate (Intermediate 14.1) as a white solid (168 mg, 14%). ¹H NMR (400 MHz, DMSO-d₆) δ 7.44 (d, J=8.5 Hz, 1H), 6.91 (d, J=10.8 Hz, 1H), 6.84 (d, J=7.3 Hz, 1H), 5.72 (d, J=5.3 Hz, 1H), 4.63 (t, J=4.3 Hz, 1H), 4.15-3.97 (m, 4H), 3.90-3.79 (m, 1H), 1.17 (t, J=7.1 Hz, 3H). LC/MS (APCI) m/z calcd. for C₁₂H₁₄ClFNO₄⁺ [M+H]⁺: 290.1; 272.1 found (M+H—H₂O).

Ethyl ((3R,4R)-6-chloro-7-fluoro-4-hydroxychroman-3-yl)carbamate (Intermediate 14.2) as a white solid (832 mg, 71%). ¹H NMR (400 MHz, DMSO-d₆) δ 7.46 (d, J=8.5 Hz, 1H), 7.25 (d, J=7.0 Hz, 1H), 6.90 (d, J=10.7 Hz, 1H), 5.84 (d, J=5.7 Hz, 1H), 4.44 (t, J=5.2 Hz, 1H), 4.17 (dd, J=11.1, 3.1 Hz, 1H), 4.01 (dq, J=18.1, 7.1, 6.7 Hz, 3H), 3.68 (qd, J=6.1, 2.9 Hz, 1H), 1.15 (t, J=7.1 Hz, 3H). LC/MS (APCI) m/z calcd. for C₁₂H₁₄ClFNO₄⁺ [M+H]⁺: 290.1; 272.1 found (M+H—H₂O).

TABLE I-14
The following intermediates were prepared according to synthetic
procedures as described in Intermediate Synthesis 14.

| Inter- | Starting | |
|---|---|---|
| mediate | Material | Structure, Name and Data |
| 14.1 | 4-chloro-3- fluorophenol | 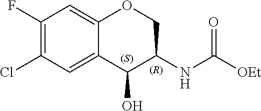 Ethyl ((3R,4S)-6-chloro-7-fluoro-4- hydroxychroman-3-yl)carbamate. ¹H NMR (400 MHz, DMSO-d₆) δ 7.44 (d, J = 8.5 Hz, 1H), 6.91 (d, J = 10.8 Hz, 1H), 6.84 (d, J = 7.3 Hz, 1H), 5.72 (d, J = 5.3 Hz, 1H), 4.63 (t, J = 4.3 Hz, 1H), 4.15-3.97 (m, 4H), 3.90-3.79 (m, 1H), 1.17 (t, J = 7.1 Hz, 3H). LC/MS (APCI) m/z calcd. for C₁₂H₁₄ClFNO₄⁺ [M + H]⁺: 290.1; 272.1 found (M + H − H₂O). |
| 14.2 | 4-chloro-3- fluorophenol | 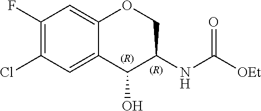 Ethyl ((3R,4R)-6-chloro-7-fluoro-4- hydroxychroman-3-yl)carbamate. ¹H NMR (400 MHz, DMSO-d₆) δ 7.46 (d, J = 8.5 Hz, 1H), 7.25 (d, J = 7.0 Hz, 1H), 6.90 (d, J = 10.7 Hz, 1H), 5.84 (d, J = 5.7 Hz, 1H), 4.44 (t, J = 5.2 Hz, 1H), 4.17 (dd, J = 11.1, 3.1 Hz, 1H), 4.01 (dq, J = 18.1, 7.1, 6.7 Hz, 3H), 3.68 (qd, J = 6.1, 2.9 Hz, 1H), 1.15 (t, J = 7.1 Hz, 3H). LC/MS (APCI) m/z calcd. for C₁₂H₁₄ClFNO₄⁺ [M + H]⁺: 290.1; 272.1 found (M + H − H₂O). |

### Example I-15. Intermediate Synthesis 15

### Preparation of Methyl (6-chloro-7-fluorochroman-3-yl)carbamate (Intermediate 15)

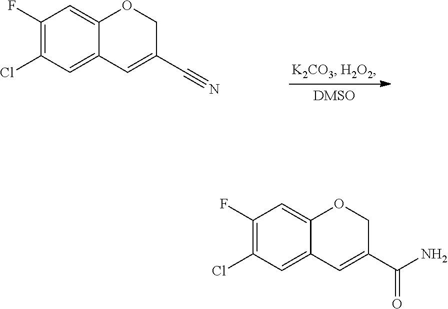 [SMILES: N#CC1=Cc2cc(Cl)c(F)cc2OC1 ; NC(=O)C1=Cc2cc(Cl)c(F)cc2OC1]

### Step 1: 6-Chloro-7-fluoro-2H-chromene-3-carboxamide

Hydrogen peroxide (4.1 mL, 47.7 mmol, 5 equiv., 35% in water) was added dropwise to a stirring solution of 6-chloro-7-fluoro-2H-chromene-3-carbonitrile (2 g, 9.54 mmol, 1 equiv.) and K₂CO₃ (2.66 g, 19.1 mmol, 2 equiv.) in DMSO (20 mL) at 0° C. before being allowed to return to rt over 3 h. The reaction was then diluted with water (200 mL), extracted with EtOAc (200 mL), the organic layer washed with water (2×150 mL) and brine (150 mL), before being dried over sodium sulfate, filtered, and solvent removed by rotary evaporation to give the product as a yellow solid (2.1 g, 97%). 1H NMR (400 MHz, DMSO-d6) δ 7.71 (s, 1H), 7.45 (d, J=8.4 Hz, 1H), 7.32 (s, 1H), 7.22 (s, 1H), 7.01 (d, J=10.4 Hz, 1H), 4.94 (d, J=1.3 Hz, 2H). LC/MS (APCI) m/z calcd. for C₁₀H₈ClFNO₂⁺ [M+H]⁺: 228.0; 228.0 found.

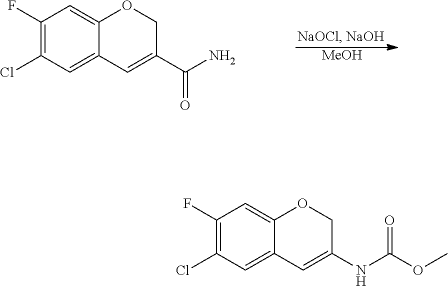 [SMILES: "MeOH" ; NC(=O)C1=Cc2cc(Cl)c(F)cc2OC1 ; COC(=O)NC1=Cc2cc(Cl)c(F)cc2OC1]

### Step 2: Methyl (6-chloro-7-fluoro-2H-chromen-3-yl)carbamate

NaOCl (14 mL, 10 mmol, 1.1 equiv, 5% in water) was added portion wise to a stirring solution of 6-chloro-7-fluoro-2H-chromene-3-carboxamide (2.11 g, 9.27 mmol, 1 equiv.) in MeOH (25 mL) at 0° C. After 30 min, NaOH (3 M, 5.9 mL, 17.6 mmol, 1.9 equiv) was added dropwise and the resulting solution warmed to rt and stirred for 14 h. The solution was made acidic (pH 4) using HCl (1 M) before being extracted with EtOAc (2×100 mL), organics combined, washed with brine, dried over sodium sulfate, filtered, and solvent removed by rotary evaporation. The product was isolated by silica chromatography (0->15% EtOAc/Hex) as a tan solid (1.7 g, 71%). ¹H NMR (400 MHz, DMSO-d₆) δ 9.42 (s, 1H), 7.25 (d, J=8.4 Hz, 1H), 6.88 (d, J=10.3 Hz, 1H), 6.56 (s, 1H), 4.76 (d, J=1.1 Hz, 2H), 3.65 (s, 3H). LC/MS (APCI) m/z calcd. for C₁₁H₁₀ClFNO₃⁺ [M+H]⁺: 258.0; 258.0 found.

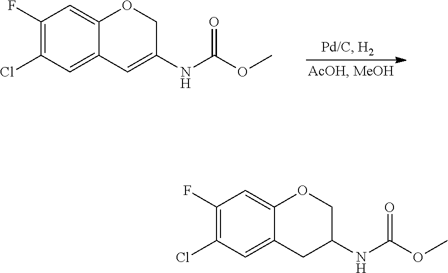 [SMILES: COC(=O)NC1=Cc2cc(Cl)c(F)cc2OC1 ; COC(=O)NC1COc2cc(F)c(Cl)cc2C1]

### Step 3: Methyl (6-chloro-7-fluorochroman-3-yl)carbamate (Intermediate 15)

Methyl (6-chloro-7-fluoro-2H-chromen-3-yl)carbamate (0.5 g, 1.941 mmol, 1 equiv.) and Pd/C (50 mg, 0.047 mmol, 0.025 equiv. 10% Pd by mass) were suspended in MeOH/AcOH (25 mL/0.1 mL) before being stirred under H₂ (balloon) at rt. After 7 h, the reaction was filtered through a pad of celite, solvent removed by rotary evaporation, and dried under high vacuum to give the desired product (Intermediate 15) as a tan solid (0.5 g, 99.8%). ¹H NMR (400 MHz, DMSO-d₆) δ 7.38 (d, J=5.0 Hz, 1H), 7.32 (d, J=8.5 Hz, 1H), 6.88 (d, J=10.7 Hz, 1H), 4.26-4.02 (m, 1H), 3.86 (q, J=6.8, 5.7 Hz, 2H), 3.55 (s, 3H), 2.97 (dd, J=15.9, 4.1 Hz, 1H), 2.77-2.58 (m, 1H). LC/MS (APCI) m/z calcd. for C₁₁H₁₂ClFNO₃⁺ [M+H]⁺: 260.1; 260.1 found.

### Example I-16. Intermediate Synthesis 16

### Preparation of Methyl (S)-(6,7-difluorochroman-3-yl)carbamate (Intermediate 16)

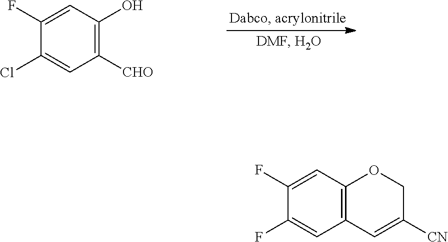 [SMILES: O=Cc1cc(Cl)c(F)cc1O ; N#CC1=Cc2cc(F)c(F)cc2OC1]

### Step 1: 6,7-Difluoro-2H-chromene-3-carbonitrile

DABCO (18.7 g, 167 mmol, 0.33 equiv) was added to a stirring solution of aldehyde (80 g, 506 mmol, 1 equiv.) and acrylonitrile (215 g, 4.05 mol, 8 equiv.) in DMF (215 mL) and water (160 mL) at rt before being heated to 80° C. for 16 h. The reaction was then cooled to 0° C., diluted with water (600 mL) and stirred for 3 h. The resulting precipitate was then filtered, washed with MeOH:H₂O (200 mL, 2:1), and dried under high vacuum to give the desired product as a pale yellow crystalline solid (76 g, 78%). ¹H NMR (400 MHz, Methylene Chloride-d₂) δ 7.11 (d, J=1.7 Hz, 1H), 6.99 (dd, J=9.9, 8.6 Hz, 1H), 6.74 (dd, J=10.9, 6.7 Hz, 1H), 4.86-4.74 (m, 2H).

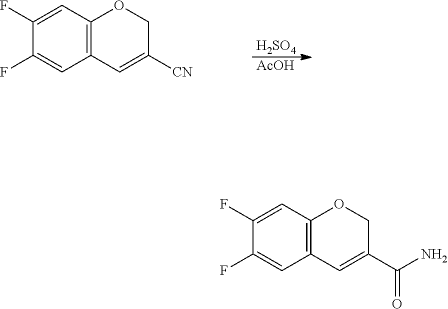 [SMILES: "AcOH" ; N#CC1=Cc2cc(F)c(F)cc2OC1 ; "H2SO4" ; NC(=O)C1=Cc2cc(F)c(F)cc2OC1]

### Step 2: 6,7-Difluoro-2H-chromene-3-carboxamide

Concentrated sulphuric acid (22 g) was added to a stirring solution of 6,7-difluoro-2H-chromene-3-carbonitrile (55 g, 284 mmol, 1 equiv.) in acetic acid (160 mL) before being heated to 100° C. for 1 h. The reaction was then cooled to rt before water:isopropanol (100 mL, 2:1) was added slowly over 20 min. The reaction was then cooled to 0° C. for 2 h, the resulting precipitate filtered, washed with cold water:isopropanol (2×20 mL, 2:1), and dried under high vacuum to give the desired product as a microcrystalline solid (51 g, 85%). ¹H NMR (400 MHz, DMSO-d₆) δ 7.79 (s, 1H), 7.47-7.31 (m, 2H), 7.27 (s, 1H), 7.08 (dd, J=11.7, 7.0 Hz, 1H), 4.96 (s, 2H).

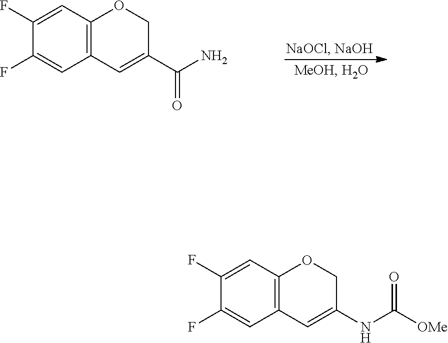 [SMILES: NC(=O)C1=Cc2cc(F)c(F)cc2OC1 ; COC(=O)NC1=Cc2cc(F)c(F)cc2OC1]

### Step 3: Methyl (6,7-difluoro-2H-chromen-3-yl)carbamate

Taking care to maintain a temperature below 0° C., aqueous sodium hypochlorite (170 mL, 341 mmol, 1.5 equiv. ˜15% w/w solution) was added to added slowly to a stirring solution of 6,7-difluoro-2H-chromene-3-carboxamide (48 g, 227 mmol, 1 equiv.) in MeOH (150 mL) at 0° C. After 30 min, NaOH (1.5 M, 200 mL) was added slowly to keep internal temperatures below 10° C. The reaction was then allowed to return to rt over 20 h before being cooled to 0° C. and HCl (1.5 M, 200 mL) was added and the resulting suspension stirred for 1 h. The precipitate was filtered, washed with MeOH:H₂O (2×100 mL, 1:1), and dried under high vacuum to give the desired product as a white microcrystalline solid (39 g, 71%). ¹H NMR (400 MHz, DMSO-d₆) δ 9.41 (s, 1H), 7.16 (dd, J=11.3, 9.1 Hz, 1H), 6.89 (dd, J=11.6, 7.1 Hz, 1H), 6.55 (s, 1H), 4.71 (s, 2H), 3.65 (s, 3H).

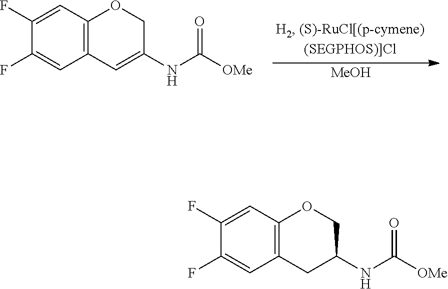 [SMILES: "MeOH" ; COC(=O)NC1=Cc2cc(F)c(F)cc2OC1 ; COC(=O)N[C@@H]1COc2cc(F)c(F)cc2C1]

### Step 4: Methyl (S)-(6,7-difluorochroman-3-yl)carbamate (Intermediate 16)

Methyl (6,7-difluoro-2H-chromen-3-yl)carbamate was dissolved in MeOH (800 mL) and resulting solution purged with N₂ for 10 min. (S)—RuCl[(p-cymene)(SEGPHOS®)]Cl was then added and the reaction stirred at 60° C. under H2 (80 psi) for 36 h. The reaction was then concentrated to −200 mL and allowed to sit undisturbed for 4 h at rt. The resulting crystals were filtered and recrystallized from MeOH twice more before being dried under high vacuum to give the desired product (Intermediate 16, 32 g, 62%, >99% ee). ¹H NMR (400 MHz, DMSO-d₆) δ 7.37 (d, J=5.4 Hz, 1H), 7.19 (dd, J=11.2, 9.3 Hz, 1H), 6.87 (dd, J=12.0, 7.2 Hz, 1H), 4.12 (d, J=9.0 Hz, 1H), 3.82 (td, J=12.7, 10.6, 4.9 Hz, 2H), 3.55 (s, 3H), 2.94 (dd, J=16.7, 4.8 Hz, 1H), 2.67 (dd, J=16.3, 7.5 Hz, 1H).

### Example I-17. Intermediate Synthesis 17

### Preparation of Methyl ((1aR,7bS)-6-chloro-5-fluoro-1,7b-dihydrocyclopropa[c]chromen-1a(2H)-yl)carbamate (Intermediate 17)

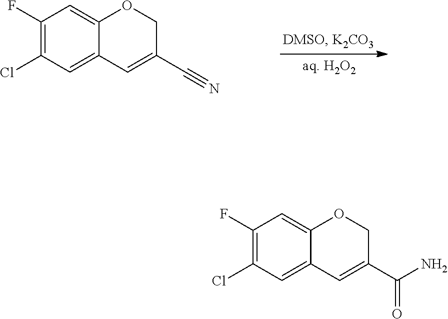 [SMILES: N#CC1=Cc2cc(Cl)c(F)cc2OC1 ; NC(=O)C1=Cc2cc(Cl)c(F)cc2OC1]

### Step 1: 6-chloro-7-fluoro-2H-chromene-3-carboxamide

6-chloro-7-fluoro-2H-chromene-3-carbonitrile (1.5 g, 7.2 mmol) was dissolved in DMSO (20 mL) and cooled to 0° C. using an ice bath. 35% Aq. H₂O₂ (3.1 mL, 35.8 mmol) was added with a syringe and the resulting mixture was stirred for 3 h., during which time it was warmed to r.t. The mixture was diluted with EtOAc (200 mL) and water (200 mL). The organic phase was washed with water (3×100 mL), saturated aq. NaCl (100 mL), dried over sodium sulfate and concentrated under reduced pressure providing 6-chloro-7-fluoro-2H-chromene-3-carboxamide (1.6 g, 6.9 mmol, 97% yield) as a yellow solid which was used in the subsequent step without additional purification. LC/MS (APCI) m/z calcd. for C₁₀H₇NO₂FCl [M+H]+: 227.0; 228.0 found.

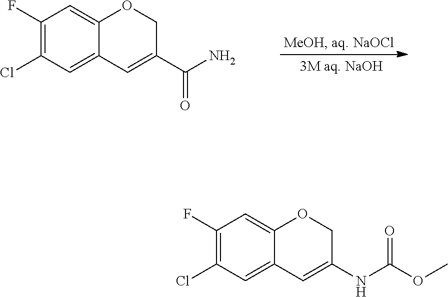 [SMILES: NC(=O)C1=Cc2cc(Cl)c(F)cc2OC1 ; COC(=O)NC1=Cc2cc(Cl)c(F)cc2OC1]

### Step 2: Methyl (6-chloro-7-fluoro-2H-chromen-3-yl)carbamate

6-chloro-7-fluoro-2H-chromene-3-carboxamide (1.6 g, 6.9 mmol) was suspended in MeOH (25 mL) and cooled to 0° C. using an ice bath. 5% aq. NaOCl (10.5 mL, 7.6 mmol) was added portionwise with a syringe and the resulting mixture was stirred at 0° C. for 30 min. 3 M aq. NaOH (4.4 mL, 13.1 mmol) was added dropwise at 0° and the reaction was warmed to r.t. and stirred at r.t. for 18 h. The pH of the reaction mixture was adjusted to 4 using 1 M aq. HCl and extracted with EtOAc (2×100 mL). The organic extracts were combined, washed with saturated aq. NaCl (100 mL), dried over sodium sulfate and concentrated in vacuo. The product was purified with silica gel using 15% EtOAc/Hex providing methyl (6-chloro-7-fluoro-2H-chromen-3-yl)carbamate (1.3 g, 5.0 mmol, 72% yield) as a tan solid. ¹H NMR (400 MHz, DMSO-d₆) δ 9.42 (s, 1H), 7.25 (d, J=8.4 Hz, 1H), 6.88 (d, J=10.3 Hz, 1H), 6.56 (s, 1H), 4.76 (d, J=0.8 Hz, 2H). LC/MS (APCI) m/z calcd. for C₁₁H₉NO₃FCl [M+H]⁺: 257.0; 258.1 found.

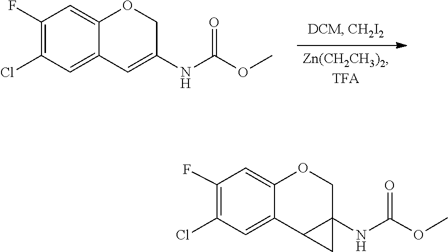 [SMILES: COC(=O)NC1=Cc2cc(Cl)c(F)cc2OC1 ; COC(=O)NC12COc3cc(F)c(Cl)cc3C1C2]

### Step 3. Methyl (6-chloro-5-fluoro-1,7b-dihydrocyclopropa[c]chromen-1a(2H)-yl)carbamate (Intermediate 17)

To anhydrous DCM (10 mL) under an atmosphere of nitrogen was added diethyl zinc (14.6 mL of 1.0 M in hexanes, 14.6 mmol). The resulting mixture was cooled to 0° C. using an ice bath and TFA (1.1 mL, 14.6 mmol) in DCM (6 mL) was added dropwise using a syringe (gas evolution.) The resulting mixture was stirred at 0° C. for 20 min. CH₂I₂ (3.9 g, 14.6 mmol, 3.0 eq.) in DCM (6 mL) was added, and the resulting mixture was stirred vigorously at 0° C. for 20 min. Methyl (6-chloro-7-fluoro-2H-chromen-3-yl)carbamate (1.3 g, 4.9 mmol) in DCM (6 mL) was added using a syringe and the ice bath was removed. The resulting mixture was stirred at r.t. for 1 h. The reaction was quenched with 0.1 M aq. HCl (40 mL) and additional DCM (20 mL) was added. The layers were shaken and separated, and the aqueous phase was extracted with additional DCM (2×25 mL). The organic phases were combined, washed with saturated aq. NaCl, dried over sodium sulfate and concentrated under reduced pressure. The product was purified with silica gel using 70% DCM/Hex providing methyl ((1aR,7bS)-6-chloro-5-fluoro-1,7b-dihydrocyclopropa[c]chromen-la(2H)-yl)carbamate (721 mg, 2.65 mmol, 55% yield) as a white solid. ¹H NMR (400 MHz, Methanol-d₄) δ 7.32 (d, J=8.1 Hz, 1H), 6.69 (d, J=10.1 Hz, 1H), 4.30 (d, J=10.0 Hz, 1H), 3.79 (d, J=10.0 Hz, 1H), 3.63 (s, 3H), 2.18 (dd, J=9.2, 5.6 Hz, 1H), 1.48-1.16 (m, 2H). LC/MS (APCI) m/z calcd. for C₁₂H₁₁NO₃FCl [M+H]⁺: 271.0; 272.1 found.

### Example I-18. Intermediate Synthesis 18

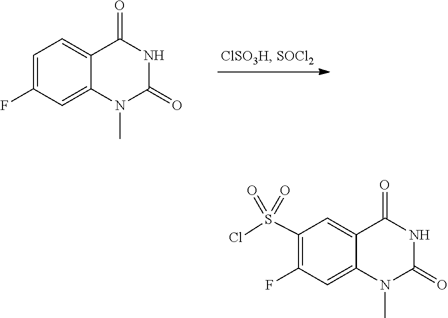 [SMILES: Cn1c(=O)[nH]c(=O)c2ccc(F)cc21 ; Cn1c(=O)[nH]c(=O)c2cc(S(=O)(=O)Cl)c(F)cc21]

### Preparation of 7-Fluoro-1-methyl-2,4-dioxo-1,2,3,4-tetrahydroquinazoline-6-sulfonyl chloride (Intermediate 18.1)

7-Fluoro-1-methylquinazoline-2,4(1H,3H)-dione (500 mg, 2.58 mmol, 1 equiv.) was suspended in chlorosulfonic acid (2 mL, 30 mmol, 12 equiv.) at 0° C. before being warmed to rt. After 10 min, thionyl chloride (1 mL, 13.8 mmol, 5.4 equiv.) was added and the reaction heated to 90° C. for 3 h. The reaction was then cooled to rt, poured into water at 0° C., filtered, and washed with Et₂O before being dried under high vacuum to give the desired product (444 mg, 59%). ¹H NMR (400 MHz, DMSO-d₆) δ 11.52 (s, 1H), 8.17 (d, J=7.9 Hz, 1H), 7.17 (d, J=11.6 Hz, 1H), 3.32 (s, 3H).

TABLE I-18
The following intermediates were prepared according to synthetic
procedures as described in Intermediate Synthesis 18.

| | Intermediate or | |
|---|---|---|
| Intermediate | Starting Material | Structure, Name and Data |
| 18.1 | 7-Fluoro-1- methylquinazoline- 2,4(1H,3H)-dione | 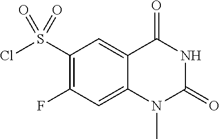 [SMILES: Cn1c(=O)[nH]c(=O)c2cc(S(=O)(=O)Cl)c(F)cc21] 7-Fluoro-1-methyl-2,4-dioxo-1,2,3,4- tetrahydroquinazoline-6-sulfonyl chloride. ¹H NMR (400 MHz, DMSO-d₆) δ 11.52 (s, 1H), 8.17 (d, J = 7.9 Hz, 1H), 7.17 (d, J = 11.6 Hz, 1H), 3.32 (s, 3H). |
| 18.2 | Ethyl 2-methyl- 1H-pyrrole-3- carboxylate | 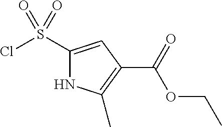 Ethyl 5-(chlorosulfonyl)-2-methyl-1H-pyrrole-3- carboxylate. ¹H NMR (400 MHz, DMSO-d₆) δ 11.46 (s, 1H), 6.94 (s, 1H), 4.13 (q, J = 7.1 Hz, 2H), 2.35 (s, 3H), 1.24 (t, J = 7.1 Hz, 3H). |
| 18.3 | 20 | 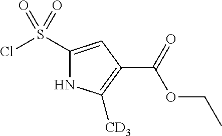 [SMILES: [2H]C([2H])([2H])c1[nH]c(S(=O)(=O)Cl)cc1C(=O)OCC] Ethyl 5-(chlorosulfonyl-2-trideuteromethyl-1H- pyrrole-3-carboxylate: ¹H NMR (500 MHz, CHLOROFORM-d) δ ppm 9.08 (1 H, br s) 7.47 (1 H, d, J = 2.72 Hz) 4.33 (2 H, q, J = 7.05 Hz) 1.38 (3 H, t, J = 7.14 Hz); ESI (NEG) m/z: 253.2 (M − H)⁺. |

### Example 1-19. Intermediate Synthesis 19

### Preparation of methyl-4-oxo-3,4-dihydroquinazoline-6-sulfonyl chloride (Intermediate 19.1)

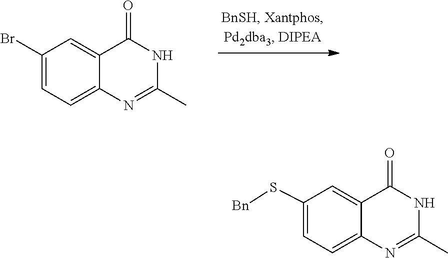 [SMILES: Cc1nc2ccc(Br)cc2c(=O)[nH]1 ; Cc1nc2ccc(SCc3ccccc3)cc2c(=O)[nH]1]

### Step 1: 6-(Benzylthio)-2-methylquinazolin-4(3H)-one

6-Bromo-2-methylquinazolin-4(3H)-one (20 g, 83.7 mmol, 1 equiv.), phenylmethanethiol (10.9 g, 87.8 mmol, 1.05 equiv.), xantphos (4 g, 192 mmol, 0.083 equiv.), Pd₂dba₃ (3.83 g, 83.7 mmol, 0.05 equiv.), and diisopropylethylamine (33.5 mL, 192.4 mmol, 2.3 equiv.) were suspended in dioxanes (165 mL) and toluene (165 mL) before being heated to 100° C. for 5 h. The reaction was cooled to rt, poured into EtOAc (1 L) and MeOH (˜200 mL) before being filtered to remove remaining solids. The organic layer was then washed with water (500 mL) and brine (500 mL) before being concentrated by rotary evaporation. The resultant semisolid was triturated with MeOH, filtered, washed with MeOH, and dried under vacuum. The crude material was dissolved in MeCN before solvent was removed by rotary evaporation and crude product dried under high vacuum. This material was used directly without further purification. ¹H NMR (400 MHz, DMSO-d₆) δ 12.23 (s, 1H), 7.92 (d, J=2.2 Hz, 1H), 7.71 (dd, J=8.6, 2.3 Hz, 1H), 7.49 (d, J=8.5 Hz, 1H), 7.40-7.34 (m, 2H), 7.30 (ddd, J=7.6, 6.7, 1.4 Hz, 2H), 7.26-7.18 (m, 1H), 4.36-4.26 (m, 2H), 2.38-2.24 (m, 3H). LC/MS (APCI) m/z calcd. for C₁₆H₁₅N₂OS⁺ [M+H]⁺: 283.1; 283.1 found.

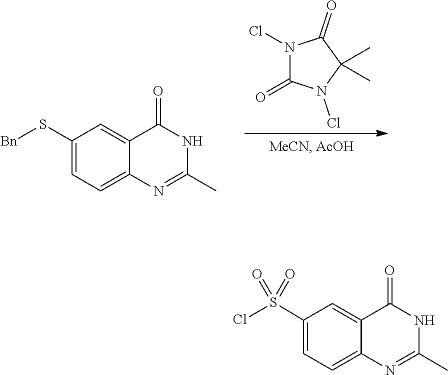 [SMILES: Cc1nc2ccc(SCc3ccccc3)cc2c(=O)[nH]1 ; CC1(C)C(=O)N(Cl)C(=O)N1Cl ; Cc1nc2ccc(S(=O)(=O)Cl)cc2c(=O)[nH]1]

### Step 2: Methyl-4-oxo-3,4-dihydroquinazoline-6-sulfonyl chloride (Intermediate 19.1)

6-(Benzylthio)-2-methylquinazolin-4(3H)-one (15 g, 53 mmol, 1 equiv.) and 1,3-dichloro-5,5-dimethylimidazolidine-2,4-dione (26.2 g, 133 mmol, 2.5 equiv.) were suspended in AcOH (16 mL), water (11 mL), and MeCN (430 mL) at rt before being cooled to 0° C. After 1 h, the product begins to precipitate, which was filtered, washed with a 25% EtOAc in hexanes solution, and dried under high vacuum to give the desired product as a white solid (11.5 g, 84%). ¹H NMR (400 MHz, DMSO-d₆) δ 14.51 (s, 1H), 8.24 (d, J=1.9 Hz, 1H), 8.06 (dd, J=8.4, 1.9 Hz, 1H), 7.67 (d, J=8.5 Hz, 1H), 2.55 (s, 3H).

TABLE I-19
The following intermediates were prepared according to synthetic
procedures as described in Intermediate Synthesis 19.

| | Intermediate or | |
|---|---|---|
| Intermediate | Starting material | Structure, Name and Data |
| 19.1 | 6-bromo-2- methylquinazolin- 4(3H)-one | 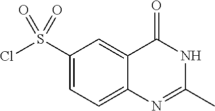 [SMILES: Cc1nc2ccc(S(=O)(=O)Cl)cc2c(=O)[nH]1] 2-Methyl-4-oxo-3,4-dihydroquinazoline-6-sulfonyl chloride. ¹H NMR (400 MHz, DMSO-d₆) δ 14.51 (s, 1H), 8.24 (d, J = 1.9 Hz, 1H), 8.06 (dd, J = 8.4, 1.9 Hz, 1H), 7.67 (d, J = 8.5 Hz, 1H), 2.55 (s, 3H). |
| 19.2 | 6-bromo-7-fluoro- 2- methylquinazolin- 4(3H)-one | 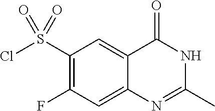 [SMILES: Cc1nc2cc(F)c(S(=O)(=O)Cl)cc2c(=O)[nH]1] 7-Fluoro-2-methyl-4-oxo-3,4-dihydroquinazoline-6- sulfonyl chloride. LC/MS (APCI) m/z calcd. for C9H7ClFN2O3S⁺ [M + H]⁺: 276.0; 276.0 found. |
| 19.3 | 22 | 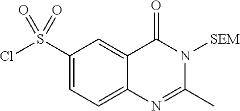 2-Methyl-4-oxo-3-((2-(trimethylsilyl)ethoxy)methyl)- 3,4-dihydroquinazoline-6-sulfonyl chloride. Aliquot quenched with 2-methoxyethan-1-amine for mass analysis: LC/MS (APCI) m/z calcd. for C₁₈H₂₉N₃O₅SSi⁺ [M + H]⁺: 428.2; 428.1 found. |

### Example 1-20. Intermediate Synthesis 20

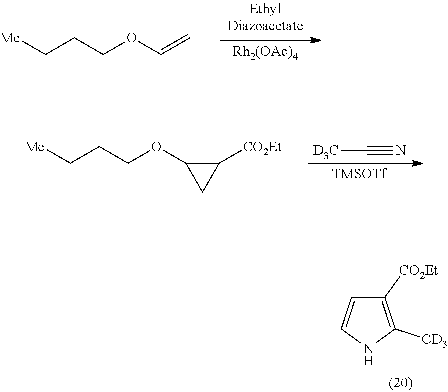

### Preparation of methyl-4-oxo-3,4-dihydroquinazoline-6-sulfonyl chloride (Intermediate 20)

### Step 1: Ethyl 2-butoxycyclopropanecarboxylate

A 2-neck 24/40-250 mL round bottom flasked was charged butyl vinyl ether (12.82 mL, 100.0 mmol) and diluted with diethyl ether (50 mL). To that solution was added rhodium(II) acetate dimer (0.44 g, 1.0 mmol). Ethyl diazoacetate (10.3 mL, 100.0 mmol) in diethyl ether (30 mL) was added via syringe pump at a rate of 0.67 mL/min placing the tip of the needle below the solvent line. After the first 10 mL of the ethyl diazoacetate was added, the addition rate was halved for the remaining time. Once complete, the mixture was filtered through a plug of celite, concentrated under reduced pressure and purified by flash chromatography using a 125-g Redi-Sep, eluting with 0-25% EtOAc/heptane to provide ethyl 2-butoxycyclopropanecarboxylate (10.0 g, 53.7 mmol, 53.8% yield) as clear liquid: ESI (POS) m/z: 187.4 (M+H)⁺.

### Step 2: Ethyl 2-trideuteromethyl-1H-pyrrole-3-carboxylate (Intermediate 20)

A 24/40-100 mL round bottom flask was charged with ethyl 2-butoxycyclopropanecarboxylate (2.0 g, 10.7 mmol) and diluted with nitromethane (21.5 ml). To that solution was added Acetonitrile-d3 (5.6 mL, 107.0 mmol) and the solution was cooled to −40° C. TMSOTf (1.9 mL, 10.7 mmol) was then added dropwise, turning the clear solution orange. After 2.5 h, the reaction mixture was allowed to warm to 22° C. and stir for 1 h. The reaction mixture was quenched with a saturated aqueous sodium bicarbonate solution. The aqueous was extracted with DCM, and the combined organic layers were washed with water and brine, dried with magnesium sulfate, and concentrated under pressure. The crude product was purified by flash chromatography using a 100 g Biotage SNAP Ultra column, eluting with 0-20% EtOAc in heptane to provide ethyl 2-trideuteromethyl-1H-pyrrole-3-carboxylate (510.0 mg, 3.3 mmol, 30.4% yield) as vicious clear oil which solidified upon drying: ¹H NMR (500 MHz, CHLOROFORM-d) δ ppm 8.09 (1H, br s) 6.56 (2H, s) 4.26 (2H, q, J=7.14 Hz) 1.33 (3H, t, J=7.14 Hz); ESI (POS) m/z: 157.4 (M+H)⁺.

### Example I-21. Intermediate Synthesis 21

### Preparation of 7-Fluoro-1-methylquinazoline-2,4(1H,3H)-dione (Intermediate 21)

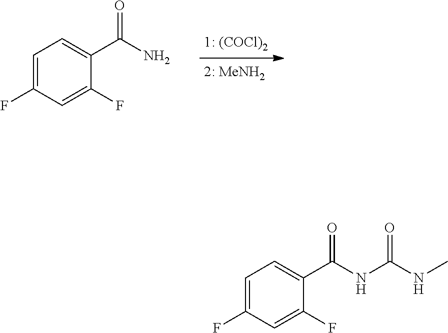

### Step 1: 2,4-Difluoro-N-(methylcarbamoyl)benzamide

Oxalyl chloride (29.5 mL, 344 mmol, 1.35 equiv.) was added slowly to a stirring solution of 2,4-difluorobenzamide (40 g, 255 mmol, 1 equiv.) in dichloroethane (360 mL) before being heated to 55° C. for 1 h. The reaction was cooled to rt and concentrated by rotary evaporation. The concentrated solution was added to a stirring solution of 2 M MeNH₂ in THF (250 mL, 500 mmol, 1.96 equiv.) at rt. After 22 h, the reaction was concentrated, the precipitate filtered, washed with water, and dried under vacuum to give the desired compound as a brown solid (51.2 g, 94%). ¹H NMR (400 MHz, DMSO-d₆) δ 10.71 (s, 1H), 8.69 (s, 1H), 8.26 (d, J=5.1 Hz, 1H), 7.38 (dddd, J=23.2, 11.0, 9.4, 2.5 Hz, 1H), 7.24-7.12 (m, 1H), 2.66 (d, J=4.9 Hz, 3H). LC/MS (APCI) m/z calcd. for C₉H₈F₂N₂O₂⁺ [M+H]⁺: 215.1; 215.1 found.

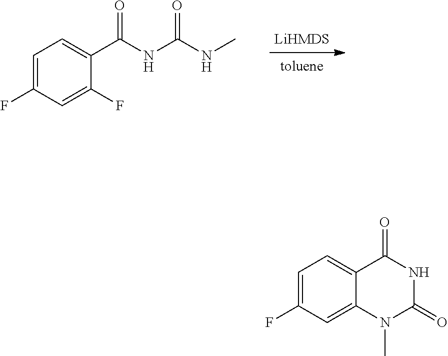

### Step 2: 7-Fluoro-1-methylquinazoline-2,4(1H,3H)-dione (Intermediate 21)

LiHMDS (690 mL, 690 mmol, 3 equiv.) was added to a solution of 2,4-difluoro-N-(methylcarbamoyl)benzamide (49.2 g, 230 mmol, 1 equiv.) in toluene (1 L) before being heated to 90° C. The reaction was then cooled to 0° C. and quenched with addition of 2 M HCl until pH reached pH 6-7. The solution was extracted with EtOAc, washed with brine, dried over sodium sulfate, filtered, and solvent removed by rotary evaporation. Trituration and drying under high vacuum yielded the desired product as a brown solid (Intermediate 21, 34.3 g, 77%). ¹H NMR (400 MHz, DMSO-d₆) δ 8.04 (dd, J=8.7, 6.5 Hz, 1H), 7.33 (dd, J=11.3, 2.3 Hz, 1H), 7.11 (td, J=8.6, 2.3 Hz, 1H), 3.41 (s, 3H), 3.33 (s, 1H). LC/MS (APCI) m/z calcd. for C₉H₈FN₂O₂⁺ [M+H]⁺: 195.1; 195.1 found.

### Example I-22. Intermediate Synthesis 22

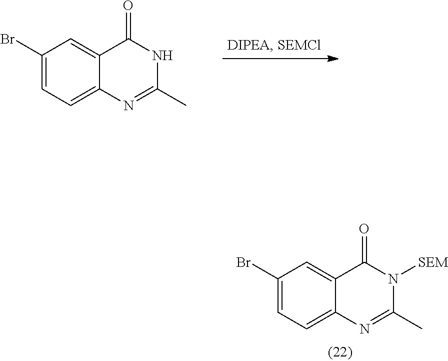 [SMILES: Cc1nc2ccc(Br)cc2c(=O)[nH]1 ; Cc1nc2ccc(Br)cc2c(=O)n1COCC[Si](C)(C)C]

### Preparation of 6-Bromo-2-methyl-3-((2-(trimethylsilyl)ethoxy)methyl)quinazolin-4(3H)-one (Intermediate 22)

SEMCl (7.5 g. 45 mmol, 1.25 equiv) was added to a stirring solution of 6-bromo-2-methylquinazolin-4(3H)-one (8 g, 36 mmol, 1 equiv.) and DIPEA (8 mL, 45 mmol, 1.25 equiv) in CH₂Cl₂ (200 mL) at rt. After 14 h, the solvent was removed by rotary evaporation and product isolated by silica chromatography (10->30% EtOAc/hexanes) as a yellow oil (Intermediate 22). LC/MS (APCI) m/z calcd. for C₁₅H₂₁BrN₂O₂Si⁺ [M+H]⁺: 369.1; 369.2 found.

### Example I-23. Intermediate Synthesis 23

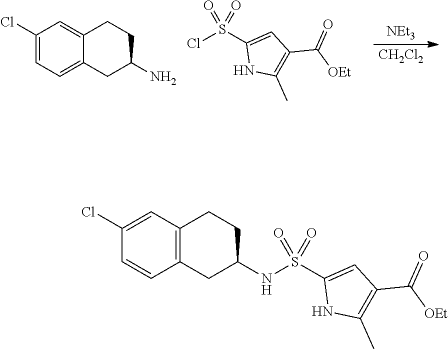

### Preparation of Ethyl (R)-5-(N-(6-chloro-1,2,3,4-tetrahydronaphthalen-2-yl)sulfamoyl)-2-methyl-1H-pyrrole-3-carboxylate (Intermediate 23.1)

Ethyl 5-(chlorosulfonyl)-2-methyl-1H-pyrrole-3-carboxylate (6.09 g, 24.2 mmol, 1.05 equiv.) was added to a stirring solution of (R)-6-chloro-1,2,3,4-tetrahydronaphthalen-2-amine (5 g, 23.1 mmol, 1 equiv.) and NEt₃ (32 mL, 231 mmol, 10 equiv.) in CH₂Cl₂ (60 mL) at rt. After 30 min, the reaction was diluted with a saturated sodium bicarbonate solution, extracted with EtOAc, the organic layer washed with brine, dried over sodium sulfate, filtered, and solvent removed by rotary evaporation. The product was then isolated by silica chromatography (20%->80% EtOAc/hex) as a white solid. ¹H NMR (400 MHz, DMSO-d₆) δ 12.30 (s, 1H), 7.63 (d, J=6.7 Hz, 1H), 7.12 (d, J=7.6 Hz, 2H), 7.03 (d, J=8.0 Hz, 1H), 6.81 (s, 1H), 4.18 (q, J=7.1 Hz, 2H), 3.52-3.38 (m, 1H), 2.82 (dd, J=17.0, 5.3 Hz, 2H), 2.77-2.66 (m, 1H), 2.60 (dd, J=16.6, 9.3 Hz, 1H), 2.44 (s, 3H), 1.88-1.74 (m, 1H), 1.69-1.51 (m, 1H), 1.26 (t, J=7.1 Hz, 3H). LC/MS (APCI) m/z calcd. for C₁₈H₂₂ClN₂O₄S⁺ [M+H]⁺: 397.1; 397.1 found.

TABLE I-23
The following intermediates were prepared according to synthetic procedures as described in Intermediate Synthesis 23.

| | | Sulfonyl | |
|---|---|---|---|
| Intermediate | Amine | Chloride | Structure, Name and Data |
| 23.1 | (R)-6-chloro- 1,2,3,4- tetrahydronaphthalen- 2-amine | 18.2 | 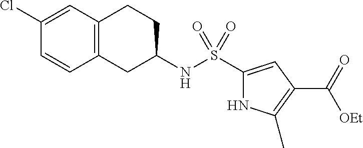 [SMILES: CCOC(=O)c1cc(S(=O)(=O)N[C@@H]2CCc3cc(Cl)ccc3C2)[nH]c1C] Ethyl (R)-5-(N-(6-chloro-1,2,3,4- tetrahydronaphthalen-2-yl)sulfamoyl)-2-methyl-1H- pyrrole-3-carboxylate. ¹H NMR (400 MHz, DMSO-d₆) δ 12.30 (s, 1H), 7.63 (d, J = 6.7 Hz, 1H), 7.12 (d, J = 7.6 Hz, 2H), 7.03 (d, J = 8.0 Hz, 1H), 6.81 (s, 1H), 4.18 (q, J = 7.1 Hz, 2H), 3.52-3.38 (m, 1H), 2.82 (dd, J = 17.0, 5.3 Hz, 2H), 2.77-2.66 (m, 1H), 2.60 (dd, J = 16.6, 9.3 Hz, 1H), 2.44 (s, 3H), 1.88-1.74 (m, 1H), 1.69-1.51 (m, 1H), 1.26 (t, J = 7.1 Hz, 3H). LC/MS (APCI) m/z calcd. for C₁₈H₂₂ClN₂O₄S⁺ [M + H]⁺: 397.1; 397.1 found. |
| 23.2 | 5.3 | 18.2 | 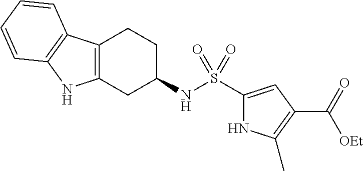 [SMILES: CCOC(=O)c1cc(S(=O)(=O)N[C@@H]2CCc3c([nH]c4ccccc34)C2)[nH]c1C] Ethyl (R)-2-methyl-5-(N-(2,3,4,9-tetrahydro-1H- carbazol-2-yl)sulfamoyl)-1H-pyrrole-3-carboxylate. ¹H NMR (400 MHz, DMSO-d₆) δ 12.34 (s, 1H), 10.63 (s, 1H), 7.70 (d, J = 7.3 Hz, 1H), 7.32 (d, J = 7.7 Hz, 1H), 7.22 (d, J = 7.9 Hz, 1H), 6.99 (t, J = 8.0 Hz, 1H), 6.92 (t, J = 7.8 Hz, 1H), 6.84 (d, J = 2.4 Hz, 1H), 4.18 (q, J = 7.1 Hz, 2H), 3.67-3.49 (m, 1H), 2.87-2.56 (m, 4H), 2.46 (s, 3H), 1.98-1.85 (m, 1H), 1.80-1.64 (m, 1H), 1.27 (t, J = 7.1 Hz, 3H). LC/MS (APCI) m/z calcd. for C₂₀H₂₃N₃O₄S⁺ [M + H]⁺: 402.1; 402.2 found. Stereochemistry arbitrarily assigned. |
| 23.3 | 3.3 | 18.2 | 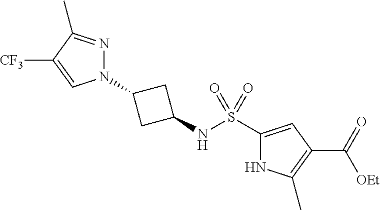 [SMILES: CCOC(=O)c1cc(S(=O)(=O)N[C@H]2C[C@H](n3cc(C(F)(F)F)c(C)n3)C2)[nH]c1C] Ethyl 2-methyl-5-(N-((trans)-3-(3-methyl-4- (trifluoromethyl)-1H-pyrazol-1- yl)cyclobutyl)sulfamoyl)-1H-pyrrole-3-carboxylate. ¹H NMR (400 MHz, DMSO-d₆) δ 12.31 (s, 1H), 8.30 (s, 1H), 7.95 (d, J = 7.6 Hz, 1H), 6.80 (d, J = 2.6 Hz, 1H), 4.83 (tt, J = 9.4, 4.7 Hz, 1H), 4.17 (q, J = 7.1 Hz, 2H), 3.98 (dd, J = 13.4, 7.1 Hz, 1H), 2.58 (ddd, J = 13.3, 8.2, 5.2 Hz, 2H), 2.43 (d, J = 1.7 Hz, 3H), 2.34 (ddd, J = 13.6, 8.7, 5.8 Hz, 2H), 2.29-2.21 (m, 3H), 1.26 (t, J = 7.1 Hz, 3H). LC/MS (APCI) m/z calcd. for C₁₇H₂₂F₃N₄O₄S⁺ [M + H]⁺: 435.1; 435.2 found. |
| 23.4 | 3.4 | 18.2 | 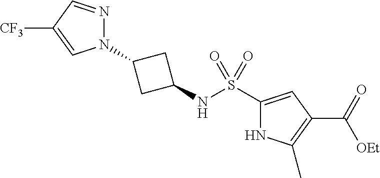 [SMILES: CCOC(=O)c1cc(S(=O)(=O)N[C@H]2C[C@H](n3cc(C(F)(F)F)cn3)C2)[nH]c1C] Ethyl 2-methyl-5-(N-((trans)-3-(4-(trifluoromethyl)- 1H-pyrazol-1-yl)cyclobutyl)sulfamoyl)-1H-pyrrole-3- carboxylate. ¹H NMR (400 MHz, DMSO-d₆) δ 12.32 (s, 1H), 8.45 (s, 1H), 7.95 (d, J = 8.6 Hz, 2H), 6.80 (s, 1H), 4.92 (tt, J = 9.0, 4.9 Hz, 1H), 4.17 (q, J = 7.1 Hz, 2H), 4.03 (s, 1H), 2.59 (ddd, J = 13.1, 8.1, 4.7 Hz, 2H), 2.43 (s, 3H), 2.38 (td, J = 8.3, 4.3 Hz, 2H), 1.26 (t, J = 7.1 Hz, 3H). LC/MS (APCI) m/z calcd. for C₁₆H₂₀F₃N₄O₄S⁺ [M + H]⁺: 421.1; 421.1 found. |
| 23.5 | 2,3,4,9- tetrahydro-1H- carbazol-2- amine | 18.2 | 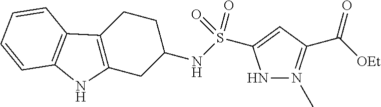 Ethyl 2-methyl-5-(N-(2,3,4,9-tetrahydro-1H-carbazol- 2-yl)sulfamoyl)-1H-pyrrole-3-carboxylate. ¹H NMR (400 MHz, DMSO-d₆) δ 11.97 (s, 1H), 10.60 (s, 1H),7.36- 7.27 (m, 2H), 7.21 (d, J = 7.9 Hz, 1H), 6.99 (ddd, J = 8.1, 7.0, 1.3 Hz, 1H), 6.91 (td, J = 7.4, 7.0, 1.1 Hz, 1H), 6.39 (d, J = 6.8 Hz, 1H), 4.14 (qd, J = 7.1, 5.1 Hz, 2H), 3.66-3.46 (m, 1H), 3.22-3.11 (m, 1H), 2.83 (dd, J = 16.2, 5.2 Hz, 1H), 2.69-2.56 (m, 2H), 2.43 (s, 3H), 1.91 (dd, J = 10.4, 5.8 Hz, 1H), 1.86-1.65 (m, 1H), 1.22 (t, J = 7.1 Hz, 3H). LC/MS (APCI) m/z calcd. for C₂₀H₂₄N₃O₄S⁺ [M + H]⁺: 402.1; 402.2 found. |
| 23.6 | 1.1 | 3-cyano- 4- fluorobenzenesulfonyl chloride | 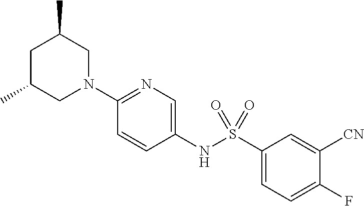 [SMILES: C[C@@H]1C[C@@H](C)CN(c2ccc(NS(=O)(=O)c3ccc(F)c(C#N)c3)cn2)C1] and |
| | | | 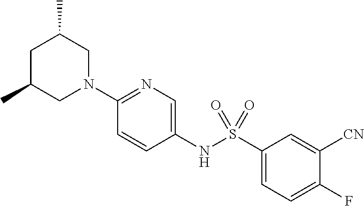 [SMILES: C[C@H]1C[C@H](C)CN(c2ccc(NS(=O)(=O)c3ccc(F)c(C#N)c3)cn2)C1] 3-Cyano-N-(6-(trans-3,5-dimethylpiperidin-1- yl)pyridin-3-yl)-4-fluorobenzenesulfonamide. LC/MS (APCI) m/z calcd. for C₁₉H₂₂FN₄O₂S⁺ [M + H]⁺: 389.1; 389.1 found. |
| 23.7 | 1- phenylpiperidin- 4-amine | 18.1 | 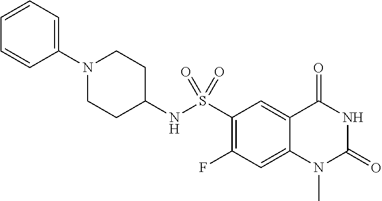 [SMILES: Cn1c(=O)[nH]c(=O)c2cc(S(=O)(=O)NC3CCN(c4ccccc4)CC3)c(F)cc21] 7-Fluoro-1-methyl-2,4-dioxo-N-(1-phenylpiperidin-4- yl)-1,2,3,4-tetrahydroquinazoline-6-sulfonamide. ¹H NMR (400 MHz, DMSO-d₆) δ 11.88 (s, 1H), 8.34 (d, J = 8.0 Hz, 1H), 8.23 (d, J = 7.7 Hz, 1H), 7.59 (d, J = 12.3 Hz, 1H), 7.17 (dd, J = 8.7, 7.2 Hz, 2H), 6.87 (d, J = 7.8 Hz, 2H), 6.72 (t, J = 7.2 Hz, 1H), 3.58 (d, J = 13.3 Hz, 2H), 3.44 (s, 3H), 3.31-3.21 (m, 1H), 2.74-2.62 (m, 2H), 1.73-1.61 (m, 2H), 1.61-1.43 (m, 2H). LC/MS (APCI) m/z calcd. for C₂₀H₂₂FN₄O₄S⁺ [M + H]⁺: 433.2; 433.2 found. |
| 23.8 | (trans)-4- phenylcyclohexan- 1-amine | 18.1 | 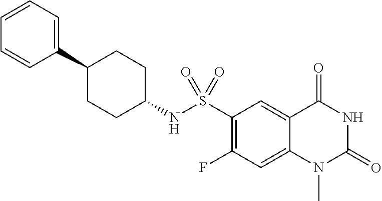 [SMILES: Cn1c(=O)[nH]c(=O)c2cc(S(=O)(=O)N[C@H]3CC[C@H](c4ccccc4)CC3)c(F)cc21] 7-Fluoro-1-methyl-2,4-dioxo-N-((trans)-4- phenylcyclohexyl)-1,2,3,4-tetrahydroquinazoline-6- sulfonamide. ¹H NMR (400 MHz, DMSO-d₆) δ 11.86 (s, 1H), 8.35 (d, J = 8.0 Hz, 1H), 8.19 (d, J = 7.8 Hz, 1H), 7.58 (d, J = 12.2 Hz, 1H), 7.30-7.09 (m, 5H), 3.44 (s, 3H), 3.19-3.12 (m, 1H), 2.37 (dd, J = 21.0, 10.0 Hz, 1H), 1.83-1.65 (m, 4H), 1.42 (q, J = 12.3 Hz, 4H). LC/MS (APCI) m/z calcd. for C₂₁H₂₃FN₃O₄S⁺ [M + H]⁺: 432.1; 432.1 found. |
| 23.9 | N¹- phenylpropane- 1,3-diamine | 18.1 | 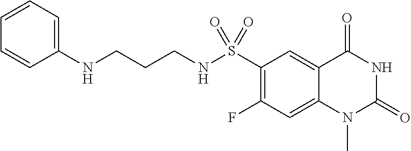 [SMILES: Cn1c(=O)[nH]c(=O)c2cc(S(=O)(=O)NCCCNc3ccccc3)c(F)cc21] 7-Fluoro-1-methyl-2,4-dioxo-N-(3- (phenylamino)propyl)-1,2,3,4-tetrahydroquinazoline- 6-sulfonamide. LC/MS (APCI) m/z calcd. for C₁₈H₂₀FN₄O₄S⁺ [M + H]⁺: 407.1; 407.3 found. |
| 23.10 | 1-(3- fluorophenyl) piperidin-4- amine | 19.2 | 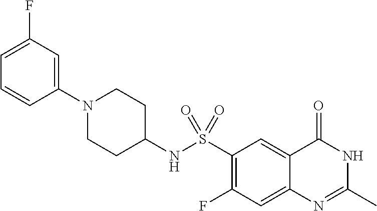 [SMILES: Cc1nc2cc(F)c(S(=O)(=O)NC3CCN(c4cccc(F)c4)CC3)cc2c(=O)[nH]1] 7-Fluoro-N-(1-(3-fluorophenyl)piperidin-4-yl)-2- methyl-4-oxo-3,4-dihydroquinazoline-6-sulfonamide. ¹H NMR (400 MHz, DMSO-d₆) δ 12.62 (s, 1H), 8.47 (d, J = 8.0 Hz, 1H), 8.26 (d, J = 7.9 Hz, 1H), 7.60 (d, J = 11.3 Hz, 1H), 7.16 (q, J = 8.1 Hz, 1H), 6.73-6.61 (m, 2H), 6.47 (td, J = 8.3, 2.3 Hz, 1H), 3.62 (d, J = 13.1 Hz, 2H), 3.32-3.26 (m, 1H), 2.79-2.66 (m, 2H), 2.40 (s, 3H), 1.62 (d, J = 12.7 Hz, 2H), 1.56-1.37 (m, 2H). LC/MS (APCI) m/z calcd. for C₂₀H₂₁F₂N₄O₃S⁺ [M + H]⁺: 435.1; 434.1 found. |
| 23.11 (Compound 30) | 1.2 | 18.2 | 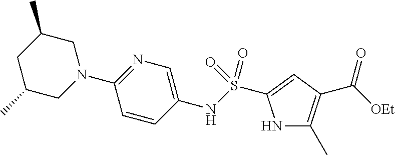 [SMILES: CCOC(=O)c1cc(S(=O)(=O)Nc2ccc(N3C[C@H](C)C[C@@H](C)C3)nc2)[nH]c1C] Ethyl 5-(N-(6-((3R,5R)-3,5-dimethylpiperidin-1- yl)pyridin-3-yl)sulfamoyl)-2-methyl-1H-pyrrole-3- carboxylate. ¹H NMR (400 MHz, DMSO-d₆) δ 12.26 (s, 1H), 9.47 (s, 1H), 7.71 (d, J = 2.6 Hz, 1H), 7.12 (dd, J = 9.1, 2.8 Hz, 1H), 6.73 (d, J = 9.1 Hz, 1H), 6.70 (d, J = 2.7 Hz, 1H), 4.16 (q, J = 7.1 Hz, 2H), 3.53 (dd, J = 12.8, 3.7 Hz, 2H), 3.33 (s, 1H), 3.11 (dd, J = 12.8, 6.8 Hz, 2H), 2.40 (s, 3H), 1.87 (ddd, J = 12.9, 8.4, 5.2 Hz, 2H), 1.41 (t, J = 5.8 Hz, 2H), 1.24 (t, J = 7.1 Hz, 3H), 0.88 (d, J = 6.8 Hz, 6H). LC/MS (APCI) m/z calcd. for C₂₀H₂₉N₄O₄S⁺ [M + H]⁺: 421.2; 421.2 found. |
| 23.12 | (trans)-3-(3- fluorophenyl) cyclobutan-1- amine | 18.2 | 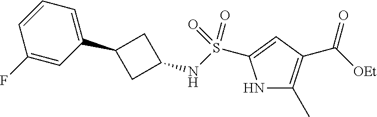 [SMILES: CCOC(=O)c1cc(S(=O)(=O)N[C@H]2C[C@H](c3cccc(F)c3)C2)[nH]c1C] Ethyl 5-(N-((trans)-3-(3- fluorophenyl)cyclobutyl)sulfamoyl)-2-methyl-1H- pyrrole-3-carboxylate. ¹H NMR (400 MHz, DMSO-d₆) δ 12.29 (s, 1H), 7.91 (d, J = 7.6 Hz, 1H), 7.35 (td, J = 8.1, 6.3 Hz, 1H), 7.14-7.06 (m, 2H), 7.01 (td, J = 8.3, 2.0 Hz, 1H), 6.78 (s, 1H), 4.17 (q, J = 7.1 Hz, 2H), 3.87 (q, J = 7.3 Hz, 1H), 3.48 (p, J = 7.3 Hz, 1H), 2.43 (s, 3H), 2.27 (t, J = 7.4 Hz, 4H), 1.26 (t, J = 7.1 Hz, 3H). LC/MS (APCI) m/z calcd. for C₁₈H₂₂FN₂O₄S⁺ [M + H]⁺: 381.1; 381.1 found. |
| 23.13 | (trans)-3-(3- chlorophenyl) cyclobutan-1- amine | 18.2 | 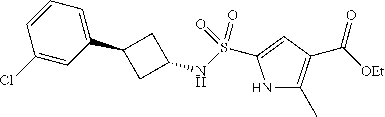 [SMILES: CCOC(=O)c1cc(S(=O)(=O)N[C@H]2C[C@H](c3cccc(Cl)c3)C2)[nH]c1C] Ethyl 5-(N-((trans)-3-(3- chlorophenyl)cyclobutyl)sulfamoyl)-2-methyl-1H- pyrrole-3-carboxylate. ¹H NMR (400 MHz, DMSO-d₆) δ 12.29 (s, 1H), 7.91 (d, J = 7.5 Hz, 1H), 7.34 (t, J = 7.8 Hz, 1H), 7.31 (d, J = 1.9 Hz, 1H), 7.28-7.18 (m, 2H), 6.78 (s, 1H), 4.17 (q, J = 7.1 Hz, 2H), 3.86 (p, J = 7.2 Hz, 1H), 3.47 (p, J = 7.4 Hz, 1H), 2.43 (s, 3H), 2.27 (t, J = 7.3 Hz, 4H), 1.26 (t, J = 7.1 Hz, 3H). |
| 23.14 | 3- phenylbicyclo [1.1.1]pentan-1- amine | 18.2 | 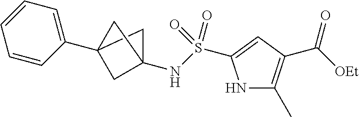 [SMILES: CCOC(=O)c1cc(S(=O)(=O)NC23CC(c4ccccc4)(C2)C3)[nH]c1C] Ethyl 2-methyl-5-(N-(3-phenylbicyclo[1.1.1]pentan-1- yl)sulfamoyl)-1H-pyrrole-3-carboxylate. ¹H NMR (400 MHz, DMSO-d₆) δ 12.30 (s, 1H), 8.48 (s, 1H), 7.31- 7.24 (m, 2H), 7.24-7.14 (m, 3H), 6.84 (s, 1H), 4.18 (q, J = 7.1 Hz, 2H), 2.46 (s, 3H), 2.04 (s, 6H), 1.26 (t, J = 7.1 Hz, 3H). LC/MS (APCI) m/z calcd. for C₁₉H₂₃N₂O₄S⁺ [M + H]⁺: 375.1; 375.1 found. |
| 23.15 | (trans)-3-(4- chlorophenyl) cyclobutan-1- amine | 18.2 | 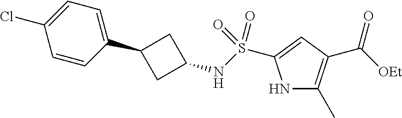 [SMILES: CCOC(=O)c1cc(S(=O)(=O)N[C@H]2C[C@H](c3ccc(Cl)cc3)C2)[nH]c1C] Ethyl 5-(N-((trans)-3-(4-chlorophenyl)cyclobutyl) sulfamoyl)-2-methyl-1H-pyrrole-3-carboxylate. ¹H NMR (400 MHz, DMSO-d₆) δ 12.30 (s, 1H), 7.92 (d, J = 7.7 Hz, 1H), 7.36 (d, J = 8.5 Hz, 2H), 7.28 (d, J = 8.5 Hz, 2H), 6.78 (s, 1H), 4.17 (q, J = 7.1 Hz, 2H), 3.86 (h, J = 7.5, 7.1 Hz, 1H), 3.45 (tt, J = 9.6, 5.5 Hz, 1H), 2.43 (s, 3H), 2.36-2.15 (m, 4H), 1.26 (t, J = 7.1 Hz, 3H). LC/MS (APCI) m/z calcd. for C₁₈H₂₂ClN₂O₄S⁺ [M + H]⁺: 397.1; 397.1 found. |
| 23.16 | (cis)-3-(3- (trifluoromethyl) phenyl)cyclobutan- 1-amine | 18.2 | 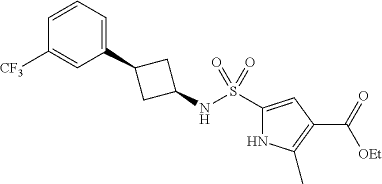 [SMILES: CCOC(=O)c1cc(S(=O)(=O)N[C@H]2C[C@@H](c3cccc(C(F)(F)F)c3)C2)[nH]c1C] Ethyl 2-methyl-5-(N-((cis)-3-(3- (trifluoromethyl)phenyl)cyclobutyl)sulfamoyl)-1H- pyrrole-3-carboxylate. LC/MS (APCI) m/z calcd. for C₁₉H₂₂F₃N₂O₄S⁺ [M + H]⁺: 431.1; 431.1 found. |
| 23.17 | 4 | 18.2 | 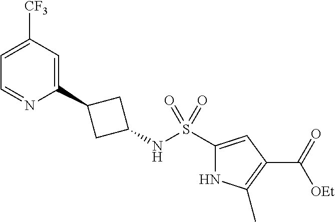 [SMILES: CCOC(=O)c1cc(S(=O)(=O)N[C@H]2C[C@H](c3cc(C(F)(F)F)ccn3)C2)[nH]c1C] Ethyl 2-methyl-5-(N-((trans)-3-(4- (trifluoromethyl)pyridin-2-yl)cyclobutyl)sulfamoyl)- 1H-pyrrole-3-carboxylate. LC/MS (APCI) m/z calcd. for C₁₈H₂₁F₃N₃O₄S⁺ [M + H]⁺: 432.1; 432.1 found. |
| 23.18 | 3-(4- chlorobenzyl) tetrahydrofuran- 3-amine | 18.2 | 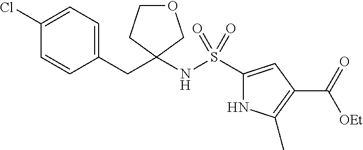 [SMILES: CCOC(=O)c1cc(S(=O)(=O)NC2(Cc3ccc(Cl)cc3)CCOC2)[nH]c1C] Ethyl 5-(N-(3-(4-chlorobenzyl)tetrahydrofuran-3- yl)sulfamoyl)-2-methyl-1H-pyrrole-3-carboxylate. ¹H NMR (400 MHz, DMSO-d₆) δ 12.23 (s, 1H), 7.60 (s, 1H), 7.36 (d, J = 8.4 Hz, 2H), 7.29 (d, J = 8.5 Hz, 2H), 6.76 (d, J = 2.4 Hz, 1H), 4.17 (q, J = 7.1 Hz, 2H), 3.78-3.63 (m, 2H), 3.60-3.47 (m, 2H), 3.08-2.94 (m, 2H), 2.43 (s, 3H), 2.05 (ddd, J = 12.3, 7.4, 4.4 Hz, 1H), 1.86 (dt, J = 13.0, 8.2 Hz, 1H), 1.26 (t, J = 7.1 Hz, 3H). LC/MS (APCI) m/z calcd. for C₁₉H₂₄ClN₂O₅S⁺ [M + H]⁺: 427.1; 427.1 found. |
| 23.19 | 1.2 | 19.2 | 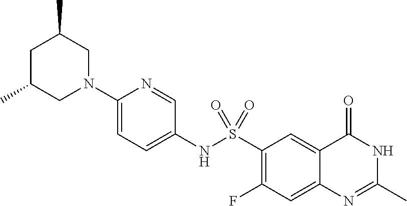 N-(6-((3R,5R)-3,5-dimethylpiperidin-1-yl)pyridin-3- yl)-7-fluoro-2-methyl-4-oxo-3,4-dihydroquinazoline-6- sulfonamide. ¹H NMR (400 MHz, Methanol-d₄) δ 8.36 (d, J = 7.7 Hz, 1H), 7.61 (d, J = 2.7 Hz, 1H), 7.30 (d, J = 11.0 Hz, 1H), 7.21 (dd, J = 9.1, 2.7 Hz, 1H), 6.55 (d, J = 9.1 Hz, 1H), 3.40 (dd, J = 12.9, 3.7 Hz, 2H), 2.99 (dd, J = 12.9, 6.8 Hz, 2H), 2.34 (s, 3H), 1.80 (pd, J = 6.4, 3.8 Hz, 2H), 1.35 (t, J = 5.8 Hz, 2H), 0.79 (d, J = 6.8 Hz, 6H). LC/MS (APCI) m/z calcd. for C₂₁H₂₅FN₅O₃S⁺ [M + H]⁺: 446.2; 446.1 found. |
| 23.2 | 1.2 | methyl 4- bromo-5- (chlorosulfonyl)- 2-fluorobenzoate | 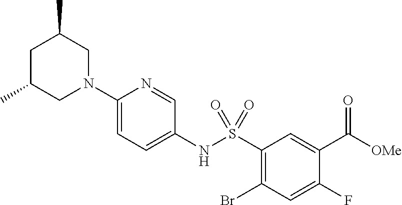 [SMILES: COC(=O)c1cc(S(=O)(=O)Nc2ccc(N3C[C@H](C)C[C@@H](C)C3)nc2)c(Br)cc1F] Methyl 4-bromo-5-(N-(6-((3R,5R)-3,5- dimethylpiperidin-1-yl)pyridin-3-yl)sulfamoyl)-2- fluorobenzoate. ¹H NMR (400 MHz, DMSO-d₆) δ 11.95 (s, 1H), 8.22 (s, 1H), 7.71 (s, 1H), 7.21-6.92 (m, 1H), 6.74-6.66 (m, 1H), 6.60 (s, 1H), 3.44-3.30 (m, 2H), 3.14- 2.98 (m, 2H), 2.29 (s, 3H), 1.94-1.72 (m, 2H), 1.47- 1.28 (m, 2H), 0.85 (s, 6H). |
| 23.21 | 3-(4- chlorobenzyl) tetrahydrofuran- 3-amine | 19.2 | 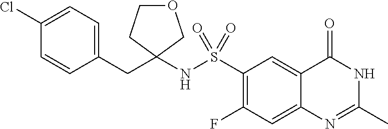 [SMILES: Cc1nc2cc(F)c(S(=O)(=O)NC3(Cc4ccc(Cl)cc4)CCOC3)cc2c(=O)[nH]1] N-(3-(4-chlorobenzyl)tetrahydrofuran-3-yl)-7-fluoro- 2-methyl-4-oxo-3,4-dihydroquinazoline-6- sulfonamide. LC/MS (APCI) m/z calcd. for C₂₀H₂₀ClFN₃O₄S⁺ [M + H]⁺: 452.0; 452.0 found. |
| 23.22 | 3-(4- chlorobenzyl) tetrahydrofuran- 3-amine | 18.1 | 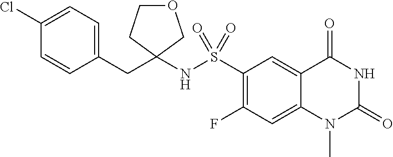 [SMILES: Cn1c(=O)[nH]c(=O)c2cc(S(=O)(=O)NC3(Cc4ccc(Cl)cc4)CCOC3)c(F)cc21] N-(3-(4-chlorobenzyl)tetrahydrofuran-3-yl)-7-fluoro- 2-methyl-4-oxo-3,4-dihydroquinazoline-6- sulfonamide. LC/MS (APCI) m/z calcd. for C₂₀H₂₀ClFN₃O₅S⁺ [M + H]⁺: 467.0; 467.0 found. |
| 23.23 | 6.0 | 18.2 | 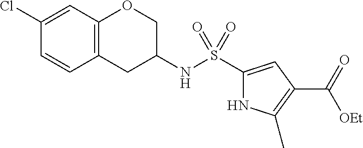 [SMILES: CCOC(=O)c1cc(S(=O)(=O)NC2COc3cc(Cl)ccc3C2)[nH]c1C] Ethyl 5-(N-(7-chlorochroman-3-yl)sulfamoyl)-2- methyl-1H-pyrrole-3-carboxylate. ¹H NMR (400 MHz, DMSO-d₆) δ 12.34 (s, 1H), 7.82 (s, 1H), 7.06 (d, J = 8.2 Hz, 1H), 6.90 (dd, J = 8.1, 2.1 Hz, 1H), 6.85 (d, J = 2.6 Hz, 2H), 4.18 (q, J = 7.1 Hz, 2H), 4.11-4.01 (m, 1H), 3.84 (dd, J = 10.7, 7.6 Hz, 1H), 3.63 (s, 1H), 2.86 (dd, J = 16.5, 5.4 Hz, 1H), 2.66 (dd, J = 16.6, 7.5 Hz, 1H), 2.45 (s, 3H), 1.26 (t, J = 7.1 Hz, 3H). LC/MS (APCI) m/z calcd. for C₁₇H₂₀ClN₂O₅S⁺ [M + H]⁺: 399.1; 399.1 found. |
| 23.24 | 11.2 | 18.2 | 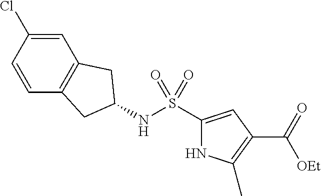 [SMILES: CCOC(=O)c1cc(S(=O)(=O)N[C@@H]2Cc3ccc(Cl)cc3C2)[nH]c1C] Ethyl (S)-5-(N-(5-chloro-2,3-dihydro-1H-inden-2- yl)sulfamoyl)-2-methyl-1H-pyrrole-3-carboxylate. LC/MS (APCI) m/z calcd. for C₁₇H₂₀ClN₂O₄S⁺ [M + H]⁺: 383.1; 383.1 found. Stereochemistry arbitrarily assigned. |
| 23.25 | 10 | 18.2 | 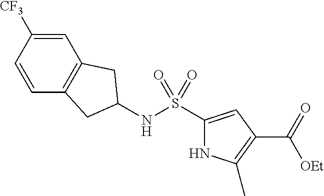 [SMILES: CCOC(=O)c1cc(S(=O)(=O)NC2Cc3ccc(C(F)(F)F)cc3C2)[nH]c1C] Ethyl 2-methyl-5-(N-(5-(trifluoromethyl)-2,3-dihydro- 1H-inden-2-yl)sulfamoyl)-1H-pyrrole-3-carboxylate. LC/MS (APCI) m/z calcd. for C₁₈H₂₀F₃N₂O₄S⁺ [M + H]⁺: 417.1; 417.1 found. |
| 23.26 | 8.0 | 18.2 | 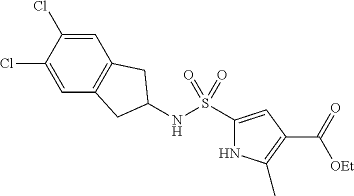 [SMILES: CCOC(=O)c1cc(S(=O)(=O)NC2Cc3cc(Cl)c(Cl)cc3C2)[nH]c1C] Ethyl 5-(N-(5,6-dichloro-2,3-dihydro-1H-inden-2- yl)sulfamoyl)-2-methyl-1H-pyrrole-3-carboxylate. LC/MS (APCI) m/z calcd. for C₁₇H₁₉Cl₂N₂O₄S⁺ [M + H]⁺: 417.0.1; 417.1 found. |
| 23.27 | 12 | 18.2 | 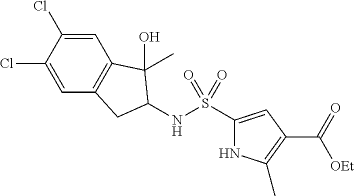 [SMILES: CCOC(=O)c1cc(S(=O)(=O)NC2Cc3cc(Cl)c(Cl)cc3C2(C)O)[nH]c1C] Ethyl 5-(N-(5,6-dichloro-1-hydroxy-1-methyl-2,3- dihydro-1H-inden-2-yl)sulfamoyl)-2-methyl-1H- pyrrole-3-carboxylate. LC/MS (APCI) m/z calcd. for C₁₈H₂₁Cl₂N₂O₅S⁺ [M + H]⁺: 447.1; 447.1 found. |
| 23.28 | 8.1 | 18.2 | 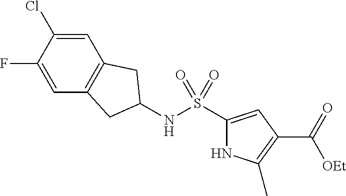 [SMILES: CCOC(=O)c1cc(S(=O)(=O)NC2Cc3cc(F)c(Cl)cc3C2)[nH]c1C] Ethyl 5-(N-(5-chloro-6-fluoro-2,3-dihydro-1H-inden- 2-yl)sulfamoyl)-2-methyl-1H-pyrrole-3-carboxylate. LC/MS cald for C₁₇H₁₉ClFN₂O₄S m/z 401.1 [M + H]+, found 401.1 [M + H]+. |
| 23.29 | 13.1 | 18.2 | 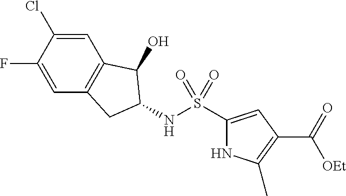 [SMILES: CCOC(=O)c1cc(S(=O)(=O)N[C@@H]2Cc3cc(F)c(Cl)cc3[C@H]2O)[nH]c1C] and |
| | | | 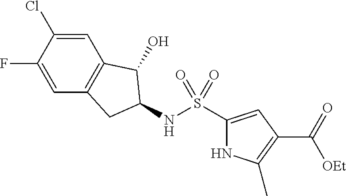 [SMILES: CCOC(=O)c1cc(S(=O)(=O)N[C@H]2Cc3cc(F)c(Cl)cc3[C@@H]2O)[nH]c1C] Ethyl 5-(N-(5-chloro-6-fluoro-2,3-dihydro-1H-inden- 2-yl)sulfamoyl)-2-methyl-1H-pyrrole-3-carboxylate. LC/MS cald for C₁₇H₁₉ClFN₂O₅S m/z 417.1 [M + H]+, found 417.1 [M + H]+. |
| 23.30 | 9.2 | 18.2 | 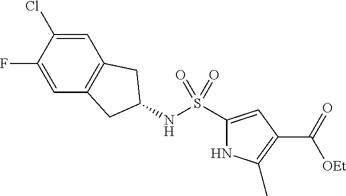 [SMILES: CCOC(=O)c1cc(S(=O)(=O)N[C@@H]2Cc3cc(F)c(Cl)cc3C2)[nH]c1C] Ethyl (R)-5-(N-(5-chloro-6-fluoro-2,3-dihydro-1H- inden-2-yl)sulfamoyl)-2-methyl-1H-pyrrole-3- carboxylate. LC/MS cald for C₁₇H₁₉ClFN₂O₄S m/z 401.1 [M + H]+, found 401.1 [M + H]+. Stereochemistry arbitrarily assigned. |
| 23.31 | 9.2 | 18.3 | 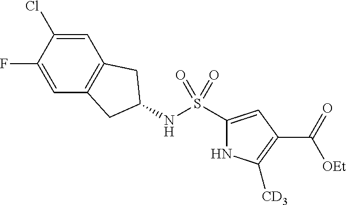 [SMILES: [2H]C([2H])([2H])c1[nH]c(S(=O)(=O)N[C@@H]2Cc3cc(F)c(Cl)cc3C2)cc1C(=O)OCC] Ethyl (R)-5-(N-(5-chloro-6-fluoro-2,3-dihydro-1H- inden-2-yl)sulfamoyl)-2-methyl-1H-pyrrole-3- carboxylate. LC/MS cald for C₁₇H₁₅D₃ClFN₂O₄S m/z 404.1 [M + H]+, found 404.1 [M + H]+. Stereochemistry arbitrarily assigned. |
| 23.32 | 6.1 | 18.2 | 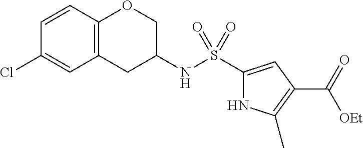 [SMILES: CCOC(=O)c1cc(S(=O)(=O)NC2COc3ccc(Cl)cc3C2)[nH]c1C] Ethyl 5-(N-(6-chlorochroman-3-yl)sulfamoyl)-2- methyl-1H-pyrrole-3-carboxylate. ¹H NMR (400 MHz, DMSO-d₆) δ 12.34 (s, 1H), 7.82 (s, 1H), 7.12 (dd, J = 6.7, 2.7 Hz, 2H), 6.85 (s, 1H), 6.81-6.77 (m, 1H), 4.18 (q, J = 7.1 Hz, 2H), 4.05 (ddd, J = 10.7, 3.3, 1.5 Hz, 1H), 3.83 (dd, J = 10.7, 7.6 Hz, 1H), 3.69-3.54 (m, 1H), 2.88 (dd, J = 16.7, 5.4 Hz, 1H), 2.74-2.62 (m, 1H), 2.45 (s, 3H), 1.26 (t, J = 7.1 Hz, 3H). LC/MS (APCI) m/z calcd. for C₁₇H₂₀ClN₂O₅S⁺ [M + H]⁺: 399.1; 399.1 found. |
| 23.33 | 13.0 | 18.2 | 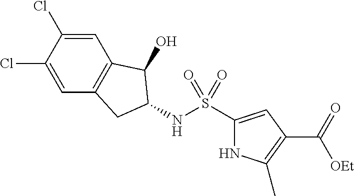 [SMILES: CCOC(=O)c1cc(S(=O)(=O)N[C@@H]2Cc3cc(Cl)c(Cl)cc3[C@H]2O)[nH]c1C] and |
| | | | 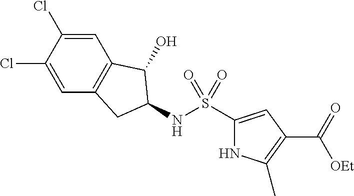 [SMILES: CCOC(=O)c1cc(S(=O)(=O)N[C@H]2Cc3cc(Cl)c(Cl)cc3[C@@H]2O)[nH]c1C] Ethyl 5-(N-(5-chloro-6-chloro-2,3-dihydro-1H-inden- 2-yl)sulfamoyl)-2-methyl-1H-pyrrole-3-carboxylate. LC/MS cald for C₁₇H₁₉Cl₂N₂O₅S m/z 433.1 [M + H]+, found 433.1 [M + H]+. |

### Example I-24. Intermediate Synthesis 24

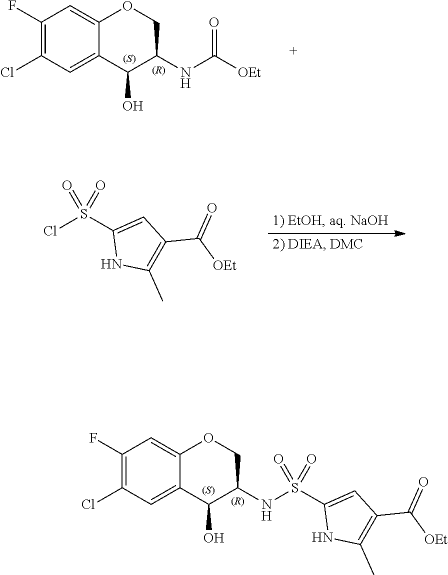

### Preparation of Ethyl 5-(N-((3R,4S)-6-chloro-7-fluoro-4-hydroxychroman-3-yl)sulfamoyl)-2-methyl-1H-pyrrole-3-carboxylate (Intermediate 24.1)

Ethyl ((3R,4S)-6-chloro-7-fluoro-4-hydroxychroman-3-yl)carbamate (157 mg, 0.54 mmol) was dissolved in ethanol (5.0 mL), and 50% aqueous NaOH (1 ml) was added. The resulting mixture was stirred at 100° C. for 16 h. It was cooled to room temperature, and ethanol removed under reduced pressure. The resulting solution was diluted with water (10 mL) and extracted with ethyl acetate (3×25 mL). The organic extracts were combined, dried over sodium sulfate and concentrated to a glassy solid. The solid was dissolved in dichloromethane (2.0 mL) and diisopropylethylamine (0.283 ml, 1.63 mmol) was added. Ethyl 5-(chlorosulfonyl)-2-methyl-1H-pyrrole-3-carboxylate (0.150 g, 0.60 mmol) was added, and the resulting mixture was stirred and room temperature for 15 min. The reaction was diluted with additional dichloromethane (20 mL), washed with 0.1 M HCl (15 mL), brine and dried over sodium sulfate. Concentration under reduced pressure provided a tan solid which was purified with silica gel using 30% ethyl acetate/hexanes, followed by reverse phase HPLC using a 40 min gradient from 10-100% acetonitrile/water with 0.1% formic acid (Phenomenex Gemini 5 micron C18 column, 150×21 mm, Axia Pack) to give 0.147 g (63%) of ethyl 5-(N-((3R,4S)-6-chloro-7-fluoro-4-hydroxychroman-3-yl)sulfamoyl)-2-methyl-1H-pyrrole-3-carboxylate as a white solid. ¹H NMR (400 MHz, Methanol-d₄) δ 7.32 (d, J=8.3 Hz, 1H), 7.03 (s, 1H), 6.69 (d, J=10.4 Hz, 1H), 4.46 (d, J=3.7 Hz, 1H), 4.26 (q, J=7.1 Hz, 2H), 4.04 (t, J=10.4 Hz, 1H), 3.94 (dd, J=10.4, 3.7 Hz, 1H), 3.70 (dt, J=10.4, 3.7 Hz, 1H), 2.52 (s, 3H), 1.34 (t, J=7.1 Hz, 3H). LC/MS (APCI) m/z calcd. for C₁₇H₁₈ClFN₂O₆S⁺ [M+H]⁺: 433.1; 415.0 found (M+H—H₂O).

TABLE I-24
The following intermediates were prepared according to synthetic
procedures as described in Intermediate Synthesis 24.

| | | Sulfonyl | |
|---|---|---|---|
| Intermediate | Carbamate | Chloride | Structure, Name and Data |
| 24.1 | 14.1 | 18.2 | 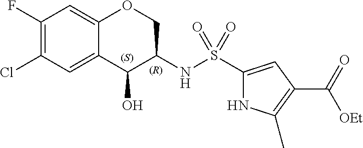 Ethyl 5-(N-((3R,4S)-6-chloro-7-fluoro-4- hydroxychroman-3-yl)sulfamoyl)-2-methyl-1H-pyrrole-3- carboxylate. ¹H NMR (400 MHz, Methanol-d₄) δ 7.32 (d, J = 8.3 Hz, 1H), 7.03 (s, 1H), 6.69 (d, J = 10.4 Hz, 1H), 4.46 (d, J = 3.7 Hz, 1H), 4.26 (q, J = 7.1 Hz, 2H), 4.04 (t, J = 10.4 Hz, 1H), 3.94 (dd, J = 10.4, 3.7 Hz, 1H), 3.70 (dt, J = 10.4, 3.7 Hz, 1H), 2.52 (s, 3H), 1.34 (t, J = 7.1 Hz, 3H). LC/MS (APCI) m/z calcd. for C₁₇H₁₉ClFN₂O₆S⁺ [M + H]⁺: 433.1; 415.0 found (M + H − H₂O). |
| 24.2 | 14.2 | 18.2 | 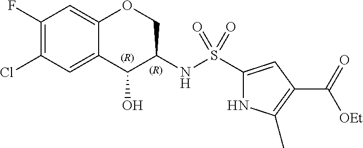 Ethyl 5-(N-((3R,4R)-6-chloro-7-fluoro-4- hydroxychroman-3-yl)sulfamoyl)-2-methyl-1H-pyrrole-3- carboxylate. ¹H NMR (400 MHz, Methanol-d₄) δ 7.29 (d, J = 8.3 Hz, 1H), 7.00 (s, 1H), 6.73 (d, J = 10.6 Hz, 1H), 4.42 (d, J = 4.3 Hz, 1H), 4.30-4.24 (m, 2H), 4.23 (dd, J = 11.0, 1.8 Hz, 1H), 4.03 (ddd, J = 11.5, 4.3, 1.3 Hz, 1H), 3.54 (td, J = 4.2, 2.4 Hz, 1H), 3.35 (s, 1H), 2.50 (s, 3H), 1.40-1.30 (m, 3H). LC/MS (APCI) m/z calcd. for C₁₇H₁₉ClFN₂O₆S⁺ [M + H]⁺: 433.1; 415.0 found (M + H − H₂O). |
| 24.3 | 14.1 | 2,6- dichloropyridine- 4- sulfonyl chloride | 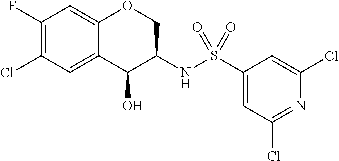 [SMILES: O=S(=O)(N[C@@H]1COc2cc(F)c(Cl)cc2[C@@H]1O)c1cc(Cl)nc(Cl)c1] 2,6-dichloro-N-((3R,4S)-6-chloro-7-fluoro-4- hydroxychroman-3-yl)pyridine-4-sulfonamide. ¹H NMR (400 MHz, Methanol-d₄) δ 7.88 (s, 2H), 7.36 (d, J = 8.3 Hz, 1H), 6.70 (d, J = 10.4 Hz, 1H), 4.60 (d, J = 4.0 Hz, 1H), 4.21- 4.03 (m, 2H), 3.88 (dt, J = 7.8, 3.8 Hz, 1H). LC/MS (APCI) m/z calcd. for C₁₄H₁₁Cl₃FN₂O₄S⁺ [M + H]⁺: 427.0; 427.0 found (M + H). |
| 24.4 | 15 | 18.2 | 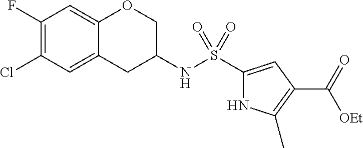 [SMILES: CCOC(=O)c1cc(S(=O)(=O)NC2COc3cc(F)c(Cl)cc3C2)[nH]c1C] Ethyl 5-(N-(6-chloro-7-fluorochroman-3-yl)sulfamoyl)-2- methyl-1H-pyrrole-3-carboxylate. ¹H NMR (400 MHz, DMSO-d₆) δ 12.30 (s, 1H), 7.83 (s, 1H), 7.28 (d, J = 8.5 Hz, 1H), 6.88 (d, J = 10.7 Hz, 1H), 6.84 (s, 1H), 4.18 (q, J = 7.1 Hz, 2H), 4.07 (dd, J = 10.7, 1.8 Hz, 1H), 3.88 (dd, J = 10.7, 7.2 Hz, 1H), 3.70-3.58 (m, 1H), 2.76 (ddd, J = 89.3, 16.6, 6.6 Hz, 2H), 2.45 (s, 3H), 1.27 (t, J = 7.1 Hz, 3H). LC/MS (APCI) m/z calcd. for C₁₇H₁₉ClFN₂O₅S+ [M + H]⁺: 417.1; 417.1 found (M + H). |
| 24.5 | 16 | 3.2 | 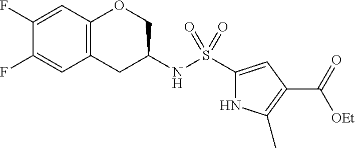 [SMILES: CCOC(=O)c1cc(S(=O)(=O)N[C@@H]2COc3cc(F)c(F)cc3C2)[nH]c1C] Ethyl (S)-5-(N-(6,7-difluorochroman-3-yl)sulfamoyl)-2- methyl-1H-pyrrole-3-carboxylate. LC/MS (APCI) m/z calcd. for C₁₇H₁₉F₂N₂O₅S+ [M + H]⁺: 401.1; 401.1 found (M + H). |
| 24.6 | 17 | 3.2 | 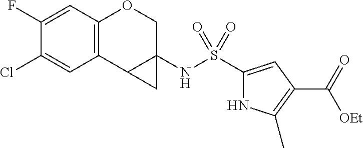 [SMILES: CCOC(=O)c1cc(S(=O)(=O)NC23COc4cc(F)c(Cl)cc4C2C3)[nH]c1C] Ethyl 5-(N-(6-chloro-5-fluoro-1,7b- dihydrocyclopropa[c]chromen-1a(2H)-yl)sulfamoyl)-2- methyl-1H-pyrrole-3-carboxylate. ¹H NMR (400 MHz, DMSO-d₆) δ 12.43 (s, 1H), 8.51 (s, 1H), 7.46 (d, J = 8.3 Hz, 1H), 6.93 (d, J = 10.3 Hz, 1H), 6.81 (d, J = 2.7 Hz, 1H), 4.26- 4.10 (m, 3H), 3.70 (d, J = 10.3 Hz, 1H), 2.45 (s, 3H), 2.12- 2.00 (m, 1H), 1.26 (t, J = 7.1 Hz, 3H), 1.20-1.10 (m, 2H). LC/MS (APCI) m/z calcd. for C₁₈H₁₈N₂O₅FSCl [M + H]⁺: 428.1; 429.1 found. |
| 24.7 | 7 | 3.2 | 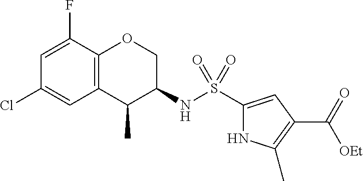 [SMILES: CCOC(=O)c1cc(S(=O)(=O)N[C@@H]2COc3c(F)cc(Cl)cc3[C@@H]2C)[nH]c1C] and |
| | | | 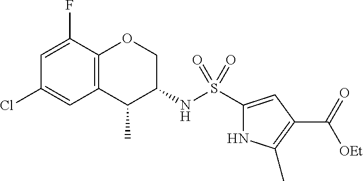 [SMILES: CCOC(=O)c1cc(S(=O)(=O)N[C@H]2COc3c(F)cc(Cl)cc3[C@H]2C)[nH]c1C] Ethyl 5-(N-((cis)-6-chloro-8-fluoro-4-methylchroman-3- yl)sulfamoyl)-2-methyl-1H-pyrrole-3-carboxylate. LC/MS (APCI) m/z calcd. for C₁₈H₂₁ClFN₂O₅S⁺ [M + H]⁺: 431.1; 431.1 found. |

### Example I-25. Intermediate Synthesis 25

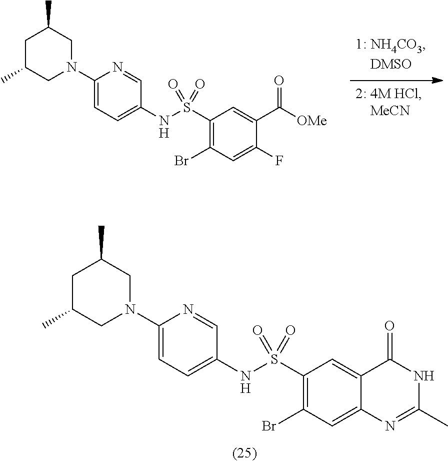

### Preparation of 7-Bromo-N-(6-((3R,5R)-3,5-dimethylpiperidin-1-yl)pyridin-3-yl)-2-methyl-4-oxo-3,4-dihydroquinazoline-6-sulfonamide (Intermediate 25)

Methyl 4-bromo-5-(N-(6-((3R,5R)-3,5-dimethylpiperidin-1-yl)pyridin-3-yl)sulfamoyl)-2-fluorobenzoate (10.9 g, 21.8 mmol, 1 equiv.) was suspended in DMSO (220 mL) before being heated to 120° C. Ammonium carbonate (10.5 g, 109 mmol, 5 equiv.) was added in small portions and the reaction stirred for 1 h. Additional ammonium carbonate (10.5 g, 109 mmol, 5 equiv.) was added and the reaction stirred for 1 h. The reaction was then cooled to rt, diluted with EtOAc, washed with brine, filtered through a pad of silica, and solvent removed by rotary evaporation. The material was then suspended in MeCN (200 mL) before 4 M HCl in dioxanes (100 mL, 400 mmol, 18 equiv.) was added and the reaction heated to 80° C. After 14 h, the reaction was cooled to rt, concentrated by rotary evaporation, diluted with EtOAc, and the pH adjusted to pH 8-9. The resulting solids were filtered and dried to give the desired product as a off-white solid (Intermediate 25, 6.6 g, 59%). ¹H NMR (400 MHz, DMSO-d₆) δ 11.94 (s, 1H), 8.16 (s, 1H), 7.63 (d, J=2.7 Hz, 1H), 7.08 (dd, J=9.1, 2.7 Hz, 1H), 6.67 (d, J=9.3 Hz, 2H), 6.31 (t, J=5.5 Hz, 1H), 3.49 (dd, J=12.8, 3.7 Hz, 2H), 3.07 (dd, J=12.8, 6.8 Hz, 2H), 2.27 (s, 3H), 1.68 (p, J=6.9 Hz, 2H), 1.37 (t, J=5.8 Hz, 2H), 0.83 (d, J=6.8 Hz, 6H).

TABLE I-25
The following intermediates were prepared according to synthetic
procedures as described in Intermediate Synthesis 26.

| Intermediate | Starting Material | Structure, Name and Data |
|---|---|---|
| 25 | Methyl 4-bromo- 5-(N-(6-((3R,5R)- 3,5- dimethylpiperidin- 1-yl)pyridin-3- yl)sulfamoyl)-2- fluorobenzoate | 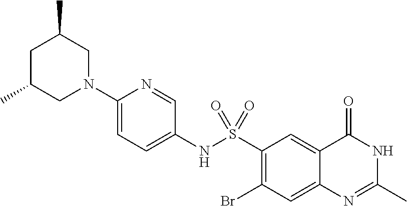 7-Bromo-N-(6-((3R,5R)-3,5-dimethylpiperidin-1- yl)pyridin-3-yl)-2-methyl-4-oxo-3,4-dihydroquinazoline- 6-sulfonamide. ¹H NMR (400 MHz, DMSO-d₆) δ 11.94 (s, 1H), 8.16 (s, 1H), 7.63 (d, J = 2.7 Hz, 1H), 7.08 (dd, J = 9.1, 2.7 Hz, 1H), 6.67 (d, J = 9.3 Hz, 2H), 6.31 (t, J = 5.5 Hz, 1H), 3.49 (dd, J = 12.8, 3.7 Hz, 2H), 3.07 (dd, J = 12.8, 6.8 Hz, 2H), 2.27 (s, 3H), 1.68 (p, J = 6.9 Hz, 2H), 1.37 (t, J = 5.8 Hz, 2H), 0.83 (d, J = 6.8 Hz, 6H). |
| 25.1 | Methyl 4-bromo- 5-(N-(6-((trans)- 3,5- dimethylpiperidin- 1-yl)pyridin-3- yl)sulfamoyl)-2- fluorobenzoate | 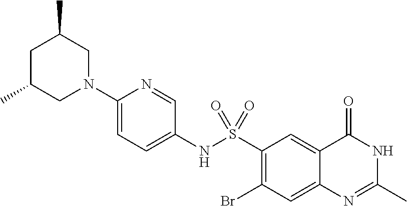 and |
| | | 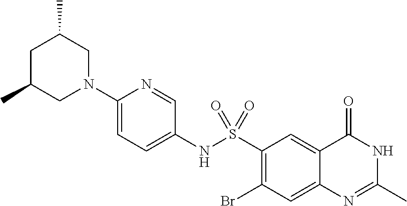 7-Bromo-N-(6-((trans)-3,5-dimethylpiperidin-1- yl)pyridin-3-yl)-2-methyl-4-oxo-3,4-dihydroquinazoline- 6-sulfonamide. ¹H NMR (400 MHz, DMSO-d₆) δ 11.94 (s, 1H), 8.16 (s, 1H), 7.63 (d, J = 2.7 Hz, 1H), 7.08 (dd, J = 9.1, 2.7 Hz, 1H), 6.67 (d, J = 9.3 Hz, 2H), 6.31 (t, J = 5.5 Hz, 1H), 3.49 (dd, J = 12.8, 3.7 Hz, 2H), 3.07 (dd, J = 12.8, 6.8 Hz, 2H), 2.27 (s, 3H), 1.68 (p, J = 6.9 Hz, 2H), 1.37 (t, J = 5.8 Hz, 2H), 0.83 (d, J = 6.8 Hz, 6H). |

### Example 1-26. Intermediate Synthesis 26

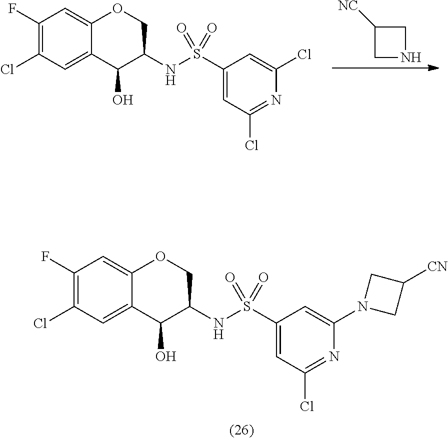 [SMILES: O=S(=O)(N[C@@H]1COc2cc(F)c(Cl)cc2[C@@H]1O)c1cc(Cl)nc(Cl)c1 ; N#CC1CNC1 ; N#CC1CN(c2cc(S(=O)(=O)N[C@@H]3COc4cc(F)c(Cl)cc4[C@@H]3O)cc(Cl)n2)C1]

### Preparation of 2-Chloro-N-((3R,4S)-6-chloro-7-fluoro-4-hydroxychroman-3-yl)-6-(3-cyanoazetidin-1-yl)pyridine-4-sulfonamide (Intermediate 26)

2,6-Dichloro-N-((3R,4S)-6-chloro-7-fluoro-4-hydroxychroman-3-yl)pyridine-4-sulfonamide (24.3) (10 g mg, 0.239 mmol, 1 equiv.) and azetidine-3-carbonitrile (196 mg, 2.39 mmol, 10 equiv) were suspended in NMP (1.5 mL) before being heated in a microwave reactor at 130° C. for 10 min. The product was then isolated by reverse phase HPLC (10->100% MeCN/H₂O with 0.1% formic acid) as a white solid (94 mg, 83%). ¹H NMR (400 MHz, Methanol-d₄) δ 7.35 (d, J=8.3 Hz, 1H), 7.12 (s, 1H), 6.78 (s, 1H), 6.70 (d, J=10.3 Hz, 1H), 4.55 (d, J=3.3 Hz, 1H), 4.41 (t, J=8.6 Hz, 2H), 4.28 (t, J=7.1 Hz, 2H), 4.09 (t, J=10.1 Hz, 1H), 4.02 (dd, J=10.7, 3.2 Hz, 1H), 3.90-3.72 (m, 2H). LC/MS (APCI) m/z calcd. for C₁₈H₁₆Cl₂FN₄O₄S⁺ [M+H]⁺: 473.0; 473.0 found.

TABLE I-26
The following intermediates were prepared according to synthetic procedures as described in Intermediate Synthesis 26.

| | Chloro | | |
|---|---|---|---|
| | pyridine | | |
| Intermediate | intermediate | Amine | Structure, Name and Data |
| 26.1 | 24.3 | azetidine-3- carbonitrile | 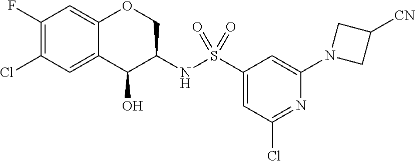 [SMILES: N#CC1CN(c2cc(S(=O)(=O)N[C@@H]3COc4cc(F)c(Cl)cc4[C@@H]3O)cc(Cl)n2)C1] 2-Chloro-N-((3R,4S)-6-chloro-7-fluoro-4- hydroxychroman-3-yl)-6-(3-cyanoazetidin-1- yl)pyridine-4-sulfonamide. ¹H NMR (400 MHz, Methanol-d₄) δ 7.35 (d, J = 8.3 Hz, 1H), 7.12 (s, 1H), 6.78 (s, 1H), 6.70 (d, J = 10.3 Hz, 1H), 4.55 (d, J = 3.3 Hz, 1H), 4.41 (t, J = 8.6 Hz, 2H), 4.28 (t, J = 7.1 Hz, 2H), 4.09 (t, J = 10.1 Hz, 1H), 4.02 (dd, J = 10.7, 3.2 Hz, 1H), 3.90-3.72 (m, 2H). LC/MS (APCI) m/z calcd. for C₁₈H₁₆Cl₂FN₄O₄S⁺ [M + H]⁺: 473.0; 473.0 found. |
| 26.2 | 24.3 | azetidin-3-ol | 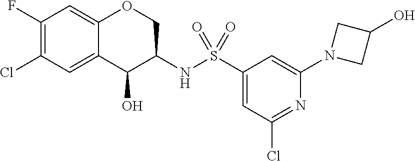 [SMILES: O=S(=O)(N[C@@H]1COc2cc(F)c(Cl)cc2[C@@H]1O)c1cc(Cl)nc(N2CC(O)C2)c1] 2-Chloro-N-((3R,4S)-6-chloro-7-fluoro-4- hydroxychroman-3-yl)-6-(3-hydroxyazetidin-1- yl)pyridine-4-sulfonamide. ¹H NMR (400 MHz, Methanol-d₄) δ 7.36 (d, J = 8.3 Hz, 1H), 7.01 (d, J = 1.2 Hz, 1H), 6.72 (s, 1H), 6.70 (d, J = 8.7 Hz, 1H), 4.72 (tt, J = 6.6, 4.4 Hz, 1H), 4.55 (d, J = 3.8 Hz, 1H), 4.33 (ddt, J = 9.2, 6.7, 1.2 Hz, 2H), 4.09 (dd, J = 10.7, 9.6 Hz, 1H), 4.00 (ddd, J = 10.8, 3.7, 1.0 Hz, 1H), 3.89 (dd, J = 9.4, 4.3 Hz, 2H), 3.77 (dt, J = 9.5, 3.8 Hz, 1H), 3.37 (s, 1H). LC/MS (APCI) m/z calcd. for C₁₇H₁₅Cl₂FN₃O₅S⁻ [M − H]⁻: 462.0; 462.1 found. |
| 26.3 | 24.3 | 3,3- dimethylazetidine | 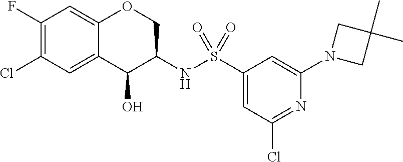 [SMILES: CC1(C)CN(c2cc(S(=O)(=O)N[C@@H]3COc4cc(F)c(Cl)cc4[C@@H]3O)cc(Cl)n2)C1] 2-Chloro-N-((3R,4S)-6-chloro-7-fluoro-4- hydroxychroman-3-yl)-6-(3,3-dimethylazetidin-1- yl)pyridine-4-sulfonamide. ¹H NMR (400 MHz, Methanol-d₄) δ 7.36 (d, J = 8.3 Hz, 1H), 6.97 (s, 1H), 6.70 (d, J = 10.4 Hz, 1H), 6.67 (s, 1H), 4.55 (d, J = 3.2 Hz, 1H), 4.09 (t, J = 10.2 Hz, 1H),4.01 (dd, J = 10.7, 3.2 Hz, 1H), 3.79 (s, 5H), 1.36 (s, 6H). LC/MS (APCI) m/z calcd. for C₁₉H₁₉Cl₂FN₃O₄S⁻ [M − H]⁻: 474.1; 474.1 found. |
| 26.4 | 24.3 | 3- (difluoromethoxy) azetidine | 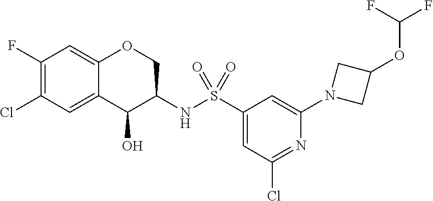 [SMILES: O=S(=O)(N[C@@H]1COc2cc(F)c(Cl)cc2[C@@H]1O)c1cc(Cl)nc(N2CC(OC(F)F)C2)c1] 2-Chloro-N-((3R,4S)-6-chloro-7-fluoro-4- hydroxychroman-3-yl)-6-(3- (difluoromethoxy)azetidin-1-yl)pyridine-4- sulfonamide. ¹H NMR (400 MHz, Methanol-d₄) δ 7.36 (d, J = 8.3 Hz, 1H), 7.06 (s, 1H), 6.76 (d, J = 1.1 Hz, 1H), 6.70 (d, J = 10.4 Hz, 1H), 6.52 (t, J = 74.0 Hz, 1H), 5.15 (ddd, J = 10.8, 6.7, 4.2 Hz, 1H), 4.56 (d, J = 3.8 Hz, 1H), 4.42 (dd, J = 9.7, 6.7 Hz, 2H), 4.14-4.05 (m, 3H), 4.01 (ddd, J = 10.8, 3.7, 1.1 Hz, 1H), 3.78 (dt, J = 9.4, 3.8 Hz, 1H). LC/MS (APCI) m/z calcd. for C₁₈H₁₅Cl₂F₃N₃O₅S⁻ [M − H]⁻: 512.0; 512.1 found. |
| 26.5 | 24.3 | 3- (difluoromethyl) azetidine | 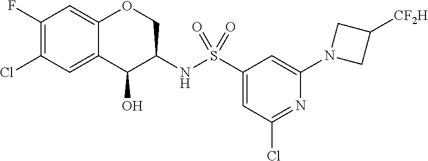 [SMILES: O=S(=O)(N[C@@H]1COc2cc(F)c(Cl)cc2[C@@H]1O)c1cc(Cl)nc(N2CC(C(F)F)C2)c1] 2-Chloro-N-((3R,4S)-6-chloro-7-fluoro-4- hydroxychroman-3-yl)-6-(3- (difluoromethyl)azetidin-1-yl)pyridine-4- sulfonamide. ¹H NMR (400 MHz, Methanol-d₄) δ 7.36 (d, J = 8.3 Hz, 1H), 7.05 (s, 1H), 6.75 (s, 1H), 6.70 (d, J = 10.4 Hz, 1H), 6.18 (td, J = 56.6, 4.3 Hz, 1H), 4.55 (d, J = 3.3 Hz, 1H), 4.21 (t, J = 8.8 Hz, 2H), 4.15-4.04 (m, 3H), 4.01 (dd, J = 10.8, 3.4 Hz, 1H), 3.78 (dt, J = 9.0, 3.9 Hz, 1H), 3.25 (ddt, J = 14.2, 9.6, 4.9 Hz, 1H). |
| 26.6 | 24.3 | (R)- pyrrolidine-3- carbonitrile | 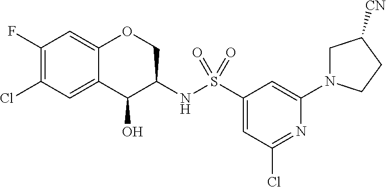 [SMILES: N#C[C@@H]1CCN(c2cc(S(=O)(=O)N[C@@H]3COc4cc(F)c(Cl)cc4[C@@H]3O)cc(Cl)n2)C1] 2-Chloro-N-((3R,4S)-6-chloro-7-fluoro-4- hydroxychroman-3-yl)-6-((R)-3-cyanopyrrolidin- 1-yl)pyridine-4-sulfonamide. ¹H NMR (400 MHz, Methanol-d₄) δ 7.35 (d, J = 8.5 Hz, 1H), 7.04 (s, 1H), 6.87 (s, 1H), 6.69 (d, J = 10.5 Hz, 1H), 4.55 (d, J = 3.8 Hz, 1H), 4.09 (t, J = 10.1 Hz, 1H), 4.01 (dd, J = 10.9, 3.6 Hz, 1H), 3.87 (dd, J = 11.0, 7.5 Hz, 1H), 3.78 (dq, J = 7.5, 3.5 Hz, 2H), 3.68 (t, J = 8.4 Hz, 1H), 3.64-3.55 (m, 1H), 3.50 (p, J = 6.6 Hz, 1H), 2.42 (ddq, J = 44.0, 13.0, 6.7 Hz, 2H). LC/MS (APCI) m/z calcd. for C₁₉H₁₆Cl₂FN₄O₄S⁻ [M − H]⁻: 485.0; 485.1 found. |

### Example I-27. Intermediate Synthesis 27

### Preparation of Ethyl 5-(N-(6-chloro-4-hydroxychroman-3-yl)sulfamoyl)-2-methyl-1H-pyrrole-3-carboxylate (Intermediate 27)

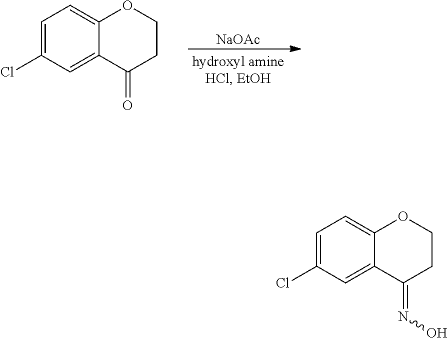 [SMILES: O=C1CCOc2ccc(Cl)cc21 ; ON=C1CCOc2ccc(Cl)cc21 ; "NaOAc"]

### Step 1: 6-chlorochroman-4-one oxime

6-chlorochroman-4-one (4.7 g, 25.8 mmol) was combined with hydroxylamine hydrochloride (3.1 g, 43.9 mmol) and EtOH (30 mL). To the mixture was added a solution of NaOAc (6.4 g, 77.5 mmol) in water (30 mL). The resulting mixture was heated at 100° C. in an oil bath for 2 h. The reaction was cooled to r.t. and aged for 30 min at r.t. The resulting suspension was filtered and the filtered solid was washed with water (25 mL) and dried under high vacuum, providing 6-chlorochroman-4-one oxime (4.6 g, 23.4 mmol, 90% yield) as a white solid. LC/MS (APCI) m/z calcd. for C₉H₈NO₂Cl [M+H]⁺: 197.0; 198.1 found.

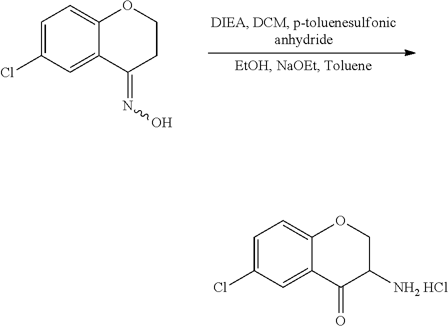

### Step 2: 3-amino-6-chlorochroman-4-one hydrochloride

6-chlorochroman-4-one oxime (4.6 g, 23.4 mmol) was dissolved in DCM (30 mL) and DIEA (6.1 mL, 35.0 mmol) was added. The resulting mixture was cooled to 0° C. with an ice bath and p-toluenesulfonic anhydride (11.4 g, 35.0 mmol) was added portionwise. The resulting mixture was stirred for 2 h, during which time it warmed to r.t. The mixture was diluted with DCM (100 mL) and washed with saturated aq. NaHCO₃ (50 mL), 0.1 M aq. HCl (50 mL), brine (30 mL), dried over sodium sulfate and concentrated under reduced pressure. The remaining solid was triturated with 20% EtOAc/Hex and filtered. The filtered solid was collected and dried under high vacuum. To the solid was added toluene (15 mL) and EtOH (4 mL), followed by NaOEt (8.3 g of 21% by weight in EtOH, 25.7 mmol) with stirring. The resulting mixture was stirred at r.t. for 18 h. The resulting suspension was filtered, and the filtered solid was washed with Et₂O (30 mL). The filtered solid was discarded and the filtrate was concentrated under reduced pressure. To the remaining residue was added 1,4-dioxane (20 mL) and 4 M hydrogen chloride in 1,4-dioxane (20 mL), followed by water (6 mL). The resulting mixture was stirred vigorously at r.t. for 30 min. and then the solvents were removed in vacuo. To the remaining solid was added Et₂O (50 mL) and the resulting suspension was triturated, sonicated and filtered. The filtered solid was dried under high vacuum, providing 3-amino-6-chlorochroman-4-one hydrochloride (3.0 g, 13.0 mmol, 56% yield) as a tan solid. ¹H NMR (400 MHz, DMSO-d₆) δ 9.04 (s, 3H), 7.76 (d, J=2.7 Hz, 1H), 7.71 (dd, J=8.9, 2.7 Hz, 1H), 7.19 (d, J=8.9 Hz, 1H), 4.87 (dd, J=10.7, 5.7 Hz, 1H), 4.77-4.65 (m, 1H), 4.53 (dd, J=13.0, 10.7 Hz, 1H). LC/MS (APCI) m/z calcd. for C₉H8NO₂Cl [M+H]⁺: 197.0; 198.1 found.

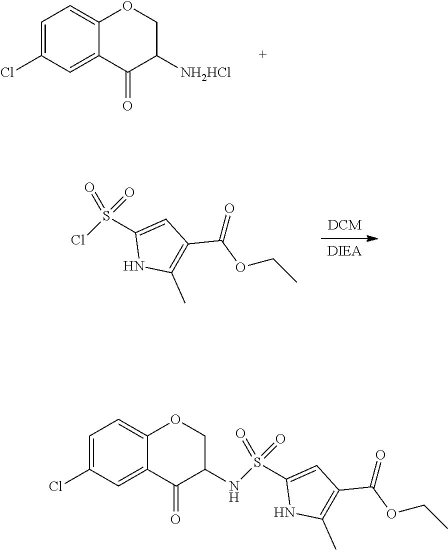

### Step 3: Ethyl 5-(N-(6-chloro-4-oxochroman-3-yl)sulfamoyl)-2-methyl-1H-pyrrole-3-carboxylate

To 3-amino-6-chlorochroman-4-one hydrochloride (778 mg, 3.34 mmol) was added DCM (5 mL) followed by DIEA (2.4 mL, 13.9 mmol). Ethyl 5-(chlorosulfonyl)-2-methyl-1H-pyrrole-3-carboxylate (700 mg, 2.78 mmol) was added and the resulting mixture was stirred at r.t. for 30 min. The reaction was diluted with additional DCM (30 mL) and washed with water (20 mL), brine (20 mL), dried over sodium sulfate and concentrated under reduced pressure. The remaining solid was triturated and sonicated in DCM/Et₂O 1:1 (10 mL) and filtered, providing ethyl 5-(N-(6-chloro-4-oxochroman-3-yl)sulfamoyl)-2-methyl-1H-pyrrole-3-carboxylate (800 mg, 1.94 mmol, 70% yield) as a white solid. ¹H NMR (400 MHz, DMSO-d₆) δ 12.30 (s, 1H), 8.09 (d, J=7.6 Hz, 1H), 7.69-7.64 (m, 1H), 7.63 (d, J=2.7 Hz, 1H), 7.13 (d, J=8.7 Hz, 1H), 6.90 (d, J=2.6 Hz, 1H), 4.68-4.56 (m, 1H), 4.46 (dd, J=11.1, 5.6 Hz, 1H), 4.30-4.22 (m, 1H), 4.18 (q, J=7.0 Hz, 2H), 2.44 (s, 3H), 1.26 (t, J=7.0 Hz, 3H). LC/MS (APCI) m/z calcd. for C₁₇H₁₇N₂O₆SCl [M+H]⁺: 412.1; 413.1 found.

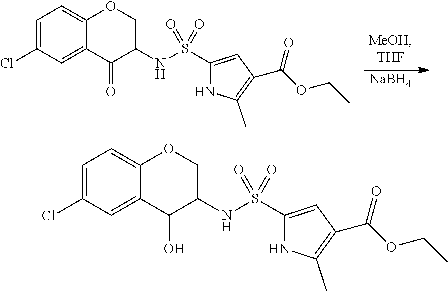

### Step 4: Ethyl 5-(N-(6-chloro-4-hydroxychroman-3-yl)sulfamoyl)-2-methyl-1H-pyrrole-3-carboxylate (Intermediate 27)

Ethyl 5-(N-(6-chloro-4-oxochroman-3-yl)sulfamoyl)-2-methyl-1H-pyrrole-3-carboxylate (416 mg, 1.01 mmol) was dissolved in MeOH (15 mL) and THF (8 mL). NaBH₄ (114 mg, 3.02 mmol) was added and the resulting mixture was stirred at r.t. for 15 min. The reaction was diluted with EtOAc (50 mL) and saturated aq. NaHCO₃ (30 mL). The layers were shaken and separated, and the organic phase was washed with brine (50 mL), dried over sodium sulfate and concentrated in vacuo providing ethyl 5-(N-(6-chloro-4-hydroxychroman-3-yl)sulfamoyl)-2-methyl-1H-pyrrole-3-carboxylate (453 mg, 0.98 mmol, 98% yield) as a white solid which was used as a mixture of diastereomers in the subsequent step. LC/MS (APCI) m/z calcd. for C₁₇H₁₉N₂O₆SCl [M+H]⁺: 414.1; 397.1 found (fragmentation).

### SYNTHETIC EXAMPLES

### Example S-1: Hydrolysis and Amide Coupling General Procedure

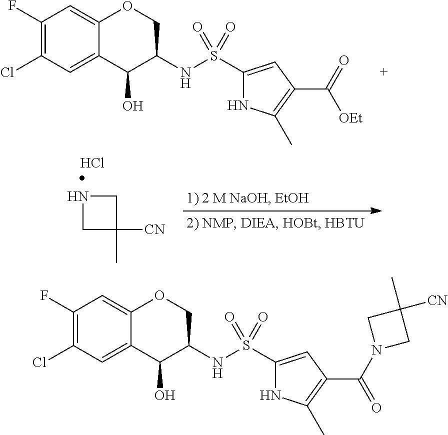

### Preparation of N-((3R,4S)-6-chloro-7-fluoro-4-hydroxychroman-3-yl)-4-(3-cyano-3-methylazetidine-1-carbonyl)-5-methyl-1H-pyrrole-2-sulfonamide

2.0 M NaOH (2.0 mL) was added to ethyl 5-(N-((3R,4S)-6-chloro-7-fluoro-4-hydroxychroman-3-yl)sulfamoyl)-2-methyl-1H-pyrrole-3-carboxylate (45 mg, 0.10 mmol, 1.0 equiv) in ethanol (2.0 mL) before being heated to 100° C. After 5 h, the reaction was returned to rt and ethanol removed by rotary evaporation. The pH was adjusted to 3 using 1.0 M HCl and extracted with ethyl acetate (3×10 mL). The organic extracts were combined, dried over sodium sulfate, filtered, and concentrated under reduced pressure. The remaining solid was dissolved in N-methylpyrrolidone (1.5 mL) before 3-methylazetidine-3-carbonitrile hydrochloride (27 mg, 0.20 mmol), HBTU (76 mg, 0.20 mmol), HOBt (27 mg, 0.20 mmol) and diisopropylethylamine (0.087 mL, 0.50 mmol) were added and the resulting solution was stirred at room temperature for 30 min. The reaction mixture was purified by reverse phase HPLC (10->100% MeCN/water with 0.1% formic acid, Phenomenex Gemini 5 micron Cis column, 150×21 mm, Axia Pack) to give the product as a white solid (31 mg, 64%). ¹H NMR (400 MHz, Methanol-d₄) δ 7.32 (d, J=8.3 Hz, 1H), 6.86 (s, 1H), 6.69 (d, J=10.3, 1H), 4.69 (s, 1H), 4.52-4.19 (m, 3H), 4.04 (td, J=10.4, 1.1 Hz, 2H), 3.93 (ddd, J=10.6, 3.9, 1.1 Hz, 1H), 3.72 (ddd, J=10.3, 4.4, 3.4 Hz, 1H), 2.49 (s, 3H), 1.69 (s, 3H). LC/MS (APCI) m/z calcd. for C₂₀H₂₁ClFN₄O₅S⁺ [M+H]⁺: 483.1; 483.0 found.

TABLE S-1
The following compounds and intermediates were prepared according to the synthetic
procedures of Example S-1.

| | Inter- | | |
|---|---|---|---|
| Compound | mediate | | |
| No | Ester | Amine | Structure, Name and Data |
| 78 | 24.1 | 3- methylazetidine- 3-carbonitrile | 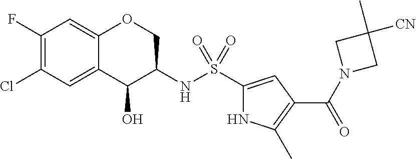 [SMILES: Cc1[nH]c(S(=O)(=O)N[C@@H]2COc3cc(F)c(Cl)cc3[C@@H]2O)cc1C(=O)N1CC(C)(C#N)C1] |
| | | | N-((3R,4S)-6-chloro-7-fluoro-4- |
| | | | hydroxychroman-3-yl)-4-(3-cyano-3- |
| | | | methylazetidine-1-carbonyl)-5-methyl-1H- |
| | | | pyrrole-2-sulfonamide. ¹H NMR (400 MHz, |
| | | | Methanol-d₄) δ 7.32 (d, J = 8.3 Hz, 1H), 6.86 (s, |
| | | | 1H), 6.69 (d, J = 10.3, 1H), 4.69 (s, 1H), 4.52- |
| | | | 4.19 (m, 3H), 4.04 (td, J = 10.4, 1.1 Hz, 2H), 3.93 |
| | | | (ddd, J = 10.6, 3.9, 1.1 Hz, 1H), 3.72 (ddd, J = |
| | | | 10.3, 4.4, 3.4 Hz, 1H), 2.49 (s, 3H), 1.69 (s, 3H). |
| | | | LC/MS (APCI) m/z calcd. for C₂₀H₂₁ClFN₄O₅S⁺ |
| | | | [M + H]⁺: 483.1; 483.0 found. |
| 77 | 24.2 | 3- methylazetidine- 3-carbonitrile | 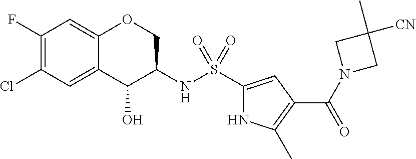 [SMILES: Cc1[nH]c(S(=O)(=O)N[C@@H]2COc3cc(F)c(Cl)cc3[C@H]2O)cc1C(=O)N1CC(C)(C#N)C1] |
| | | | N-((3R,4R)-6-chloro-7-fluoro-4- |
| | | | hydroxychroman-3-yl)-4-(3-cyano-3- |
| | | | methylazetidine-1-carbonyl)-5-methyl-1H- |
| | | | pyrrole-2-sulfonamide. ¹H NMR (400 MHz, |
| | | | Methanol-d₄) δ 7.32 (d, J = 8.2 Hz, 1H), 6.82 (s, |
| | | | 1H), 6.74 (d, J = 10.4 Hz, 1H), 4.82-3.81 (m, |
| | | | 7H), 3.55 (td, J = 4.5, 2.4 Hz, 1H), 2.48 (s, 3H), |
| | | | 1.70 (s, 3H). LC/MS (APCI) m/z calcd. for |
| | | | C₂₀H₂₁ClFN₄O₅S⁺ [M + H]⁺: 483.1; 483.1 found. |
| Chiral Purification Intermediate 1 | 24.4 | 3- methylazetidine- 3-carbonitrile | 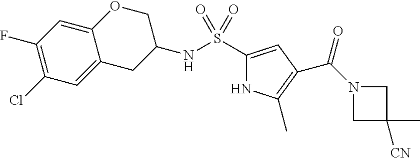 [SMILES: Cc1[nH]c(S(=O)(=O)NC2COc3cc(F)c(Cl)cc3C2)cc1C(=O)N1CC(C)(C#N)C1] |
| | | | N-(6-chloro-7-fluorochroman-3-yl)-4-(3-cyano- |
| | | | 3-methylazetidine-1-carbonyl)-5-methyl-1H- |
| | | | pyrrole-2-sulfonamide. ¹H NMR (400 MHz, |
| | | | DMSO-d₆) δ 12.22 (s, 1H), 7.74 (d, J = 6.2 Hz, |
| | | | 1H), 7.29 (d, J = 8.5 Hz, 1H), 6.88 (d, J = 10.7 |
| | | | Hz, 1H), 6.75 (s, 1H), 4.42 (d, J = 147.8 Hz, 3H), |
| | | | 4.11-3.91 (m, 2H), 3.86 (dd, J = 10.7, 7.5 Hz, |
| | | | 1H), 3.74-3.53 (m, 1H), 2.86 (dd, J = 16.5, 5.2 |
| | | | Hz, 1H), 2.64 (dd, J = 16.3, 7.4 Hz, 1H), 2.41 (s, |
| | | | 3H), 1.62 (s, 3H). LC/MS (APCI) m/z calcd. for |
| | | | C₂₀H₂₁ClFN₄O₄S⁺ [M + H]⁺: 467.1; 467.1 found. |
| 70 | 24.5 | 3- methylazetidine- 3-carbonitrile | 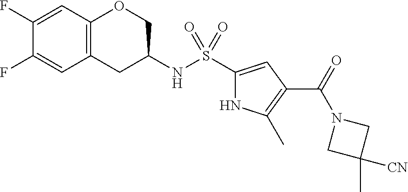 [SMILES: Cc1[nH]c(S(=O)(=O)N[C@@H]2COc3cc(F)c(F)cc3C2)cc1C(=O)N1CC(C)(C#N)C1] |
| | | | (S)-4-(3-cyano-3-methylazetidine-1-carbonyl)- |
| | | | N-(6,7-difluorochroman-3-yl)-5-methyl-1H- |
| | | | pyrrole-2-sulfonamide. ¹H NMR (400 MHz, |
| | | | DMSO-d₆) δ 7.75 (d, J = 6.0 Hz, 1H), 7.23-7.12 |
| | | | (m, 1H), 6.89 (dd, J = 11.9, 7.1 Hz, 1H), 6.75 (s, |
| | | | 1H), 4.35 (t, J = 5.1 Hz, 1H), 4.03 (d, J = 9.6 Hz, |
| | | | 1H), 3.87-3.76 (m, 1H), 3.63 (s, 1H), 3.49-3.38 |
| | | | (m, 1H), 3.33 (s, 3H), 2.84 (dd, J = 15.8, 5.2 Hz, |
| | | | 1H), 2.71-2.58 (m, 1H), 2.41 (s, 3H), 1.62 (s, |
| | | | 2H), 1.06 (t, J = 7.0 Hz, 1H). LC/MS (APCI) m/z |
| | | | calcd. for C₂₀H₂₁F₂N₄O₄S⁺ [M + H]⁺: 451.1; 451.1 |
| | | | found. |
| 67 | 23.1 | 3- methylazetidine- 3-carbonitrile | 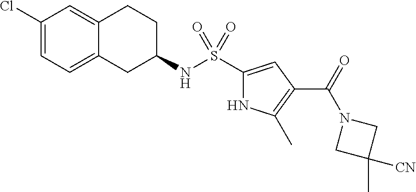 [SMILES: Cc1[nH]c(S(=O)(=O)N[C@@H]2CCc3cc(Cl)ccc3C2)cc1C(=O)N1CC(C)(C#N)C1] |
| | | | (R)-N-(6-chloro-1,2,3,4-tetrahydronaphthalen- |
| | | | 2-yl)-4-(3-cyano-3-methylazetidine-1-carbonyl)- |
| | | | 5-methyl-1H-pyrrole-2-sulfonamide. 1H NMR |
| | | | (400 MHz, DMSO-d6) δ 12.12 (s, 1H), 7.55 (d, |
| | | | J = 7.0 Hz, 1H), 7.12 (d, J = 7.2 Hz, 2H), 7.03 (d, |
| | | | J = 8.6 Hz, 1H), 6.66 (d, J = 2.4 Hz, 1H), 4.25 (s, |
| | | | 2H), 3.94 (s, 2H), 3.51-3.38 (m, 2H), 2.81 (d, J = |
| | | | 17.7 Hz, 2H), 2.77-2.65 (m, 1H), 2.59 (dd, J = |
| | | | 16.6, 9.2 Hz, 1H), 2.40 (s, 3H), 1.79 (d, J = 9.0 |
| | | | Hz, 1H), 1.69-1.51 (m, 1H), 1.06 (t, J = 7.0 Hz, |
| | | | 2H). LC/MS (APCI) m/z calcd. for |
| | | | C₂₁H₂₄ClN₄O₃S⁺ [M + H]⁺: 447.1; 447.1 found. |
| 35 | 23.5 | Methylamine | 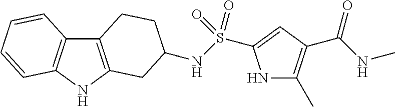 [SMILES: CNC(=O)c1cc(S(=O)(=O)NC2CCc3c([nH]c4ccccc34)C2)[nH]c1C] |
| | | | N,2-dimethyl-5-(N-(2,3,4,9-tetrahydro-1H- |
| | | | carbazol-2-yl)sulfamoyl)-1H-pyrrole-3- |
| | | | carboxamide. ¹H NMR (400 MHz, DMSO-d₆) δ |
| | | | 11.95 (s, 1H), 10.60 (s, 1H), 7.79 (d, J = 4.6 Hz, |
| | | | 1H), 7.53 (d, J = 7.5 Hz, 1H), 7.29 (d, J = 7.5 Hz, |
| | | | 1H), 7.19 (d, J = 7.9 Hz, 1H), 7.01 (d, J = 2.6 Hz, |
| | | | 1H), 6.95 (ddd, J = 8.1, 7.0, 1.3 Hz, 1H), 6.88 (td, |
| | | | J = 7.4, 1.1 Hz, 1H), 3.62-3.47 (m, 1H), 2.82- |
| | | | 2.70 (m, 2H), 2.64 (d, J = 4.5 Hz, 3H), 2.42 (s, |
| | | | 3H), 1.92-1.82 (m, 2H), 1.73-1.61 (m, 2H). |
| | | | LC/MS (APCI) m/z calcd. for C₁₉H₂₃N₄O₃S⁺ |
| | | | [M + H]⁺: 387.1; 387.1 found. |
| 36 | 23.5 | dimethylamine | 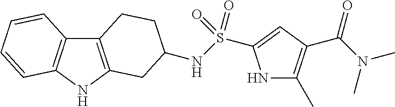 [SMILES: Cc1[nH]c(S(=O)(=O)NC2CCc3c([nH]c4ccccc34)C2)cc1C(=O)N(C)C] |
| | | | N,N,2-trimethyl-5-(N-(2,3,4,9-tetrahydro-1H- |
| | | | carbazol-2-yl)sulfamoyl)-1H-pyrrole-3- |
| | | | carboxamide. ¹H NMR (400 MHz, DMSO-d₆) δ |
| | | | 12.03 (s, 1H), 10.63 (s, 1H), 7.59 (d, J = 7.3 Hz, |
| | | | 1H), 7.31 (d, J = 7.7 Hz, 1H), 7.22 (d, J = 7.9 Hz, |
| | | | 1H), 6.99 (ddd, J = 8.1, 7.0, 1.3 Hz, 1H), 6.91 |
| | | | (ddd, J = 8.0, 7.1, 1.1 Hz, 1H), 6.64 (d, J = 2.0 Hz, |
| | | | 1H), 3.64-3.50 (m, 1H), 2.97 (s, 6H), 2.82 (dd, |
| | | | J = 16.1, 5.5 Hz, 1H), 2.77-2.55 (m, 3H), 2.27 (s, |
| | | | 3H), 1.90 (d, J = 9.2 Hz, 1H), 1.79-1.61 (m, 1H). |
| | | | LC/MS (APCI) m/z calcd. for C₂₀H₂₅N₄O₃S⁺ |
| | | | [M + H]⁺: 401.2; 401.1 found. |
| 37 | 23.5 | 2-methoxyethan- 1-amine | 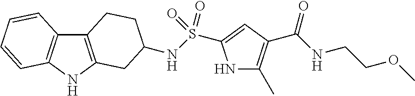 [SMILES: COCCNC(=O)c1cc(S(=O)(=O)NC2CCc3c([nH]c4ccccc34)C2)[nH]c1C] |
| | | | N-(2-methoxyethyl)-2-methyl-5-(N-(2,3,4,9- |
| | | | tetrahydro-1H-carbazol-2-yl)sulfamoyl)-1H- |
| | | | pyrrole-3-carboxamide. ¹H NMR (400 MHz, |
| | | | DMSO-d₆) δ 11.99 (s, 1H), 10.63 (s, 1H), 7.92 (t, |
| | | | J = 5.6 Hz, 1H), 7.55 (d, J = 7.5 Hz, 1H), 7.32 (d, |
| | | | J = 7.7 Hz, 1H), 7.22 (d, J = 7.9 Hz, 1H), 7.11 (d, |
| | | | J = 2.5 Hz, 1H), 6.99 (ddd, J = 8.1, 7.0, 1.3 Hz, |
| | | | 1H), 6.92 (ddd, J = 8.0, 7.0, 1.1 Hz, 1H), 3.67- |
| | | | 3.51 (m, 1H), 3.44-3.35 (m, 2H), 3.33-3.27 (m, |
| | | | 2H), 3.25 (s, 3H), 2.88-2.54 (m, 4H), 2.45 (s, |
| | | | 3H), 1.97-1.84 (m, 1H), 1.70 (qd, J = 11.8, 11.3, |
| | | | 5.6 Hz, 1H). LC/MS (APCI) m/z calcd. for |
| | | | C₂₁H₂₇N₄O₄S⁺ [M + H]⁺: 431.2; 431.1 found. |
| 49 | 23.11 | 3,3- difluoroazetidine | 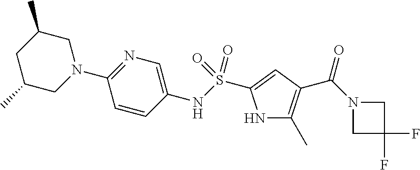 [SMILES: Cc1[nH]c(S(=O)(=O)Nc2ccc(N3C[C@H](C)C[C@@H](C)C3)nc2)cc1C(=O)N1CC(F)(F)C1] |
| | | | 4-(3,3-Difluoroazetidine-1-carbonyl)-N-(6- |
| | | | ((3R,5R)-3,5-dimethylpiperidin-1-yl)pyridin-3- |
| | | | yl)-5-methyl-1H-pyrrole-2-sulfonamide. ¹H |
| | | | NMR (400 MHz, DMSO-d₆) δ 12.08 (s, 1H), 9.25 |
| | | | (s, 1H), 7.49 (d, J = 2.6 Hz, 1H), 6.94 (dd, J = 9.1, |
| | | | 2.7 Hz, 1H), 6.54 (d, J = 9.1 Hz, 1H), 6.30 (s, 1H), |
| | | | 4.25 (s, 4H), 3.34 (dd, J = 12.7, 3.7 Hz, 2H), 2.91 |
| | | | (dd, J = 12.8, 6.8 Hz, 2H), 2.19 (s, 3H), 1.69 (pd, |
| | | | J = 6.6, 3.7 Hz, 2H), 1.22 (t, J = 5.8 Hz, 2H), 0.69 |
| | | | (d, J = 6.8 Hz, 6H). LC/MS (APCI) m/z calcd. |
| | | | for C₂₁H₂₈F₂N₅O₃S⁺ [M + H]⁺: 468.2; 468.2 found. |
| 50 | 23.13 | 3- methylazetidine- 3-carbonitrile | 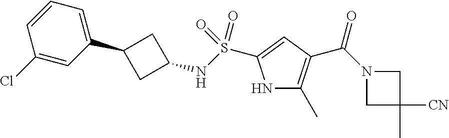 [SMILES: Cc1[nH]c(S(=O)(=O)N[C@H]2C[C@H](c3cccc(Cl)c3)C2)cc1C(=O)N1CC(C)(C#N)C1] |
| | | | N-((trans)-3-(3-chlorophenyl)cyclobutyl)-4-(3- |
| | | | cyano-3-methylazetidine-1-carbonyl)-5-methyl- |
| | | | 1H-pyrrole-2-sulfonamide. ¹H NMR (400 MHz, |
| | | | DMSO-d₆) δ 12.21 (s, 1H), 7.84 (d, J = 6.9 Hz, |
| | | | 1H), 7.34 (t, J = 7.8 Hz, 1H), 7.31 (d, J = 1.9 Hz, |
| | | | 1H), 7.28-7.19 (m, 2H), 6.66 (s, 1H), 4.78-4.07 |
| | | | (m, 4H), 3.87 (d, J = 6.8 Hz, 1H), 3.47 (p, J = 7.7 |
| | | | Hz, 1H), 2.39 (s, 3H), 2.36-2.17 (m, 4H), 1.62 |
| | | | (s, 3H). LC/MS (APCI) m/z calcd. for |
| | | | C₂₁H₂₄ClN₄O₃S⁺ [M + H]⁺: 447.1; 447.1 found. |
| 51 | 23.12 | 3- methylazetidine- 3-carbonitrile | 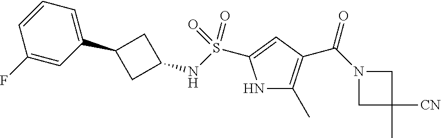 [SMILES: Cc1[nH]c(S(=O)(=O)N[C@H]2C[C@H](c3cccc(F)c3)C2)cc1C(=O)N1CC(C)(C#N)C1] |
| | | | 4-(3-Cyano-3-methylazetidine-1-carbonyl)-N- |
| | | | ((trans)-3-(3-fluorophenyl)cyclobutyl)-5- |
| | | | methyl-1H-pyrrole-2-sulfonamide. ¹H NMR |
| | | | (400 MHz, DMSO-d₆) δ 12.19 (s, 1H), 7.83 (d, |
| | | | J = 7.5 Hz, 1H), 7.35 (td, J = 8.1, 6.3 Hz, 1H), 7.13- |
| | | | 7.06 (m, 2H), 7.05-6.96 (m, 1H), 6.66 (s, 1H), |
| | | | 4.75-4.02 (m, 4H), 3.87 (h, J = 7.9 Hz, 1H), 3.46 |
| | | | (td, J = 8.8, 4.4 Hz, 1H), 2.39 (s, 3H), 2.36-2.17 |
| | | | (m, 4H), 1.62 (s, 3H). LC/MS (APCI) m/z calcd. |
| | | | for C₂₁H₂₄FN₄O₃S⁺ [M + H]⁺: 431.2; 431.1 found. |
| 52 | 23.14 | 3- methylazetidine- 3-carbonitrile | 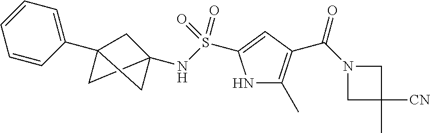 [SMILES: Cc1[nH]c(S(=O)(=O)NC23CC(c4ccccc4)(C2)C3)cc1C(=O)N1CC(C)(C#N)C1] |
| | | | 4-(3-Cyano-3-methylazetidine-1-carbonyl)-5- |
| | | | methyl-N-(3-phenylbicyclo[1.1.1]pentan-l-yl)- |
| | | | 1H-pyrrole-2-sulfonamide. ¹H NMR (400 MHz, |
| | | | DMSO-d₆) δ 12.21 (s, 1H), 8.38 (s, 1H), 7.28 (dd, |
| | | | J = 8.0, 6.5 Hz, 2H), 7.24-7.14 (m, 3H), 6.70 (s, |
| | | | 1H), 4.81-4.40 (m, 2H), 4.33-4.07 (m, 2H), |
| | | | 2.41 (s, 3H), 2.05 (s, 6H), 1.63 (s, 3H). LC/MS |
| | | | (APCI) m/z calcd. for C₂₂H₂₃N₄O₃S⁻ [M − H]⁻: |
| | | | 423.1; 423.1 found. |
| 53 | 23.15 | 3- methylazetidine- 3-carbonitrile | 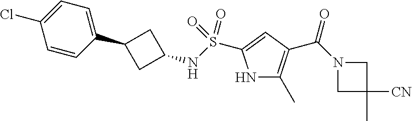 [SMILES: Cc1[nH]c(S(=O)(=O)N[C@H]2C[C@H](c3ccc(Cl)cc3)C2)cc1C(=O)N1CC(C)(C#N)C1] |
| | | | N-((trans)-3-(4-chlorophenyl)cyclobutyl)-4-(3- |
| | | | cyano-3-methylazetidine-1-carbonyl)-5-methyl- |
| | | | 1H-pyrrole-2-sulfonamide. ¹H NMR (400 MHz, |
| | | | DMSO-d₆) δ 12.19 (s, 1H), 7.83 (d, J = 7.8 Hz, |
| | | | 1H), 7.42-7.32 (m, 2H), 7.32-7.21 (m, 2H), |
| | | | 6.65 (s, 1H), 4.72-4.00 (m, 4H), 3.85 (q, J = 7.0 |
| | | | Hz, 1H), 3.44 (dq, J = 9.3, 5.3, 4.7 Hz, 1H), 2.39 |
| | | | (s, 3H), 2.35-2.15 (m, 4H), 1.62 (s, 3H). LC/MS |
| | | | (APCI) m/z calcd. for C₂₁H₂₄ClN₄O₃S⁺ [M + H]⁺: |
| | | | 447.1; 447.1 found. |
| 55 | 23.4 | azetidine-3- carbonitrile | 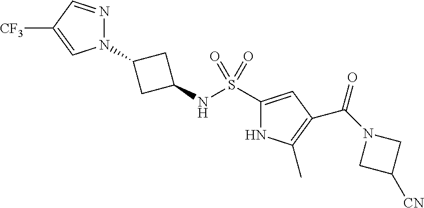 [SMILES: Cc1[nH]c(S(=O)(=O)N[C@H]2C[C@H](n3cc(C(F)(F)F)cn3)C2)cc1C(=O)N1CC(C#N)C1] |
| | | | 4-(3-cyanoazetidine-1-carbonyl)-5-methyl-N- |
| | | | ((trans)-3-(4-(trifluoromethyl)-1H-pyrazol-1- |
| | | | yl)cyclobutyl)-1H-pyrrole-2-sulfonamide. ¹H |
| | | | NMR (400 MHz, DMSO-d₆) δ 12.23 (s, 1H), 8.45 |
| | | | (s, 1H), 7.92 (d, J = 11.8 Hz, 2H), 6.65 (s, 1H), |
| | | | 4.92 (tt, J = 8.9, 4.8 Hz, 1H), 4.79-4.07 (m, 4H), |
| | | | 4.04 (d, J = 7.2 Hz, 1H), 3.80 (ddd, J = 15.3, 9.0, |
| | | | 6.2 Hz, 1H), 2.59 (ddd, J = 13.1, 8.1, 4.8 Hz, 2H), |
| | | | 2.49-2.19 (m, 5H). LC/MS (APCI) m/z calcd. |
| | | | for C₁₈H₂₀F₃N₆O₃S⁺ [M + H]⁺: 457.1; 457.1 found. |
| 56 | 23.3 | azetidine-3- carbonitrile | 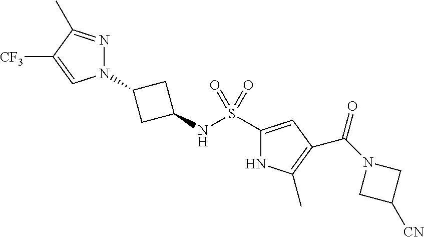 [SMILES: Cc1nn([C@H]2C[C@H](NS(=O)(=O)c3cc(C(=O)N4CC(C#N)C4)c(C)[nH]3)C2)cc1C(F)(F)F] |
| | | | 4-(3-Cyanoazetidine-1-carbonyl)-5-methyl-N- |
| | | | ((trans)-3-(3-methyl-4-(trifluoromethyl)-1H- |
| | | | pyrazol-1-yl)cyclobutyl)-1H-pyrrole-2- |
| | | | sulfonamide. ¹H NMR (400 MHz, DMSO-d₆) δ |
| | | | 12.21 (s, 1H), 8.30 (s, 1H), 7.89 (d, J = 7.8 Hz, |
| | | | 1H), 6.65 (s, 1H), 4.82 (dd, J = 8.9, 4.7 Hz, 1H), |
| | | | 4.57-4.14 (m, 4H), 4.01 (q, J = 6.7 Hz, 1H), 3.86- |
| | | | 3.75 (m, 1H), 2.57 (ddd, J = 13.2, 8.1, 4.9 Hz, |
| | | | 2H), 2.39 (s, 3H), 2.37-2.29 (m, 2H), 2.26 (s, |
| | | | 3H). LC/MS (APCI) m/z calcd. for |
| | | | C₁₉H₂₂F₃N₆O₃S⁺ [M + H]⁺: 471.1; 471.2 found. |
| 38 | 23.2 | ammonia | 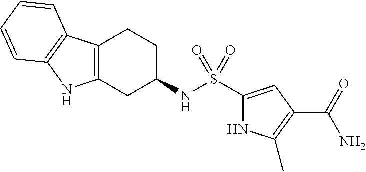 [SMILES: Cc1[nH]c(S(=O)(=O)N[C@@H]2CCc3c([nH]c4ccccc34)C2)cc1C(N)=O] |
| | | | (R)-2-methyl-5-(N-(2,3,4,9-tetrahydro-1H- |
| | | | carbazol-2-yl)sulfamoyl)-1H-pyrrole-3- |
| | | | carboxamide. ¹H NMR (400 MHz, DMSO-d₆) δ |
| | | | 12.01 (s, 1H), 10.64 (s, 1H), 7.57 (d, J = 7.5 Hz, |
| | | | 1H), 7.39 (s, 1H), 7.32 (d, J = 7.7 Hz, 1H), 7.22 |
| | | | (d, J = 7.9 Hz, 1H), 7.09 (s, 1H), 6.98 (t, J = 8.1 |
| | | | Hz, 1H), 6.91 (t, J = 7.4 Hz, 1H), 6.78 (s, 1H), |
| | | | 3.64-3.50 (m, 1H), 2.90-2.53 (m, 4H), 2.45 (s, |
| | | | 3H), 1.98-1.80 (m, 1H), 1.71 (tt, J = 10.5, 6.6 |
| | | | Hz, 1H). LC/MS (APCI) m/z calcd. for |
| | | | C₁₈H₂₁N₄O₃S⁺ [M + H]:⁺: 373.1; 373.1 found. |
| 39 | 23.2 | ethanolamine | 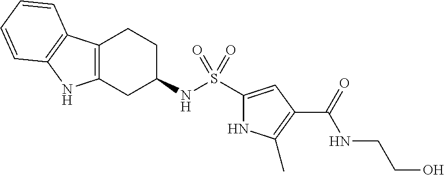 [SMILES: Cc1[nH]c(S(=O)(=O)N[C@@H]2CCc3c([nH]c4ccccc34)C2)cc1C(=O)NCCO] |
| | | | (R)-N-(2-hydroxyethyl)-2-methyl-5-(N-(2,3,4,9- |
| | | | tetrahydro-1H-carbazol-2-yl)sulfamoyl)-1H- |
| | | | pyrrole-3-carboxamide.¹H NMR (400 MHz, |
| | | | DMSO-d₆) δ 12.02 (s, 1H), 10.63 (s, 1H), 7.84 (t, |
| | | | J = 5.6 Hz, 1H), 7.57 (d, J = 7.5 Hz, 1H), 7.32 (d, |
| | | | J = 7.7 Hz, 1H), 7.22 (d, J = 7.9 Hz, 1H), 7.10 (s, |
| | | | 1H), 6.98 (t, J = 7.5 Hz, 1H), 6.92 (t, J = 7.4 Hz, |
| | | | 1H), 4.66 (s, 1H), 3.67-3.52 (m, 1H), 3.45 (t, J = |
| | | | 6.2 Hz, 2H), 3.22 (q, J = 6.1 Hz, 2H), 2.90-2.54 |
| | | | (m, 4H), 2.45 (s, 3H), 1.91 (d, J = 12.7 Hz, 1H), |
| | | | 1.70 (tq, J = 10.2, 5.7 Hz, 1H). LC/MS (APCI) |
| | | | m/z calcd. for C₂₀H₂₅N₄O₄S⁺ [M + H]: 417.2: |
| | | | 417.1 found. |
| 40 | 23.2 | 3,3- dimethylazetidine | 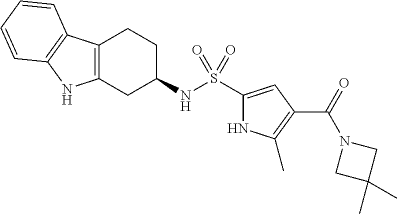 [SMILES: Cc1[nH]c(S(=O)(=O)N[C@@H]2CCc3c([nH]c4ccccc34)C2)cc1C(=O)N1CC(C)(C)C1] |
| | | | (R)-4-(3,3-dimethylazetidine-1-carbonyl)-5- |
| | | | methyl-N-(2,3,4,9-tetrahydro-1H-carbazol-2- |
| | | | yl)-1H-pyrrole-2-sulfonamide. ¹H NMR (400 |
| | | | MHz, DMSO-d₆) δ 12.16 (s, 1H), 10.63 (s, 1H), |
| | | | 7.57 (d, J = 7.4 Hz, 1H), 7.31 (d, J = 7.7 Hz, 1H), |
| | | | 7.22 (d, J = 7.9 Hz, 1H), 6.98 (ddd, J = 8.1, 7.0, |
| | | | 1.3 Hz, 1H), 6.95-6.83 (m, 1H), 6.69 (d, J = 2.2 |
| | | | Hz, 1H), 3.94 (s, 2H), 3.70-3.50 (m, 3H), 2.84- |
| | | | 2.55 (m, 4H), 2.42 (s, 3H), 1.98-1.84 (m, 1H), |
| | | | 1.70 (tq, J = 12.3, 5.6 Hz, 1H), 1.22 (s, 6H). |
| | | | LC/MS (APCI) m/z calcd. for C₂₃H₂₉N₄O₃S⁺ |
| | | | [M + H]⁺: 441.2: 441.2 found. |
| 41 | 23.2 | azetidine | 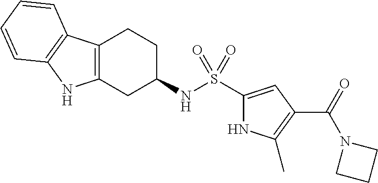 [SMILES: Cc1[nH]c(S(=O)(=O)N[C@@H]2CCc3c([nH]c4ccccc34)C2)cc1C(=O)N1CCC1] |
| | | | (R)-4-(azetidine-1-carbonyl)-5-methyl-N- |
| | | | (2,3,4,9-tetrahydro-1H-carbazol-2-yl)-1H- |
| | | | pyrrole-2-sulfonamide. ¹H NMR (400 MHz, |
| | | | Methanol-d₄) δ 7.31 (d, J = 7.8 Hz, 1H), 7.19 (d, |
| | | | J = 8.0 Hz, 1H), 6.98 (td, J = 7.5, 1.3 Hz, 1H), 6.95- |
| | | | 6.84 (m, 1H), 6.77 (s, 1H), 4.31 (d, J = 8.5 Hz, |
| | | | 2H), 4.09 (t, J = 7.9 Hz, 2H), 3.70 (dddd, J = 10.2, |
| | | | 8.4, 5.4, 2.9 Hz, 1H), 2.85 (dd, J = 16.0, 5.3 Hz, |
| | | | 1H), 2.76 (dt, J = 15.7, 5.2 Hz, 1H), 2.62 (dddd, |
| | | | J = 17.8, 16.2, 7.6, 3.5 Hz, 2H), 2.46 (s, 3H), 2.37- |
| | | | 2.22 (m, 2H), 2.01-1.87 (m, 1H), 1.78 (dtd, J = |
| | | | 12.8, 9.4, 5.8 Hz, 1H). LC/MS (APCI) m/z calcd. |
| | | | for C₂₁H₂₅N₄O₃S⁺ [M + H]⁺: 413.2: 413.2 found. |
| 42 | 23.2 | 3'3- difluoroazetidine | 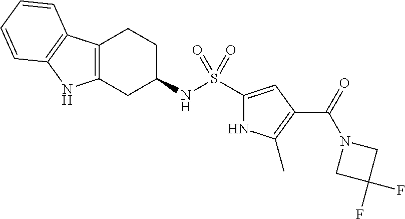 [SMILES: Cc1[nH]c(S(=O)(=O)N[C@@H]2CCc3c([nH]c4ccccc34)C2)cc1C(=O)N1CC(F)(F)C1] |
| | | | (R)-4-(3,3-difluoroazetidine-1-carbonyl)-5- |
| | | | methyl-N-(2,3,4,9-tetrahydro-1H-carbazol-2- |
| | | | yl)-1H-pyrrole-2-sulfonamide. ¹H NMR (400 |
| | | | MHz, Methanol-d₄) δ 7.34 (dd. J = 7.6, 1.2 Hz, |
| | | | 1H), 7.28-7.15 (m, 1H). 7.02 (ddd, J, 8.1, 6.9, |
| | | | 1.3 Hz, 1H). 6.95 (td, J = 7.5, 1.2 Hz, 1H), 6.75 (s, |
| | | | 1H), 3.81 (t. J = 5.1 Hz, 4H), 3.71 (dtd, J = 9.9, |
| | | | 7.8. 4.9 Hz, 1H), 2.86-2.74 (m, 2H), 2.74-2.64 |
| | | | (m, 3H), 2.37 (s, 2H), 1.99 (dq, J = 13.5, 4.7 Hz, |
| | | | 1H). 1.82 (dtd, J = 12.9, 9.4, 5.7 Hz. 1H), LCMS- |
| | | | ESI (POS.) m/z: 449.2 (M + H). LC/MS (APCI) |
| | | | m/z calcd. for C₂₁H₂₃F₂N₄O₃S⁺ [M + H]⁺: 449.1: |
| | | | 449.2 found. |
| 43 | 23.2 | 3- methylazetidin- 3-ol | 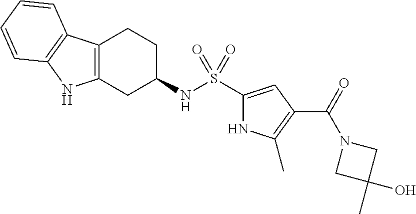 [SMILES: Cc1[nH]c(S(=O)(=O)N[C@@H]2CCc3c([nH]c4ccccc34)C2)cc1C(=O)N1CC(C)(O)C1] |
| | | | (R)-4-(3-cyano-3-methylazetidine-1-carbonyl)- |
| | | | 5-methyl-N-(2,3,4,9-tetrahydro-1H-carbazol-2- |
| | | | yl)-1H-pyrrole-2-sulfonamide. ¹H NMR (400 |
| | | | MHz, DMSO-d₆) δ 12.15 (s, 1H), 10.63 (s, 1H), |
| | | | 7.58 (d, J = 7.4 Hz, 1H), 7.32 (d, J = 7.7 Hz, 1H), |
| | | | 7.26-7.17 (m, 1H), 7.02-6.95 (m, 1H), 6.92 (td, |
| | | | J = 7.4, 1.1 Hz, 1H), 6.68 (d, J = 2.4 Hz, 1H), 5.60 |
| | | | (s, 1H), 4.18-4.03 (m, 2H), 3.90-3.73 (m, 2H), |
| | | | 3.67-3.49 (m, 1H), 2.87-2.54 (m, 4H), 2.42 (s, |
| | | | 3H), 1.90 (dd, J = 9.9, 5.1 Hz, 1H), 1.78-1.60 |
| | | | (m, 1H), 1.37 (s, 3H). LC/MS (APCI) m/z calcd. |
| | | | for C₂₃H₂₇N₅O₃S⁺ [M + H]⁺: 443.2: 443.2 found. |
| 44 | 23.2 | 2-fluoroethan-1- amine | 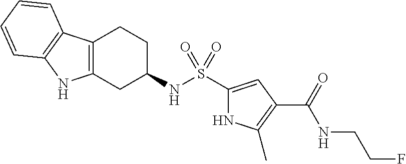 [SMILES: Cc1[nH]c(S(=O)(=O)N[C@@H]2CCc3c([nH]c4ccccc34)C2)cc1C(=O)NCCF] |
| | | | (R)-N-(2-fluoroethyl)-2-methyl-5-(N-(2,3,4,9- |
| | | | tetrahydro-1H-carbazol-2-yl)sulfamoyl)-1H- |
| | | | pyrrole-3-carboxamide. ¹H NMR (400 MHz, |
| | | | Methanol-d₄) δ 7.40-7.27 (m, 1H), 7.27-7.18 |
| | | | (m, 1H), 7.08 (s, 1H), 7.05-6.98 (m, 1H), 6.94 |
| | | | (td, J = 7.5, 1.2 Hz, 1H), 4.58 (td, J = 5.3, 1.6 Hz, |
| | | | 1H), 4.46 (td, J = 5.2, 1.6 Hz, 1H), 3.74 (dtt, J = |
| | | | 11.0, 5.3, 2.6 Hz, 1H), 3.69-3.60 (m, 1H), 3.60- |
| | | | 3.51 (m, 1H), 2.92 (dd, J = 15.8, 5.4 Hz, 1H), 2.85- |
| | | | 2.74 (m, 1H), 2.74-2.58 (m, 2H), 2.55 (s, 3H), |
| | | | 2.03-1.91 (m, 1H), 1.81 (dtd, J = 13.0, 9.4, 5.8 |
| | | | Hz, 1H). LC/MS (APCI) m/z calcd. for |
| | | | C₂₀H₂₄FN₄O₃S⁺ [M + H]⁺: 419.2: 419.2 found. |
| 45 | 23.2 | 2,2,2- trifluoroethan-1- amine | 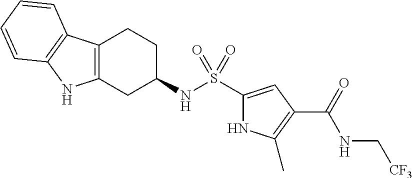 [SMILES: Cc1[nH]c(S(=O)(=O)N[C@@H]2CCc3c([nH]c4ccccc34)C2)cc1C(=O)NCC(F)(F)F] |
| | | | (R)-2-methyl-5-(N-(2,3,4,9-tetrahydro-1H- |
| | | | carbazol-2-yl)sulfamoyl)-N-(2,2,2- |
| | | | trifluoroethyl)-1H-pyrrole-3-carboxamide. ¹H |
| | | | NMR (400 MHz, Methanol-d₄) δ 7.38-7.30 (m, |
| | | | 1H), 7.22 (dd, J = 8.1, 1.0 Hz, 1H), 7.11 (s, 1H), |
| | | | 7.01 (ddd, J = 8.1, 7.0, 1.3 Hz, 1H), 6.95 (td, J = |
| | | | 7.5, 7.0, 1.1 Hz, 1H), 4.08-3.90 (m, 2H), 3.83- |
| | | | 3.65 (m, 1H), 2.92 (dd, J = 15.8, 5.4 Hz, 1H), 2.86- |
| | | | 2.73 (m, 1H), 2.74-2.57 (m, 2H), 2.54 (s, 3H), |
| | | | 2.02-1.90 (m, 1H), 1.82 (dtd, J = 12.9, 9.4, 5.8 |
| | | | Hz, 1H). LC/MS (APCI) m/z calcd. for |
| | | | C₂₀H₂₂F₃N₄O₃S⁺ [M + H]⁺: 455.1; 455.2 found. |
| 46 | 23.2 | pyrrolidine | 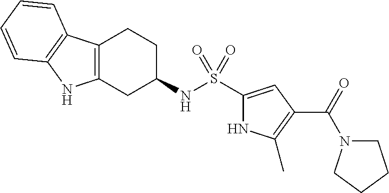 [SMILES: Cc1[nH]c(S(=O)(=O)N[C@@H]2CCc3c([nH]c4ccccc34)C2)cc1C(=O)N1CCCC1] |
| | | | (R)-5-methyl-4-(pyrrolidine-1-carbonyl)-N- |
| | | | (2,3,4,9-tetrahydro-1H-carbazol-2-yl)-1H- |
| | | | pyrrole-2-sulfonamide. ¹H NMR (400 MHz, |
| | | | Methanol-d₄) δ 7.34 (d, J = 7.8 Hz, 1H), 7.22 (d, |
| | | | J = 7.8 Hz, 1H), 7.02 (td, J = 8.0, 7.5, 1.4 Hz, 1H), |
| | | | 6.95 (td, J = 7.5, 1.2 Hz, 1H), 6.88 (s, 1H), 3.72 |
| | | | (dtd, J = 11.0, 5.5, 2.7 Hz, 1H), 3.58 (dt, J = 13.1, |
| | | | 6.8 Hz, 4H), 2.92-2.74 (m, 2H), 2.74-2.54 (m, |
| | | | 2H), 2.43 (s, 3H), 1.95 (ddq, J = 17.3, 12.0, 6.2, |
| | | | 5.2 Hz, 5H), 1.82 (dtd, J = 13.0, 9.5, 5.9 Hz, 1H). |
| | | | LC/MS (APCI) m/z calcd. for C₂₂H₂₇N₄O₃S⁺ |
| | | | [M + H]⁺: 427.2; 427.2 found. |
| 47 | 23.2 | piperidine | 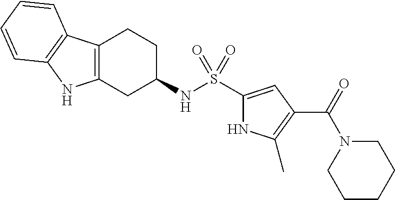 [SMILES: Cc1[nH]c(S(=O)(=O)N[C@@H]2CCc3c([nH]c4ccccc34)C2)cc1C(=O)N1CCCCC1] |
| | | | (R)-5-methyl-4-(piperidine-1-carbonyl)-N- |
| | | | (2,3,4,9-tetrahydro-1H-carbazol-2-yl)-1H- |
| | | | pyrrole-2-sulfonamide. ¹H NMR (400 MHz, |
| | | | Methanol-d₄) δ 7.32 (d, J = 7.9 Hz, 1H), 7.20 (d, |
| | | | J = 8.0 Hz, 1H), 7.04-6.95 (m, 1H), 6.97-6.88 |
| | | | (m, 1H), 6.63 (s, 1H), 3.69 (dtd, J = 11.3, 5.4, 2.6 |
| | | | Hz, 1H), 3.59 (s, 4H), 2.86 (dd, J = 15.9, 5.3 Hz, |
| | | | 1H), 2.77 (dt, J = 15.6, 5.1 Hz, 1H), 2.72-2.54 |
| | | | (m, 2H), 2.32 (s, 3H), 2.01-1.91 (m, 1H), 1.86- |
| | | | 1.74 (m, 1H), 1.78-1.64 (m, 2H), 1.58-1.43 (m, |
| | | | 4H). LC/MS (APCI) m/z calcd. for C₂₃H₂₉N₄O₃S⁺ |
| | | | [M + H]⁺: 441.2; 441.2 found. |
| 48 | 23.2 | 3- methylazetidine- 3-carbonitrile | 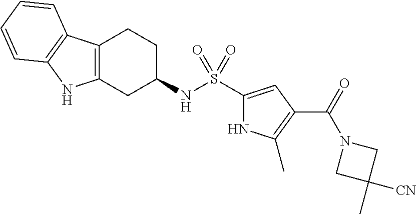 [SMILES: Cc1[nH]c(S(=O)(=O)N[C@@H]2CCc3c([nH]c4ccccc34)C2)cc1C(=O)N1CC(C)(C#N)C1] |
| | | | (R)-4-(3-cyano-3-methylazetidine-1-carbonyl)- |
| | | | 5-methyl-N-(2,3,4,9-tetrahydro-1H-carbazol-2- |
| | | | yl)-1H-pyrrole-2-sulfonamide. ¹H NMR (400 |
| | | | MHz, DMSO-d₆) δ 12.23 (s, 1H), 10.62 (s, 1H), |
| | | | 7.59 (d, J = 7.5 Hz, 1H), 7.32 (d, J = 7.7 Hz, 1H), |
| | | | 7.22 (d, J = 7.9 Hz, 1H), 6.99 (ddd, J = 8.1, 7.1, |
| | | | 1.3 Hz, 1H), 6.92 (td, J = 7.5, 1.1 Hz, 1H), 6.72 |
| | | | (d, J = 2.4 Hz, 1H), 4.74-3.93 (m, 4H), 3.66- |
| | | | 3.51 (m, 1H), 2.87-2.56 (m, 4H), 2.42 (s, 3H), |
| | | | 1.91 (d, J = 12.6 Hz, 1H), 1.71 (tq, J = 12.4, 5.7 |
| | | | Hz, 1H), 1.62 (s, 3H). LC/MS (APCI) m/z calcd. |
| | | | for C₂₃H₂₆N₅O₃S⁺ [M + H]⁺: 452.2; 452.2 found. |
| 54 | 23.16 | 3- methylazetidine- 3-carbonitrile | 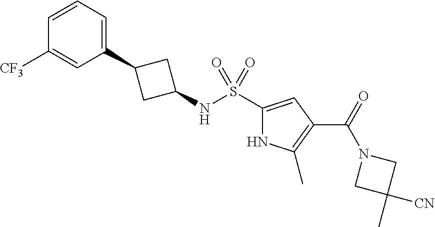 [SMILES: Cc1[nH]c(S(=O)(=O)N[C@H]2C[C@@H](c3cccc(C(F)(F)F)c3)C2)cc1C(=O)N1CC(C)(C#N)C1] |
| | | | 4-(3-Cyano-3-methylazetidine-1-carbonyl)-5- |
| | | | methyl-N-((cis)-3-(3- |
| | | | (trifluoromethyl)phenyl)cyclobutyl)-1H- |
| | | | pyrrole-2-sulfonamide. ¹H NMR (400 MHz, |
| | | | DMSO-d₆) δ 12.23 (s, 1H), 7.77 (d, J = 8.9 Hz, |
| | | | 1H), 7.58 (s, 1H), 7.55-7.46 (m, 3H), 6.67 (s, |
| | | | 1H), 4.25 (t, J = 123.7 Hz, 6H), 3.73 (h, J = 8.4 |
| | | | Hz, 1H), 3.16 (tt, J = 10.2, 7.6 Hz, 1H), 2.40 (s, |
| | | | 3H), 1.94 (qd, J = 9.1, 2.7 Hz, 2H), 1.61 (s, 3H). |
| | | | LC/MS (APCI) m/z calcd. for C₂₂H₂₄F₃N₄O₃S⁺ |
| | | | [M + H]⁺: 481.1; 481.1 found. |
| 57 | 23.17 | azetidine-3- carbonitrile | 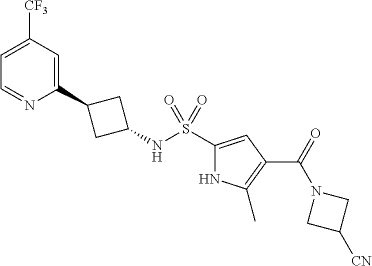 [SMILES: Cc1[nH]c(S(=O)(=O)N[C@H]2C[C@H](c3cc(C(F)(F)F)ccn3)C2)cc1C(=O)N1CC(C#N)C1] |
| | | | 4-(3-Cyanoazetidine-1-carbonyl)-5-methyl-N- |
| | | | ((trans)-3-(4-(trifluoromethyl)pyridin-2- |
| | | | yl)cyclobutyl)-1H-pyrrole-2-sulfonamide. ¹H |
| | | | NMR (400 MHz, Methanol-d₄) δ 8.83-8.74 (m, |
| | | | 1H), 7.51 (d, J = 3.0 Hz, 2H), 6.78 (s, 1H), 4.40 |
| | | | (d, J = 104.5 Hz, 5H), 4.18 (p, J = 7.7 Hz, 1H), |
| | | | 3.76 (tt, J = 9.2, 6.1 Hz, 1H), 3.67 (tt, J = 9.5, 4.5 |
| | | | Hz, 1H), 2.55-2.45 (m, 4H), 2.38 (tdd, J = 9.8, |
| | | | 7.2, 2.3 Hz, 2H). LC/MS (APCI) m/z calcd. for |
| | | | C₂₀H₂₁F₃N₅O₃S⁺ [M + H]⁺: 468.1; 468.2 found. |
| 58 | 23.17 | 3- methylazetidine- 3-carbonitrile | 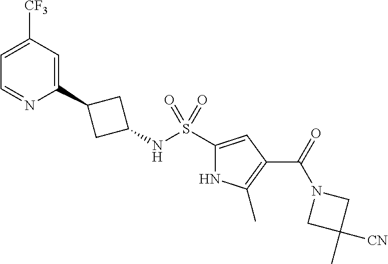 [SMILES: Cc1[nH]c(S(=O)(=O)N[C@H]2C[C@H](c3cc(C(F)(F)F)ccn3)C2)cc1C(=O)N1CC(C)(C#N)C1] |
| | | | 4-(3-Cyano-3-methylazetidine-1-carbonyl)-5- |
| | | | methyl-N-(trans)-3-(4-(trifluoromethyl)pyridin- |
| | | | 2-yl)cyclobutyl)-1H-pyrrole-2-sulfonamide. ¹H |
| | | | NMR (400 MHz, DMSO-d₆) δ 12.19 (s, 1H), 8.83 |
| | | | (d, J = 5.0 Hz, 1H), 7.84 (d, J = 8.5 Hz, 1H), 7.60 |
| | | | (s, 1H), 6.64 (d, J = 1.3 Hz, 1H), 4.81-3.77 (m, |
| | | | 6H), 3.63 (tt, J = 9.0, 4.5 Hz, 1H), 2.41-2.25 (m, |
| | | | 7H), 1.61 (s, 3H). LC/MS (APCI) m/z calcd. for |
| | | | C₂₁H₂₃F₃N₅O₃S⁺ [M + H]⁺: 482.1; 482.2 found. |
| Chiral Purification Intermediate 2 | 23.18 | 3- methylazetidine- 3-carbonitrile | 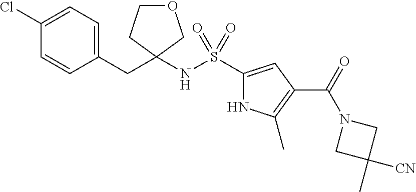 [SMILES: Cc1[nH]c(S(=O)(=O)NC2(Cc3ccc(Cl)cc3)CCOC2)cc1C(=O)N1CC(C)(C#N)C1] |
| | | | N-(3-(4-chlorobenzyl)tetrahydrofuran-3-yl)-4- |
| | | | (3-cyano-3-methylazetidine-1-carbonyl)-5- |
| | | | methyl-1H-pyrrole-2-sulfonamide. ¹H NMR |
| | | | (400 MHz, DMSO-d₆) δ 12.16 (s, 1H), 7.53 (s, |
| | | | 1H), 7.36 (d, J = 8.4 Hz, 2H), 7.29 (d, J = 8.5 Hz, |
| | | | 2H), 6.65 (s, 1H), 4.47-3.91 (m, 4H), 3.79-3.64 |
| | | | (m, 2H), 3.62-3.49 (m, 2H), 3.12-2.89 (m, 2H), |
| | | | 2.39 (s, 3H), 2.04 (ddd, J = 12.2, 7.4, 4.4 Hz, 1H), |
| | | | 1.85 (dt, J = 13.0, 8.2 Hz, 1H), 1.62 (s, 3H). |
| | | | LC/MS (APCI) m/z calcd. for C₂₂H₂₆ClN₄O₄S⁺ |
| | | | [M + H]⁺: 477.1; 477.1 found. |
| Chiral Purification Intermediate 3 | 23.18 | 3,3- difluoroazetidine | 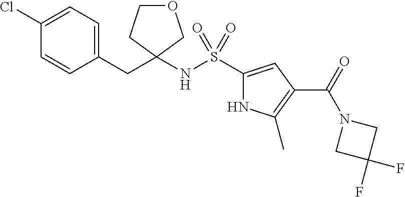 [SMILES: Cc1[nH]c(S(=O)(=O)NC2(Cc3ccc(Cl)cc3)CCOC2)cc1C(=O)N1CC(F)(F)C1] |
| | | | N-(3-(4-Chlorobenzyl)tetrahydrofuran-3-yl)-4- |
| | | | (3,3-difluoroazetidine-1-carbonyl)-5-methyl- |
| | | | 1H-pyrrole-2-sulfonamide. ¹H NMR (400 MHz, |
| | | | DMSO-d₆) δ 12.19 (s, 1H), 7.54 (s, 1H), 7.36 (d, |
| | | | J = 8.4 Hz, 2H), 7.29 (d, J = 8.5 Hz, 2H), 6.68 (s, |
| | | | 1H), 4.50 (s, 4H), 3.79-3.65 (m, 2H), 3.54 (dd, |
| | | | J = 16.8, 8.5 Hz, 2H), 3.13-2.91 (m, 2H), 2.40 (s, |
| | | | 3H), 2.05 (ddd, J = 12.2, 7.4, 4.3 Hz, 1H), 1.86 |
| | | | (dt, J = 13.0, 8.3 Hz, 1H). LC/MS (APCI) m/z |
| | | | calcd. for C₂₀H₂₃ClF₂N₃O₄S⁺ [M + H]⁺: 474.1; |
| | | | 474.1 found. |
| Chiral Purification Intermediate 4 | 23.18 | 2,2- difluoroethan-1- amine | 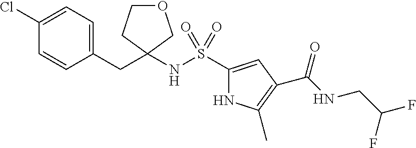 [SMILES: Cc1[nH]c(S(=O)(=O)NC2(Cc3ccc(Cl)cc3)CCOC2)cc1C(=O)NCC(F)F] |
| | | | 5-(N-(3-(4-chlorobenzyl)tetrahydrofuran-3- |
| | | | yl)sulfamoyl)-N-(2,2-difluoroethyl)-2-methyl- |
| | | | 1H-pyrrole-3-carboxamide. 1H NMR (400 |
| | | | MHz, DMSO-d6) δ 12.00 (s, 1H), 8.25 (t, J = 5.9 |
| | | | Hz, 1H), 7.51 (s, 1H), 7.41-7.33 (m, 2H), 7.33- |
| | | | 7.23 (m, 2H), 7.09 (s, 1H), 6.05 (t, J = 4.2 Hz, |
| | | | 1H), 3.78-3.62 (m, 2H), 3.62-3.45 (m, 4H), |
| | | | 3.01 (s, 2H), 2.43 (s, 3H), 2.08 (ddd, J = 12.4, 7.5, |
| | | | 4.5 Hz, 1H), 1.82 (dt, J = 12.9, 8.2 Hz, 1H). |
| | | | LC/MS (APCI) m/z calcd. for C18H21F3N5O3S+ |
| | | | [M + H]+: 462.1; 462.1 found. |
| 81 | 24.6 | azetidine-3- carbonitrile | 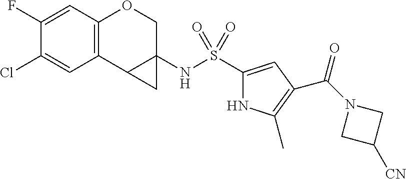 [SMILES: Cc1[nH]c(S(=O)(=O)NC23COc4cc(F)c(Cl)cc4C2C3)cc1C(=O)N1CC(C#N)C1] |
| | | | N-(6-chloro-5-fluoro-1,7b- |
| | | | dihydrocyclopropa[c]chromen-1a(2H)-yl)-4-(3- |
| | | | cyanoazetidine-1-carbonyl)-5-methyl-1H- |
| | | | pyrrole-2-sulfonamide. ¹H NMR (400 MHz, |
| | | | Methanol-d₄) δ 7.24 (d, J = 8.1 Hz, 1H), 6.76 (s, |
| | | | 1H), 6.68 (d, J = 10.0 Hz, 1H), 4.63-4.16 (m, |
| | | | 5H), 3.82-3.62 (m, 2H), 2.47 (s, 3H), 2.06-1.93 |
| | | | (m, 1H), 1.35-1.10 (m, 2H). LC/MS (APCI) m/z |
| | | | calcd. for C₂₀H₁₈N₄O₄FSCl [M + H]⁺: 464.1; 465.1 |
| | | | found. |
| 31 | 24.6 | 3- methylazetidine- 3-carbonitrile | 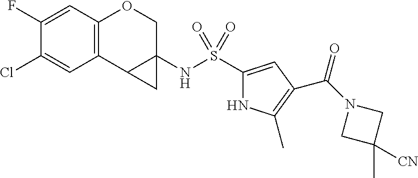 [SMILES: Cc1[nH]c(S(=O)(=O)NC23COc4cc(F)c(Cl)cc4C2C3)cc1C(=O)N1CC(C)(C#N)C1] |
| | | | N-(6-chloro-5-fluoro-1,7b- |
| | | | dihydrocyclopropa[c]chromen-1a(2H)-yl)-4-(3- |
| | | | cyano-3-methylazetidine-1-carbonyl)-5-methyl- |
| | | | 1H-pyrrole-2-sulfonamide. ¹H NMR (400 MHz, |
| | | | Methanol-d₄) δ 7.20 (d, J = 8.1 Hz, 1H), 6.73 (s, |
| | | | 1H), 6.63 (d, J = 10.0 Hz, 1H), 4.61-4.28 (m, |
| | | | 2H), 4.19 (d, J = 10.3 Hz, 1H), 4.16-3.89 (m, |
| | | | 2H), 3.64 (d, J = 10.3 Hz, 1H), 2.44 (s, 3H), 2.06- |
| | | | 1.92 (m, 1H), 1.64 (s, 3H), 1.25-1.09 (m, 2H). |
| | | | LC/MS (APCI) m/z calcd. for C₂₁H₂₀N₄O₄FSCl |
| | | | [M + H]⁺: 478.1; 479.1 found. |
| 16 (Chiral Purification Intermediate 6) | 23.23 | 3- methylazetidine- 3-carbonitrile | 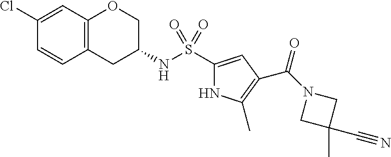 [SMILES: Cc1[nH]c(S(=O)(=O)N[C@H]2COc3cc(Cl)ccc3C2)cc1C(=O)N1CC(C)(C#N)C1] |
| | | | N-(7-chlorochroman-3-yl)-4-(3-cyano-3- |
| | | | methylazetidine-1-carbonyl)-5-methyl-1H- |
| | | | pyrrole-2-sulfonamide. ¹H NMR (400 MHz, |
| | | | DMSO-d₆) δ 12.25 (s, 1H), 7.74 (s, 1H), 7.07 (d, |
| | | | J = 8.2 Hz, 1H), 6.90 (dd, J = 8.1, 2.2 Hz, 1H), 6.85 |
| | | | (d, J = 2.1 Hz, 1H), 6.75 (s, 1H), 4.05 (d, J = 11.5 |
| | | | Hz, 2H), 3.83 (dd, J = 10.7, 7.8 Hz, 1H), 3.63 (s, |
| | | | 1H), 3.44 (dd, J = 7.0, 4.9 Hz, 1H), 2.85 (dd, J = |
| | | | 16.5, 5.4 Hz, 1H), 2.70-2.59 (m, 1H), 2.41 (s, |
| | | | 3H), 2.33 (dt, J = 4.6, 2.3 Hz, 1H), 1.62 (s, 3H), |
| | | | 1.06 (t, J = 7.0 Hz, 1H). LC/MS (APCI) m/z |
| | | | calcd. for C₂₀H₂₂N₄O₄SCl⁺ [M + H]⁺: 448.1; 449.1 |
| | | | found. |
| 27 | 24.7 | methylazetidine- 3-carbonitrile | 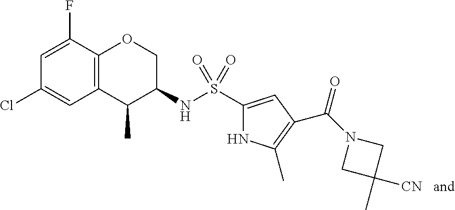 [SMILES: Cc1[nH]c(S(=O)(=O)N[C@@H]2COc3c(F)cc(Cl)cc3[C@@H]2C)cc1C(=O)N1CC(C)(C#N)C1] |
| | | | 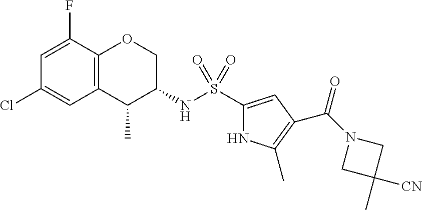 [SMILES: Cc1[nH]c(S(=O)(=O)N[C@H]2COc3c(F)cc(Cl)cc3[C@H]2C)cc1C(=O)N1CC(C)(C#N)C1] |
| | | | N-((cis)-6-chloro-8-fluoro-4-methylchroman-3- |
| | | | yl)-4-(3-cyano-3-methylazetidine-1-carbonyl)-5- |
| | | | methyl-1H-pyrrole-2-sulfonamide ¹H NMR, |
| | | | (400 MHz, DMSO-d₆) δ 12.25 (s, 1H). 7.80 (s, |
| | | | 1H), 7.26 (dd, J = 10.8. 2.5 Hz. 1H). 7.12 (d, J = |
| | | | 1.9 Hz, 1H), 6.75 (s. 1H), 4.61 (s. 1H), 424 (s, |
| | | | 3H), 4.13-3.83 (m, 2H), 3.37 (s, 1H), 2.86-2.69 |
| | | | (m, 1H), 2.41 (s, 3H), 1.62 (s, 3H), 1.11 (d, J = 7.0 |
| | | | Hz, 3H). LC/MS (APCI) m/z calcd. for |
| | | | C₂₁H₂₂ClFN₄O₄S⁺ [M + H]⁺: 481.1; 481.2 found. |
| 69 | 23 24 | methylazetidine- 3-carbonitrile | 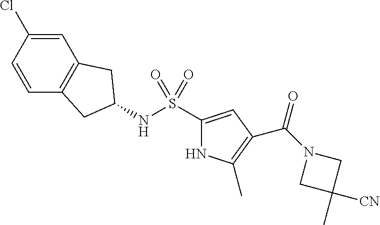 [SMILES: Cc1[nH]c(S(=O)(=O)N[C@@H]2Cc3ccc(Cl)cc3C2)cc1C(=O)N1CC(C)(C#N)C1] |
| | | | (R)-N-(5-chloro-2,3-dihydro-1H-inden-2-yl)-4- |
| | | | (3-cyano-3-methylazetidine-1-carbonyl)-5- |
| | | | methyl-1H-pyrrole-2-sulfonamide. 1H NMR |
| | | | (400 MHz, DMSO-d6) δ 12.20 (br s, 1H), 7.76 (d, |
| | | | J = 7.05 Hz, 1H), 7.24 (s, 1H), 7.14-7.22 (m, 2H), |
| | | | 6.71 (d, J = 1.66 Hz, 1H), 4.09-4.77 (m, 3H), 4.01 |
| | | | (qd, J = 7.07, 14.45 Hz, 2H), 2.94-3.10 (m, 2H), |
| | | | 2.65-2.81 (m, 2H), 2.42 (s, 3H), 1.62 (s, 3H). |
| | | | HRMS cald for C20H21N4O3S m/z 433.1102 |
| | | | [M + H]+, found 433.1102 [M + H]+. |
| Chiral Purification Intermediate 7 | 23.32 | 3- methylazetidine- 3-carbonitrile | 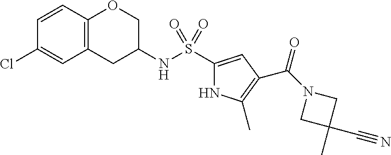 [SMILES: Cc1[nH]c(S(=O)(=O)NC2COc3ccc(Cl)cc3C2)cc1C(=O)N1CC(C)(C#N)C1] |
| | | | N-(6-chlorochroman-3-yl)-4-(3-cyano-3- |
| | | | methylazetidine-1-carbonyl)-5-methyl-1H- |
| | | | pyrrole-2-sulfonamide. ¹H NMR (400 MHz, |
| | | | DMSO-d₆) δ 12.08 (s, 1H), 7.57 (s, 1H), 7.00- |
| | | | 6.91 (m, 2H), 6.66-6.60 (m, 1H), 6.58 (s, 1H), |
| | | | 4.56-4.33 (m, 1H), 4.32-3.93 (m, 3H), 3.92- |
| | | | 3.83 (m, 1H), 3.65 (dd, J = 10.6, 7.7 Hz, 1H), 3.47 |
| | | | (s, 1H), 2.71 (dd, J = 16.7, 5.4 Hz, 1H), 2.51 (dd, |
| | | | J = 16.5, 8.0 Hz, 1H), 2.25 (s, 3H), 1.46 (s, 3H). |
| | | | LC/MS (APCI) m/z calcd. for C₂₀H₂₂ClN₄O₄S⁺ |
| | | | [M + H]⁺: 449.1; 449.1 found. |
| Chiral Purification Intermediate 8 | 27 | azetidine-3- carbonitrile | 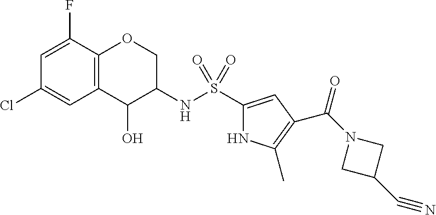 [SMILES: Cc1[nH]c(S(=O)(=O)NC2COc3c(F)cc(Cl)cc3C2O)cc1C(=O)N1CC(C#N)C1] |
| | | | N-(6-chloro-4-hydroxychroman-3-yl)-4-(3- |
| | | | cyanoazetidine-l-carbonyl)-5-methyl-1H- |
| | | | pyrrole-2-sulfonamide. ¹H NMR (400 MHz, |
| | | | DMSO-d₆) δ 12.24 (s, 1H), 7.41-7.25 (m, 2H), |
| | | | 7.22 (dd, J = 8.7, 2.7 Hz, 1H), 6.80 (d, J = 8.7 Hz, |
| | | | 1H), 6.77 (s, 1H), 5.84 (s, 1H), 4.63-3.91 (m, |
| | | | 6H), 3.89-3.75 (m, 2H), 3.62 (dt, J = 10.1, 3.7 |
| | | | Hz, 1H), 2.40 (s, 3H). LC/MS (APCI) m/z calcd. |
| | | | for C₁₉H₁₉N₄O₅SCl [M + H]⁺: 450.1; 451.1 found. |
| Chiral Purification Intermediate 9 | 27 | 3- methylazetidine- 3-carbonitrile | 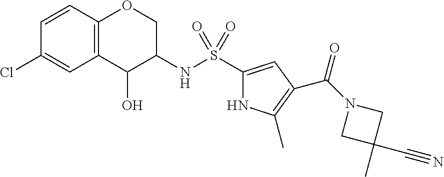 [SMILES: Cc1[nH]c(S(=O)(=O)NC2COc3ccc(Cl)cc3C2O)cc1C(=O)N1CC(C)(C#N)C1] |
| | | | N-(6-chloro-4-hydroxychroman-3-yl)-4-(3- |
| | | | cyano-3-methylazetidine-1-carbonyl)-5-methyl- |
| | | | 1H-pyrrole-2-sulfonamide. ¹H NMR (400 MHz, |
| | | | DMSO-d₆) δ 12.18 (s, 1H), 7.66 (s, 1H), 7.28 (d, |
| | | | J = 2.6 Hz, 1H), 7.22 (dd, J = 8.7, 2.7 Hz, 1H), 6.83 |
| | | | (d, J = 8.7 Hz, 1H), 6.75 (s, 1H), 5.87 (d, J = 4.5 |
| | | | Hz, 1H), 4.77-3.73 (m, 7H), 3.44-3.36 (m, 1H), |
| | | | 2.40 (s, 3H), 1.63 (s, 3H). ). LC/MS (APCI) m/z |
| | | | calcd. for C₂₀H₂₁N₄O₅SCl [M + H]⁺: 464.1; 465.1 |
| | | | found. |
| Chiral Purification Intermediate 10 | 23.25 | 3- methylazetidine- 3-carbonitrile | 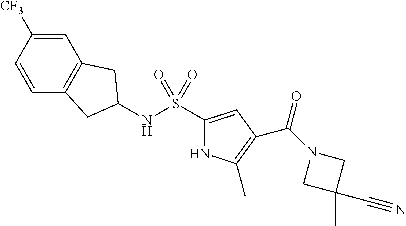 [SMILES: Cc1[nH]c(S(=O)(=O)NC2Cc3ccc(C(F)(F)F)cc3C2)cc1C(=O)N1CC(C)(C#N)C1] |
| | | | 4-(3-cyano-3-methylazetidine-l-carbonyl)-5- |
| | | | methyl-N-(5-(trifluoromethyl)-2,3-dihydro-1H- |
| | | | inden-2-yl)-1H-pyrrole-2-sulfonamide. LC/MS |
| | | | cald for C₂₁H₂₂F₃N₄O₃S m/z 467.1 [M + H]⁺, |
| | | | found 467.1 [M + H]⁺. |
| 79 | 23.26 | 3- methylazetidine- 3-carbonitrile | 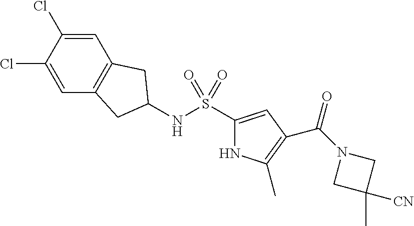 [SMILES: Cc1[nH]c(S(=O)(=O)NC2Cc3cc(Cl)c(Cl)cc3C2)cc1C(=O)N1CC(C)(C#N)C1] |
| | | | 4-(3-cyano-3-methylazetidine-1-carbonyl)-N- |
| | | | (5,6-dichloro-2,3-dihydro-1H-inden-2-yl)-5- |
| | | | methyl-1H-pyrrole-2-sulfonamide. 1H NMR |
| | | | (400 MHz, DMSO-d6) δ 12.22 (br s, 1H), 7.79 (d, |
| | | | J = 6.43 Hz, 1H), 7.46 (s, 2H), 6.70 (s, 1H), 3.86- |
| | | | 4.70 (m, 5H), 3.03 (dd, J = 7.67, 16.48 Hz, 2H), |
| | | | 2.74 (dd, J = 6.74, 16.59 Hz, 2H), 2.42 (s, 3H), 1.62 |
| | | | (s, 3H). HRMS cald for C20H21Cl2N4O3S m/z |
| | | | 467.0706 [M + H]+, found 467.0722 [M +H]+. |
| Chiral Purification Intermediate 11 | 23.27 | 3- methylazetidine- 3-carbonitrile | 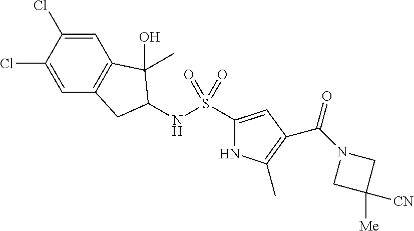 [SMILES: Cc1[nH]c(S(=O)(=O)NC2Cc3cc(Cl)c(Cl)cc3C2(C)O)cc1C(=O)N1CC(C)(C#N)C1] |
| | | | 4-(3-cyano-3-methylazetidine-1-carbonyl)-N- |
| | | | (5,6-dichloro-1-hydroxy-1-methyl-2,3-dihydro- |
| | | | 1H-inden-2-yl)-5-methyl-1H-pyrrole-2- |
| | | | sulfonamide. LC/MS cald for C₂₁H₂₃Cl₂N₄O₄S |
| | | | m/z 497.1 [M + H]⁺, found 497.1 [M + H]⁺. |
| Chiral Purification Intermediate 12 | 23.28 | 3- methylazetidine- 3-carbonitrile | 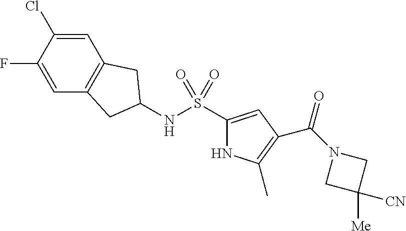 [SMILES: Cc1[nH]c(S(=O)(=O)NC2Cc3cc(F)c(Cl)cc3C2)cc1C(=O)N1CC(C)(C#N)C1] |
| | | | N-(5-chloro-6-fluoro-2,3-dihydro-1H-inden-2- |
| | | | yl)-4-(3-cyano-3-methylazetidine-1-carbonyl)-5- |
| | | | methyl-1H-pyrrole-2-sulfonamide. LC/MS cald |
| | | | for C₂₀H₂₁ClFN₄O₃S m/z 451.1 [M + H] |
| | | | 451.1 [M + H]+. |
| 89 | 23.29 | 3- methylazetidine- 3-carbonitrile | 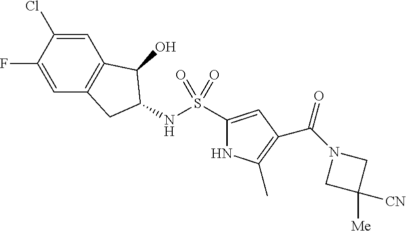 [SMILES: Cc1[nH]c(S(=O)(=O)N[C@@H]2Cc3cc(F)c(Cl)cc3[C@H]2O)cc1C(=O)N1CC(C)(C#N)C1] |
| | | | 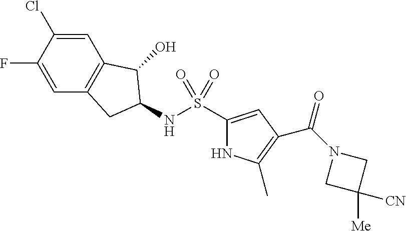 [SMILES: Cc1[nH]c(S(=O)(=O)N[C@H]2Cc3cc(F)c(Cl)cc3[C@@H]2O)cc1C(=O)N1CC(C)(C#N)C1] |
| | | | Ethyl 5-(N-((trans)-6-chloro-5-fluoro-1- |
| | | | hydroxy-2,3-dihydro-1H-inden-2-yl)sulfamoyl)- |
| | | | 2-methyl-1H-pyrrole-3-carboxylate. 1H NMR |
| | | | (500 MHz, DMSO-d6) δ 11.83-12.53 (m, 1H), |
| | | | 7.79-8.04 (m, 1H), 7.38 (d, J = 7.01 Hz, 1H), 7.24 |
| | | | (d, J = 9.34 Hz, 1H), 6.67-6.82 (m, 1H), 5.60-6.01 |
| | | | (m, 1H), 4.82 (d, J = 6.62 Hz, 1H), 3.86-4.70 (m, |
| | | | 4H), 3.64-3.75 (m, 2H), 2.93 (dd, J = 7.72, 16.15 |
| | | | Hz, 1H), 2.41 (s, 3H), 1.62 (s, 3H). HRMS cald |
| | | | for C₂₀H₂₁ClFN₄O₄S m/z 467.0951 [M + H]+, |
| | | | found 467.0964 [M + H]+. |
| 93 | 23.30 | 3- (trifluoromethyl) azetidine | 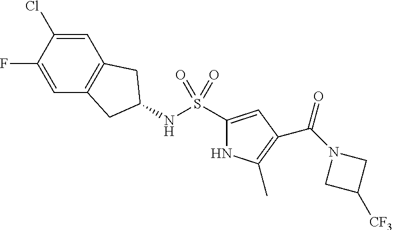 [SMILES: Cc1[nH]c(S(=O)(=O)N[C@@H]2Cc3cc(F)c(Cl)cc3C2)cc1C(=O)N1CC(C(F)(F)F)C1] |
| | | | (R)-N-(5-chloro-6-fluoro-2,3-dihydro-1H-inden- |
| | | | 2-yl)-5-methyl-4-(3-(trifluoromethyl)azetidine-1- |
| | | | carbonyl)-1H-pyrrole-2-sulfonamide. 1H NMR |
| | | | (500 MHz, DMSO-d6) δ 12.23 (br s, 1H), 7.77 (d, |
| | | | J = 7.14 Hz, 1H), 7.38 (d, J = 7.14 Hz, 1H), 7.24 (d, |
| | | | J = 9.47 Hz, 1H), 6.73 (d, J = 2.47 Hz, 1H), 3.93- |
| | | | 4.56 (m, 6H), 3.02 (td, J = 8.13, 16.32 Hz, 2H), |
| | | | 2.64-2.80 (m, 2H), 2.42 (s, 3H). HRMS cald for |
| | | | C₁₉H₁₉ClF₄N₃O₃S m/z 480.0767 [M + H]+, found |
| | | | 480.0781[M + H]+. |
| | | | Stereochemistry arbitrarily assigned. |
| 94 | 23.30 | 3- (trifluoromethoxy) azetidine | 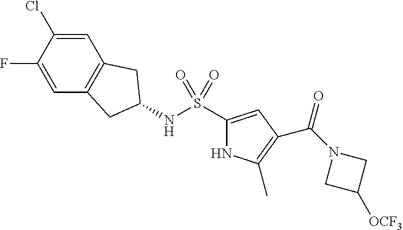 [SMILES: Cc1[nH]c(S(=O)(=O)N[C@@H]2Cc3cc(F)c(Cl)cc3C2)cc1C(=O)N1CC(OC(F)(F)F)C1] |
| | | | (R)-N-(5-chloro-6-fluoro-2,3-dihydro-1H- |
| | | | inden-2-yl)-5-methyl-4-(3- |
| | | | (trifluoromethoxy)azetidine-1-carbonyl)-1H- |
| | | | pyrrole-2-sulfonamide. 1H NMR (500 MHz, |
| | | | DMSO-d6) δ 12.21 (br s, 1H), 7.80 (br d, J = 7.01 |
| | | | Hz, 1H), 7.38 (d, J = 7.14 Hz, 1H), 7.24 (d, J = 9.47 |
| | | | Hz, 1H), 6.70 (s, 1H), 5.18-5.26 (m, 1H), 4.42 (br |
| | | | s, 3H), 4.03 (qd, J = 7.10, 14.26 Hz, 2H), 3.02 (td, |
| | | | J = 8.16, 16.25 Hz, 2H), 2.64-2.81 (m, 2H), 2.41 (s, |
| | | | 3H). HRMS cald for C₁₉H₁₉ClF₄N₃O₄S m/z |
| | | | 496.0716 [M + H] |
| | | | Stereochemistry arbitrarily assigned. |
| 95 | 23.30 | 3- methylazetidin- 3-ol | 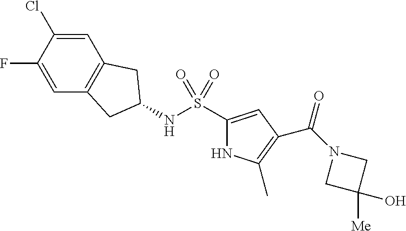 [SMILES: Cc1[nH]c(S(=O)(=O)N[C@@H]2Cc3cc(F)c(Cl)cc3C2)cc1C(=O)N1CC(C)(O)C1] |
| | | | (R)-N-(5-chloro-6-fluoro-2,3-dihydro-1H- |
| | | | inden-2-yl)-4-(3-hydroxy-3-methylazetidine-1- |
| | | | carbonyl)-5-methyl-1H-pyrrole-2-sulfonamide. |
| | | | 1H NMR (500 MHz, DMSO-d6) δ 12.14 (br s, |
| | | | 1H), 7.77 (d, J = 7.01 Hz, 1H), 7.38 (d, J = 7.14 Hz, |
| | | | 1H), 7.24 (d, J = 9.47 Hz, 1H), 6.66 (d, J = 2.21 Hz, |
| | | | 1H), 5.60 (s, 1H), 3.77-4.18 (m, 5H), 2.94-3.08 |
| | | | (m, 2H), 2.64-2.81 (m, 2H), 2.41 (s, 3H), 1.38 (s, |
| | | | 3H). HRMS cald for C₁₉H₂₂ClFN₃O₄S m/z |
| | | | 442.0998 [M + H]+, found 442.0997 [M + H]+. |
| | | | Stereochemistry arbitrarily assigned. |
| 96 | 23.30 | 2-oxa-6- azaspiro[3.3] heptane | 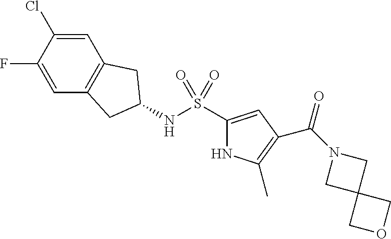 [SMILES: Cc1[nH]c(S(=O)(=O)N[C@@H]2Cc3cc(F)c(Cl)cc3C2)cc1C(=O)N1CC2(COC2)C1] |
| | | | (R)-N-(5-chloro-6-fluoro-2,3-dihydro-1H- |
| | | | inden-2-yl)-5-methyl-4-(2-oxa-6- |
| | | | azaspiro[3.3]heptane-6-carbonyl)-1H-pyrrole- |
| | | | 2-sulfonamide. 1H NMR (500 MHz, DMSO-d6) |
| | | | δ 12.15 (br s, 1H), 7.79 (d, J = 7.01 Hz, 1H), 7.39 |
| | | | (d, J = 7.27 Hz, 1H), 7.25 (d, J = 9.47 Hz, 1H), 6.68 |
| | | | (s, 1H), 4.67 (s, 4H), 3.98-4.50 (m, 5H), 3.01 (td, |
| | | | J = 8.13, 16.32 Hz, 2H), 2.68-2.76 (m, 2H), 2.39 (s, |
| | | | 3H). HRMS cald for C₂₀H₂₂ClFN₃O₄S m/z |
| | | | 454.0998 [M + H]+, found 454.1018 [M + H]+. |
| | | | Stereochemistry arbitrarily assigned. |
| 97 | 23.30 | 6,6-difluoro-2- azaspiro[3.3] heptane | 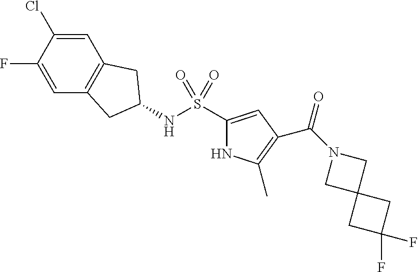 [SMILES: Cc1[nH]c(S(=O)(=O)N[C@@H]2Cc3cc(F)c(Cl)cc3C2)cc1C(=O)N1CC2(C1)CC(F)(F)C2] |
| | | | (R)-N-(5-chloro-6-fluoro-2,3-dihydro-1H- |
| | | | inden-2-yl)-4-(6,6-difluoro-2- |
| | | | azaspiro[3.3]heptane-2-carbonyl)-5-methyl-1H- |
| | | | pyrrole-2-sulfonamide. 1H NMR (500 MHz, |
| | | | DMSO-d6) δ 12.16 (br s, 1H), 7.80 (br d, J = 7.14 |
| | | | Hz, 1H), 7.39 (d, J = 7.14 Hz, 1H), 7.24 (d, J = 9.47 |
| | | | Hz, 1H), 6.68 (s, 1H), 4.40 (br s, 2H), 3.97-4.17 |
| | | | (m, 3H), 3.01 (td, J = 8.01, 16.15 Hz, 2H), 2.79- |
| | | | 2.90 (m, 4H), 2.66-2.79 (m, 2H), 2.41 (s, 3H). |
| | | | HRMS cald for C₂₁H₂₂ClF₃N₃O₃S m/z 488.1017 |
| | | | [M + H]+, found 488.1025 [M + H]+. |
| | | | Stereochemistry arbitrarily assigned. |
| 98 | 23.31 | 3- (trifluoromethoxy) azetidine | 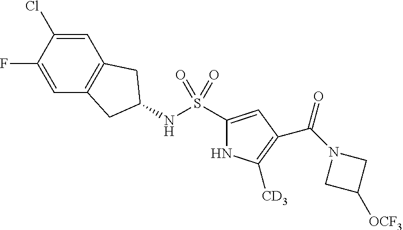 [SMILES: [2H]C([2H])([2H])c1[nH]c(S(=O)(=O)N[C@@H]2Cc3cc(F)c(Cl)cc3C2)cc1C(=O)N1CC(OC(F)(F)F)C1] |
| | | | (R)-N-(5-chloro-6-fluoro-2,3-dihydro-1H- |
| | | | inden-2-yl)-5-(methyl-d3)-4-(3- |
| | | | (trifluoromethoxy)azetidine-l-carbonyl)-1H- |
| | | | pyrrole-2-sulfonamide. 1H NMR (500 MHz, |
| | | | DMSO-d6) δ 12.22 (br s, 1H), 7.80 (d, J = 5.97 Hz, |
| | | | 1H), 7.37 (d, J = 7.14 Hz, 1H), 7.23 (d, J = 9.47 Hz, |
| | | | 1H), 6.70 (s, 1H), 5.17-5.26 (m, 1H), 4.22-4.72 |
| | | | (m, 4H), 3.99-4.08 (m, 1H), 3.01 (td, J = 8.11, |
| | | | 16.22 Hz, 2H), 2.67-2.78 (m, 2H). HRMS cald for |
| | | | C₁₉H₁₆D₃ClF₄N₃O₄S m/z 499.0904 [M + H]+, |
| | | | found 499.0915 [M + H]+. |
| | | | Stereochemistry arbitrarily assigned. |
| 99 | 23.33 | 3- methylazetidine- 3-carbonitrile | 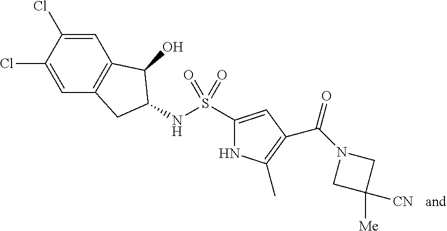 [SMILES: Cc1[nH]c(S(=O)(=O)N[C@@H]2Cc3cc(Cl)c(Cl)cc3[C@H]2O)cc1C(=O)N1CC(C)(C#N)C1 ; "and"] |
| | | | 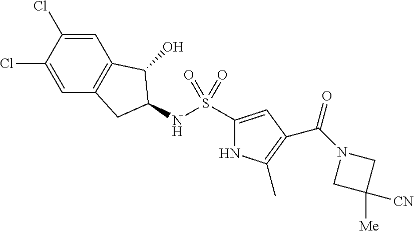 [SMILES: Cc1[nH]c(S(=O)(=O)N[C@H]2Cc3cc(Cl)c(Cl)cc3[C@@H]2O)cc1C(=O)N1CC(C)(C#N)C1] |
| | | | 4-(3-cyano-3-methylazetidine-1-carbonyl)-N- |
| | | | (trans)-5,6-dichloro-1-hydroxy-2,3-dihydro-1H- |
| | | | inden-2-yl)-5-methyl-1H-pyrrole-2- |
| | | | sulfonamide. ¹H NMR (500 MHz, DMSO-d₆) δ |
| | | | 12.20 (br s, 1H), 7.90 (d, J = 4.93 Hz, 1H), 7.46 (s, |
| | | | 1H), 7.43 (s, 1H), 6.73 (s, 1H), 5.84 (br s, 1H), |
| | | | 4.83 (d, J = 6.75 Hz, 1H), 3.84-4.69 (m, 4H), 3.63- |
| | | | 3.74 (m, 1H), 2.94 (dd, J = 7.85, 16.15 Hz, 1H), |
| | | | 2.52-2.54 (m, 1H), 2.41 (s, 3H), 1.62 (s, 3H). |
| | | | HRMS cald for C₂₀H₂₁Cl₂N₄O₄S m/z 483.0655 |
| | | | [M + H]⁺, found 483.0676 [M + H]⁺ |

### Example S-2. Sulfonamide Formation and SNAr General Procedure

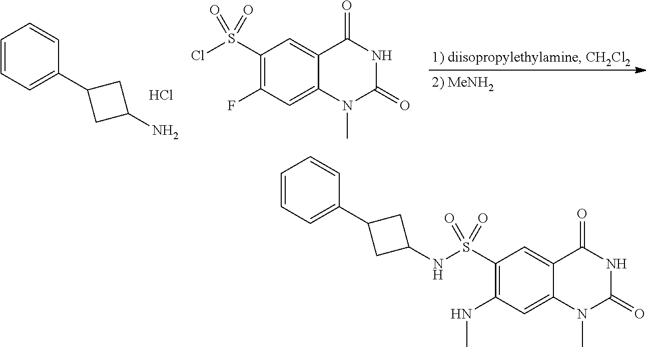

1-Methyl-7-(methylamino)-2,4-dioxo-N-(3-phenylcyclobutyl)-1,2,3,4-tetrahydroquinazoline-6-sulfonamide. 7-Fluoro-1-methyl-2,4-dioxo-1,2,3,4-tetrahydroquinazoline-6-sulfonyl chloride (188 mg, 1.025 mmol, 1 equiv.) was added to a stirring solution of 3-phenylcyclobutan-1-amine hydrochloride (300 mg, 1.25 mmol, 1 equiv.), and diisopropylamine (2 mL, 11.5 mmol, 11 equiv.) in CH₂Cl₂ (12 mL) at rt. After 1.5 h, methyl amine (2 M in THF, 10 mL, 20 mmol, 20 equiv.) was added and the reaction heated to 50° C. The reaction was then cooled to rt, solvent removed by rotary evaporation, and before residue was triturated with CH₂Cl₂/MeOH to give the product as a white solid (139 mg, 33%). H NMR (400 MHz, DMSO-d₆) δ 11.32 (s, 1H), 8.26 (s, 1H), 8.17 (s, 1H), 7.28 (t, J=7.5 Hz, 2H), 7.17 (dd, J=15.8, 7.3 Hz, 3H), 6.65 (q, J=4.6 Hz, 1H), 6.31 (s, 1H), 3.75 (d, J=7.5 Hz, 1H), 3.44 (s, 4H), 2.99 (d, J=4.8 Hz, 3H), 2.21 (qdd, J=12.5, 8.7, 6.0 Hz, 4H). LC/MS (APCI) m/z calcd. for C₂₀H₂₃N₄O₄S⁺ [M+H]⁺: 415.1; 415.3 found.

TABLE S-2
The following compounds and intermediates were prepared according to the synthetic
procedures of Example S-2.

| | Inter- | | | |
|---|---|---|---|---|
| | mediate | | | |
| | Sulfonyl | | | |
| Compound | chloride | Amine 1 | Amine 2 | Structure, Name and Data |
| 20 | 18.1 | 3- phenyl cyclobutan-1- amine | Methyl- amine | 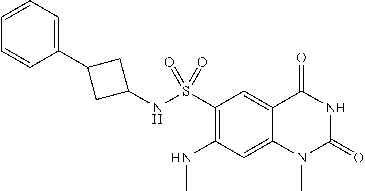 [SMILES: CNc1cc2c(cc1S(=O)(=O)NC1CC(c3ccccc3)C1)c(=O)[nH]c(=O)n2C] |
| | | | | 1-Methyl-7-(methylamino)-2,4-dioxo-N- |
| | | | | (3-phenylcyclobutyl)-1,2,3,4- |
| | | | | tetrahydroquinazoline-6-sulfonamide. ¹H |
| | | | | NMR (400 MHz, DMSO-d₆) δ 11.32 (s, 1H), |
| | | | | 8.26 (s, 1H), 8.17 (s, 1H), 7.28 (t, J = 7.5 |
| | | | | Hz, 2H), 7.17 (dd, J = 15.8, 7.3 Hz, 3H), |
| | | | | 6.65 (q, J = 4.6 Hz, 1H), 6.31 (s, 1H), 3.75 |
| | | | | (d, J = 7.5 Hz, 1H), 3.44 (s, 4H), 2.99 (d, J = |
| | | | | 4.8 Hz, 3H), 2.21 (qdd, J = 12.5, 8.7, 6.0 Hz, |
| | | | | 4H). LC/MS (APCI) m/z calcd. for |
| | | | | C₂₀H₂₃N₄O₄S⁺ [M + H]⁺: 415.1; 415.3 found. |
| 21 | 18.1 | 2-(1H-indo1-2- yl)ethan-1- amine | Methyl- amine | 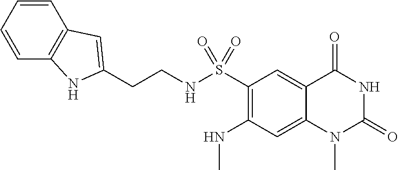 [SMILES: CNc1cc2c(cc1S(=O)(=O)NCCc1cc3ccccc3[nH]1)c(=O)[nH]c(=O)n2C] |
| | | | | N-(2-(1H-indo1-2-ypethyl)-1-methyl-7- |
| | | | | (methylamino)-2,4-dioxo-1,2,3,4- |
| | | | | tetrahydroquinazoline-6-sulfonamide. ¹H |
| | | | | NMR (400 MHz, DMSO-d₆) δ 12.13 (s, 1H), |
| | | | | 8.18 (s, 1H), 7.41 (s, 2H), 7.09 (dd, J = 6.0, |
| | | | | 3.2 Hz, 2H), 6.49 (q, J = 4.8 Hz, 1H), 6.15 |
| | | | | (s, 1H), 3.40 (s, 3H), 3.34 (s, 3H), 3.25 (t, |
| | | | | J = 7.0 Hz, 2H), 2.93 (t, J = 7.0 Hz, 2H), 2.87 |
| | | | | (d, J = 4.6 Hz, 3H). LC/MS (APCI) m/z |
| | | | | calcd. for C₂₀H₂₂N₅O₄S⁺ [M + H]⁺: 428.1; |
| | | | | 428.3 found. |
| 22 | 18.1 | (1R,5S,6s)- 3-(3- fluorophenyl)- 3-azabicyclo [3.1.0]hexan- 6-amine | Methyl- amine | 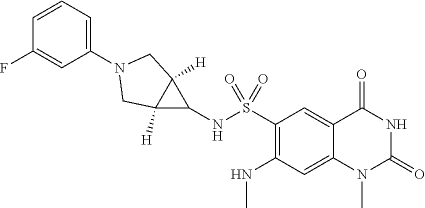 [SMILES: CNc1cc2c(cc1S(=O)(=O)NC1[C@H]3CN(c4cccc(F)c4)C[C@@H]13)c(=O)[nH]c(=O)n2C] |
| | | | | N-((1R,5S,6s)-3-(3-Fluorophenyl)-3- |
| | | | | azabicyclo[3.1.0]hexan-6-yl)-1-methyl-7- |
| | | | | (methylamino)-2,4-dioxo-1,2,3,4- |
| | | | | tetrahydroquinazoline-6-sulfonamide. ¹H |
| | | | | NMR (400 MHz, DMSO-d₆) δ 11.29 (s, 1H), |
| | | | | 8.21 (s, 1H), 7.10 (q, J = 7.8 Hz, 1H), 6.89 |
| | | | | (s, 2H), 6.73 (s, 1H), 6.44-6.33 (m, 1H), |
| | | | | 6.33-6.19 (m, 2H), 4.00 (s, 3H), 3.44 (s, |
| | | | | 3H), 2.97 (d, J = 4.7 Hz, 2H), 2.72 (t, J = 7.6 |
| | | | | Hz, 2H), 2.55 (t, J = 7.6 Hz, 2H), 1.26 (d, |
| | | | | J = 3.7 Hz, 1H). LC/MS (APCI) m/z calcd. |
| | | | | for C₂₁H₂₃FN₅O₄S⁺ [M + H]⁺: 460.1; 460.3 |
| | | | | found. |
| 23 | 18.1 | (cis)-N¹-(3- fluorophenyl)-3- methyl- cylcobutane- 1,3- diamine | Methyl- amine | 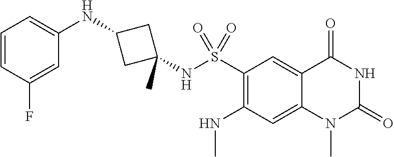 [SMILES: CNc1cc2c(cc1S(=O)(=O)N[C@]1(C)C[C@H](Nc3cccc(F)c3)C1)c(=O)[nH]c(=O)n2C] |
| | | | | N-((cis)-3-((3-fluorophenyl)amino)-1- |
| | | | | methylcyclobutyl)-1-methyl-7- |
| | | | | (methylamino)-2,4-dioxo-1,2,3,4- |
| | | | | tetrahydroquinazoline-6-sulfonamide. ¹H |
| | | | | NMR (400 MHz, DMSO-d₆) δ 11.28 (s, 1H), |
| | | | | 8.20 (s, 1H), 8.02 (s, 1H), 7.02 (q, J = 8.0 |
| | | | | Hz, 1H), 6.55 (q, J = 4.6 Hz, 1H), 6.35- |
| | | | | 6.13 (m, 5H), 3.66-3.50 (m, 1H), 3.45 (s, |
| | | | | 3H), 2.99 (d, J = 4.8 Hz, 3H), 2.25 (ddd, J = |
| | | | | 9.4, 7.1, 2.8 Hz, 2H), 1.97 (dd, J = 11.2, 8.4 |
| | | | | Hz, 2H), 1.26 (s, 3H). LC/MS (APCI) m/z |
| | | | | calcd. for C₂₁H₂₅FN₅O₄S⁺ [M + H]⁺: 462.2; |
| | | | | 462.3 found. |
| 24 | 19.2 | 3-(3- fluorophenyl) cyclobutan- 1-amine | Methyl- amine | 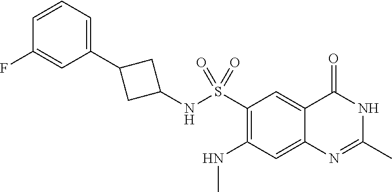 [SMILES: CNc1cc2nc(C)[nH]c(=O)c2cc1S(=O)(=O)NC1CC(c2cccc(F)c2)C1] |
| | | | | N-(3-(3-fluorophenyl)cyclobutyl)-2- |
| | | | | methyl-7-(methylamino)-4-oxo-3,4- |
| | | | | dihydroquinazoline-6-sulfonamide. ¹H |
| | | | | NMR (400 MHz, DMSO-d₆) δ 11.95 (s, 1H), |
| | | | | 8.37-8.24 (m, 2H), 7.31 (td, J = 8.0, 6.5 |
| | | | | Hz, 1H), 7.09-6.91 (m, 3H), 6.64 (s, 1H), |
| | | | | 6.43 (q, J = 4.7 Hz, 1H), 3.76 (q, J = 7.4 Hz, |
| | | | | 1H), 3.47 (tt, J = 9.6, 5.5 Hz, 1H), 2.92 (d, |
| | | | | J = 4.7 Hz, 3H), 2.30 (s, 3H), 2.27-2.11 (m, |
| | | | | 4H). LC/MS (APCI) m/z calcd. for |
| | | | | C₂₀H₂₂FN₄O₃S⁺ [M + H]⁺: 417.1; 417.3 |
| | | | | found. |
| 19 | 18.1 | 2.10 | Methyl- amine | 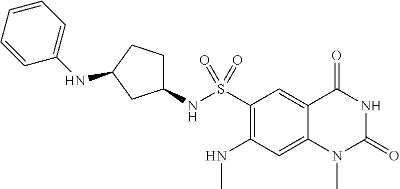 [SMILES: CNc1cc2c(cc1S(=O)(=O)N[C@@H]1CC[C@H](Nc3ccccc3)C1)c(=O)[nH]c(=O)n2C] |
| | | | | 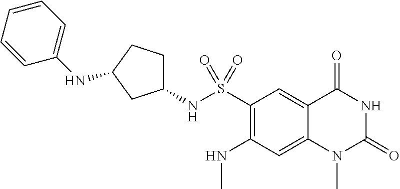 [SMILES: CNc1cc2c(cc1S(=O)(=O)N[C@H]1CC[C@@H](Nc3ccccc3)C1)c(=O)[nH]c(=O)n2C] |
| | | | | 1-Methyl-7-(methylamino)-2,4-dioxo-N- |
| | | | | ((cis)-3-(phenylamino)cyclopentyl)-1,2,3,4- |
| | | | | tetrahydroquinazoline-6-sulfonamide. ¹H |
| | | | | NMR (400 MHz, DMSO-d₆) δ 11.33 (s, 1H), |
| | | | | 8.21 (s, 1H), 7.86 (d, J = 7.5 Hz, 1H), 7.02 |
| | | | | (dd, J = 8.6, 7.1 Hz, 2H), 6.57 (d, J = 5.0 Hz, |
| | | | | 1H), 6.48 (dd, J = 8.1, 6.8 Hz, 3H), 6.30 (s, |
| | | | | 1H), 5.50 (d, J = 6.8 Hz, 1H), 3.58 (dq, J = |
| | | | | 13.4, 7.2, 6.7 Hz, 1H), 3.45 (s, 4H), 2.96 (d, |
| | | | | J = 4.8 Hz, 3H), 2.12 (dt, J = 13.4, 7.0 Hz, |
| | | | | 1H), 1.82 (dt, J = 11.6, 7.0 Hz, 1H), 1.73- |
| | | | | 1.56 (m, 1H), 1.45 (ddd, J = 21.0, 17.1, 10.0 |
| | | | | Hz, 2H), 1.22 (dt, J = 12.8, 7.9 Hz, 1H). |
| | | | | LC/MS (APCI) m/z calcd. for C₂₁H₂₆N₅O₄S ⁺ |
| | | | | [M + H]⁺: 444.2; 444.2 found. |
| 59 | 19.2 | 4 | Methyl- amine | 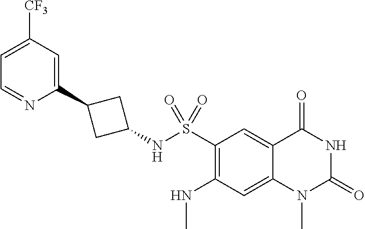 [SMILES: CNc1cc2c(cc1S(=O)(=O)N[C@H]1C[C@H](c3cc(C(F)(F)F)ccn3)C1)c(=O)[nH]c(=O)n2C] |
| | | | | 1-methyl-7-(methylamino)-2,4-dioxo-N- |
| | | | | (trans)-3-(4-(trifluoromethyl)pyridin-2- |
| | | | | yl)cyclobutyl)-1,2,3,4- |
| | | | | tetrahydroquinazoline-6-sulfonamide. ¹H |
| | | | | NMR (400 MHz, DMSO-d₆) δ 12.28-11.58 |
| | | | | (m, 1H), 8.80 (d, J = 5.1 Hz, 1H), 8.28 (s, |
| | | | | 2H), 7.54 (d, J = 3.0 Hz, 1H), 6.63 (s, 1H), |
| | | | | 6.40 (d, J = 5.0 Hz, 1H), 4.02 (q, J = 7.7 Hz, |
| | | | | 1H), 3.59 (q, J = 6.9 Hz, 1H), 3.13 (s, 1H), |
| | | | | 2.92 (d, J = 4.8 Hz, 3H), 2.28 (s, 3H), 2.24 |
| | | | | (t, J = 7.3 Hz, 4H). LC/MS (APCI) m/z |
| | | | | calcd. for C20H21F3N5O4S+ [M + H]+: |
| | | | | 484.1; 484.1 found. |
| 60 | 18.1 | 4 | Methyl- amine | 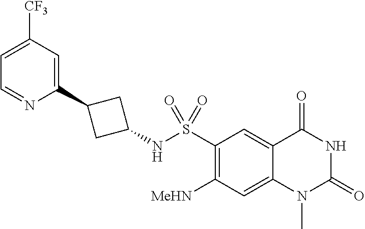 [SMILES: CNc1cc2c(cc1S(=O)(=O)N[C@H]1C[C@H](c3cc(C(F)(F)F)ccn3)C1)c(=O)[nH]c(=O)n2C] |
| | | | | 1-Methyl-7-(methylamino)-2,4-dioxo-N- |
| | | | | ((trans)-3-(4-(trifluoromethyl)pyridin-2- |
| | | | | yl)cyclobutyl)-1,2,3,4- |
| | | | | tetrahydroquinazoline-6-sulfonamide. ¹H |
| | | | | NMR (400 MHz, methanol-d) δ 8.80 (d, J = |
| | | | | 5.0 Hz, 1H), 8.16 (s, 1H), 7.56 (s, 1H), 6.62 |
| | | | | (q, J = 4.8 Hz, 1H), 6.30 (s, 1H), 3.99 (p, J = |
| | | | | 7.7 Hz, 1H), 3.62 (td, J = 8.2, 4.4 Hz, 1H), |
| | | | | 3.43 (s, 3H), 2.99 (d, J = 4.8 Hz, 3H), 2.27 |
| | | | | (t, J = 6.5 Hz, 4H). LC/MS (APCI) m/z |
| | | | | calcd. for C₂₀H₂₁F₃N₅O₃S⁺ [M + H]⁺: 468.1; |
| | | | | 468.1 found. |
| 14 | 18.1 | 2.6 | Methyl- amine | 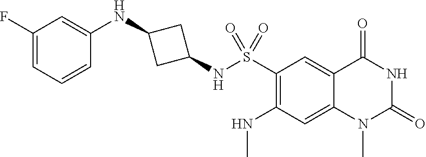 [SMILES: CNc1cc2c(cc1S(=O)(=O)N[C@H]1C[C@@H](Nc3cccc(F)c3)C1)c(=O)[nH]c(=O)n2C] |
| | | | | N-((cis)-3-((3- |
| | | | | fluorophenyl)amino)cyclobutyl)-1-methyl- |
| | | | | 7-(methylamino)-2,4-dioxo-1,2,3,4- |
| | | | | tetrahydroquinazoline-6-sulfonamide. ¹H |
| | | | | NMR (400 MHz, DMSO-d₆) δ 8.19 (s, 1H), |
| | | | | 7.01 (td, J = 8.2, 7.0 Hz, 2H), 6.58 (t, J = 4.9 |
| | | | | Hz, 2H), 6.31 (s, 1H), 6.29-6.21 (m, 2H), |
| | | | | 6.20-6.13 (m, 2H), 3.45 (s, 3H), 2.98 (d, |
| | | | | J = 4.7 Hz, 3H), 2.48-2.37 (m, 3H), 1.66- |
| | | | | 1.50 (m, 3H). LC/MS (APCI) m/z calcd. for |
| | | | | C₂₀H₂₁FN₅O₄S⁻ [M − H]⁻ : 446.1; 446.2 found. |
| 15 | 19 2 | 2.6 | Methyl- amine | 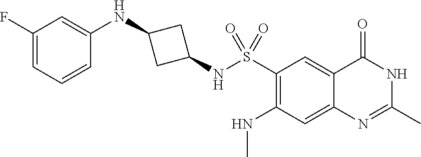 [SMILES: CNc1cc2nc(C)[nH]c(=O)c2cc1S(=O)(=O)N[C@H]1C[C@@H](Nc2cccc(F)c2)C1] |
| | | | | N-((cis)-3-((3- |
| | | | | fluorophenyl)amino)cyclobutyl)-2-methyl- |
| | | | | 7-(methylamino)-4-oxo-3,4- |
| | | | | dihydroquinazoline-6-sulfonamide. ¹H |
| | | | | NMR (400 MHz, DMSO-d₆) δ 11.68 (s, 1H), |
| | | | | 8.31 (s, 1H), 8.18 (s, 1H), 7.00 (q, J = 8.1 |
| | | | | Hz, 1H), 6.64 (s, 1H), 6.37 (q, J = 4.7 Hz, |
| | | | | 1H), 6.24 (td, J = 8.3, 2.3 Hz, 2H), 6.14 (dt, |
| | | | | J = 11.8, 2.2 Hz, 2H), 3.34 (q, J = 3.0, 2.5 |
| | | | | Hz, 2H), 2.92 (d, J = 4.8 Hz, 3H), 2.39 (dtd, |
| | | | | J = 9.6, 7.1, 2.9 Hz, 2H), 2.34-2.23 (m, |
| | | | | 3H), 1.56 (qd, J = 8.9, 2.6 Hz, 2H). LC/MS |
| | | | | (APCI) m/z calcd. for C₂₀H₂₃FN₅O₃S⁺ |
| | | | | [M + H]⁺: 432.1.2; 432.3 found. |
| Chiral Purification Intermediate 5 | 18.1 | N¹- phenyl- cyclobutane- 1,3- diamine | Methyl- amine | 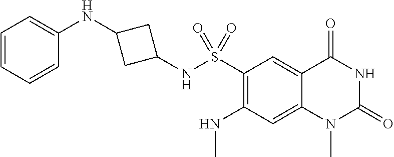 [SMILES: CNc1cc2c(cc1S(=O)(=O)NC1CC(Nc3ccccc3)C1)c(=O)[nH]c(=O)n2C] |
| | | | | 1-Methyl-7-(methylamino)-2,4-dioxo-N- |
| | | | | (3-(phenylamino)cyclobutyl)-1,2,3,4- |
| | | | | tetrahydroquinazoline-6-sulfonamide. ¹H |
| | | | | NMR (400 MHz, DMSO-d₆) δ 11.25 (s, 1H), |
| | | | | 8.30-8.11 (m, 2H), 7.02 (q, J = 8.0 Hz, |
| | | | | 2H), 6.58 (dd, J = 8.1, 4.8 Hz, 1H), 6.50 (dt, |
| | | | | J = 11.4, 7.2 Hz, 1H), 6.39 (dd, J = 16.1,7.9 |
| | | | | Hz, 2H), 6.31 (d, J = 1.4 Hz, 1H), 5.84 (dd, |
| | | | | J = 34.5, 5.9 Hz, 1H), 3.81-3.64 (m, 1H), |
| | | | | 3.45 (s, 3H), 2.98 (d, J = 4.7 Hz, 3H), 2.42 |
| | | | | (dt, J = 9.7, 7.0 Hz, 1H), 2.20-2.04 (m, |
| | | | | 1H), 2.00-1.84 (m, 1H), 1.57 (dt, J = 11.4, |
| | | | | 8.7 Hz, 1H), 1 proton under solvent peak. |
| | | | | LC/MS (APCI) m/z calcd. for C₂₀H₂₄N₅O₄S⁺ |
| | | | | [M + H]⁺: 430.2; 430.3 found. |
| 18 | 18.1 | 2.8 | Methyl- amine | 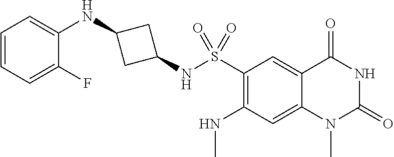 [SMILES: CNc1cc2c(cc1S(=O)(=O)N[C@H]1C[C@@H](Nc3ccccc3F)C1)c(=O)[nH]c(=O)n2C] |
| | | | | N-((cis)-3-((2- |
| | | | | fluorophenyl)amino)cyclobutyl)-1-methyl- |
| | | | | 7-(methylamino)-2,4-dioxo-1,2,3,4- |
| | | | | tetrahydroquinazoline-6-sulfonamide. ¹H |
| | | | | NMR (400 MHz, DMSO-d₆) δ 11.33 (s, 1H), |
| | | | | 8.19 (s, 1H), 8.07 (d, J = 7.8 Hz, 1H), 7.02- |
| | | | | 6.93 (m, 1H), 6.90 (t, J = 7.7 Hz, 1H), 6.59 |
| | | | | (q, J = 4.9 Hz, 1H), 6.56-6.46 (m, 2H), |
| | | | | 6.31 (s, 1H), 5.58 (dd, J = 6.4, 1.9 Hz, 1H), |
| | | | | 3.45 (s, 5H), 2.99 (d, J = 4.8 Hz, 3H), 2.44 |
| | | | | (dtd, J = 9.9, 7.1, 2.8 Hz, 2H), 1.71 (qd, J = |
| | | | | 8.8, 2.8 Hz, 2H). LC/MS (APCI) m/z calcd. |
| | | | | for C₂₀H₂₁FN₅O₄S⁺ [M − H]⁻: 446.1; 446.1 |
| | | | | found. |
| 25 | 18.1 | 2.9 | Methyl- amine | 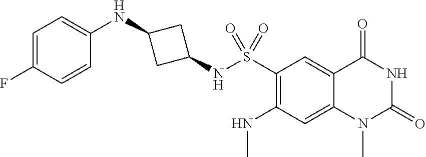 [SMILES: CNc1cc2c(cc1S(=O)(=O)N[C@H]1C[C@@H](Nc3ccc(F)cc3)C1)c(=O)[nH]c(=O)n2C] |
| | | | | N-((cis)-3-((4- |
| | | | | fluorophenyl)amino)cyclobutyl)-1-methyl- |
| | | | | 7-(methylamino)-2,4-dioxo-1,2,3,4- |
| | | | | tetrahydroquinazoline-6-sulfonamide. ¹H |
| | | | | NMR (400 MHz, DMSO-d₆) δ 11.33 (s, 1H), |
| | | | | 8.19 (s, 1H), 8.11 (d, J = 8.2 Hz, 1H), 6.86 |
| | | | | (t, J = 8.9 Hz, 2H), 6.58 (q, J = 4.5 Hz, 1H), |
| | | | | 6.46-6.35 (m, 2H), 6.31 (s, 1H), 5.73 (d, |
| | | | | J = 6.3 Hz, 1H), 3.45 (s, 3H), 3.34-3.24 (m, |
| | | | | 2H), 2.98 (d, J = 4.8 Hz, 3H), 2.42 (dtd, J = |
| | | | | 9.6, 7.1, 2.9 Hz, 2H), 1.55 (qd, J = 8.9, 2.8 |
| | | | | Hz, 2H). LC/MS (APCI) m/z calcd. for |
| | | | | C₂₀H₂₃FN₅O₄S⁺ [M + H]⁺: 448.1; 448.2 |
| | | | | found. |
| 26 | 18.1 | (R)-1-(6- fluoro-1H- indo1-2- yl)propan- 2-amine | Methyl- amine | 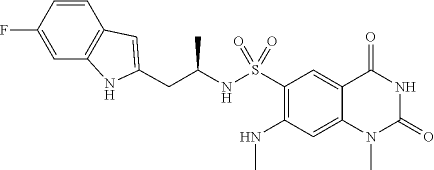 [SMILES: CNc1cc2c(cc1S(=O)(=O)N[C@H](C)Cc1cc3ccc(F)cc3[nH]1)c(=O)[nH]c(=O)n2C] |
| | | | | (R)-N-(1-(6-fluoro-1H-indo1-2-yl)propan- |
| | | | | 2-yl)-1-methyl-7-(methylamino)-2,4-dioxo- |
| | | | | 1,2,3,4-tetrahydroquinazoline-6- |
| | | | | sulfonamide. ¹H NMR (400 MHz, DMSO- |
| | | | | d₆) δ 11.21 (s, 1H), 10.73 (s, 1H), 8.05 (s, |
| | | | | 1H), 7.76 (d, J = 8.1 Hz, 1H), 7.26 (dd, J = |
| | | | | 8.4, 5.5 Hz, 1H), 6.78-6.62 (m, 2H), 6.23 |
| | | | | (q, J = 4.8 Hz, 1H), 5.99 (s, 1H), 5.77 (s, |
| | | | | 1H), 3.56 (p, J = 7.2 Hz, 1H), 3.27 (s, 3H), |
| | | | | 2.79 (d, J = 4.8 Hz, 3H), 2.70-2.60 (m, |
| | | | | 2H), 1.11 (d, J = 6.5 Hz, 3H). LC/MS |
| | | | | (APCI) m/z calcd. for C₂₁H₂₁FN₅O₄S⁻ [M − H]⁻: |
| | | | | 458.1; 458.3 found. |
| 28 | 19.2 | 2,3,4,9- tetrahydro- 1H- carbazol-2- amine | Methyl- amine | 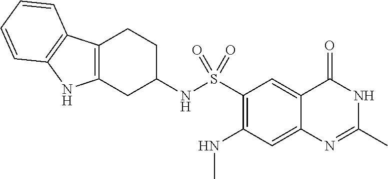 [SMILES: CNc1cc2nc(C)[nH]c(=O)c2cc1S(=O)(=O)NC1CCc2c([nH]c3ccccc23)C1] |
| | | | | 2-Methyl-7-(methylamino)-4-oxo-N- |
| | | | | (2,3,4,9-tetrahydro-1H-carbazol-2-yl)-3,4- |
| | | | | dihydroquinazoline-6-sulfonamide. ¹H |
| | | | | NMR (400 MHz, DMSO-d₆) δ 11.95 (s, 1H), |
| | | | | 10.60 (s, 1H), 8.37 (s, 1H), 8.03 (d, J = 7.6 |
| | | | | Hz, 1H), 7.30 (d, J = 7.7 Hz, 1H), 7.20 (d, |
| | | | | J = 7.9 Hz, 1H), 6.98 (ddd, J = 8.1, 7.0, 1.3 |
| | | | | Hz, 1H), 6.91 (ddd, J = 8.0, 7.0, 1.1 Hz, 1H), |
| | | | | 6.68 (s, 1H), 6.40 (q, J = 4.6 Hz, 1H), 3.46 |
| | | | | (td, J = 9.5, 9.0, 4.5 Hz, 1H), 2.91 (d, J = 4.8 |
| | | | | Hz, 3H), 2.84-2.64 (m, 2H), 2.57 (dd, J = |
| | | | | 16.6, 7.1 Hz, 2H), 2.31 (s, 3H), 1.91-1.76 |
| | | | | (m, 1H), 1.67 (ddt, J = 12.7, 8.5, 4.2 Hz, |
| | | | | 1H). LC/MS (APCI) m/z calcd. for |
| | | | | C₂₂H₂₄N₅O₃S⁺ [M + H]⁺: 438.2; 438.2 found. |
| 33 | 19.2 | 5.3 | Methyl- amine | 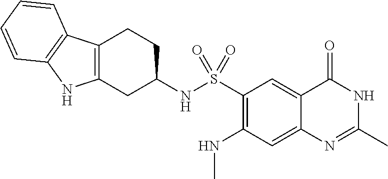 [SMILES: CNc1cc2nc(C)[nH]c(=O)c2cc1S(=O)(=O)N[C@@H]1CCc2c([nH]c3ccccc23)C1] |
| | | | | (R)-2-Methyl-7-(methylamino)-4-oxo-N- |
| | | | | (2,3,4,9-tetrahydro-1H-carbazol-2-yl)-3,4- |
| | | | | dihydroquinazoline-6-sulfonamide. ¹H |
| | | | | NMR (400 MHz, DMSO-d₆) δ 11.96 (s, 1H), |
| | | | | 10.60 (s, 1H), 8.36 (s, 1H), 8.03 (d, J = 7.6 |
| | | | | Hz, 1H), 7.30 (dd, J = 7.6, 1.1 Hz, 1H), 7.25- |
| | | | | 7.14 (m, 1H), 6.98 (ddd, J = 8.0, 7.0, 1.3 |
| | | | | Hz, 1H), 6.91 (ddd, J = 8.1, 7.0, 1.1 Hz, 1H), |
| | | | | 6.68 (s, 1H), 6.40 (q, J = 4.8 Hz, 1H), 3.46 |
| | | | | (td, J = 9.5, 8.9, 4.7 Hz, 1H), 2.91 (d, J = 4.8 |
| | | | | Hz, 3H), 2.85-2.64 (m, 2H), 2.64-2.52 |
| | | | | (m, 2H), 2.31 (s, 3H), 1.91-1.75 (m, 1H), |
| | | | | 1.67 (ddt, J = 12.7, 8.5, 4.2 Hz, 1H). |
| | | | | LC/MS (APCI) m/z calcd. for C₂₂H₂₄N₅O₃S⁺ |
| | | | | [M + H]⁺: 438.2; 438.2 found. |
| | | | | Stereochemistry arbitrarily assigned. |
| 32 | 19.2 | 5.4 | Methyl- amine | 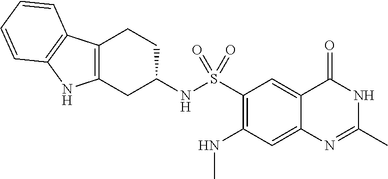 [SMILES: CNc1cc2nc(C)[nH]c(=O)c2cc1S(=O)(=O)N[C@H]1CCc2c([nH]c3ccccc23)C1] |
| | | | | (S)-2-Methyl-7-(methylamino)-4-oxo-N- |
| | | | | (2,3,4,9-tetrahydro-1H-carbazol-2-yl)-3,4- |
| | | | | dihydroquinazoline-6-sulfonamide. ¹H |
| | | | | NMR (400 MHz, DMSO-d₆) δ 11.84 (s, 1H), |
| | | | | 10.48 (s, 1H), 8.24 (s, 1H), 7.91 (d, J = 7.1 |
| | | | | Hz, 1H), 7.18 (d, J = 7.7 Hz, 1H), 7.13- |
| | | | | 7.02 (m, 1H), 6.86 (ddd, J = 8.1, 7.0, 1.3 Hz, |
| | | | | 1H), 6.79 (td, J = 7.4, 1.1 Hz, 1H), 6.56 (s, |
| | | | | 1H), 6.28 (q, J = 4.8 Hz, 1H), 3.35 (s, 1H), |
| | | | | 2.79 (d, J = 4.7 Hz, 3H), 2.73-2.51 (m, |
| | | | | 2H), 2.51-2.41 (m, 2H), 2.19 (s, 3H), 1.80- |
| | | | | 1.63 (m, 1H), 1.63-1.42 (m, 1H). LC/MS |
| | | | | (APCI) m/z calcd. for C₂₂H₂₄N₅O₃S⁺ |
| | | | | [M + H]⁺: 438.2; 438.2 found. |
| | | | | Stereochemistry arbitrarily assigned. |
| 29 | 19.2 | 1-(3- methyl-1H- indo1-2- yl)propan- 2-amine | Methyl- amine | 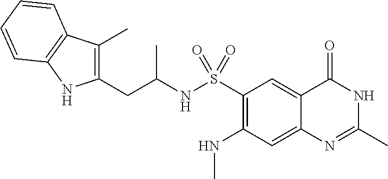 [SMILES: CNc1cc2nc(C)[nH]c(=O)c2cc1S(=O)(=O)NC(C)Cc1[nH]c2ccccc2c1C] |
| | | | | 2-Methyl-N-(1-(3-methyl-1H-indo1-2- |
| | | | | yl)propan-2-yl)-7-(methylamino)-4-oxo- |
| | | | | 3,4-dihydroquinazoline-6-sulfonamide. |
| | | | | ¹H NMR (400 MHz, Acetone-d₆) δ 9.59 (s, |
| | | | | 1H), 8.40 (s, 1H), 8.10 (s, 1H), 7.28 (d, J = |
| | | | | 7.8 Hz, 1H), 7.09 (d, J = 7.9 Hz, 1H), 6.93 |
| | | | | (td, J = 8.0, 7.5, 1.4 Hz, 1H), 6.88 (td, J = |
| | | | | 7.4, 1.2 Hz, 1H), 6.55 (d, J = 8.0 Hz, 1H), |
| | | | | 6.45 (s, 1H), 6.21 (d, J = 4.7 Hz, 1H), 3.62 |
| | | | | (p, J = 6.7 Hz, 1H), 2.75 (s, 3H), 2.38 (s, |
| | | | | 3H), 2.07 (s, 3H), 2.03-1.99 (m, 2H), 1.07 |
| | | | | (dd, J = 6.6, 1.0 Hz, 3H). LC/MS (APCI) |
| | | | | m/z calcd. for C₂₂H₂₆N₅O₃S⁺ [M + H]⁺: 440.2; |
| | | | | 440.2 found. |
| 4 | 19.2 | [1,1′- biphenyl]- 4-amine | Methyl- amine | 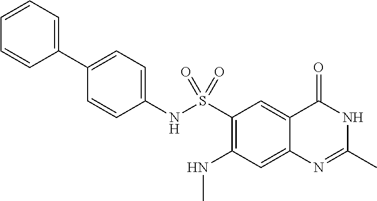 [SMILES: CNc1cc2nc(C)[nH]c(=O)c2cc1S(=O)(=O)Nc1ccc(-c2ccccc2)cc1] |
| | | | | N-([1,1′-biphenyl]-4-yl)-2-methyl-7- |
| | | | | (methylamino)-4-oxo-3,4- |
| | | | | dihydroquinazoline-6-sulfonamide. ¹H |
| | | | | NMR (400 MHz, Methanol-d₄) δ 8.74 (s, |
| | | | | 1H), 7.48 (d, J = 7.9 Hz, 2H), 7.42 (d, J = |
| | | | | 8.6 Hz, 2H), 7.37-7.34 (m, 2H), 7.33- |
| | | | | 7.22 (m, 2H), 7.18 (d, J = 8.6 Hz, 2H), 2.93 |
| | | | | (s, 3H), 2.41 (s, 3H). LC/MS (APCI) m/z |
| | | | | calcd. for C₂₂H₁₉N₄O₃S⁻ [M − H]⁻: 419.1; |
| | | | | 419.1 found. |
| 34 | 19.2 | 2-(1H- indo1-2- yl)ethan-1- amine | Methyl- amine | 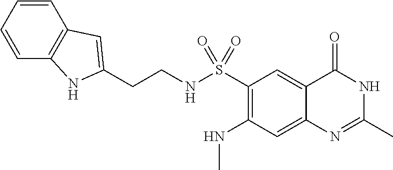 [SMILES: CNc1cc2nc(C)[nH]c(=O)c2cc1S(=O)(=O)NCCc1cc2ccccc2[nH]1] |
| | | | | N-(2-(1H-indo1-2-ypethyl)-2-methyl-7- |
| | | | | (methylamino)-4-oxo-3,4- |
| | | | | dihydroquinazoline-6-sulfonamide. ¹H |
| | | | | NMR (400 MHz, DMSO-d₆) δ 11.95 (s, 1H), |
| | | | | 11.05-10.74 (m, 1H), 8.32 (d, J = 1.6 Hz, |
| | | | | 1H), 7.94 (s, 1H), 7.38 (d, J = 7.7 Hz, 1H), |
| | | | | 7.23 (d, J = 7.9 Hz, 1H), 7.02-6.94 (m, |
| | | | | 1H), 6.91 (t, J = 7.3 Hz, 1H), 6.63 (s, 1H), |
| | | | | 6.36 (d, J = 49.8 Hz, 1H), 6.09 (s, 1H), 3.11 |
| | | | | (t, J = 7.3 Hz, 2H), 2.85 (d, J = 4.4 Hz, 3H), |
| | | | | 2.81 (t, J = 7.3 Hz, 2H), 2.30 (s, 3H). |
| | | | | LC/MS (APCI) m/z calcd. for C₂₀H₂₁N₅O₃S⁻ |
| | | | | [M − H]⁻: 412.1; 412.1 found. |
| 61 | 18.1 | 3.3 | Methyl- amine | 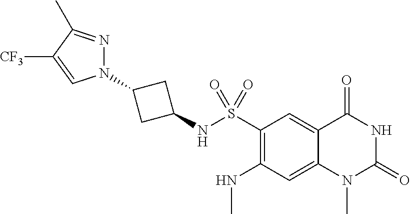 [SMILES: CNc1cc2c(cc1S(=O)(=O)N[C@H]1C[C@H](n3cc(C(F)(F)F)c(C)n3)C1)c(=O)[nH]c(=O)n2C] |
| | | | | 1-Methyl-N-(trans)-3-(3-methyl-4- |
| | | | | (trifluoromethyl)-1H-pyrazol-1- |
| | | | | yl)cyclobutyl)-7-(methylamino)-2,4-dioxo- |
| | | | | 1,2,3,4-tetrahydroquinazoline-6- |
| | | | | sulfonamide. ¹H NMR (400 MHz, DMSO- |
| | | | | d₆) δ 11.34 (s, 1H), 8.29 (d, J = 13.4 Hz, |
| | | | | 2H), 8.16 (d, J = 1.8 Hz, 1H), 6.62 (d, J = |
| | | | | 5.0 Hz, 1H), 6.31 (s, 1H), 4.88-4.77 (m, |
| | | | | 1H), 3.90 (dd, J = 13.0, 6.3 Hz, 1H), 3.44 (s, |
| | | | | 3H), 2.99 (d, J = 4.8 Hz, 3H), 2.48-2.31 |
| | | | | (m, 4H), 2.24 (d, J = 14.3 Hz, 3H). LC/MS |
| | | | | (APCI) m/z calcd. for C₁₉H₂₂F₃N₆O₄S⁺ |
| | | | | [M + H]⁺: 487.1; 487.2 found. |
| 1 | 3-cyano- 4- fluoro- benzene- sulfonyl chloride | 1.1 | Methyl- amine | 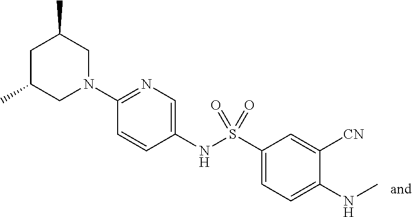 |
| | | | | 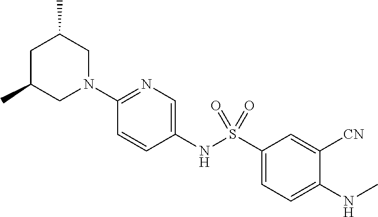 |
| | | | | 3-Cyano-N-(6-(trans-3,5- |
| | | | | dimethylpiperidin-1-yl)pyridin-3-yl)-4- |
| | | | | (methylamino) benzenesulfonamide. ¹H |
| | | | | NMR (400 MHz, Chloroform-d) δ 7.71 (d, |
| | | | | J = 2.1 Hz, 1H), 7.68 (dd, J = 9.0, 2.3 Hz, |
| | | | | 1H), 7.63 (d, J = 2.6 Hz, 1H), 7.29 (dd, J = |
| | | | | 9.1, 2.8 Hz, 1H), 6.58 (dd, J = 35.2, 9.0 Hz, |
| | | | | 2H), 6.41 (s, 1H), 5.22 (q, J = 5.0 Hz, 1H), |
| | | | | 3.57 (dd, J = 12.8, 3.8 Hz, 2H), 3.11 (dd, J = |
| | | | | 12.8, 6.9 Hz, 2H), 2.97 (d, J = 5.0 Hz, 3H), |
| | | | | 1.96 (pd, J = 6.5, 3.8 Hz, 2H), 1.45 (t, J = |
| | | | | 5.9 Hz, 2H), 0.93 (d, J = 6.8 Hz, 6H). |
| | | | | LC/MS (APCI) m/z calcd. for C₂₀H₂₄N₅O₂S⁻ |
| | | | | [M − H]⁻: 398.2; 398.2 found. |
| 8 | 19.2 | 1.2 | Methyl- amine | 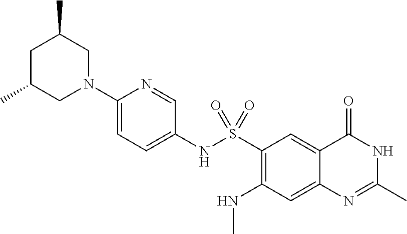 |
| | | | | N-(6-((3R,5R)-3,5-dimethylpiperidin-1- |
| | | | | yl)pyridin-3-yl)-2-methyl-7- |
| | | | | (methylamino)-4-oxo-3,4- |
| | | | | dihydroquinazoline-6-sulfonamide. ¹H |
| | | | | NMR (400 MHz, DMSO-d₆) δ 11.93 (s, 1H), |
| | | | | 8.08 (s, 1H), 7.61 (d, J = 2.7 Hz, 1H), 7.07 |
| | | | | (dd, J = 9.1, 2.7 Hz, 1H), 6.66 (d, J = 9.1 Hz, |
| | | | | 1H), 6.61 (s, 1H), 6.33 (q, J = 4.8 Hz, 1H), |
| | | | | 5.76 (s, 1H), 3.48 (dd, J = 12.8, 3.7 Hz, 2H), |
| | | | | 3.06 (dd, J = 12.9, 6.8 Hz, 2H), 2.88 (d, J = |
| | | | | 4.7 Hz, 3H), 2.27 (s, 3H), 1.82 (dq, J = 10.2, |
| | | | | 6.4 Hz, 2H), 1.37 (t, J = 5.8 Hz, 2H), 0.82 |
| | | | | (d, J = 6.8 Hz, 6H). LC/MS (APCI) m/z |
| | | | | calcd. for C₂₂H₂₉N₆O₃S⁺ [M + H]⁺: 457.2; |
| | | | | 457.2 found. |
| 10 | 18 1 | 1- phenyl- piperidin-4- amine | Methyl- amine | 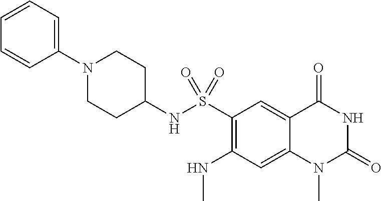 [SMILES: CNc1cc2c(cc1S(=O)(=O)NC1CCN(c3ccccc3)CC1)c(=O)[nH]c(=O)n2C] |
| | | | | 1-Methyl-7-(methylamino)-2,4-dioxo-N- |
| | | | | (1-phenylpiperidin-4-yl)-1,2,3,4- |
| | | | | tetrahydroquinazoline-6-sulfonamide. ¹H |
| | | | | NMR (400 MHz, DMSO-d₆) δ 11.34 (s, 1H), |
| | | | | 8.23 (s, 1H), 7.89 (d, J = 6.8 Hz, 1H), 7.16 |
| | | | | (dd, J = 8.7, 7.2 Hz, 2H), 6.87 (d, J = 7.8 Hz, |
| | | | | 2H), 6.72 (t, J = 7.2 Hz, 1H), 6.55 (q, J = |
| | | | | 5.3, 4.8 Hz, 1H), 6.33 (s, 1H), 3.46 (s, 4H), |
| | | | | 3.20-3.03 (m, 2H), 2.98 (d, J = 4.8 Hz, |
| | | | | 3H), 2.79-2.64 (m, 2H), 1.64 (d, J = 12.8 |
| | | | | Hz, 2H), 1.52-1.31 (m, 2H). LC/MS |
| | | | | (APCI) m/z calcd. for C₂₁H₂₆N₅O₄S⁺ |
| | | | | [M + H]⁺: 444.2; 444.2 found. |
| 12 | 18.1 | (trans)-4- phenyl- cyclohexan-1- amine | Methyl- amine | 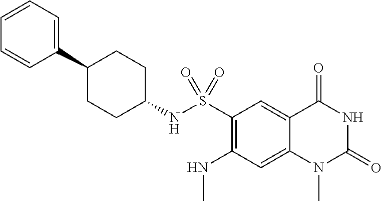 [SMILES: CNc1cc2c(cc1S(=O)(=O)N[C@H]1CC[C@H](c3ccccc3)CC1)c(=O)[nH]c(=O)n2C] |
| | | | | 1-Methyl-7-(methylamino)-2,4-dioxo-N-4- |
| | | | | phenylcyclohexyl)-1,2,3,4- |
| | | | | tetrahydroquinazoline-6-sulfonamide. ¹H |
| | | | | NMR (400 MHz, DMSO-d₆) δ 11.39 (s, 1H), |
| | | | | 8.29 (s, 1H), 7.89 (d, J = 7.1 Hz, 1H), 7.35- |
| | | | | 7.14 (m, 5H), 6.61 (d, J = 4.9 Hz, 1H), 6.38 |
| | | | | (s, 1H), 3.52 (s, 3H), 3.39 (s, 3H), 3.10- |
| | | | | 3.02 (m, 1H), 2.50-2.36 (m, 1H), 1.88- |
| | | | | 1.67 (m, 4H), 1.61-1.24 (m, 4H). LC/MS |
| | | | | (APCI) m/z calcd. for C₂₂H₂₇N₄O₄S⁺ |
| | | | | [M + H]⁺: 443.2; 443.3 found. |
| 13 | 18.1 | N¹- phenyl- propane-1,3- diamine | Methyl- amine | 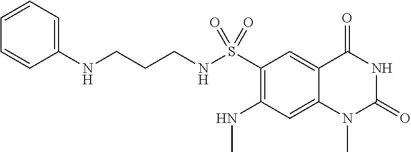 [SMILES: CNc1cc2c(cc1S(=O)(=O)NCCCNc1ccccc1)c(=O)[nH]c(=O)n2C] |
| | | | | 1-Methyl-7-(methylamino)-2,4-dioxo-N- |
| | | | | (3-(phenylamino)propyl)-1,2,3,4- |
| | | | | tetrahydroquinazoline-6-sulfonamide. ¹H |
| | | | | NMR (400 MHz, DMSO-d₆) δ 11.32 (s, 1H), |
| | | | | 8.17 (s, 1H), 7.77 (t, J = 5.7 Hz, 1H), 7.01 |
| | | | | (dd, J = 8.4, 7.2 Hz, 2H), 6.58 (q, J = 4.7 Hz, |
| | | | | 1H), 6.47 (td, J = 7.2, 1.0 Hz, 1H), 6.45- |
| | | | | 6.38 (m, 2H), 6.25 (s, 1H), 5.42 (t, J = 5.5 |
| | | | | Hz, 1H), 3.41 (s, 3H), 2.95 (d, J = 4.8 Hz, |
| | | | | 3H), 2.87 (dq, J = 13.3, 6.5 Hz, 4H), 1.60 (p, |
| | | | | J = 6.8 Hz, 2H). LC/MS (APCI) m/z calcd. |
| | | | | for C₁₉H₂₄N₅O₄S⁺ [M + H]⁺: 418.2; 418.2 |
| | | | | found. |
| 11 | 19.2 | 1-(3- fluorophenyl) piperidin- 4-amine | Methyl- amine | 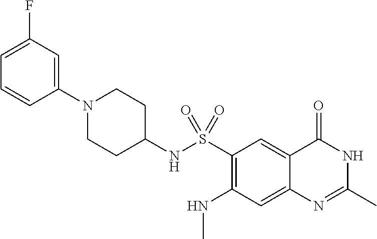 [SMILES: CNc1cc2nc(C)[nH]c(=O)c2cc1S(=O)(=O)NC1CCN(c2cccc(F)c2)CC1] |
| | | | | N-(1-(3-fluorophenyl)piperidin-4-yl)-2- |
| | | | | methyl-7-(methylamino)-4-oxo-3,4- |
| | | | | dihydroquinazoline-6-sulfonamide. ¹H |
| | | | | NMR (400 MHz, DMSO-d₆) δ 11.98 (s, 1H), |
| | | | | 8.34 (s, 1H), 7.94 (d, J = 7.7 Hz, 1H), 7.15 |
| | | | | (q, J = 8.1 Hz, 1H), 6.67 (q, J = 5.8 Hz, 3H), |
| | | | | 6.47 (td, J = 8.3, 2.3 Hz, 1H), 6.32 (q, J = |
| | | | | 4.8 Hz, 1H), 3.55 (d, J = 13.1 Hz, 2H), 3.26- |
| | | | | 3.06 (m, 1H), 2.91 (d, J = 4.7 Hz, 3H), |
| | | | | 2.76 (t, J = 11.5 Hz, 2H), 2.31 (s, 3H), 1.59 |
| | | | | (d, J = 12.5 Hz, 2H), 1.47-1.25 (m, 2H). |
| | | | | LC/MS (APCI) m/z calcd. for C₂₁H₂₅FN₅O₃S⁺ |
| | | | | [M + H]⁺: 446.2, 446.3 found. |
| 2 | 3-cyano- 4- fluoro- benzene- sulfonyl chloride | 1.1 | — | 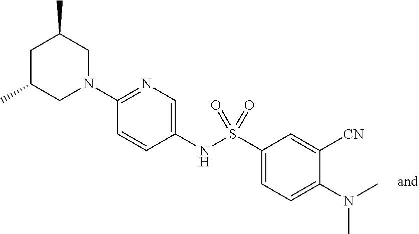 |
| | | | | 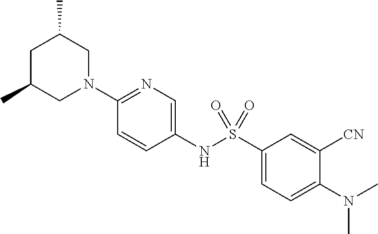 |
| | | | | 3-Cyano-4-(dimethylamino)-N-(6-(trans- |
| | | | | 3,5-dimethylpiperidin-l-yl)pyridin-3- |
| | | | | yl)benzenesulfonamide. ¹H NMR (400 |
| | | | | MHz, Methanol-d₄) δ 7.69 (d, J = 2.3 Hz, |
| | | | | 1H), 7.65-7.52 (m, 2H), 7.26 (dd, J = 9.2, |
| | | | | 2.7 Hz, 1H), 6.97 (d, J = 9.2 Hz, 1H), 6.71 |
| | | | | (d, J = 9.2 Hz, 1H), 3.54 (dd, J = 12.9, 3.7 |
| | | | | Hz, 2H), 3.20 (s, 6H), 3.14 (dd, J = 12.9, 6.9 |
| | | | | Hz, 2H), 1.95 (pd, J = 6.5, 3.7 Hz, 2H), 1.48 |
| | | | | (t, J = 5.9 Hz, 2H), 0.94 (d, J = 6.9 Hz, 6H). |
| | | | | LC/MS (APCI) m/z calcd. for C₂₁H₂₆N₅O₂S⁻ |
| | | | | [M − H]⁻: 412.2; 412.1 found. |

### Example S-3. Palladium Catalyzed Methylation General Procedure

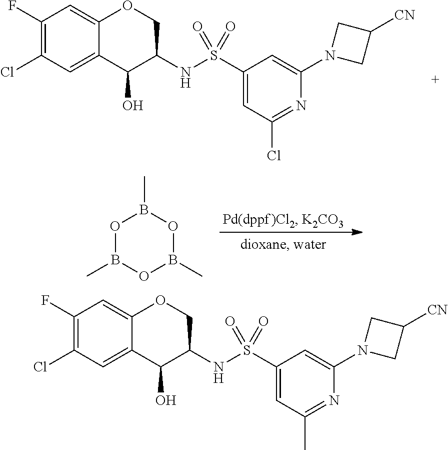

N-((3R,4S)-6-chloro-7-fluoro-4-hydroxychroman-3-yl)-2-(3-cyanoazetidin-1-yl)-6-methylpyridine-4-sulfonamide. Pd(dppf)Cl2 (9 mg, 0.013 mmol, 0.2 equiv.) and K2CO3 (26 mg, 0.19 mmol, 3 equiv.) were added to a solution of 2-chloro-N-((3R,4S)-6-chloro-7-fluoro-4-hydroxychroman-3-yl)-6-(3-cyanoazetidin-1-yl)pyridine-4-sulfonamide in dioxane (2 mL) before trimethylboroxine (27 mg, 0.19 mmol, 3 equiv.) and water (0.5 mL) were added and the reaction heated to 130° C. in a microwave reactor for 15 min. The solvents were removed by rotary evaporation, the crude residue suspended in NMP, filtered through a 0.4 μm syringe filter, and product isolated by reverse phase HPLC (10->100% MeCN/H₂O with 0.1% formic acid) as a white solid (13 mg, 45%). ¹H NMR (400 MHz, Methanol-d₄) δ 7.34 (d, J=8.3 Hz, 1H), 7.04 (s, 1H), 6.70 (t, J=5.3 Hz, 2H), 4.51 (d, J=3.8 Hz, 1H), 4.38 (t, J=8.4 Hz, 2H), 4.24 (t, J=6.9 Hz, 2H), 4.07 (t, J=10.3 Hz, 1H), 3.97 (dd, J=10.6, 3.3 Hz, 1H), 3.78 (dtd, J=30.6, 9.1, 4.9 Hz, 2H), 2.47 (s, 3H). LC/MS (APCI) m/z calcd. for C₁₉H₁₉ClFN₄O₄S⁺ [M+H]⁺: 453.1; 453.1 found.

TABLE S-3
The following compounds were prepared according to synthetic procedures as described in Example S-3.

| Compound | Intermediate | Structure, Name and Data |
|---|---|---|
| 85 | 26.1 | 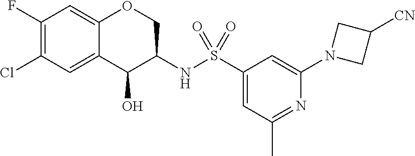 [SMILES: Cc1cc(S(=O)(=O)N[C@@H]2COc3cc(F)c(Cl)cc3[C@@H]2O)cc(N2CC(C#N)C2)n1] |
| | | N-((3R,4S)-6-chloro-7-fluoro-4-hydroxychroman-3-yl)-2-(3- |
| | | cyanoazetidin-1-yl)-6-methylpyridine-4-sulfonamide. ¹H NMR |
| | | (400 MHz, Methanol-d₄) δ 7.34 (d, J = 8.3 Hz, 1H), 7.04 (s, 1H), |
| | | 6.70 (t, J = 5.3 Hz, 2H), 4.51 (d, J = 3.8 Hz, 1H), 4.38 (t, J = 8.4 Hz, |
| | | 2H), 4.24 (t, J = 6.9 Hz, 2H), 4.07 (t, J = 10.3 Hz, 1H), 3.97 (dd, J = |
| | | 10.6, 3.3 Hz, 1H), 3.78 (dtd, J = 30.6, 9.1, 4.9 Hz, 2H), 2.47 (s, 3H). |
| | | LC/MS (APCI) m/z calcd. for C₁₉H₁₉ClFN₄O₄S⁺ [M + H]⁺: 453.1; |
| | | 453.1 found. |
| 86 | 26.2 | 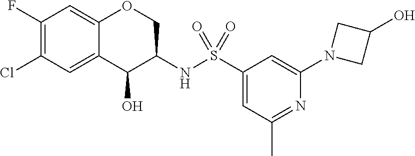 [SMILES: Cc1cc(S(=O)(=O)N[C@@H]2COc3cc(F)c(Cl)cc3[C@@H]2O)cc(N2CC(O)C2)n1] |
| | | N-((3R,4S)-6-chloro-7-fluoro-4-hydroxychroman-3-yl)-2-(3- |
| | | hydroxyazetidin-1-yl)-6-methylpyridine-4-sulfonamide. 1H |
| | | NMR (400 MHz, Methanol-d₄) δ 7.34 (d, J = 8.2 Hz, 1H), 6.93 (s, |
| | | 1H), 6.70 (d, J = 10.4 Hz, 1H), 6.64 (s, 1H), 4.72 (tt, J = 6.5, 4.4 Hz, |
| | | 1H), 4.52 (d, J = 3.7 Hz, 1H), 4.32 (ddt, J = 9.1, 6.6, 1.3 Hz, 2H), |
| | | 4.13-4.01 (m, 1H), 3.96 (ddd, J = 10.7, 3.8, 1.0 Hz, 1H), 3.86 (ddt, |
| | | J = 8.9, 4.4, 1.6 Hz, 2H), 3.72 (dt, J = 9.9, 3.8 Hz, 1H), 2.46 (s, 3H). |
| | | LC/MS (APCI) m/z calcd. for C₁₈H₁₈ClFN₃O₅S⁻ [M − H]⁻: 442.1; |
| | | 442.1 found. |
| 87 | 26.3 | 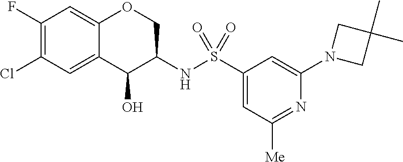 [SMILES: Cc1cc(S(=O)(=O)N[C@@H]2COc3cc(F)c(Cl)cc3[C@@H]2O)cc(N2CC(C)(C)C2)n1] |
| | | N-((3R,4S)-6-chloro-7-fluoro-4-hydroxychroman-3-yl)-2-(3,3- |
| | | dimethylazetidin-1-yl)-6-methylpyridine-4-sulfonamide. ¹H |
| | | NMR (400 MHz, Methanol-d₄) δ 7.34 (d, J = 8.4 Hz, 1H), 6.90 (s, |
| | | 1H), 6.70 (d, J = 10.4 Hz, 1H), 6.59 (s, 1H), 4.52 (d, J = 3.7 Hz, 1H), |
| | | 4.07 (t, J = 10.3 Hz, 1H), 3.97 (dd, J = 10.6, 3.8 Hz, 1H), 3.77 (s, |
| | | 4H), 3.73 (dd, J = 9.1, 4.7 Hz, 1H), 2.44 (s, 3H), 1.36 (s, 6H). LC/MS |
| | | (APCI) m/z calcd. for C₂₀H₂₂ClFN₃O₄S⁻[M − H]⁻: 454.1; 454.1 found. |
| 88 | 26.4 | 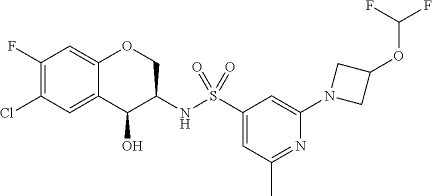 [SMILES: Cc1cc(S(=O)(=O)N[C@@H]2COc3cc(F)c(Cl)cc3[C@@H]2O)cc(N2CC(OC(F)F)C2)n1] |
| | | N-((3R,4S)-6-chloro-7-fluoro-4-hydroxychroman-3-yl)-2-(3- |
| | | (difluoromethoxy)azetidin-1-yl)-6-methylpyridine-4- |
| | | sulfonamide. ¹H NMR (400 MHz, Methanol-d₄) δ 7.35 (d, J = 8.3 |
| | | Hz, 1H), 6.98 (d, J = 1.3 Hz, 1H), 6.70 (d, J = 10.4 Hz, 1H), 6.68 (s, |
| | | 1H), 6.51 (t, J = 74.1 Hz, 1H), 5.15 (tt, J = 6.6, 4.2 Hz, 1H), 4.52 (d, |
| | | J = 3.8 Hz, 1H), 4.46-4.36 (m, 2H), 4.13-4.03 (m, 3H), 3.97 (ddd, |
| | | J = 10.7, 3.8, 1.2 Hz, 1H), 3.74 (dt, J = 9.8, 3.8 Hz, 1H), 2.47 (s, 3H). |
| | | LC/MS (APCI) m/z calcd. for C₁₉H₁₈ClF₃N₃O₅S⁻ [M − H]⁻: 492.1; |
| | | 492.1 found. |
| 90 | 26.5 | 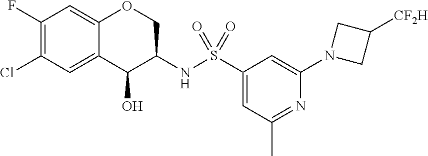 [SMILES: Cc1cc(S(=O)(=O)N[C@@H]2COc3cc(F)c(Cl)cc3[C@@H]2O)cc(N2CC(C(F)F)C2)n1] |
| | | N-((3R,4S)-6-Chloro-7-fluoro-4-hydroxychroman-3-yl)-2-(3- |
| | | (difluoromethyDazetidin-1-yl)-6-methylpyridine-4-sulfonamide. |
| | | ¹H NMR (400 MHz, Methanol-d₄) δ 7.34 (d, J = 8.3 Hz, 1H), 6.97 |
| | | (s, 1H), 6.70 (d, J = 10.4 Hz, 1H), 6.66 (s, 1H), 6.18 (td, J = 56.4, 4.2 |
| | | Hz, 1H), 4.51 (d, J = 3.7 Hz, 1H), 4.19 (t, J = 8.6 Hz, 2H), 4.13- |
| | | 4.01 (m, 3H), 3.97 (dd, J = 10.6, 3.3 Hz, 1H), 3.73 (dt, J = 9.7, 3.7 |
| | | Hz, 1H), 3.29-3.13 (m, 1H), 2.46 (s, 3H). LC/MS (APCI) m/z calcd. |
| | | for C₁₉H₁₈ClF₃N₃O₄S⁻ [M − H]⁻: 476.1; 476.1 found. |
| 91 | 26.6 | 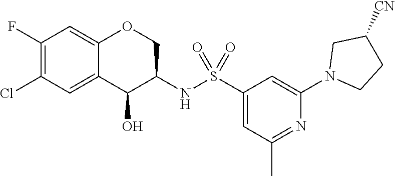 [SMILES: Cc1cc(S(=O)(=O)N[C@@H]2COc3cc(F)c(Cl)cc3[C@@H]2O)cc(N2CC[C@@H](C#N)C2)n1] |
| | | N-((3R,4S)-6-chloro-7-fluoro-4-hydroxychroman-3-yl)-2-((R)-3- |
| | | cyanopyrrolidin-1-yl)-6-methylpyridine-4-sulfonamide. ¹H |
| | | NMR (400 MHz, Methanol-d₄) δ 7.34 (d, J = 8.3 Hz, 1H), 6.94 (s, |
| | | 1H), 6.77 (s, 1H), 6.69 (d, J = 10.4 Hz, 1H), 4.52 (d, J = 3.7 Hz, 1H), |
| | | 4.12-4.02 (m, 1H), 3.97 (dd, J = 10.7, 3.8 Hz, 1H), 3.87 (ddd, J = |
| | | 10.8, 7.2, 2.1 Hz, 1H), 3.80 (ddd, J = 10.8, 5.6, 2.2 Hz, 1H), 3.73 (dt, |
| | | J = 9.8, 3.8 Hz, 1H), 3.71-3.64 (m, 1H), 3.64-3.54 (m, 1H), 3.48 |
| | | (p, J = 6.7 Hz, 1H), 2.47 (s, 4H), 2.36 (dq, J = 12.8, 6.5 Hz, 1H). |
| | | LC/MS (APCI) m/z calcd. for C₂₀H₁₉ClFN₄O₄S⁻[M − H]⁻: 465.1; |
| | | 465.1 found. |
| 9 | 25.1 | 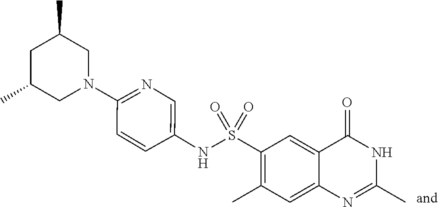 |
| | | 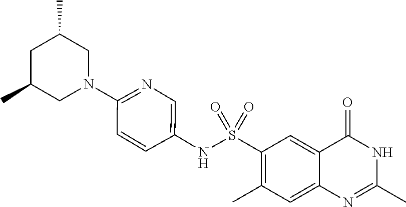 |
| | | N-(6-((trans)-3,5-dimethylpiperidin-1-yl)pyridin-3-yl)-2,7- |
| | | dimethyl-4-oxo-3,4-dihydroquinazoline-6-sulfonamide. ¹H NMR |
| | | (400 MHz, DMSO-d₆) δ 12.48 (s, 1H), 9.91 (s, 1H), 8.40 (s, 1H), |
| | | 7.70 (d, J = 2.7 Hz, 1H), 7.62 (s, 1H), 7.17 (dd, J = 9.1, 2.8 Hz, 1H), |
| | | 6.73 (d, J = 9.1 Hz, 1H), 3.54 (dd, J = 12.8, 3.7 Hz, 2H), 3.12 (dd, |
| | | J = 12.8, 6.8 Hz, 2H), 2.73 (s, 3H), 2.41 (s, 3H), 1.89 (pd, J = 6.3, |
| | | 3.7 Hz, 2H), 1.43 (t, J = 5.8 Hz, 2H), 0.88 (d, J = 6.8 Hz, 6H). LC/MS |
| | | (APCI) m/z calcd. for C₂₂H₂₈N₅O3S⁺ [M + H]⁺: 442.2; 442.5 found. |

### Example S-4. Base Catalyzed SNAr General Procedure

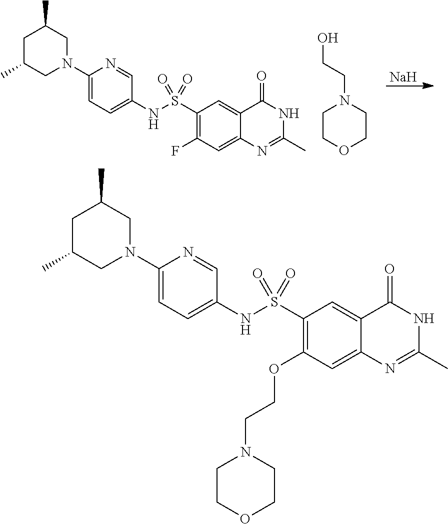

N-(6-((3R,5R)-3,5-dimethylpiperidin-1-yl)pyridin-3-yl)-2-methyl-7-(2-morpholinoethoxy)-4-oxo-3,4-dihydroquinazoline-6-sulfonamide. NaH (2.9 g, 72.g mmol, 10 equiv., 60% dispersion in mineral oil) was added to stirring solution of 2-morpholinoethan-1-ol (15 mL, 113 mmol, 15 equiv.) in THF at 0° C. After 30 min, the reaction was warmed to rt, THF removed by rotary evaporation, and the contents transferred to a microwave vial. N-(6-((3R,5R)-3,5-dimethylpiperidin-1-yl)pyridin-3-yl)-7-fluoro-2-methyl-4-oxo-3,4-dihydroquinazoline-6-sulfonamide (3.2 mg, 7.183 mmol, 1 equiv.) was added before the vial was sealed and the reaction heated to 130° C. for 30 min. The reaction was then cooled to rt, quenched with saturated sodium bicarbonate, extracted with CH₂Cl₂, the organic layer washed with brine, dried over sodium sulfate, filtered, and solvent removed by rotary evaporation. The crude material was resolved by reverse phase HPLC to give the product as the formate salt. The formate salt product was suspended in a solution of saturated sodium bicarbonate and extracted with CH₂Cl₂, the organic layer dried over sodium sulfate, filtered, and solvent removed by rotary evaporation to give the product as a white solid (1.55, 39%). ¹H NMR (400 MHz, Methanol-d₄) δ 8.26 (s, 1H), 7.47 (d, J=2.7 Hz, 1H), 7.14 (s, 1H), 7.11 (dd, J=9.1, 2.7 Hz, 1H), 6.54 (d, J=9.2 Hz, 1H), 4.40 (t, J=5.0 Hz, 2H), 3.53-3.43 (m, 4H), 3.40 (dd, J=12.9, 3.7 Hz, 2H), 2.99 (dd, J=12.9, 6.9 Hz, 2H), 2.84 (t, J=5.0 Hz, 2H), 2.54 (s, 4H), 2.34 (s, 3H), 1.85-1.67 (m, 2H), 1.34 (t, J=5.8 Hz, 2H), 0.78 (d, J=6.8 Hz, 6H). LC/MS (APCI) m/z calcd. for C₂₇H₃₇N₆O₅S⁺ [M+H]⁺: 557.3; 557.3 found.

TABLE S-4
The following compounds were prepared according to synthetic procedures as described in Example S-4.

| Compound | Intermediate | Structure, Name and Data |
|---|---|---|
| 6 | 23.19 | 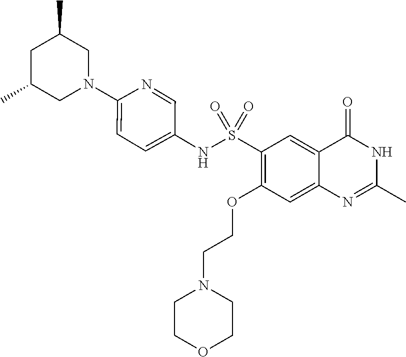 |
| | | N-(6-((3R,5R)-3,5-dimethylpiperidin-1-yl)pyridin-3-yl)-2- |
| | | methyl-7-(2-morpholinoethoxy)-4-oxo-3,4-dihydroquinazoline- |
| | | 6-sulfonamide. ¹H NMR (400 MHz, Methanol-d₄) δ 8.26 (s, 1H), |
| | | 7.47 (d, J = 2.7 Hz, 1H), 7.14 (s, 1H), 7.11 (dd, J = 9.1, 2.7 Hz, |
| | | 1H), 6.54 (d, J = 9.2 Hz, 1H), 4.40 (t, J = 5.0 Hz, 2H), 3.53-3.43 |
| | | (m, 4H), 3.40 (dd, J = 12.9, 3.7 Hz, 2H), 2.99 (dd, J = 12.9, 6.9 Hz, |
| | | 2H), 2.84 (t, J = 5.0 Hz, 2H), 2.54 (s, 4H), 2.34 (s, 3H), 1.85-1.67 |
| | | (m, 2H), 1.34 (t, J = 5.8 Hz, 2H), 0.78 (d, J = 6.8 Hz, 6H). LC/MS |
| | | (APCI) m/z calcd. for C₂₇H₃₇N₆O₅S⁺ [M + H]⁺: 557.3; 557.3 found. |
| 62 | 18.1, 3.3, sodium methoxide | 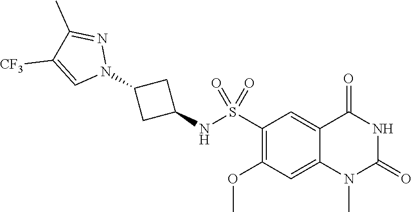 [SMILES: COc1cc2c(cc1S(=O)(=O)N[C@H]1C[C@H](n3cc(C(F)(F)F)c(C)n3)C1)c(=O)[nH]c(=O)n2C] |
| | | 7-Methoxy-1-methyl-N-((trans)-3-(3-methyl-4- |
| | | (trifluoromethyl)-1H-pyrazol-1-yl)cyclobutyl)-2,4-dioxo-1,2,3,4- |
| | | tetrahydroquinazoline-6-sulfonamide. ¹H NMR (400 MHz, |
| | | DMSO-d₆) δ 11.65 (s, 1H), 8.27 (d, J = 3.4 Hz, 2H), 8.03 (d, J = 8.2 |
| | | Hz, 1H), 6.93 (s, 1H), 4.89-4.71 (m, 1H), 4.10 (s, 3H), 4.02 (q, J = |
| | | 7.4 Hz, 1H), 3.49 (s, 3H), 2.48-2.33 (m, 4H), 2.23 (s, 3H). |
| | | LC/MS (APCI) m/z calcd. for C₁₉H₂₁F₃N₅O₅S⁺ [M + H]⁺: 488.1; |
| | | 488.1 found. |

### Example S5. Chiral Purifications

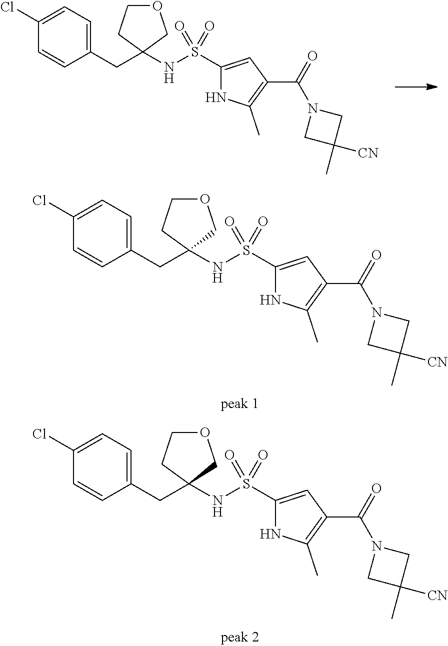 [SMILES: Cc1[nH]c(S(=O)(=O)NC2(Cc3ccc(Cl)cc3)CCOC2)cc1C(=O)N1CC(C)(C#N)C1 ; Cc1[nH]c(S(=O)(=O)N[C@@]2(Cc3ccc(Cl)cc3)CCOC2)cc1C(=O)N1CC(C)(C#N)C1 ; Cc1[nH]c(S(=O)(=O)N[C@]2(Cc3ccc(Cl)cc3)CCOC2)cc1C(=O)N1CC(C)(C#N)C1]

(R)—N-(3-(4-chlorobenzyl)tetrahydrofuran-3-yl)-4-(3-cyano-3-methylazetidine-1-carbonyl)-5-methyl-1H-pyrrole-2-sulfonamide (CK3762429). N-(3-(4-chlorobenzyl)tetrahydrofuran-3-yl)-4-(3-cyano-3-methylazetidine-1-carbonyl)-5-methyl-1H-pyrrole-2-sulfonamide was resolved by chiral chromotography (chiral pack AD-H column, 15% iPrOH/hexanes at 15 mL/min) to give peak 1 and peak 2 (CK3762429). ¹H NMR (400 MHz, DMSO-d₆) δ 12.13 (s, 1H), 7.52 (s, 1H), 7.36 (d, J=8.4 Hz, 2H), 7.29 (d, J=8.4 Hz, 2H), 6.64 (s, 1H), 4.46-4.14 (m, 4H), 3.72 (d, J=9.0 Hz, 2H), 3.54 (dd, J=15.7, 8.4 Hz, 2H), 3.08-2.93 (m, 2H), 2.39 (s, 3H), 2.07-2.01 (m, 1H), 1.88-1.83 (m, 1H), 1.62 (s, 3H). LC/MS (APCI) m/z calcd. for C₂₂H₂₆ClN₄O₄S⁺ [M+H]⁺: 477.1; 477.1 found.

NB: Stereochemistry Assigned Randomly and not Confirmed by x-Ray Crystallography

TABLE S-5
The following compounds were prepared according to synthetic procedures as described in Example S-5.

| Compound | Racemate | Structure, Name and Data |
|---|---|---|
| 63 | Chiral Purification Intermediate 2 | 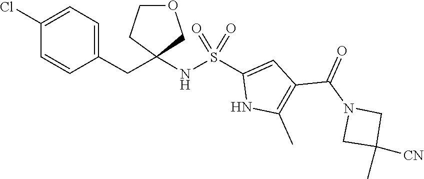 [SMILES: Cc1[nH]c(S(=O)(=O)N[C@]2(Cc3ccc(Cl)cc3)CCOC2)cc1C(=O)N1CC(C)(C#N)C1] |
| | | (R)-N-(3-(4-chlorobenzyl)tetrahydrofuran-3-yl)-4-(3- |
| | | cyano-3-methylazetidine-1-carbonyl)-5-methyl-1H- |
| | | pyrrole-2-sulfonamide. ¹H NMR (400 MHz, DMSO-d₆) δ |
| | | 12.13 (s, 1H), 7.52 (s, 1H), 7.36 (d, J = 8.4 Hz, 2H), 7.29 (d, |
| | | J = 8.4 Hz, 2H), 6.64 (s, 1H), 4.46-4.14 (m, 4H), 3.72 (d, J = |
| | | 9.0 Hz, 2H), 3.54 (dd, J = 15.7, 8.4 Hz, 2H), 3.08-2.93 (m, |
| | | 2H), 2.39 (s, 3H), 2.07-2.01 (m, 1H), 1.88-1.83 (m, 1H), |
| | | 1.62 (s, 3H). LC/MS (APCI) m/z calcd. for C₂₂H₂₆ClN₄O₄S⁺ |
| | | [M + H]⁺: 477.1; 477.1 found. |
| | | NB: Stereochemistry assigned randomly and not confirmed by |
| | | x-ray crystallography |
| 64 | Chiral Purification Intermediate 3 | 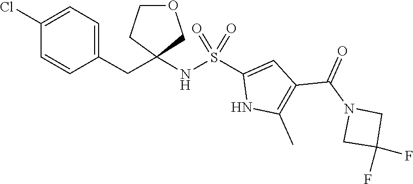 [SMILES: Cc1[nH]c(S(=O)(=O)N[C@]2(Cc3ccc(Cl)cc3)CCOC2)cc1C(=O)N1CC(F)(F)C1] |
| | | (R)-N-(3-(4-chlorobenzyl)tetrahydrofuran-3-yl)-4-(3,3- |
| | | difluoroazetidine-1-carbonyl)-5-methyl-1H-pyrrole-2- |
| | | sulfonamide. ¹H NMR (400 MHz, DMSO-d₆) δ 12.19 (s, |
| | | 1H), 7.53 (s, 1H), 7.36 (d, J = 8.3 Hz, 2H), 7.29 (d, J = 8.5 Hz, |
| | | 2H), 6.67 (s, 1H), 4.61-4.40 (m, 4H), 3.79-3.68 (m, 2H), |
| | | 3.54 (dd, J = 19.4, 8.5 Hz, 2H), 3.08-2.93 (m, 2H), 2.40 (s, |
| | | 3H), 2.05 (ddd, J = 17.1, 8.2, 4.5 Hz, 1H), 1.89-1.80 (m, |
| | | 1H). LC/MS (APCI) m/z calcd. for C₂₀H₂₃ClF₂N₃O₄S⁺ |
| | | [M + H]⁺: 474.1; 474.1 found. |
| | | NB: Stereochemistry assigned randomly and not confirmed by |
| | | x-ray crystallography |
| 65 | Chiral Purification Intermediate 4 | 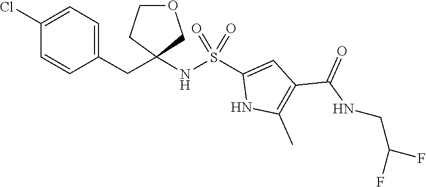 [SMILES: Cc1[nH]c(S(=O)(=O)N[C@]2(Cc3ccc(Cl)cc3)CCOC2)cc1C(=O)NCC(F)F] |
| | | (R)-5-(N-(3-(4-chlorobenzyl)tetrahydrofuran-3- |
| | | yl)sulfamoyl)-N-(2,2-difluoroethyl)-2-methyl-1H-pyrrole-3- |
| | | carboxamide. 1H NMR (400 MHz, DMSO-d6) δ 12.00 (s, |
| | | 1H), 8.25 (t, J = 5.9 Hz, 1H), 7.51 (s, 1H), 7.41-7.33 (m, |
| | | 2H), 7.33-7.23 (m, 2H), 7.09 (s, 1H), 6.05 (t, J = 4.2 Hz, |
| | | 1H), 3.78-3.62 (m, 2H), 3.62-3.45 (m, 4H), 3.01 (s, 2H), |
| | | 2.43 (s, 3H), 2.08 (ddd, J = 12.4, 7.5, 4.5 Hz, 1H), 1.82 (dt, J = |
| | | 12.9, 8.2 Hz, 1H). LC/MS (APCI) m/z calcd. for |
| | | C18H21F3N5O3S⁺ [M + H]+: 462.1; 462.1 found. |
| | | NB: Stereochemistry assigned randomly and not confirmed by |
| | | x-ray crystallography |
| 100 | Chiral Purification Intermediate 5 | 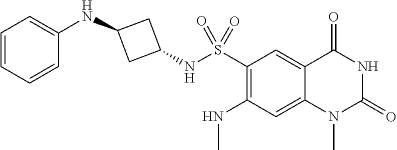 [SMILES: CNc1cc2c(cc1S(=O)(=O)N[C@H]1C[C@H](Nc3ccccc3)C1)c(=O)[nH]c(=O)n2C] |
| | | 1-Methyl-7-(methylamino)-2,4-dioxo-N-((1r,3r)-3- |
| | | (phenylamino)cyclobutyl)-1,2,3,4-tetrahydroquinazoline-6- |
| | | sulfonamide. ¹H NMR (400 MHz, DMSO-d₆) δ 11.33 (s, |
| | | 1H), 8.36-8.11 (m, 2H), 7.03 (t, J = 7.8 Hz, 2H), 6.65-6.55 |
| | | (m, 1H), 6.51 (t, J = 7.2 Hz, 1H), 6.37 (d, J = 7.7 Hz, 2H), |
| | | 6.31 (s, 1H), 5.87 (d, J = 5.6 Hz, 1H), 3.82-3.64 (m, 2H), |
| | | 3.45 (s, 3H), 2.99 (d, J = 4.6 Hz, 3H), 2.19-2.04 (m, 2H), |
| | | 2.01-1.85 (m, 2H). LC/MS (APCI) m/z calcd. for |
| | | C₂₀H₂₄N₅O₄S⁺ [M + H]⁺: 430.2; 430.3 found. |
| | | NB: Stereochemistry assigned randomly and not confirmed by |
| | | x-ray crystallography |
| 17 | Chiral Purification Intermediate 5 | 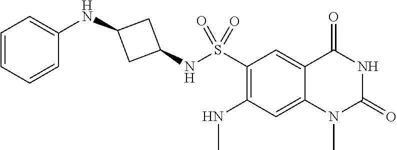 [SMILES: CNc1cc2c(cc1S(=O)(=O)N[C@H]1C[C@@H](Nc3ccccc3)C1)c(=O)[nH]c(=O)n2C] |
| | | 1-Methyl-7-(methylamino)-2,4-dioxo-N-(cis)-3- |
| | | (phenylamino)cyclobutyl)-1,2,3,4-tetrahydroquinazoline-6- |
| | | sulfonamide. ¹H NMR (400 MHz, DMSO-d₆) δ 11.34 (s, |
| | | 1H), 8.19 (s, 1H), 8.09 (s, 1H), 7.01 (dd, J = 8.5, 7.2 Hz, 2H), |
| | | 6.59 (d, J = 5.0 Hz, 1H), 6.48 (t, J = 7.3 Hz, 1H), 6.41 (d, J = |
| | | 7.4 Hz, 2H), 6.31 (s, 1H), 5.78 (d, J = 6.2 Hz, 1H), 3.45 (s, |
| | | 3H), 3.36 (dd, J = 12.0, 5.0 Hz, 2H), 2.98 (d, J = 4.8 Hz, 3H), |
| | | 2.48-2.34 (m, 2H), 1.58 (dt, J = 10.9, 8.4 Hz, 2H). LC/MS |
| | | (APCI) m/z calcd. for C₂₀H₂₄N₅O₄S⁺ [M + H]⁺: 430.2; 430.1 found. |
| | | NB: Stereochemistry assigned randomly and not confirmed by |
| | | x-ray crystallography |
| 72 | Chiral Purification Intermediate 6 | 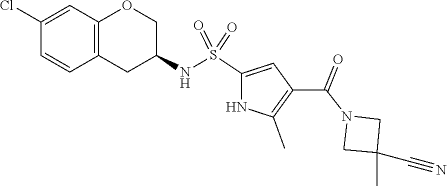 [SMILES: Cc1[nH]c(S(=O)(=O)N[C@@H]2COc3cc(Cl)ccc3C2)cc1C(=O)N1CC(C)(C#N)C1] |
| | | (S)-N-(7-chlorochroman-3-yl)-4-(3-cyano-3- |
| | | methylazetidine-1-carbonyl)-5-methyl-1H-pyrrole-2- |
| | | sulfonamide. ¹H NMR (400 MHz, DMSO-d₆) δ 12.25 (s, |
| | | 1H), 7.74 (s, 1H), 7.07 (d, J = 8.2 Hz, 1H), 6.90 (dd, J = 8.1, |
| | | 2.2 Hz, 1H), 6.85 (d, J = 2.1 Hz, 1H), 6.75 (s, 1H), 4.05 (d, J = |
| | | 11.5 Hz, 2H), 3.83 (dd, J = 10.7, 7.8 Hz, 1H), 3.63 (s, 1H), |
| | | 3.44 (dd, J = 7.0, 4.9 Hz, 1H), 2.85 (dd, J = 16.5, 5.4 Hz, 1H), |
| | | 2.70-2.59 (m, 1H), 2.41 (s, 3H), 2.33 (dt, J = 4.6, 2.3 Hz, |
| | | 1H), 1.62 (s, 3H), 1.06 (t, J = 7.0 Hz, 1H). LC/MS (APCI) m/z |
| | | calcd. for C₂₀H₂₂N₄O₄SCl⁺ [M + H]⁺: 448.1; 449.1 found. |
| | | NB: Stereochemistry assigned randomly and not confirmed by |
| | | x-ray crystallography |
| 73 | Chiral Purification Intermediate 1 | 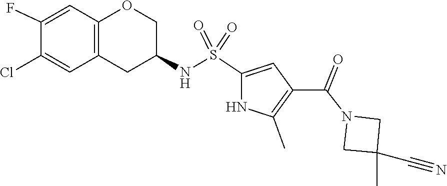 [SMILES: Cc1[nH]c(S(=O)(=O)N[C@@H]2COc3cc(F)c(Cl)cc3C2)cc1C(=O)N1CC(C)(C#N)C1] |
| | | (S)-N-(6-chloro-7-fluorochroman-3-yl)-4-(3-cyano-3- |
| | | methylazetidine-1-carbonyl)-5-methyl-1H-pyrrole-2- |
| | | sulfonamide. ¹H NMR (400 MHz, DMSO-d₆) δ 12.22 (s, 1H), |
| | | 7.74 (d, J = 6.2 Hz, 1H) 7.29 (d, J = 8.5 Hz, 1H), 6.88 (d J = |
| | | 10.7 Hz, 1H), 6.75 (s, 1H), 4.42 (d, J = 147.8 Hz, 3H), 4.11- |
| | | 3.91 (m, 2H), 3.86 (dd, J = 10.7, 7.5 Hz, 1H), 3.74-3.53 (m, |
| | | 1H), 2.86 (dd, J = 16.5, 5.2 Hz, 1H), 2.64 (dd, J = 16.3, 7.4 |
| | | Hz, 1H), 2.41 (s, 3H), 1.62 (s, 3H). LC/MS (APCI) m/z calcd. |
| | | for C₂₀H₂₁ClFN₄O₄S⁺ [M + H]⁺: 467.1; 467.1 found. |
| | | NB: Stereochemistry assigned randomly and not confirmed by |
| | | x-ray crystallography |
| 80 | Chiral Purification Intermediate 1 | 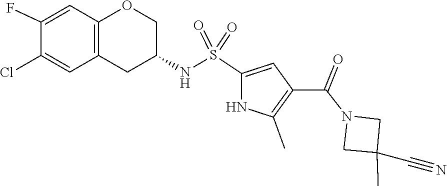 [SMILES: Cc1[nH]c(S(=O)(=O)N[C@H]2COc3cc(F)c(Cl)cc3C2)cc1C(=O)N1CC(C)(C#N)C1] |
| | | (R)-N-(6-chloro-7-fluorochroman-3-yl)-4-(3-cyano-3- |
| | | methylazetidine-1-carbonyl)-5-methyl-1H-pyrrole-2- |
| | | sulfonamide. ¹H NMR (400 MHz, DMSO-d₆) δ 12.22 (s, 1H), |
| | | 7.74 (d, J = 6.2 Hz, 1H) 7.29 (d, J = 8.5 Hz, 1H), 6.88 (d J = |
| | | 10.7 Hz, 1H), 6.75 (s, 1H), 4.42 (d, J = 147.8 Hz, 3H), 4.11- |
| | | 3.91 (m, 2H), 3.86 (dd, J = 10.7, 7.5 Hz, 1H), 3.74-3.53 (m, |
| | | 1H), 2.86 (dd, J = 16.5, 5.2 Hz, 1H), 2.64 (dd, J = 16.3, 7.4 |
| | | Hz, 1H), 2.41 (s, 3H), 1.62 (s, 3H). LC/MS (APCI) m/z calcd. |
| | | for C₂₀H₂₁ClFN₄O₄S⁺ [M + H]: 467.1; 467.1 found. |
| | | NB: Stereochemistry assigned randomly and not confirmed by |
| | | x-ray crystallography |
| 71 | Chiral Purification Intermediate 7 | 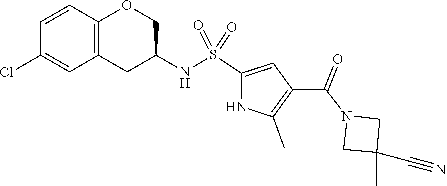 [SMILES: Cc1[nH]c(S(=O)(=O)N[C@@H]2COc3ccc(Cl)cc3C2)cc1C(=O)N1CC(C)(C#N)C1] |
| | | (S)-N-(6-chlorochroman-3-yl)-4-(3-cyano-3- |
| | | methylazetidine-1-carbonyl)-5-methyl-1H-pyrrole-2- |
| | | sulfonamide. ¹H NMR (400 MHz, DMSO-d₆) δ 12.08 (s, 1H), |
| | | 7.57 (s, 1H) 7.00-6.91 (m 2H) 6.66-6.60 (m 1H) 6.58 (s, |
| | | 1H), 4.56-4.33 (m, 1H), 4.32-3.93 (m, 3H), 3.92-3.83 (m, |
| | | 1H), 3.65 (dd, J = 10.6, 7.7 Hz, 1H), 3.47 (s, 1H), 2.71 (dd, J = |
| | | 16.7, 5.4 Hz, 1H), 2.51 (dd, J = 16.5, 8.0 Hz, 1H), 2.25 (s, |
| | | 3H), 1.46 (s, 3H). LC/MS (APCI) m/z calcd. for |
| | | C₂₀H₂₂ClN₄O₄S⁺ [M + H]: 449.1; 449.1 found. |
| | | NB: Stereochemistry assigned randomly and not confirmed by |
| | | x-ray crystallography |
| 74 | Chiral Purification Intermediate 8 | 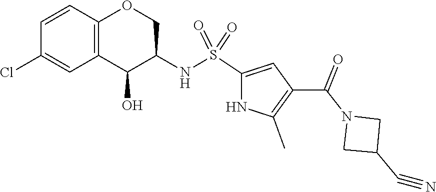 [SMILES: Cc1[nH]c(S(=O)(=O)N[C@@H]2COc3ccc(Cl)cc3[C@@H]2O)cc1C(=O)N1CC(C#N)C1] |
| | | N-((3R,4S)-6-chloro-4-hydroxychroman-3-yl)-4-(3- |
| | | cyanoazetidine-1-carbonyl)-5-methyl-1H-pyrrole-2- |
| | | sulfonamide. ¹H NMR (400 MHz, DMSO-d₆) δ 12.24 (s, 1H), |
| | | 7.41-7.25 (m, 2H), 7.22 (dd, J = 8.7, 2.7 Hz, 1H), 6.80 (d, |
| | | J = 8.7 Hz, 1H), 6.77 (s, 1H), 5.84 (s, 1H), 4.63-3.91 (m, 6H), |
| | | 3.89-3.75 (m, 2H), 3.62 (dt, J = 10.1, 3.7 Hz, 1H), 2.40 (s, |
| | | 3H). LC/MS (APCI) m/z calcd. for C₁₉H₂₀N₄O₅SCl [M + H]⁺: |
| | | 451.1; 451.1 found. |
| | | NB: Stereochemistry assigned randomly and not confirmed by |
| | | x-ray crystallography |
| 75 | Chiral Purification Intermediate 9 | 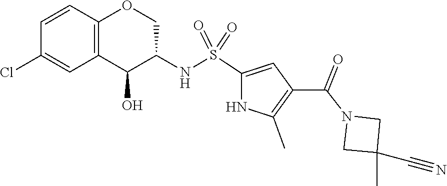 [SMILES: Cc1[nH]c(S(=O)(=O)N[C@H]2COc3ccc(Cl)cc3[C@@H]2O)cc1C(=O)N1CC(C)(C#N)C1] |
| | | N-((3S,4S)-6-chloro-4-hydroxychroman-3-yl)-4-(3-cyano-3- |
| | | methylazetidine-1-carbonyl)-5-methyl-1H-pyrrole-2- |
| | | sulfonamide. ¹H NMR (400 MHz, DMSO-d₆) δ 12.18 (s, 1H), |
| | | 7.66 (s, 1H), 7.28 (d, J = 2.6 Hz, 1H), 7.22 (dd, J = 8.7, 2.7 |
| | | Hz, 1H), 6.83 (d, J = 8.7 Hz, 1H), 6.75 (s, 1H), 5.87 (d, J = 4.5 |
| | | Hz, 1H), 4.77-3.73 (m, 7H), 3.44-3.36 (m, 1H), 2.40 (s, |
| | | 3H), 1.63 (s, 3H). ). LC/MS (APCI) m/z calcd. for |
| | | C₂₀H₂₂N₄O₅SCl [M + H]⁺: 465.1; 465.1 found. |
| | | NB: Stereochemistry assigned randomly and not confirmed by |
| | | x-ray crystallography |
| 76 | Chiral Purification Intermediate 10 | 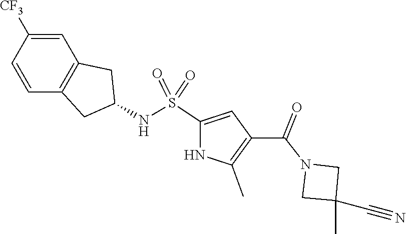 [SMILES: Cc1[nH]c(S(=O)(=O)N[C@@H]2Cc3ccc(C(F)(F)F)cc3C2)cc1C(=O)N1CC(C)(C#N)C1] |
| | | (R)-4-(3-cyano-3-methylazetidine-1-carbonyl)-5-methyl-N- |
| | | (5-(trifluoromethyl)-2,3-dihydro-1H-inden-2-yl)-1H- |
| | | pyrrole-2-sulfonamide. 1H NMR (500 MHz, |
| | | ACETONITRILE-d3) δ 10.15 (s, 1H), 7.51 (s, 1H), 7.48 (d, |
| | | J = 7.96 Hz, 1H), 7.36 (d, J = 8.17 Hz, 1H), 6.76 (s, 1H), 5.90 (d, |
| | | J = 7.40 Hz, 1H), 3.89-4.68 (m, 5H), 3.18 (d, J = 7.53 Hz, 1H), |
| | | 3.15 (d, J = 7.40 Hz, 1H), 2.84-2.88 (m, 1H), 2.81-2.84 (m, 1H), |
| | | 2.50 (s, 3H), 1.67 (s, 3H). HRMS cald for C₂₁H₂₂F₃N₄O₃S m/z |
| | | 467.1359 [M + H]+ , found 467.1368 [M + H]+ . |
| 82 | Chiral Purification Intermediate 11 | 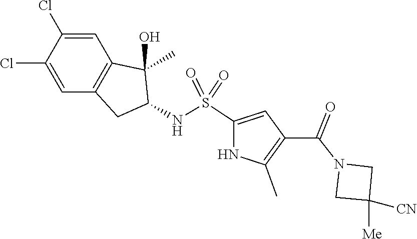 [SMILES: Cc1[nH]c(S(=O)(=O)N[C@@H]2Cc3cc(Cl)c(Cl)cc3[C@@]2(C)O)cc1C(=O)N1CC(C)(C#N)C1] |
| | | 4-(3-cyano-3-methylazetidine-1-carbonyl)-N-((1R,2R)-5,6- |
| | | dichloro-1-hydroxy-1-methyl-2,3-dihydro-1H-inden-2-yl)- |
| | | 5-methyl-1H-pyrrole-2-sulfonamide. 1H NMR (400 MHz, |
| | | DMSO-d6) δ 12.18 (br s, 1H), 7.69 (d, J = 9.02 Hz, 1H), 7.44 |
| | | (s, 2H), 6.72 (s, 1H), 5.50 (br s, 1H), 3.93-4.56 (m, 4H), 3.83 |
| | | (q, J = 8.50 Hz, 1H), 2.80 (br dd, J = 7.72, 16.02 Hz, 1H), 2.52-2.62 |
| | | (m, 1H), 2.37-2.48 (m, 3H), 1.63 (s, 3H), 1.20 (s, 3H). |
| | | HRMS cald for C₂₁H₂₃Cl₂N₄O₄S m/z 497.0812 [M + H]+ , |
| | | found 497.0730 [M + H]+ . |
| | | Stereochemistry arbitrarily assigned. |
| 92 | Chiral Purification Intermediate 11 | 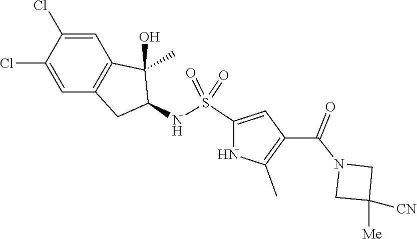 [SMILES: Cc1[nH]c(S(=O)(=O)N[C@H]2Cc3cc(Cl)c(Cl)cc3[C@@]2(C)O)cc1C(=O)N1CC(C)(C#N)C1] |
| | | 4-(3-cyano-3-methylazetidine-1-carbonyl)-N-((1R,2S)-5,6- |
| | | dichloro-1-hydroxy-1-methyl-2,3-dihydro-1H-inden-2-yl)- |
| | | 5-methyl-1H-pyrrole-2-sulfonamide. 1H NMR (400 MHz, |
| | | DMSO-d6) δ 12.20 (br s, 1H), 7.51 (d, J = 8.71 Hz, 1H), 7.41- |
| | | 7.47 (m, 1H), 6.75 (s, 1H), 5.27 (br s, 1H), 3.88-4.66 (m, 4H), |
| | | 3.58 (br d, J = 5.70 Hz, 1H), 2.68-2.80 (m, 2H), 2.38-2.46 (m, |
| | | 4H), 1.62 (s, 3H), 1.33 (s, 3H). HRMS cald for |
| | | C₂₁H₂₃Cl₂N₄O₄S m/z 497.0812 [M + H]+ , found 497.0835 [M + H]+. |
| 83 | Chiral Purification Intermediate 12 | 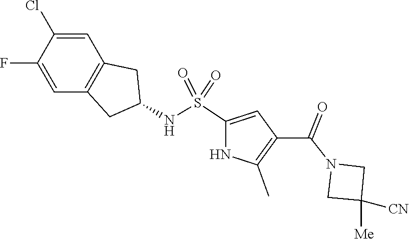 [SMILES: Cc1[nH]c(S(=O)(=O)N[C@@H]2Cc3cc(F)c(Cl)cc3C2)cc1C(=O)N1CC(C)(C#N)C1] |
| | | (R)-N-(5-chloro-6-fluoro-2,3-dihydro-1H-inden-2-yl)-4-(3- |
| | | cyano-3-methylazetidine-1-carbonyl)-5-methyl-1H- |
| | | pyrrole-2-sulfonamide. 1H NMR (500 MHz, DMSO-d6) δ |
| | | 12.23 (br s, 1H), 7.80 (d, J = 7.14 Hz, 1H), 7.39 (d, J = 7.14 Hz, |
| | | 1H), 7.25 (d, J = 9.47 Hz, 1H), 6.71 (d, J = 2.47 Hz, 1H), 3.78- |
| | | 4.77 (m, 5H), 3.02 (td, J = 8.13, 16.32 Hz, 2H), 2.63-2.82 (m, |
| | | 2H), 2.42 (s, 3H), 1.62 (s, 3H). HRMS cald for |
| | | C₂₀H₂₁ClFN₄O₃S miz 451.1002 [M + H]+ , found 451.1017 [M + H]+. |
| 84 | Chiral Purification Intermediate 12 | 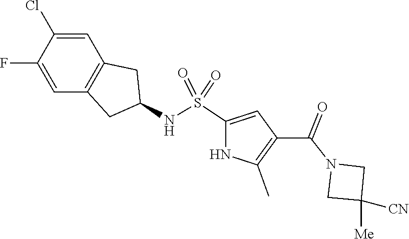 [SMILES: Cc1[nH]c(S(=O)(=O)N[C@H]2Cc3cc(F)c(Cl)cc3C2)cc1C(=O)N1CC(C)(C#N)C1] |
| | | (S)-N-(5-chloro-6-fluoro-2,3-dihydro-1H-inden-2-yl)-4-(3- |
| | | cyano-3-methylazetidine-1-carbonyl)-5-methyl-1H- |
| | | pyrrole-2-sulfonamide. H NMR (500 MHz, DMSO-d6) δ |
| | | 12.23 (br s, 1H), 7.80 (d, J = 7.14 Hz, 1H), 7.39 (d, J = 7.14 Hz, |
| | | 1H), 7.25 (d, J = 9.60 Hz, 1H), 6.71 (d, J = 2.47 Hz, 1H), 3.75- |
| | | 4.16 (m, 5H), 2.94-3.08 (m, 2H), 2.67-2.78 (m, 2H), 2.42 (s, |
| | | 3H), 1.62 (s, 3H). HRMS cald for C20H21ClFN4O3S m/z |
| | | 451.1002 [M + H]+ , found 451.1021 [M + H]+ . |

### Example S-6. Custom Syntheses

### Preparation of 5-Methyl-N-((trans)-3-(3-methyl-4-(trifluoromethyl)-1H-pyrazol-1-yl)cyclobutyl)-4-(5-methyloxazol-2-yl)-1H-pyrrole-2-sulfonamide (Compound 66)

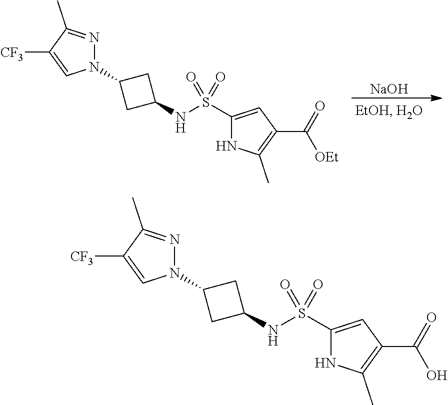

### Step 1: 2-Methyl-5-(N-(trans)-3-(3-methyl-4-(trifluoromethyl)-1H-pyrazol-1-yl)cyclobutyl)sulfamoyl)-1H-pyrrole-3-carboxylic acid

NaOH (1.84 g, 46.0 mmol, 10 equiv.) was added to a stirring solution of ethyl 2-methyl-5-(N-((1r,3r)-3-(3-methyl-4-(trifluoromethyl)-1H-pyrazol-1-yl)cyclobutyl)sulfamoyl)-1H-pyrrole-3-carboxylate (2 g, 4.6 mmol, 1 equiv.) in EtOH/H₂O (46 mL/23 mL) at rt before being heated to 90° C. for 4 h. The reaction was then cooled to rt, acidified with 2 M HCl, the precipitate filtered, and dried under high vacuum to give the desired compound as a peach colored solid (1.7 g, 91%). ¹H NMR (400 MHz, DMSO-d₆) δ 12.22 (s, 1H), 12.08 (s, 1H), 8.30 (s, 1H), 7.95 (d, J=7.5 Hz, 1H), 6.76 (d, J=2.7 Hz, 1H), 4.83 (ddd, J=14.0, 8.5, 5.0 Hz, 1H), 4.00 (dq, J=14.4, 7.6 Hz, 1H), 2.58 (ddd, J=13.3, 8.2, 5.2 Hz, 2H), 2.42 (s, 3H), 2.40-2.30 (m, 2H), 2.30-2.23 (m, 3H). LC/MS (APCI) m/z calcd. for C₁₅H₁₈F₃N₄O₄S⁺ [M+H]⁺: 407.1; 407.1 found.

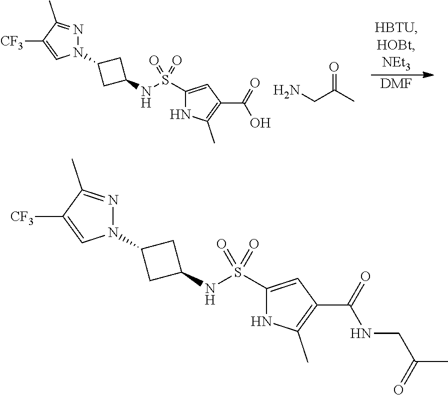

### Step 2: 2-Methyl-5-(N-(trans)-3-(3-methyl-4-(trifluoromethyl)-1H-pyrazol-1-yl)cyclobutyl)sulfamoyl)-N-(2-oxopropyl)-1H-pyrrole-3-carboxamide

HBTU (350 mg, 0.923 mmol, 1.5 equiv.) was added to a stirring solution of HOBt (125 mg, 0.923, 1.5 equiv.), 2-methyl-5-(N-((1r,3r)-3-(3-methyl-4-(trifluoromethyl)-1H-pyrazol-1-yl)cyclobutyl)sulfamoyl)-1H-pyrrole-3-carboxylic acid (250 mg, 0.615 mmol, 1 equiv.), 1-aminopropan-2-one (67 mg, 0.923 mmol, 1.5 equiv.), and NEt₃ (386 μL, 2.77 mmol, 4.5 equiv.) in DMF (1 mL) at rt. After 10 min, the reaction was diluted with water, extracted with EtOAc, the organic layer washed with brine, dried over sodium sulfate, filtered, and solvent removed by rotary evaporation. The product was isolated by silica chromatography (80% EtOAc/hexanes) as a yellow solid (155 mg, 55%). ¹H NMR (400 MHz, DMSO-d₆) δ 12.04 (s, 1H), 8.30 (s, 1H), 8.21 (t, J=5.7 Hz, 1H), 7.86 (d, J=7.6 Hz, 1H), 7.08 (d, J=2.5 Hz, 1H), 4.89-4.76 (m, 1H), 3.93 (d, J=5.7 Hz, 1H), 2.75-2.67 (m, 3H), 2.60 (ddd, J=13.1, 8.2, 4.8 Hz, 2H), 2.42 (s, 3H), 2.40-2.30 (m, 2H), 2.26 (d, J=1.2 Hz, 3H), 2.08 (s, 2H). LC/MS (APCI) m/z calcd. for C₁₈H₂₂F₃N₅O₄S⁺ [M+H]⁺: 462.1; 462.1 found.

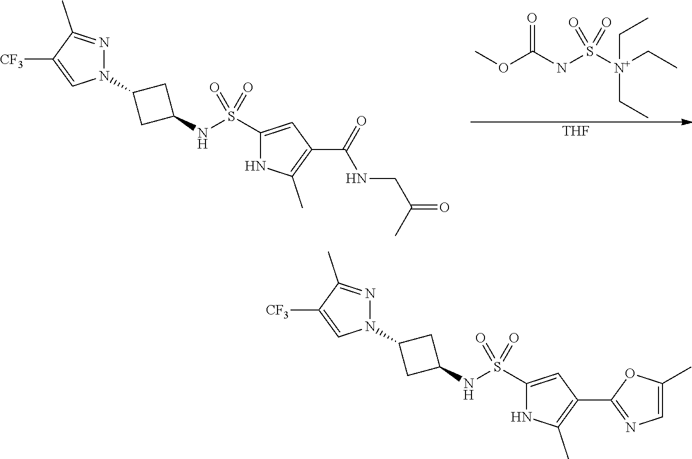 [SMILES: CC(=O)CNC(=O)c1cc(S(=O)(=O)N[C@H]2C[C@H](n3cc(C(F)(F)F)c(C)n3)C2)[nH]c1C ; CC[N+](CC)(CC)S(=O)(=O)NC(=O)OC ; Cc1cnc(-c2cc(S(=O)(=O)N[C@H]3C[C@H](n4cc(C(F)(F)F)c(C)n4)C3)[nH]c2C)o1 ; "THF"]

### Step 3: 5-Methyl-N-(trans)-3-(3-methyl-4-(trifluoromethyl)-1H-pyrazol-1-yl)cyclobutyl)-4-(5-methyloxazol-2-yl)-1H-pyrrole-2-sulfonamide

Burgess reagent (392 mg, 1.646 mmol, 4.9 equiv.) was added to a stirring solution of 2-Methyl-5-(N-((1r,3r)-3-(3-methyl-4-(trifluoromethyl)-1H-pyrazol-1-yl)cyclobutyl)sulfamoyl)-N-(2-oxopropyl)-1H-pyrrole-3-carboxamide (155 mg, 0.336 mmol, 1 equiv.) in THF (1.6 mL) before being heated to 60° C. for 1 h. The reaction was cooled to rt, diluted with a saturated sodium bicarbonate solution, extracted with EtOAc, organic layer washed with brine, dried over sodium sulfate, filtered, and solvent removed by rotary evaporation. The product was then isolated by reverse phase HPLC (15->50% MeCN/H₂O with 0.1% formic acid) as a white solid (60 mg, 40%). ¹H NMR (400 MHz, DMSO-d₆) δ 12.18 (s, 1H), 8.29 (s, 1H), 7.94 (d, J=7.7 Hz, 1H), 6.82 (dd, J=6.1, 1.8 Hz, 2H), 4.87-4.78 (m, 1H), 4.35 (t, J=5.1 Hz, 1H), 4.01 (dd, J=15.0, 7.3 Hz, 1H), 3.47-3.41 (m, 2H), 2.58 (ddd, J=13.3, 8.3, 5.2 Hz, 2H), 2.41-2.33 (m, 2H), 2.32 (d, J=1.2 Hz, 3H), 2.25 (s, 3H). LC/MS (APCI) m/z calcd. for C₁₈H₂₁F₃N₅O₃S⁺ [M+H]⁺: 444.1; 444.2 found.

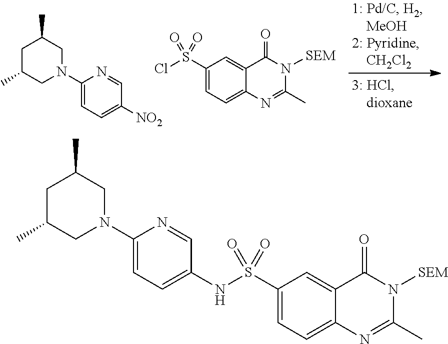

### Preparation of N-(6-((3R,5R)-3,5-dimethylpiperidin-1-yl)pyridin-3-yl)-2-methyl-4-oxo-3,4-dihydroquinazoline-6-sulfonamide (Compound 5)

2-((3R,5R)-3,5-dimethylpiperidin-1-yl)-5-nitropyridine (Enantiomer 2, 100 mg, 0.487 mmol, 1 equiv.) and Pd/C (28 mg, 10% Pd by mass, 0.026 mmol, 0.05 equiv.) were suspended in MeOH (5 mL) before being stirred under H₂ (50 psi) for 30 min. The reaction was then filtered and concentrated by rotary evaporation before being dried under high vacuum. The crude aniline and pyridine (0.08 g, 1.01 mmol, 2 equiv.) were suspended in CH₂Cl₂ before 2-methyl-4-oxo-3-((2-(trimethylsilyl)ethoxy)methyl)-3,4-dihydroquinazoline-6-sulfonyl chloride (83 mg, 0.215 mmol, 0.45 equiv) was added and the reaction stirred for 1 h. The crude reaction was filtered through a plug of silica (10->30% EtOAc/hexanes) and solvent removed by rotary evaporation. The crude residue was suspended in MeOH (0.5 mL) before 4 M HCl in dioxanes (3 mL) was added and the reaction heated to 90° C. for 10 min. The reaction was then cooled to rt and solvent removed by rotary evaporation and product isolated by silica chromatography (0->10% MeOH/CH2Cl2) as a tan solid (19 mg, 20% over 3 steps). ¹H NMR (400 MHz, Methanol-d₄) δ 8.33 (d, J=2.2 Hz, 1H), 7.88 (dd, J=8.7, 2.1 Hz, 1H), 7.57 (d, J=8.6 Hz, 1H), 7.46 (d, J=2.7 Hz, 1H), 7.14 (dd, J=9.2, 2.7 Hz, 1H), 6.57 (d, J=9.2 Hz, 1H), 3.41 (dd, J=12.9, 3.7 Hz, 2H), 3.00 (dd, J=12.9, 6.8 Hz, 2H), 2.36 (s, 3H), 1.82 (pd, J=6.5, 4.2 Hz, 2H), 1.36 (t, J=5.8 Hz, 2H), 0.81 (d, J=6.9 Hz, 6H). LC/MS (APCI) m/z calcd. for C₂₁H₂₆N₅O₃S⁺ [M+H]⁺: 428.2; 428.2 found.

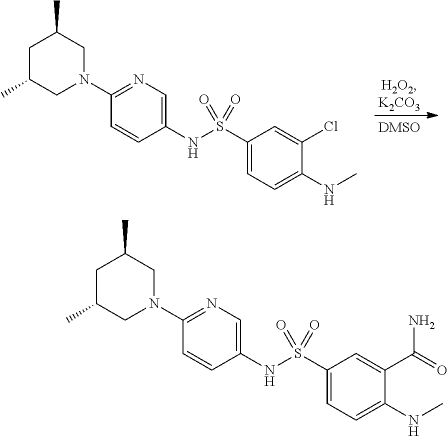

### Preparation of 5-(N-(6-(trans-3,5-dimethylpiperidin-1-yl)pyridin-3-yl)sulfamoyl)-2-(methylamino)benzamide (Compound 3)

H₂O₂ was added to a stirring solution of 3-cyano-N-(6-((trans)-3,5-dimethylpiperidin-1-yl)pyridin-3-yl)-4-(methylamino)benzenesulfonamide (58 mg, 0.145 mmol, 1 equiv.) in DMSO (1.5 mL) at rt. After 10 min, the reaction was filtered through a 0.4 μm syringe filter and product isolated by reverse phase HPLC (5->100% MeCN/H₂O with 0.1% formic acid) as a white glassy solid (12 mg, 20%). ¹H NMR (400 MHz, Chloroform-d) δ 8.32 (d, J=4.9 Hz, 1H), 8.05 (s, 1H), 7.72 (dd, J=21.9, 2.4 Hz, 2H), 7.50 (dd, J=9.0, 2.2 Hz, 1H), 7.21-7.16 (m, 1H), 6.50 (dd, J=27.3, 9.2 Hz, 2H), 6.06 (s, 2H), 3.48 (dd, J=12.8, 3.8 Hz, 2H), 3.03 (dd, J=12.8, 6.9 Hz, 2H), 2.82 (d, J=4.7 Hz, 3H), 1.89 (pd, J=6.4, 3.8 Hz, 2H), 1.38 (t, J=5.9 Hz, 2H), 0.86 (d, J=6.8 Hz, 6H). LC/MS (APCI) m/z calcd. for C₂₀H₂₈N₅O₃S⁺ [M+H]⁺: 418.2; 418.2 found.

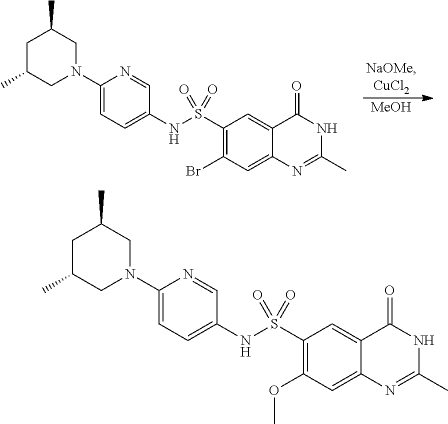

### Intermediate 25

### Preparation of N-(6-((3R,5R)-3,5-dimethylpiperidin-1-yl)pyridin-3-yl)-7-methoxy-2-methyl-4-oxo-3,4-dihydroquinazoline-6-sulfonamide

7-Bromo-N-(6-((3R,5R)-3,5-dimethylpiperidin-1-yl)pyridin-3-yl)-2-methyl-4-oxo-3,4-dihydroquinazoline-6-sulfonamide (Intermediate 25) and CuCl₂ were suspended in a solution of 30% NaOMe in MeOH (4 mL, 22 mmol, 56 equiv.) in a microwave vial before being heated to 120° C. for 30 min. The reaction was then cooled to rt, quenched with AcOH, solvent removed by rotary evaporation, and product isolated by reverse phase HPLC as an off-white solid (37 mg, 20%). ¹H NMR (400 MHz, DMSO-d₆) δ 12.40 (s, 1H), 9.61 (s, 1H), 8.28 (d, J=0.9 Hz, 1H), 7.74 (d, J=2.7 Hz, 1H), 7.28 (s, 1H), 7.22 (dd, J=9.0, 2.3 Hz, 1H), 6.71 (d, J=9.2 Hz, 1H), 4.10 (s, 3H), 3.52 (dd, J=12.8, 3.7 Hz, 2H), 3.10 (dd, J=12.8, 6.8 Hz, 2H), 2.39 (s, 3H), 1.88 (td, J=6.6, 4.1 Hz, 2H), 1.42 (t, J=5.8 Hz, 2H), 0.88 (d, J=6.7 Hz, 6H). LC/MS (APCI) m/z calcd. for C₂₂H₂₈N₅O₄S⁺ [M+H]⁺: 458.2; 458.1 found.

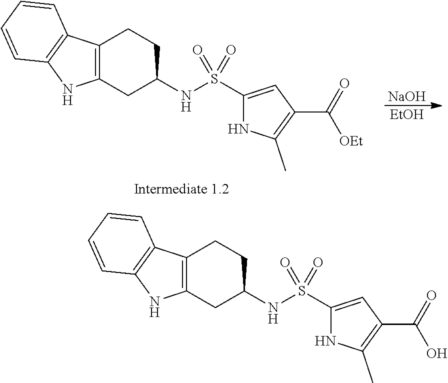

### Preparation of (R)-4-(5,5-dimethyl-4,5-dihydrooxazol-2-yl)-5-methyl-N-(2,3,4,9-tetrahydro-1H-carbazol-2-yl)-1H-pyrrole-2-sulfonamide (Compound 68)

### Step 1: (R)-2-methyl-5-(N-(2,3,4,9-tetrahydro-1H-carbazol-2-yl)sulfamoyl)-1H-pyrrole-3-carboxylic acid

NaOH (2M, 30 mL, 60 mmol, 9.6 equiv) was added to a stirring solution of ethyl (R)-2-methyl-5-(N-(2,3,4,9-tetrahydro-1H-carbazol-2-yl)sulfamoyl)-1H-pyrrole-3-carboxylate (2.5 g, 6.23 mmol, 1 equiv) in EtOH (30 mL) before being heated to 90° C. After 5 h, the reaction was cooled to rt before EtOH was removed by rotary evaporation. The solution was diluted with water before being washed with diethyl ether (50 mL), acidified with HCl (3M) to pH ˜2. The resultant precipitate was filtered, washed with water, and dried overnight to give (R)-2-methyl-5-(N-(2,3,4,9-tetrahydro-1H-carbazol-2-yl)sulfamoyl)-1H-pyrrole-3-carboxylic acid (2.05 g, 88%) as a white solid. ¹H NMR (400 MHz, DMSO-d₆) δ 12.24 (s, 1H), 12.07 (s, 1H), 10.63 (s, 1H), 7.68 (d, J=7.3 Hz, 1H), 7.32 (d, J=7.8 Hz, 1H), 7.22 (dt, J=8.1, 1.0 Hz, 1H), 6.99 (ddd, J=8.1, 7.0, 1.3 Hz, 1H), 6.92 (ddd, J=8.0, 7.1, 1.1 Hz, 1H), 6.82 (d, J=2.7 Hz, 1H), 3.66-3.52 (m, 1H), 2.82 (dd, J=16.0, 5.4 Hz, 1H), 2.77-2.69 (m, 1H), 2.60 (ddd, J=22.1, 15.8, 7.0 Hz, 2H), 2.45 (s, 3H), 1.99-1.86 (m, 1H), 1.73 (ddt, J=20.3, 10.3, 6.5 Hz, 1H).

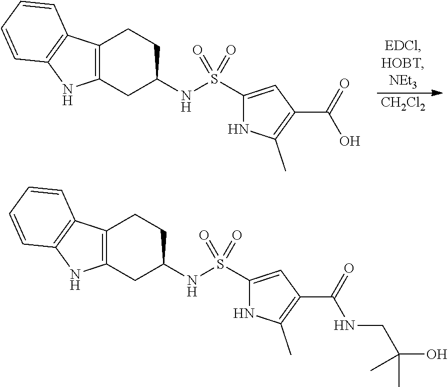

### Step 2: (R)—N-(2-hydroxy-2-methylpropyl)-2-methyl-5-(N-(2,3,4,9-tetrahydro-1H-carbazol-2-yl)sulfamoyl)-1H-pyrrole-3-carboxamide

1-Amino-2-methylpropan-2-ol (107 mg, 1.21 mmol, 3 equiv) and triethylamine (111 μL, 0.8 mmol, 2 equiv) were added to a stirring solution of (R)-2-methyl-5-(N-(2,3,4,9-tetrahydro-1H-carbazol-2-yl)sulfamoyl)-1H-pyrrole-3-carboxylic acid (150 mg, 0.40 mmol, 1 equiv), hydroxybenzotriazole (165 mg, 1.21 mmol, 3 equiv), and 1-Ethyl-3-(3-dimethylaminopropyl)carbodiimide hydrochloride (231 mg, 1.21 mmol, 3 equiv) in CH₂Cl₂ (2 mL) at rt. After 3 h, the reaction was subjected to an aqueous workup and product isolated by reverse phase HPLC (136 mg, 76%). ¹H NMR (400 MHz, DMSO-d₆) δ 12.03 (s, 1H), 10.63 (s, 1H), 7.69 (t, J=6.1 Hz, 1H), 7.56 (d, J=7.6 Hz, 1H), 7.32 (d, J=7.7 Hz, 1H), 7.22 (dt, J=8.0, 0.9 Hz, 1H), 7.16 (d, J=1.7 Hz, 1H), 6.99 (ddd, J=8.2, 7.1, 1.3 Hz, 1H), 6.92 (ddd, J=8.0, 7.1, 1.1 Hz, 1H), 4.56 (s, 1H), 3.69-3.51 (m, 1H), 3.23-3.06 (m, 2H), 2.83 (dd, J=16.0, 5.4 Hz, 1H), 2.78-2.68 (m, 1H), 2.68-2.54 (m, 2H), 2.45 (s, 3H), 1.91 (d, J=12.7 Hz, 1H), 1.78-1.62 (m, 1H), 1.07 (s, 6H). LC/MS (APCI) m/z calcd. for C₂₂H₂₉N₄O₄S⁺ [M+H]⁺: 445.1; 445.1 found.

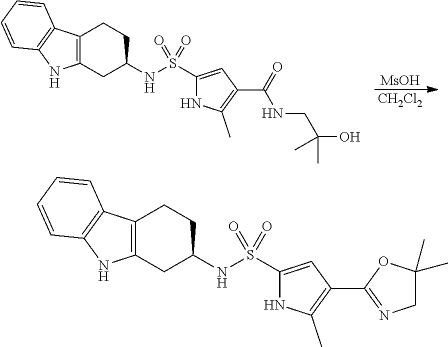 [SMILES: "MsOH" ; Cc1[nH]c(S(=O)(=O)N[C@@H]2CCc3c([nH]c4ccccc34)C2)cc1C(=O)NCC(C)(C)O ; "CH2Cl2" ; Cc1[nH]c(S(=O)(=O)N[C@@H]2CCc3c([nH]c4ccccc34)C2)cc1C1=NCC(C)(C)O1]

### Step 3: Preparation of (R)-4-(5,5-dimethyl-4,5-dihydrooxazol-2-yl)-5-methyl-N-(2,3,4,9-tetrahydro-1H-carbazol-2-yl)-1H-pyrrole-2-sulfonamide (Compound 68)

Methanesulfonic acid was added to a stirring solution of (R)—N-(2-hydroxy-2-methylpropyl)-2-methyl-5-(N-(2,3,4,9-tetrahydro-1H-carbazol-2-yl)sulfamoyl)-1H-pyrrole-3-carboxamide (129 mg, 0.291 mmol, 1 equiv) in CH₂Cl₂ at 0° C. before being warmed to 50° C. After 60 h, the reaction was quenched with saturated sodium bicarbonate, extracted with EtOAc, organics combined, dried over sodium sulfate, filtered, and solvent removed by rotary evaporation. The product was isolated by reverse phase HPLC as a white solid (75 mg, 60%). ¹H NMR (400 MHz, DMSO-d₆) δ 12.15 (s, 1H), 10.63 (s, 1H), 8.15 (s, 1H), 7.62 (d, J=7.2 Hz, 1H), 7.32 (d, J=7.7 Hz, 1H), 7.22 (dt, J=8.0, 0.9 Hz, 1H), 6.99 (ddd, J=8.0, 7.0, 1.3 Hz, 1H), 6.92 (ddd, J=8.0, 7.0, 1.1 Hz, 1H), 3.59 (s, 3H), 2.86-2.55 (m, 4H), 2.45 (s, 3H), 1.92 (d, J=12.7 Hz, 1H), 1.77-1.63 (m, 1H), 1.37 (s, 6H). LC/MS (APCI) m/z calcd. for C₂₂H₂₇N₄O₃S⁺ [M+H]⁺: 427.2; 427.1 found.

### BIOLOGICAL EXAMPLES

### Example B-1. In Vitro Model of Dose-Dependent Myofibril ATPase Modulation

Dose responses were measured using a calcium-buffered, pyruvate kinase and lactate dehydrogenase-coupled ATPase assay containing the following reagents (concentrations expressed are final assay concentrations): Potassium PIPES (12 mM), MgCl₂ (2 mM), ATP (1 mM), DTT (1 mM), BSA (0.1 mg/ml), NADH (0.5 mM), PEP (1.5 mM), pyruvate kinase (4 U/ml), lactate dehydrogenase (8 U/ml), and antifoam (90 ppm). The pH was adjusted to 6.80 at 22° C. by addition of potassium hydroxide. Calcium levels were controlled by a buffering system containing 0.6 mM EGTA and varying concentrations of calcium, to achieve a free calcium concentration of 1×10⁻⁴ M to 1×10⁻⁸ M.

Bovine cardiac myofibrils were obtained by homogenizing the appropriate tissue in the presence of detergent. Such treatment removes membranes and majority of soluble cytoplasmic proteins but leaves intact cardiac sarcomeric acto-myosin apparatus. Concentrations of myofibrils were adjusted to achieve the necessary rate of ATP hydrolysis (typically 0.25-1.0 mg/ml).

Chemical entity dose responses were measured at the calcium concentration corresponding to 25% of maximal ATPase activity (pCa₂₅), so a preliminary experiment was performed to test the response of the ATPase activity to free calcium concentrations in the range of 1×10⁻⁴ M to 1×10⁻⁸ M. Subsequently, the assay mixture was adjusted to the pCa₂₅. Assays were performed by first preparing a dilution series of test chemical entity, each with an assay mixture containing potassium Pipes, MgCl₂, BSA, DTT, pyruvate kinase, lactate dehydrogenase, myofibrils, antifoam, EGTA, CaCl₂), and water. The assay was started by adding an equal volume of solution containing potassium Pipes, MgCl₂, BSA, DTT, ATP, NADH, PEP, antifoam, and water. ATP hydrolysis was monitored by absorbance at 340 nm. The resulting dose response curve was fit by the 4 parameter equation y=Bottom+((Top−Bottom)/(1+((EC50/X)\hat{ }Hill))). The AC1.4 is defined as the concentration at which ATPase activity was 1.4-fold higher than the bottom of the dose curve. AC1.4 values reported in the table below are mean values based on a minimum of two independent tests. For compounds for which two independent tests were performed, the individual values were within two-fold of each other. For compounds for which more than two independent tests were performed, the typical error is mean+/−20-30%.

TABLE 2

| Compound | AC1.4 (μM) |
|---|---|
| 1 | 14.3998 |
| 2 | 12.20975 |
| 3 | 11.94175 |
| 4 | 0.18325 |
| 5 | 4.4808 |
| 6 | 0.7385 |
| 7 | 1.3592 |
| 8 | 0.6695 |
| 9 | 9.2363 |
| 10 | 2.8354 |
| 11 | 5.25155 |
| 12 | 1.6528 |
| 13 | 9.78345 |
| 14 | 0.20065 |
| 15 | 1.01935 |
| 16 | 8.4932 |
| 17 | 0.69305 |
| 18 | 0.8832 |
| 19 | 0.6379 |
| 20 | 3.9537 |
| 21 | 4.8236 |
| 22 | 0.4479 |
| 23 | 0.6045 |
| 24 | 5.6964 |
| 25 | 1.8965 |
| 26 | 2.21545 |
| 27 | 1.2273 |
| 28 | 0.2504 |
| 29 | 8.20385 |
| 30 | 0.4832 |
| 31 | 1.27375 |
| 32 | 5.8178 |
| 33 | 0.0762 |
| 34 | 13.2484 |
| 35 | 6.2537 |
| 36 | 6.92235 |
| 37 | 2.9421 |
| 38 | 4.3985 |
| 39 | 1.72245 |
| 40 | 0.0608 |
| 41 | 0.24345 |
| 42 | 0.06395 |
| 43 | 0.1254 |
| 44 | 0.906 |
| 45 | 0.46275 |
| 46 | 0.2277 |
| 47 | 1.9857 |
| 48 | 0.04675 |
| 49 | 2.9024 |
| 50 | 0.54665 |
| 51 | 2.8889 |
| 52 | 1.4557 |
| 53 | 0.6222 |
| 54 | 2.9934 |
| 55 | 5.7638 |
| 56 | 0.9674 |
| 57 | 0.8572 |
| 58 | 1.0081 |
| 59 | 0.41845 |
| 60 | 2.06245 |
| 61 | 0.3215 |
| 62 | 0.86915 |
| 63 | 0.081 |
| 64 | 0.3886 |
| 65 | 3.2638 |
| 66 | 1.1207 |
| 67 | 0.05605 |
| 68 | 0.12135 |
| 69 | 0.166 |
| 70 | 0.3162 |
| 71 | 0.30545 |
| 72 | 0.4237 |
| 73 | 0.0793 |
| 74 | 0.56835 |
| 75 | 0.58765 |
| 76 | 0.11215 |
| 77 | 0.1896 |
| 78 | 0.1127 |
| 79 | 0.0599 |
| 80 | 13.1993 |
| 81 | 1.19415 |
| 82 | 0.6173 |
| 83 | 0.06985 |
| 84 | 0.303 |
| 85 | 1.3618 |
| 86 | 13.53385 |
| 87 | 3.78315 |
| 88 | 2.20895 |
| 89 | 0.2521 |
| 90 | 5.31165 |
| 91 | 1.41665 |
| 92 | 0.62945 |
| 93 | 0.1626 |
| 94 | 0.6847 |
| 95 | 0.95775 |
| 96 | 1.21815 |
| 97 | 0.2269 |
| 98 | 0.66675 |
| 99 | 0.26347 |
| 100 | 5.57134 |

1. A compound of Formula (I) 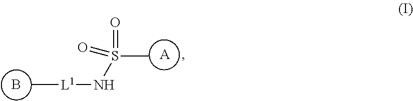
or a pharmaceutically acceptable salt thereof, wherein:
A is selected from the group consisting of 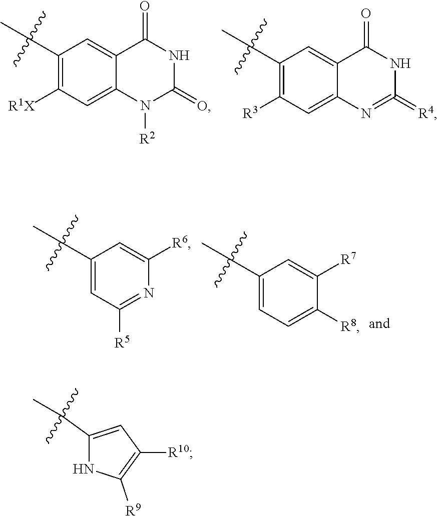
L¹ is a bond, C₁₋₆alkylene, or —NH—C₁₋₆alkylene-; and
B is selected from the group consisting of C₄₋₆cycloalkyl, tetrahydrofuranyl, piperidinyl, phenyl, pyridyl, indolyl, 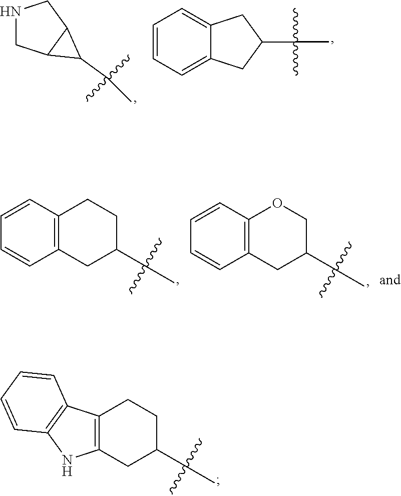
wherein
X is O or NH;
R¹, R², R⁴, and R⁵ are each independently C₁₋₆alkyl;
R³ is H, C₁₋₆alkyl, —NH—C₁₋₆alkyl, or —O—C₁₋₆alkyl, wherein the —O—C₁₋₆alkyl of R³ is optionally substituted with heterocyclyl;
R⁶ is 4- to 5-membered nitrogen-containing heterocyclyl substituted with one or more independently selected —CN, —OH, C₁₋₆alkyl, or —O—C₁₋₆alkyl substituents, wherein each C₁₋₆alkyl or —O—C₁₋₆alkyl substituent is optionally substituted with one or more independently selected halo substituents;
R⁷ is —CN or —C(O)—NH₂;
R⁸ is —NH—C₁₋₆alkyl or —N(C₁₋₆alkyl)₂;
R⁹ is C₁₋₆alkyl;
R¹⁰ is —C(O)—R^{a}, 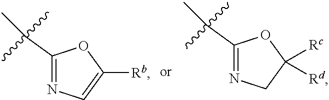
wherein
R^{a} is selected from the group consisting of —O—C₁₋₆alkyl, —NR^{a1}R^{a2}, and a 4- to 7-membered nitrogen-containing heterocyclyl optionally substituted with one or more independently selected halo, —OH, —CN, C₁₋₆alkyl, C₁₋₆haloalkyl, or —O—C₁₋₆haloalkyl substituents;
R^{a1} is H or C₁₋₆alkyl;
R^{a2} is H or C₁₋₆alkyl optionally substituted with one or more independently selected halo, —OH, C₁₋₆haloalkyl, —O—C₁₋₆alkyl, or —NH—C₁₋₆haloalkyl substituents;
R^{b}, R^{c}, and R^{d} are independently selected C₁₋₆ alkyl; and
B is optionally substituted with one or more substituents independently selected from the group consisting of: halo; —OH; C₁₋₆alkyl optionally substituted with phenyl, wherein the phenyl is optionally substituted with one or more independently selected halo substituents; C₁₋₆haloalkyl; C₃₋₆cycloalkyl; 3- to 6-membered heterocyclyl optionally substituted with one or more independently selected C₁₋₆alkyl substituents; phenyl optionally substituted with one or more independently selected halo, C₁₋₆alkyl, or C₁₋₆haloalkyl substituents; —NH-phenyl optionally substituted with one or more independently selected halo substituents; pyrazolyl optionally substituted with one or more independently selected C₁₋₆alkyl or C₁₋₆haloalkyl substituents; and pyridyl optionally substituted with one or more independently selected C₁₋₆alkyl or C₁₋₆haloalkyl substituents.

2. The compound or pharmaceutically acceptable salt thereof of claim 1, wherein A is selected from the group consisting of

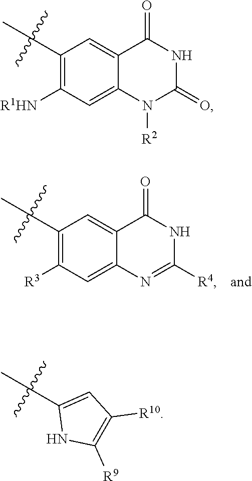

3. The compound or pharmaceutically acceptable salt thereof of claim 1, wherein A is 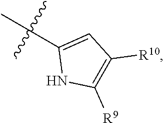
R⁹ is CH₃, and R¹⁰ is —C(O)—R^{a}.

4. (canceled)

5. The compound or pharmaceutically acceptable salt thereof of claim 3, wherein R^{a} is 4- to 7-membered nitrogen-containing heterocyclyl selected from the group consisting of 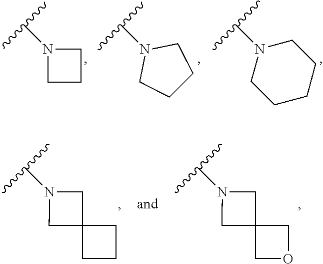
wherein each 4- to 7-membered nitrogen-containing heterocyclyl of R^{a} is optionally substituted with one or more independently selected fluoro, —OH, —CN, —CH₃, —CF₃, or —OCF₃ substituents.

6. (canceled)

7. The compound or pharmaceutically acceptable salt thereof of claim 1, wherein A is 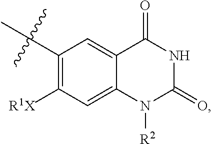
X is NH, R¹ is —CH₃, and R² is —CH₃.

8. The compound or pharmaceutically acceptable salt thereof of claim 1, wherein A is 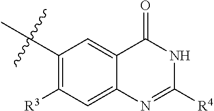
and R³ is selected from the group consisting of H, —CH₃, —OCH₃, —NHCH₃, and —O—(CH₂)₂—N(CH₂CH₂)₂O.

9. (canceled)

10. The compound or pharmaceutically acceptable salt thereof of claim 1, wherein B is selected from the group consisting of C₄₋₆cycloalkyl, pyridyl,

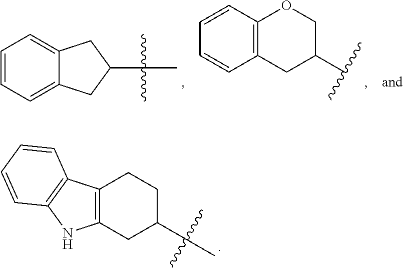

11. The compound or pharmaceutically acceptable salt thereof of claim 1, wherein B is selected from the group consisting of, 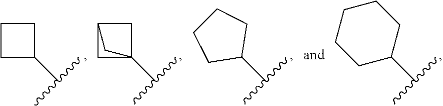
wherein the C₄₋₆cycloalkyl of B is substituted with one or more substituents from the group consisting of: C₁₋₆alkyl; phenyl optionally substituted with one or more independently selected halo or C₁₋₆haloalkyl substituents; —NH-phenyl optionally substituted with one or more independently selected halo substituents; pyrazolyl substituted with one or more independently selected C₁₋₆alkyl or C₁₋₆haloalkyl substituents; and pyridyl substituted with one or more independently selected C₁₋₆haloalkyl substituents.

12. (canceled)

13. The compound or pharmaceutically acceptable salt thereof of claim 1, wherein B is 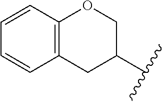 [SMILES: *C1COc2ccccc2C1]
substituted with one or more substituents independently selected from the group consisting of halo, —OH, C₁₋₆alkyl, and C₃₋₆cycloalkyl.

14. (canceled)

15. The compound or pharmaceutically acceptable salt thereof of claim 13, wherein B is 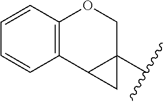 [SMILES: *C12COc3ccccc3C1C2]
substituted with one to two independently selected F or Cl substituents.

16. The compound or pharmaceutically acceptable salt thereof of claim 1, wherein B is unsubstituted

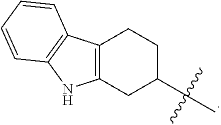 [SMILES: *C1CCc2c([nH]c3ccccc23)C1]

17. The compound or pharmaceutically acceptable salt thereof of claim 1, wherein B is 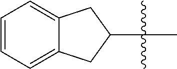 [SMILES: *C1Cc2ccccc2C1]
substituted with one or more substituents independently selected from the group consisting of halo, —OH, C₁₋₆alkyl, and C₁₋₆haloalkyl.

18. (canceled)

19. The compound or pharmaceutically acceptable salt thereof of claim 1, wherein B is pyridyl substituted with one or more independently selected 3-6 membered heterocyclyl substituents substituted with one or more independently selected C₁₋₆alkyl substituents.

20. The compound or pharmaceutically acceptable salt thereof of claim 1, wherein B is

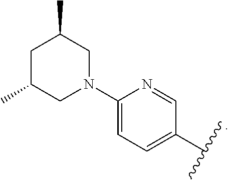 [SMILES: *c1ccc(N2C[C@H](C)C[C@@H](C)C2)nc1]

21. The compound or pharmaceutically acceptable salt thereof of claim 1, wherein
A is 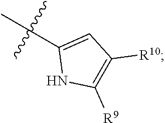
L¹ is a bond; and
B is selected from the group consisting of C₄₋₆cycloalkyl, 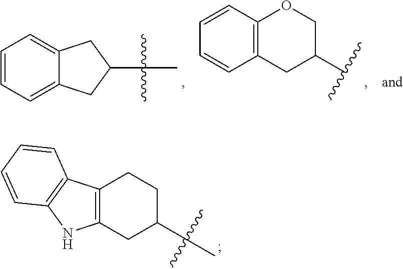
wherein
B is optionally substituted with one or more substituents independently selected from the group consisting of: halo; —OH; C₁₋₆alkyl; C₁₋₆haloalkyl; C₃₋₆cycloalkyl; phenyl optionally substituted with one or more independently selected halo or C₁₋₆haloalkyl substituents; pyrazolyl optionally substituted with one or more independently selected C₁₋₆alkyl or C₁₋₆haloalkyl substituents; and pyridyl optionally substituted with one or more independently selected C₁₋₆haloalkyl substituents.

22. The compound or pharmaceutically acceptable salt thereof of claim 1, wherein
A is 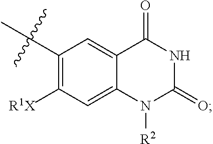
L¹ is a bond; and
B is C₄₋₆cycloalkyl;
wherein
X is NH or O;
R¹ is CH₃;
R² is CH₃; and
B is optionally substituted with one or more substituents from the group consisting of: C₁₋₆alkyl; unsubstituted phenyl; —NH-phenyl optionally substituted with one or more independently selected halo substituents; pyrazolyl substituted with one or more independently selected C₁₋₆alkyl or C₁₋₆haloalkyl substituents; and pyridyl substituted with one or more independently selected C₁₋₆haloalkyl substituents.

23. (canceled)

24. A pharmaceutical composition comprising a pharmaceutically acceptable excipient, carrier or adjuvant and at least one compound of claim 1.

25-27. (canceled)

28. A method of treating heart disease in a mammal which method comprises administering to the mammal a therapeutically effective amount of at least one compound of claim 1, or a pharmaceutical salt thereof or a pharmaceutical composition thereof.

29-31. (canceled)

32. A method for modulating the cardiac sarcomere in a mammal which method comprises administering to the mammal an amount of at least one compound of claim 1, or a pharmaceutical salt thereof or a pharmaceutical composition thereof to modulate the cardiac sarcomere in the mammal.

33. A method for potentiating cardiac myosin in a mammal which method comprises administering to the mammal an amount of at least one compound of claim 1, or a pharmaceutical salt thereof or a pharmaceutical composition thereof to potentiate cardiac myosin in the mammal.

34-35. (canceled)
